(19) United States
(12) Patent Application Publication
(10) Pub. No.: US 2011/0160280 A1
(43) Pub. Date: Jun. 30, 2011

(54) CANCER-RELATED GENES, CDCA5, EPHA7, STK31 AND WDHD1

(75) Inventors: Yusuke Nakamura, Tokyo (JP); Yataro Daigo, Tokyo (JP); Shuichi Nakatsuru, Kanagawa (JP)

(73) Assignee: Oncotherapy Science, Inc., Kanagawa (JP)

(21) Appl. No.: 12/674,659

(22) PCT Filed: Aug. 21, 2008
(86) PCT No.: PCT/JP2008/065353

§ 371 Date: Mar. 17, 2011

Related U.S. Application Data
(60) Provisional application No. 60/957,934, filed on Aug. 24, 2007.
(60) Provisional application No. 60/977,335, filed on Oct. 3, 2007.

(51) Int. Cl. A61K 31/7052; C07H 21/00; C12N 15/63; C12N 5/00; C12Q 1/68; G01N 33/68; C12Q 1/02; G01N 33/53; A61P 35/00

(52) U.S. Cl. 514/44 A; 536/24.5; 435/320.1; 435/375; 435/6.14; 436/86; 435/29; 435/7.92; 435/6.13; 436/501

(57) ABSTRACT

The invention features methods for detecting cancers, especially lung cancer and/or esophageal cancer, using over-expressed gene; CDCA5, EPHA7, STK31 or WDHD1 compared the normal organs. Also disclosed are methods of identifying compounds for treating and preventing cancers, based on the over-expression or the biological activity of CDCA5, EPHA7, STK31 or WDHD1 in the cancers, especially the interaction between EPHA7 and EGFR. Also, features are a method for treating cancers by administering a double-stranded molecule against CDCA5, EPHA7, STK31 or WDHD1 gene. The invention also features products, including the double-stranded molecules and vectors encoding them, as well as compositions comprising the molecules or vectors, useful in the provided methods.

### CROSS REFERENCE TO RELATED APPLICATIONS

This application claims the benefit of U.S. Provisional Application No. 60/957,934, filed on Aug. 24, 2007, and U.S. Provisional Application No. 60/977,335, filed on Oct. 3, 2007. The entire contents of both applications are hereby incorporated herein by reference for all purposes.

### TECHNICAL FIELD

The present invention relates to the field of biological science, more specifically to the field of cancer research. In particular, the present invention relates to methods for detecting and diagnosing cancers as well as methods for treating and preventing cancer. Moreover, the present invention relates to methods for screening for agents useful for treating and preventing cancers.

### BACKGROUND

Lung cancer and Esophagus Cancer

Aerodigestive tract cancer including carcinomas of lung, esophagus, and nasopharynx accounts for nearly one-forth of all cancer deaths in Japan. Lung cancer is the leading cause of cancer-related death in the world, and 1.3 million patients die annually (WHO Cancer World Health Organization. 2006). Two major histologically-distinct types of lung cancer, non-small cell lung cancer (NSCLC) and small-cell lung cancer (SCLC) have different pathophysiological and clinical features. NSCLC accounts for nearly 80% of lung cancers, whereas SCLC accounts for 20% of them (Morita T & Sugano H. Acta Pathol Jpn. 1990 September; 40(9):665-75; Simon G R, et al., Chest. 2003 January; 123(1 Suppl):259S-271S). In spite of applying surgical techniques combined with various treatment modalities for example, radiotherapy and chemotherapy, the overall 5-year survival rate of lung cancer is still low at about 15% (Parkin D M. Lancet Oncol. 2001 September; 2(9):533-43). Esophageal squamous cell carcinoma (ESCC) is one of the most lethal malignancies of the digestive tract, and the overall 5-years survival rate of lung cancer is only 15% (Shimada H, et al., Surgery. 2003 May; 133(5):486-94). The highest incidence of esophageal cancer was reported in the area called “Asian esophageal cancer belt”, which covers from the eastern shores of the Caspian Sea to central China (Mosavi-Jarrahi A & Mohagheghi M A. Asian Pac J Cancer Prey. 2006 July-September; 7(3):375-80). Although many genetic alterations involved in development and/or progression of lung and esophagus cancer have been reported, the precise molecular mechanism remains unclear (Sozzi G. Eur J Cancer. 2001 October; 37 Suppl 7:S63-73).

In spite of the use of modern surgical techniques combined with various treatment modalities, for example, radiotherapy and chemotherapy, lung cancer and ESCC are known to reveal the worst prognosis among malignant tumors. Five-year survival rates for lung cancer patients including all disease stages still remain at 15% and those for ESCC patients are 10% to 16% (Parkin Dm et al., CA Cancer J Clin 2005; 55:74-108 Global cancer statistics, 2002). Therefore, improved therapeutic strategies, including the development of molecular-targeted agents and antibodies, as well as cancer vaccines, are eagerly awaited. An increased understanding of the molecular basis of lung cancer has identified targeted strategies that inhibit specific key molecules in tumor growth and progression. For example, epidermal growth factor receptor (EGFR) is commonly overexpressed in NSCLC and its expression frequently correlates with a poor prognosis (Brabender J, et al., Clin Cancer Res. 2001 July; 7(7):1850-5). Recently, two main classes of EGFR inhibitors have been developed; small molecules that act as tyrosine kinase inhibitors (TKI), e.g., gefitinib and erlotinib, and monoclonal antibodies to the extracellular domain of EGFR, e.g., cetuximab. Although the aforementioned targeted therapies are expected to improve the prognosis of NSCLC, the result has yet to be sufficient. Erlotinib showed a survival benefit as compared to placebo, wherein the median survival was 6.7 months for erlotinib compared to 4.7 months for placebo (Shepherd F A. et al., N Engl J Med. 2005 Jul. 14; 353(2):123-32). On the other hand, gefitinib only showed a superior response rate and symptom control (Giaccone G, et al., J Clin Oncol. 2004 Mar. 1; 22(5):777-84; Baselga J. J Clin Oncol. 2004 Mar. 1; 22(5):759-61). In the case of cetuximab, the current Phase-2 data are not mature enough to make any definitive conclusions about the role of this agent in NSCLC (Azim H A & Ganti A K. Cancer Treat Rev. 2006 December; 32(8):630-6. Epub 2006 Oct. 10). Therefore, effective therapeutic strategies, including development of molecular-targeted agents and antibodies, as well as cancer vaccines, are eagerly awaited.

### Tumor Markers

Tumor markers that are currently available for lung cancer, for example, carcinoembryonic antigen (CEA), serum cytokeratin 19 fragment (CYFRA 21-1), and progastrin-releasing peptide (pro-GRP), are not satisfactory for diagnosis at an early stage or for monitoring the disease because of their relatively low sensitivity and specificity in detecting the presence of cancer cells (Shinkai T, et al., Cancer. 1986 Apr. 1; 57(7):1318-23; Pujol J L, et al., Cancer Res. 1993 Jan. 1; 53(1):61-6). In the same way, tumor markers that are currently available for esophageal cancer, for example, squamous cell carcinoma-related antigen (SCC), carcinoembryonic antigen (CEA), serum cytokeratin 19 fragment (CYFRA 21-1) are not satisfactory for diagnosis at an early stage or for monitoring the disease. Although the precise pathways involved in lung and esophageal tumorigenesis remain unclear, some evidence indicates that tumor cells express cell surface markers unique to each histologic type at particular stages of differentiation (Mahomed F, et al., Oral Dis. 2007 July; 13(4):386-92). Because cell surface proteins are considered more accessible to immune mechanisms and drug delivery systems, identification of cancer-specific cell surface and secretory proteins will be an effective approach to development of effective diagnostic markers and therapeutic strategies.

cDNA Microarray Analysis

Systematic analysis of expression levels of thousands of genes on a cDNA microarray is an effective approach for identifying molecules involved in pathways of carcinogenesis, some of these genes or their products will become targets for development of efficacious anti-cancer drugs and tumor markers that are reliable indicators of disease. To isolate such molecules we have analyzed genome-wide expression profiles of lung cancers and ESCCs, using pure populations of tumor cells prepared by laser microdissection (Kikuchi T, et al., Oncogene. 2003 Apr. 10; 22(14):2192-205; Kakiuchi S, et al., Mol Cancer Res. 2003 May; 1(7):485-99; Kakiuchi S, et al., Hum Mol Genet. 2004 Dec. 15; 13(24):3029-43. Epub 2004 Oct. 20; Kikuchi T, et al., Int J Oncol. 2006 April; 28(4):799-805; Taniwaki M, et al., Int J Oncol. 2006 September; 29(3):567-75; Yamabuki T, et al., Int J Oncol. 2006 June; 28(6):1375-84).

siRNA

For example, in recent years, a new approach of cancer therapy using gene-specific siRNA was attempted in clinical trials (Bumcrot D et al., Nat Chem Biol 2006 Dec., 2(12): 711-9). RNAi has already earned a place among the major technology platforms (Putral L N et al., Drug News Perspect 2006 Jul.-Aug., 19(6): 317-24; Frantz S, Nat Rev Drug Discov 2006 Jul., 5(7): 528-9; Dykxhoorn D M et al., Gene Ther 2006 March, 13(6): 541-52). Nevertheless, there are several challenges that need to be faced before RNAi can be applied in clinical use. These challenges include poor stability of RNA in vivo (Hall A H et al., Nucleic Acids Res 2004 Nov. 15, 32(20): 5991-6000, Print 2004; Amarzguioui M et al., Nucleic Acids Res 2003 Jan. 15, 31(2): 589-95), toxicity as an agent (Frantz S, Nat Rev Drug Discov 2006 Jul., 5(7): 528-9), mode of delivery, the precise sequence of the siRNA or shRNA used, and cell type specificity.

It is a well-known fact that there are possible toxicities related to silencing of partially homologous genes or induction of universal gene suppression by activating the interferon response (Judge A D et al., Nat Biotechnol 2005 Apr., 23(4): 457-62, Epub 2005 Mar. 20; Jackson A L & Linsley P S, Trends Genet 2004 Nov., 20(11): 521-4). So double-stranded molecules targeting cancer-specific genes, which molecules are devoid of adverse side-effects, are needed for the development of anticancer drugs.

### Gene Function

### (1) CDCA5

CDCA5 was identified as a regulator of sister chromatid cohesion, a cell cycle-controlled proteins. This 35-kDa protein is degraded through anaphase promoting complex (APC)-dependent ubiquitination in G1 phase. Previous studies have demonstrated that CDCA5 interacts with cohesion on chromatin and functions during interphase to support sister chromatid cohesion. Sister chromatids are further separated than normally in most G2 cells, demonstrating that CDCA5 is already required for establishment of cohesion during S phase (Schmitz J, et al., Curr Biol. 2007 Apr. 3; 17(7):630-6. Epub 2007 Mar. 8). So far only one other protein is known to be specifically required for cohesion establishment: the budding yeast acetyltransferase Eco1/Ctf7 (Skibbens R V, et al., Genes Dev. 1999 Feb. 1; 13(3):307-19; Tóth A, et al., Genes Dev. 1999 Feb. 1; 13(3):320-33; Ivanov D, et al., Curr Biol. 2002 Feb. 19; 12(4):323-8). Homologs of this enzyme are also required for cohesion in Drosophila and human cells (Williams B C, et al., Curr Biol. 2003 Dec. 2; 13(23):2025-36; Hou F & Zou H. Mol Biol Cell. 2005 August; 16(8):3908-18. Epub 2005 Jun. 15), although it is not yet known whether these proteins also function in S phase. It is therefore of interest to address whether CDCA5 and Eco1/Ctf7 homologs collaborate to establish cohesion in cancer cells.

Sister chromatid cohesion must be established and dismantled at the appropriate times in the cell cycle to effectively ensure accurate chromosome segregation. It has previously been shown that the activation of APCCdc20 controls the dissolution of cohesion by targeting the anaphase inhibitor securin for degradation. This allows the separase-dependent cleavage of Scc1/Rad21, triggering anaphase. The degradation of most cell cycle substrates of the APC is logical in terms of their function; degradation prevents the untimely presence of activity and in a ratchet-like way promotes cell cycle progression.

The function of CDCA5 is also redundant with that of other factors that regulate cohesion, with their combined activities ensuring the fidelity of chromosome replication and segregation (Rankin S, et al., Mol Cell. 2005 Apr. 15; 18(2):185-200). According to our microarray data, APC and CDC20 are also expressed highly in lung and esophageal cancers; although their expressions in normal tissues are low. Furthermore, CDC20 was confirmed with high expression in clinical small cell lung cancer using semi-quantitative RT-PCR and immunohistochemical analysis (Taniwaki M, et al, Int J Oncol. 2006 September; 29(3):567-75).

These data are consistent with the conclusion that CDCA5 in collaboration with CDC20 enhances the growth of cancer cells, by promoting cell cycle progression, although, no evidence shows that these molecules could interact directly with CDCA5. The protein is localized at nucleus in interphase cells, dispersed from the chromatid in mitosis, and interacts with the cohesion complex in anaphase (Rankin S, et al., Mol Cell. 2005 Apr. 15; 18(2):185-200). CDCA5 was reported to be required for stable binding of cohesion to chromatid and for sister chromatid cohesion in interphase (Schmitz J, et al., Curr Biol. 2007 Apr. 3; 17(7):630-6. Epub 2007 Mar. 8). In spite of these biological studies, there has been no report prior to the present invention describing the significance of activation of CDCA5 in human carcinogenesis and its use as a diagnostic and therapeutic target.

### (2) EPHA7

The EPH receptors comprise the largest group of receptor tyrosine kinases and are found in a wide variety of cell types in developing and mature tissues. One prominent function of the EPH proteins includes establishing cell positioning and maintaining cellular organization. In many developing regions of the central nervous system, EPH receptors and ephrins show complementary patterns of expression (Murai K K & Pasquale E B. J Cell Sci. 2003 Jul. 15; 116(Pt 14):2823-32). EPH receptors have been divided into two groups based on the nature of their corresponding ligands and their sequence homology: EphA and EphB receptors (Eph Nomenclature Committee, 1997).

Of all the receptor tyrosine kinases (RTKs) that are found in the human genome, the Eph-receptor family has 13 members and constitutes the largest family. The EPH receptors are divided on the basis of sequence similarity and ligand affinity into an A-subclass, which contains eight members (EPHA1-EPHA8), and a B-subclass, which in mammals contains five members (EPHB1-EPHB4, EPHB6). Their ligands, the ephrins, are divided into two subclasses, the A-subclass (ephrinA1-ephrinA5), which are tethered to the cell membrane by a glycosylphosphatidylinositol (GPI) ANCHOR, and the B-subclass (ephrinB1-ephrinB3), members of which have a transmembrane domain that is followed by a short cytoplasmic region (Kullander K & Klein R. Nat Rev Mol Cell Biol. 2002 July; 3(7):475-86).

Several signal transduction pathways are known about EPH/ephrin axis. For example, EPHA4 was involved in the JAK/Stat pathway (Lai K O, et al., J Biol Chem. 2004 Apr. 2; 279(14):13383-92. Epub 2004 Jan. 15), and EPHB4 receptor signaling mediates endothelial cell migration and proliferation via the PI3K pathway (Steinle J J, et al., J Biol Chem. 2002 Nov. 15; 277(46):43830-5. Epub 2002 Sep. 13). Furthermore, EPH/ephrin axis regulates the activities of Rho signalling or small GTPases of the Ras family (Lawrenson I D, et al., J Cell Sci. 2002 Mar. 1; 115(Pt 5):1059-72: Murai K K & Pasquale E B. J Cell Sci. 2003 Jul. 15; 116(Pt 14):2823-32).

In spite of several reports about the importance of EPH receptor family proteins in signaling pathways for cell proliferation and transformation, EPHA7 was only reported to be expressed during limb development and in nervous system (Salsi V & Zappavigna V. J Biol Chem. 2006 Jan. 27; 281(4):1992-9. Epub 2005 Nov. 28; Rogers J H et al., Brain Res Mol Brain Res. 1999 Dec. 10; 74(1-2):225-30; Araujo M & Nieto M A. Mech Dev. 1997 November; 68(1-2):173-7). Among the Eph family genes, relatively less attention has been directed toward EPHA7 in human tumors, and prior to the present invention, the role of EPHA7 in human oncology was unclear.

### (3) STK31

STK31 is a member of the Ser/Thr-kinase protein family and encodes a 115-kDa protein that contains a Tudor domain on its N-terminus, which was known to be involved in RNA binding, and Ser/Thr-kinase protein kinase domain on the C-terminus, however its physiological function remains unclear. STK31 is classified into a very unique category by the phylogenetic tree of Kinome (on the worldwide web at cellsignal.com/reference/kinase/kinome.jsp). PKR is considered as a structural homolog of STK31.

PKR protein kinase, also binds to double-strand RNA with its N-terminal domain, and has a C-terminal Ser/Thr-kinase domain. When bound to an activating RNA and ATP, PKR undergoes autophosphorylation reactions and phosphorylates the alpha-subunit of eukaryotic initiation factor 2 (elF2 alpha), inhibiting the function of the elF2 complex and continued initiation of translation (Manche L, et al., Mol Cell Biol. 1992 November; 12(11): 5238-48; Jammi N V & Beal P A. Nucleic Acids Res. 2001 Jul. 15; 29(14):3020-9; Kwon H C, et al., Jpn J Clin Oncol. 2005 September; 35(9):545-50. Epub 2005 Sep. 7).

Recently, several serine threonine kinases are considered to be a good therapeutic target for cancer. Protein kinase C beta (PKC beta), which belongs to the member of serine threonine kinases, was found to be overexpressed in fatal/refractory diffuse large B-cell lymphoma (DLBCL) and to be as a target for anti-tumor therapy (Goekjian P G & Jirousek M R. Expert Opin Investig Drugs. 2001 December; 10(12):2117-40). A phase II study was conducted with the inhibitor of PKC beta, enzastaurin, in patients with relapsed or refractory DLBCL (Goekjian P G & Jirousek M R. Expert Opin Investig Drugs. 2001 December; 10(12):2117-40). STK31 is known to associate with meiosis/germ cell differentiation in mice (Wang P J, et al., Nat Genet. 2001 April; 27(4):422-6; Olesen C, et al., Cell Tissue Res. 2007 April; 328(1):207-21. Epub 2006 Nov. 25). However, prior to the present invention its precise physiological function and its relevance to carcinogenesis was unknown.

### (4) WDHD1

WDHD1 encodes a 1129-amino acid protein with high-mobility-group (HMG) box domains and WD repeats domain. The HMG box is well conserved and consists of three alpha-helices arranged in an L-shape, which binds the DNA minor groove (Thomas J O & Travers A A. Trends Biochem Sci. 2001 March; 26(3):167-74). The HMG proteins bind DNA in a sequence-specific or non-sequence-specific way to induce DNA bending, and regulate chromatin function and gene expression (Sessa L & Bianchi M E. Gene. 2007 Jan. 31; 387(1-2):133-40. Epub 2006 Nov. 10).

In general, HMG proteins have been known to bind nucleosomes, repress transcription by interacting with the basal transcriptional machinery, act as transcriptional coactivator, or determine whether a specific regulator functions as an activator or a repressor of transcription (Ge H & Roeder R G. J Biol Chem. 1994; 269:17136-40; Paranjape S M, et al., Genes Dev 1995; 9:1978-91; Sutrias-Grau M, et al., J Biol Chem. 1999; 274: 1628-34; Shykind B M, et al., Genes Dev 1995; 9:354-65; Lehming N, et al., Nature 1994; 371:175-79). This broad spectrum of functions can be achieved in part by protein-protein interaction in addition to DNA binding activity conferred by the HMG domain. In the case of WDHD1, the candidate domain for protein-protein interaction is the WD-repeats.

WD repeat proteins contribute to cellular functions ranging from signal transduction to cell cycle control and are conserved across eukaryotes as well as prokaryotes (Li D & Roberts R. Cell Mol Life Sci. 2001; 58:2085-97). AND-1 is a nuclear protein with a conserved WD-repeats domain that was commonly found as a protein-protein interaction domain as well as HMG-box domain that was determined to be a DNA- or chromatin-binding domain in oocytes and various other cells of Xenopus laevis (Köhler A, et al., J Cell Sci. 1997 May; 110 (Pt 9):1051-62). The DNA-binding capability of the protein was demonstrated by DNA affinity chromatography and electrophoretic mobility shift assays using four-way junction DNA (Köhler A, et al., J Cell Sci. 1997 May; 110 (Pt 9):1051-62). Structural analysis has clarified that WD-repeat proteins form a propeller-like structure with several blades that is composed of a four-stranded antiparallel beta-sheet. This beta-propeller-like structure serves as a platform to which proteins can bind either stably or reversibly (Li D & Roberts R. Cell Mol Life Sci. 2001; 58:2085-97). Evidence of interacting proteins with WDHD1 aids in the understanding of the WDHD1 function(s). However, prior to the present invention, no report has clarified the physiological function of WDHD1/AND-1 and the significance of WDHD1 transactivation in human cancer progression.

### SUMMARY OF THE INVENTION

The present invention relates to cancer-related genes, in particular CX genes, including CDCA5, EPHA7, STK31 and WDHD1, which are commonly up-regulated in tumors, and strategies for the development of molecular targeted drugs and cancer vaccines for cancer treatment using CX genes.

In one aspect, the present invention provides a method for diagnosing cancer, e.g. a cancer mediated by a CX gene, e.g., lung and/or esophagus cancer, using the expression level or biological activity of the CX genes as an index. The present invention also provides a method for predicting the progress of cancer, e.g. lung and/or esophagus cancer, therapy in a patient, using the expression level or biological activity of the CX genes as an index. Furthermore, the present invention provides a method for predicting the prognosis of the cancer, e.g. lung and/or esophagus cancer, patient using the expression level or biological activity of the CX genes as an index. In some embodiments, the cancer is mediated or promoted by a CX gene. In some embodiments, the cancer is lung and/or esophagus cancer.

In another embodiment, the present invention provides a method for screening an agent for treating or preventing cancers, e.g. a cancer mediated by a CX gene, e.g., lung and/or esophagus cancer, using the expression level or biological activity of the CX genes as an index. Particularly, the present invention provides a method for screening an agent for treating or preventing cancers expressing CDCA5, e.g. lung and/or esophagus cancer, using the interaction between CDCA5 polypeptide and CDC2 polypeptide or between CDCA5 polypeptide and ERK polypeptide as an index.

In a further embodiment, the present invention provides double-stranded molecules, e.g. siRNA, against the CX genes, CDCA5, EPHA7, STK31 and WDHD1, that was screened by the methods of the present invention. The double-stranded molecules of the present invention are useful for treating or preventing cancers, e.g. a cancer mediated by a CX gene or resulting from overexpression of a CX gene, e.g., lung and/or esophagus cancer. So the present invention further relates to a method for treating cancer comprising contacting a cancerous cell with an agent screened by the methods of present invention, e.g. siRNA.

### BRIEF DESCRIPTION OF THE DRAWINGS

FIG. 1. CDCA5 expression in lung and esophageal cancers and normal tissues.

A, Expression of CDCA5 gene in lung cancer samples, examined by semiquantitative RT-PCR and western blotting. B, Expression of CDCA5 gene in esophageal cancer samples, examined by semiquantitative RT-PCR and western blotting. C, Localization of exogenous CDCA5 protein in COS-7 cells. The cells were immunocytochemically stained with affinity-purified anti-c-Myc rabbit polyclonal antibody (green) and DAPI (blue) to discriminate nucleus (see Materials and Methods). D, Northern blot analysis of the CDCA5 transcript in various normal human tissues. CDCA5 was exclusively expressed in testis.

FIG. 2. Growth inhibitory effects of siRNA against CDCA5 on lung cancer cells and growth promoting effects of exogenous CDCA5.

Two lung cancer cell lines A549 and LC319 were transfected with siRNAs for CDCA5 (A, B). Upper panels, knockdown effect of CDCA5 expression by siRNAs was confirmed by semiquantitative RT-PCR analyses. Expression of ACTB served as a quantity control at transcriptional levels. Middle panels, Colony formation assays of A549 and LC319 cells transfected with specific oligonucleotide siRNAs for CDCA5 (si-#1 and -#2) or control oligonucleotides. Lower panels, viability of A549 and LC319 cells evaluated by MTT assay in response to both si-#1 and si-#2, in comparison with that to controls. C, MTT assay shows growth promoting effect of CDCA5 on mammalian cells, compared with mock vector.

FIG. 3. EPHA7 expression in lung and esophageal cancers, and normal tissues.

A, upper panels, expression of EPHA7 in clinical lung cancers and normal lung tissues, examined by semi-quantitative RT-PCR. Lower panels, expression of EPHA7 in lung-cancer cell lines, examined by semiquantitative RT-PCR. The present inventors prepared appropriate dilutions of each single-stranded cDNA prepared from mRNAs of lung-cancer samples, taking the level of beta-actin (ACTS) expression as a quantitative control. B, upper panels, expression of EPHA7 in clinical samples of ESCC and normal esophagus tissues, examined by semiquantitative RT-PCR. Lower panels, expression of EPHA7 in esophageal cancer cell lines, examined by semiquantitative RT-PCR. C, expression of EPHA7 in normal human tissues, detected by northern-blot analysis. D, expression of EPHA7 in lung cancer cells and fetal tissues, detected by northern-blot analysis. E, expression of EPHA7 protein in normal human tissues, detected by immunohistochemical staining (×200). F, upper panels, subcellular localization of endogenous EPHA7 protein in SBC-3 cells. Lower panels, EPHA7 was stained at the cytoplasm and cytoplasmic membrane of the cell by anti-EPHA7 antibody to N-terminal of EPHA7. EPHA7 was stained at the cytoplasm and nucleus of the cell by anti-EPHA7 antibody to C-terminal of EPHA7. G, EPHA7 protein expression levels in EPHA7 positive and negative lung cancer cell lines, examined by immunocytochemistry and ELISA of culture media.

FIG. 4. Expression of EPHA7 protein in lung and esophageal cancer tissues.

A, immunohistochemical evaluation of EPHA7 protein expression using lung and esophageal cancer tissues. Left panels, expression of EPHA7 in SCLCs, lung ADCs and lung SCCs, detected by immunohistochemical staining and of no expression in normal lung (upper, ×100; lower, ×200). Positive staining appeared predominantly in the cytoplasm and cytoplasmic membrane. Right panels, expression of EPHA7 in ESCCs detected by immunohistochemical staining and of no expression in normal esophagus (upper, ×100; lower, ×200). B, association of EPHA7 overexpression with poor clinical outcomes for NSCLC patients. Kaplan-Meier analysis of tumor-specific survival in patients with NSCLC according to EPHA7 expression (P=0.006; Log-rank test). C, association of EPHA7 overexpression with poor clinical outcomes for ESCC patients. Kaplan-Meier analysis of tumor-specific survival in patients with NSCLC according to EPHA7 expression (P=0.0263; Log-rank test).

FIG. 5. Serum levels of EPHA7.

A, serum levels of EPHA7 in lung, esophageal, and cervical cancer patients, as well as COPD patients and healthy donor. B, left panel, receiver-operating characteristic (ROC) curves drawn with the data of these 439 cancer (NSCLC+SCLC+ESCC) patients and 127 healthy controls. Right panel, the concentration of serum EPHA7 before and after surgical resection of primary tumors. C, upper panel, ROC curves of EPHA7 and CEA. Lower panel, ROC curves of EPHA7 and ProGRP.

FIG. 6. Growth-promoting and invasive effects of EPHA7.

A, Left and right panels, inhibition of growth of NCI-H520 or SBC-5 cells by siRNA against EPHA7. Expression of EPHA7 in response to si-EPHA7 or control siRNAs in the cancer cells, analyzed by semi-quantitative RT-PCR (Top panels). Colony-formation assays of the cells transfected with specific siRNAs for EPHA7 or control siRNAs (Middle panels). Viability of the cells evaluated by MTT assay in response to si-EPHA7s or control siRNAs (Bottom panels). All assays were performed three times, and in triplicate wells.

FIG. 7. Phosphorylation of EGFR, p44/42 MAPK, and CDC25 as downstream targets for EPHA7. A, growth-promoting effect of EPHA7 on COS-7 cells transfected with EPHA7-expressing plasmids. Upper panels, transient expression of EPHA7 in COS-7 cells detected by Western-Blotting. Lower panels, the cell viability of COS-7 cells was measured by MTT assay. B, assays demonstrating the invasive nature of NIH3T3 and COS-7 cells in Matrigel matrix after transfection of expression plasmids for human EPHA7. Top panels, transient expression of EPHA7 in COS-7 and NIH-3T3 cells detected by Western-Blotting. Middle and bottom panels, giemsa staining (×100), and the relative number of cells migrating through the Matrigel-coated filters. Assays were performed three times and in triplicate wells.

FIG. 8. A, Tyr-845 of EGFR, Tyr-783 of PLCgamma, and Ser-216 of CDC25 were significantly phosphorylated in the cells transfected with the EPHA7-expression vector, compared with those with mock vector. B, the cognate interaction between endogenous EGFR and exogenous EPHA7, by immunoprecipitation experiment.

FIG. 9. Expression of STK31 in tumor samples and normal tissues.

A, Expression of STK31 in a normal lung tissue and 15 clinical lung cancer samples (lung ADC, lung SCC, and SCLC; upper panels) and 23 lung-cancer cell lines (lower panels), detected by semiquantitative RT-PCR analysis. B, Expression of STK31 in a normal esophagus and 10 clinical ESCC tissue samples, and 10 ESCC cell lines, detected by semiquantitative RT-PCR analysis. C, Subcellular localization of endogenous STK31 protein in lung cancer cells of NCI-H2170. STK31 was stained at the cytoplasm and nucleolus of cancer cells. D, Northern-blot analysis of the STK31 transcript in 23 normal adult human tissues. A strong signal was observed in testis.

FIG. 10. Expression of STK31 protein in normal human tissues and association of STK31 overexpression with poor prognosis for NSCLC patients.

A, Expression of STK31 in normal tissues (heart, lung, kidney, liver, testis). B, Examples for positive and negative STK31 expression in lung cancer tissues and normal lung tissue (original magnification ×100). C, Kaplan-Meier analysis of survival of patients with NSCLC (P=0.0178 by the Log-rank test) according to expression of STK31.

FIG. 11. Growth suppression of lung cancer cells by siRNA against STK31 and growth promoting effects of exogenous STK31.

A, Gene knockdown effect in response to si-STK31-#1, si-STK31-#2, or control siRNAs (si-EGFP and si-LUC) in LC319 cells, analyzed by semiquantitative RT-PCR. B, C, results of colony formation and MTT assays of LC319 cells transfected with specific siRNAs or controls. Bars, SD of triplicate assays. D, upper panels, transient expression of STK31 in COS-7, detected by Western blot analysis. Lower panel, MTT assay shows growth promoting effect of a transient expression of STK31, compared with mock vector.

FIG. 12. Kinase activity of STK31 recombinant protein and downstream targets of STK31.

A, in vitro kinase assay was done with GST fusion recombinant protein of STK31 kinase and MBP as a substrate. Phosphorylated MBP was detected. B, Levels of phosphorylation of EGFR (Ser1046/1047) and ERK (ERK1/2, P44/42 MAPK) (Thr202/Tyr204) after transient expression of STK31 in COS-7 cells, detected by Western blot analysis. C, In vitro kinase assay performed with recombinant STK31 and whole extracts prepared from COS-7 cells. Phosphorylation of ERK (ERK1/2, P44/42 MAPK) induced by STK31 was detected in a dose-dependent manner. D, Levels of phosphorylation of MEK (MEK1/2) (Ser217/Ser221) after transient expression of STK31 in COS-7 cells, detected by Western blot analysis. E, Dephosphorylation of ERK1/2 and MEK1/2 when STK31 expression was knocked down by siRNA against STK31. F, Interaction of STK31 and MAPK cascade.

FIG. 13. Expression of WDHD1 in lung and esophageal cancers and normal tissues.

A, expression of WDHD1 in a normal lung tissue and 15 clinical lung cancer samples (lung ADC, lung SCC, and SCLC; upper panels) and 23 lung-cancer cell lines (lower panels), detected by semiquantitative RT-PCR analysis. B, expression of WDHD1 in a normal esophagus and 10 clinical ESCC tissue samples, and 10 ESCC cell lines, detected by semiquantitative RT-PCR analysis. C, expression of WDHD1 protein in 5 lung-cancer and 4 esophageal cancer cell lines, examined by western-blot analysis. D, subcellular localization of endogenous WDHD1 protein in LC319 cells. WDHD1 was stained strongly at the nucleus and weakly cytoplasm throughout the cell cycle. During mitotic phase WDHD1 was stained on mitotic chromatin.

FIG. 14. Expression of WDHD1 in normal tissues and association of WDHD1 overexpression with poor prognosis for NSCLC and ESCC patients.

A, northern-blot analysis of the WDHD1 transcript in 23 normal adult human tissues. A strong signal was observed in testis. B, immunohistochemical analysis of WDHD1 protein expressions in 5 normal tissues (liver, heart, kidney, lung, and testis) with those in lung cancers. WDHD1 expressed abundantly in testis (mainly in nucleus and/or cytoplasm of primary spermatocytes) and lung cancers, but its expression was hardly detectable in the remaining four normal tissues. C, D, association of WDHD1 expression with poor prognosis. Upper panels Examples for positive and negative staining of WDHD1 expression in cancer tissues (original magnification ×100); C, lung SCC, D, ESCC. Lower panels, Kaplan-Meier analysis of survival of patients with NSCLC (C; P=0.0208 by the Log-rank test) and ESCC (D; P=0.0285 by the Log-rank test) according to expression of WDHD1.

FIG. 15. Growth promotive effect of WDHD1.

A, B, inhibition of growth of lung cancer cell lines A549 (A, left panel) and LC319 (A, right panel) and an esophageal cancer TE9 (B) by siRNAs against WDHD1. Top panels, gene knockdown effect on WDHD1 protein expression in A549, LC319 and TE9 cells by two si-WDHD1 (si-WDHD1-#1 and si-WDHD1-#2) and two control siRNAs (si-EGFP and si-SCR), analyzed by RT-PCR. Middle and bottom panels, colony formation and MTT assays of A549, LC319 and TE9 cells transfected with si-WDHD1s or control siRNAs. Columns, relative absorbance of triplicate assays; bars, SD. C, Flow cytometric analysis of NSCLC cells treated with si-WDHD1. LC319 cells were transfected with si-WDHD1-#2, collected at 72 h after transfection, for flow cytometry. The numbers besides the panels indicate the percentage of total cells at each phase. D, Enhanced growth of mammalian cells transiently transfected with WDHD1-expressing plasmids. Assays showing the growth nature of COS-7 cells after transfection with expression plasmids for hWDHD1. MTT assays of COS-7 cells transfected with hWDHD1 or control plasmids were performed. E, F, Flow cytometric analysis of NSCLC cells treated with si-WDHD1. A549 cells were transfected with si-WDHD1-#2 or si-LUC (Luciferase) and collected at 24, 48, and 72 hours after transfection for flow cytometry (E). A549 cells transfected with si-WDHD1-#2 or si-LUC were synchronized in G0/G1 phase and collected at 0, 4.5, and 9 hours after the cell cycle release for flow cytometry (F). The numbers besides the panels indicate the percentage of cells at each phase. G, Time-lapse imaging analysis of NSCLC cells treated with si-WDHD1. A549 cells were transfected with si-WDHD1-#2 or si-Luciferase and the images were captured every 30 minutes. The appearance of cells at every 12 hour is shown (From 24 to 108 hours). H, Mitotic failure and cell death induced by WDHD1 knockdown.

FIG. 16. Regulation of WDHD1 stability by its phosphorylation through PI3K signaling. A, phosphorylation of WDHD1 at serine and tyrosine residues. Left panels, dephosphorylation of endogenous WDHD1 protein in A549 cells by treatment with λ-phosphatase. Right panels, phosphorylation of WDHD1 at its serine and tyrosine residues was indicated by immunoprecipitation with anti-WDHD1 antibody followed by immunoblotting with pan-phospho-specific antibodies. B, expression of WDHD1 protein throughout the cell cycle. LC319 cells were synchronized at G0/G1 with RPMI1640 containing 1% FBS and 4 μg/ml of aphidicolin for 24 hours and released from G1 arrest by the removal of aphidicolin. Flow cytometric analysis (upper panels) and western blotting (lower panels) were done at 0, 4, and 9 hours (h) after removal of aphidicolin. C, A549 cells were also synchronized at G0/G1 with RPMI1640 containing 1% FBS and 1 μg/ml of aphidicolin for 18 hours and released from G1 arrest by the removal of aphidicolin. Flow cytometric analysis (upper panels) and western blotting (lower panels) were done at 0, 2, 4, 6, and 8 hours (h) after removal of aphidicolin. D, Reduction of WDHD1 protein by PI3K inhibition with LY294002. LC319 were treated with LY294002 in concentrations ranging from 0 and 20 μM for 24 hours and served for western-blot analysis. E, Reduction of WDHD1 protein by AKT1 inhibition with siRNA against AKT1. LC319 were transferred with siRNA for AKT1 or EGFP and served for western-blot analysis. F, G, Phosphorylation of WDHD1 protein by AKT1. Immunoprecipitant of WDHD1 was detected with anti-phospho AKT substrate (PAS) antibody (F). In vitro phosphorylation of WDHD1 protein by recombinant human AKT1 (rhAKT1) (G). H, I Phosphorylation status of Serine-374 on WDHD1 protein by AKT1. Immunoprecipitant of WDHD1 whose serine 374 was replaced with alanine (S374A) was immunoblotted with PAS antibody (H), and applied to in vitro kinase assay with rhAKT1 (I).

FIG. 17. In vitro phosphorylation of CDCA5 by CDC2 and ERK. A, Consensus phosphorylation sites on CDCA5 for CDC2 and ERK. Upper panel, homology of phosphorylation site of human CDCA5 (amino acid residues 68-82) for CDC2 (S/T-P-x-R/K) with homologues of other species. Middle and Lower panels, homology of phosphorylation site (amino acid residues 76-86 and 109-122) for ERK (x-x-S/T-P) with homologues of other species. B-C, In vitro phosphorylation of CDCA5 by CDC2 and ERK. D, MALDI-TOF mass spectrometric analysis of in vitro phosphorylated CDCA5. 8 sites were identified to be directly phosphorylated by ERK, while 3 were determined to be CDC2-dependent phosphorylation sites.

FIG. 18. Identification of ERK-dependent phosphorylation sites on CDCA5 in cultured cells. A, Endogenous CDCA5 was phosphorylated by ERK in Hela cells after EGF stimulation with or without MEK inhibitor U0126. B, In Hela cells, exogenous CDCA5 was sifted to acidic pI values in EGF stimulation. However, it was inhibited in cells with U0126 treatment, likely to the spots pattern in none treated cells.

FIG. 19. Identification of CDK1/CDC2-dependent phosphorylation sites on CDCA5 in cultured cells. A, Lung cancer cell lines A549 and LC319 were synchronized at G1/S phase with aphidicolin treatment. After release from G1/S phase, the phosphorylation status of endogenous CDCA5 protein throughout the cell cycle was detected by western-blotting. B, TE8 cell line was synchronized at G1/S phase with Aphidicolin. The cells were collected every 2 hours for 12 hours. To prevent mitosis exit, Nocodazole was added at 5 hours after release from G1/S phase. At the same time, CDK1/CDC2 inhibitors were added. C, None-tagged wild type CDCA5 and S21A, S75A and T159A alanine substituents were transfected to Hela cells. 24 hours after release from G1/S phase, and subsequent synchronization with nocodazole. D, Endogenous CDCA5 was sifted in esophageal cancer cell line TE8 and small cell lung cancer cell line SBC3 with nocodazole treatment. E. TE8 cell line was treated with CDK1/CDC2 inhibitor alsterpaullon with 1, 2, 3, 4 mM after release from G1/S phase at 5 hours while using nocodazole for mitosis synchronization.

FIG. 20. Identification of EGFR and MET as novel interacting proteins for EPHA7.

A, B, Identification of MET as an EPHA7-interacting protein. Extracts from COS-7 cells exogenously expressed EPHA7, MET, and/or mock were immunoprecipitated by either anti-myc agarose or anti-Flag agarose and immunoblotted with anti-Flag antibody or anti-myc antibody. Immunoblot with the same antibodies as immunoprecipitation was performed for evaluation of immunoprecipitation efficiency by striping and re-immunoblotting the same membrane. IP, immunoprecipitation; IB, immunoblot. C, D, Identification of EGFR as an EPHA7-interacting protein. IP, immunoprecipitation; IB, immunoblot. E, Expression profiles of EPHA7, EGFR, and MET proteins in lung cancer cells. ACTB, beta-actin.

FIG. 21. Tyrosine phosphorylation of EGFR and MET by EPHA7 kinase.

A, Schematic representation of recombinant EGFR and MET. Numbers indicate amino acid number. TM, transmembrane lesion. B, In vitro kinase assay using recombinant EPHA7 and EGFR followed by immunoblotting with anti-pan phospho-Tyr antibody. #1, #2, and #3 indicate full cytoplasmic region EGFR and partial fragment EGFR described in A. Arrowhead, phosphorylation of cytoplasmic region EGFR. Arrow, phosphorylation of #3 EGFR. C, In vitro kinase assay of EPHA7 and EGFR using [gamma-³²P] ATE Arrow, phosphorylation of #3 EGFR. D, In vitro kinase assay of EPHA7 and MET using [gamma-³²P] ATP. Arrowhead, phosphorylation of cytoplasmic region MET. E, Enhancement of EGFR/MET phosphorylation in COS-7 cells exogenously expressing EPHA7. All extracts were obtained 48 hours after transfection of EPHA7 expressing vector or mock vector.

FIG. 22. Enhancement of downstream of EGFR and MET which are important for cellular proliferation/survival signaling by EPHA7. All extracts were obtained 48 hours after transfection of EPHA7 expressing vector or mock vector.

### DISCLOSURE OF THE INVENTION

### Definitions

The words “a”, “an”, and “the” as used herein mean “at least one” unless otherwise specifically indicated.

The terms “isolated” and “purified” used in relation with a substance (e.g., polypeptide, antibody, polynucleotide, etc.) indicates that the substance is substantially free from at least one substance that can be included in the natural source. Thus, an isolated or purified antibody refers to antibodies that is substantially free of cellular material for example, carbohydrate, lipid, or other contaminating proteins from the cell or tissue source from which the protein (antibody) is derived, or substantially free of chemical precursors or other chemicals when chemically synthesized. The term “substantially free of cellular material” includes preparations of a polypeptide in which the polypeptide is separated from cellular components of the cells from which it is isolated or recombinantly produced.

Thus, a polypeptide that is substantially free of cellular material includes preparations of polypeptide having less than about 30%, 20%, 10%, or 5% (by dry weight) of heterologous protein (also referred to herein as a “contaminating protein”). When the polypeptide is recombinantly produced, in some embodiments it is also substantially free of culture medium, which includes preparations of polypeptide with culture medium less than about 20%, 10%, or 5% of the volume of the protein preparation. When the polypeptide is produced by chemical synthesis, in some embodiments it is substantially free of chemical precursors or other chemicals, which includes preparations of polypeptide with chemical precursors or other chemicals involved in the synthesis of the protein less than about 30%, 20%, 10%, 5% (by dry weight) of the volume of the protein preparation. That a particular protein preparation contains an isolated or purified polypeptide can be shown, for example, by the appearance of a single band following sodium dodecyl sulfate (SDS)-polyacrylamide gel electrophoresis of the protein preparation and Coomassie Brilliant Blue staining or the like of the gel. In one embodiment, proteins including antibodies of the present invention are isolated or purified.

An “isolated” or “purified” nucleic acid molecule, for example, a cDNA molecule, can be substantially free of other cellular material or culture medium when produced by recombinant techniques, or substantially free of chemical precursors or other chemicals when chemically synthesized. In one embodiment, nucleic acid molecules encoding proteins of the present invention are isolated or purified.

The terms “polypeptide”, “peptide”, and “protein” are used interchangeably herein to refer to a polymer of amino acid residues. The terms apply to amino acid polymers in which one or more amino acid residue is a modified residue, or a non-naturally occurring residue, for example, an artificial chemical mimetic of a corresponding naturally occurring amino acid, as well as to naturally occurring amino acid polymers.

The term “amino acid” refers to naturally occurring and synthetic amino acids, as well as amino acid analogs and amino acid mimetics that similarly functions to the naturally occurring amino acids. Naturally occurring amino acids are those encoded by the genetic code, as well as those modified after translation in cells (e.g., hydroxyproline, gamma-carboxyglutamate, and O-phosphoserine). The phrase “amino acid analog” refers to compounds that have the same basic chemical structure (an alpha carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical compounds that have different structures but similar functions to general amino acids.

Amino acids can be referred to herein by their commonly known three letter symbols or the one-letter symbols recommended by the IUPAC-IUB Biochemical Nomenclature Commission.

The terms “polynucleotides”, “oligonucleotide”, “nucleotides”, “nucleic acids”, and “nucleic acid molecules” are used interchangeably unless otherwise specifically indicated and are similarly to the amino acids referred to by their commonly accepted single-letter codes. Similar to the amino acids, they encompass both naturally-occurring and non-naturally occurring nucleic acid polymers. The polynucleotide, oligonucleotide, nucleotides, nucleic acids, or nucleic acid molecules can be composed of DNA, RNA or a combination thereof.

As used herein, the term “biological sample” refers to a whole organism or a subset of its tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid and semen). “Biological sample” further refers to a homogenate, lysate, extract, cell culture or tissue culture prepared from a whole organism or a subset of its cells, tissues or component parts, or a fraction or portion thereof. Lastly, “biological sample” refers to a medium, for example, a nutrient broth or gel in which an organism has been propagated, which contains cellular components, for example, proteins or polynucleotides.

### (1) Cancer-Related Genes and Cancer-Related Protein, and Functional Equivalent Thereof.

The words “cancer-related gene(s)”, “cancer-related polynucleotide(s)”, “CX gene(s)” and “CX polynucleotide(s)” as used herein interchangeably refer to a gene selected from the group consisted of CDCA5, EPHA7, STK31 and WDHD1.

The words “cancer-related protein(s)”, “cancer-related polypeptide(s)”, “CX protein(s)” and “CX polypeptide(s)” as used herein is a protein or polypeptide encoded by a gene selected from the group consisted of CDCA5, EPHA7, STK31 and WDHD1.

### (i) CDCA5

The nucleotide sequence of human CDCA5 gene is shown in SEQ ID NO: 1 and is also available as GenBank Accession No. NM_{—}080668 or BC011000. Herein, the phrase “CDCA5 gene” encompasses the human CDCA5 gene as well as those of other animals including non-human primate, mouse, rat, dog, cat, horse, and cow but is not limited thereto, and includes allelic mutants and genes found in other animals as corresponding to the CDCA5 gene.

The amino acid sequence encoded by the human CDCA5 gene is shown as SEQ ID NO: 2 and is also available as GenBank Accession No. AAH11000. In the present invention, the polypeptide encoded by the CDCA5 gene is referred to as “CDCA5”, and sometimes as “CDCA5 polypeptide” or “CDCA5 protein”.

According to an aspect of the present invention, functional equivalents are also included in the CDCA5. Herein, a “functional equivalent” of a protein is a polypeptide that has a biological activity equivalent to the protein. Namely, any polypeptide that retains at least one biological activity of CDCA5 can be used as such a functional equivalent in the present invention. For example, the functional equivalent of CDCA5 retains promoting activity of cell proliferation. In addition, the biological activity of CDCA5 contains binding activity to CDC2 (GenBank Accession No.: NM_{—}001786, SEQ ID NO: 48) or ERK (GenBank Accession No.: NM_{—}001040056, SEQ ID NO: 50) and/or CDC2-mediated or ERK-mediated phosphorylation. The functional equivalent of CDCA5 can contain a CDC2 binding region, ERK binding region and/or at least one of phosphorylation motifs, e.g. consensus phosphorylation motif for CDC2 (S/T-P-x-R/K) at amino acid residues 68-82 of SEQ ID NO: 2, wherein phosphorylated site is at Serine-21, Serine-75 and Threonine-159 of SEQ ID NO: 2 and/or consensus phosphorylation motif for ERK (x-x-S/T-P) at amino acid residues 76-86 or 109-122 of SEQ ID NO: 2, wherein phosphorylated site is Serine-21, Threonine-48, Serine-75, Serine-79, Threonine-111, Threonine-115, Threonine-159 and Serin-209 of SEQ ID NO: 2.

Functional equivalents of CDCA5 include those wherein one or more amino acids, e.g., 1-5 amino acids, e.g., up to 5% of amino acids, are substituted, deleted, added, or inserted to the natural occurring amino acid sequence of the CDCA5 protein.

### (ii) EPHA7

The nucleotide sequence of human EPHA7 gene is shown in SEQ ID NO: 3 and is also available as GenBank Accession No. NM_{—}004440.2. Herein, the phrase “EPHA7 gene” encompasses the human EPHA7 gene as well as those of other animals including non-human primate, mouse, rat, dog, cat, horse, and cow but is not limited thereto, and includes allelic mutants and genes found in other animals as corresponding to the EPHA7 gene.

The amino acid sequence encoded by the human EPHA7 gene is shown as SEQ ID NO: 4 and is also available as GenBank Accession No. NP_{—}004431.1. In the present invention, the polypeptide encoded by the EPHA7 gene is referred to as “EPHA7”, and sometimes as “EPHA7 polypeptide” or “EPHA7 protein”.

According to an aspect of the present invention, functional equivalents are also included in the EPHA7. Herein, a “functional equivalent” of a protein is a polypeptide that has a biological activity equivalent to the protein. Namely, any polypeptide that retains at least one biological activity of EPHA7 can be used as such a functional equivalent in the present invention. Exemplary biological activity of EPHA7 is a promoting activity of cell proliferation, tyrosine kinase activity or binding activity for EGFR. In some embodiments, the functional equivalent of EPHA7 contains Tyr kinase domain (633aa-890aa of SEQ ID NO: 4) and/or EGFR binding domain.

Functional equivalents of EPHA7 include those wherein one or more amino acids, e.g., 1-5 amino acids, e.g., up to 5% of amino acids, are substituted, deleted, added, or inserted to the natural occurring amino acid sequence of the EPHA7 protein.

(iii) STK31

The nucleotide sequence of human STK31 gene is shown in SEQ ID NO: 5 and is also available as GenBank Accession No. NM_{—}031414.2. Herein, the phrase “STK31 gene” encompasses the human STK31 gene as well as those of other animals including non-human primate, mouse, rat, dog, cat, horse, and cow but is not limited thereto, and includes allelic mutants and genes found in other animals as corresponding to the STK31 gene.

The amino acid sequence encoded by the human STK31 gene is shown as SEQ ID NO: 6 and is also available as GenBank Accession No. NP_{—}116562.1. In the present invention, the polypeptide encoded by the STK31 gene is referred to as “STK31”, and sometimes as “STK31 polypeptide” or “STK31 protein”.

According to an aspect of the present invention, functional equivalents are also included in the STK31. Herein, a “functional equivalent” of a protein is a polypeptide that has a biological activity equivalent to the protein. Namely, any polypeptide that retains at least one biological activity of STK31 can be used as such a functional equivalent in the present invention. Exemplary biological activity of STK31 is a promoting activity of cell proliferation, Ser/Thr-kinase activity or promoting activity for the phosphorylation of EGFR (Ser1046/1047), ERK (p44/42 MAPK) (Thr202/Tyr204) (SEQ ID NO.: 50, GenBank Accession No.: NM_{—}001040056) and MEK (MEK1/2) (SEQ ID NO.: 72 or SEQ ID NO.: 74, NM_{—}002755 or NM_{—}030662). In some embodiments, the functional equivalent of STK31 contains Ser/Thr-kinase domain (745aa-972aa of SEQ ID NO: 6) and/or c-raf (GenBank Accession No.: NM_{—}002880, SEQ ID NO.: 50), MEK1/2 and/or ERK (p44/42 MAPK) binding domain.

Functional equivalents of STK31 include those wherein one or more amino acids, e.g., 1-5 amino acids, e.g., up to 5% of amino acids, are substituted, deleted, added, or inserted to the natural occurring amino acid sequence of the STK31 protein.

### (iv) WDHD1

The nucleotide sequence of human WDHD1 gene is shown in SEQ ID NO: 7 and is also available as GenBank Accession No. NM_{—}007086.2. Herein, the phrase “WDHD1 gene” encompasses the human WDHD1 gene as well as those of other animals including non-human primate, mouse, rat, dog, cat, horse, and cow but is not limited thereto, and includes allelic mutants and genes found in other animals as corresponding to the WDHD1 gene.

The amino acid sequence encoded by the human WDHD1 gene is shown as SEQ ID NO: 8 also available as GenBank Accession No. NP_{—}009017.1. In the present invention, the polypeptide encoded by the WDHD1 gene is referred to as “WDHD1”, and sometimes as “WDHD1 polypeptide” or “WDHD1 protein”.

According to an aspect of the present invention, functional equivalents are also included in the WDHD1. Herein, a “functional equivalent” of a protein is a polypeptide that has a biological activity equivalent to the protein. Namely, any polypeptide that retains at least one biological activity of WDHD1 can be used as such a functional equivalent in the present invention. Exemplary biological activity of WDHD1 is a promoting activity of cell proliferation. In some embodiments, the functional equivalent of WDHD1 contains phosphorylation sites.

Functional equivalents of WDHD1 include those wherein one or more amino acids, e.g., 1-5 amino acids, e.g., up to 5% of amino acids, are substituted, deleted, added, or inserted to the natural occurring amino acid sequence of the STK31 protein.

Generally, it is known that modifications of one or more amino acid in a protein do not influence the function of the protein (Mark D F, et al., Proc Natl Acad Sci USA. 1984 September; 81(18):5662-6; Zoller M J & Smith M. Nucleic Acids Res. 1982 Oct. 25; 10(20):6487-500; Wang A, et al., Science. 1984 Jun. 29; 224(4656):1431-3; Dalbadie-McFarland G, et. al., Proc Natl Acad Sci USA. 1982 November; 79(21):6409-13). One of skill in the art will recognize that individual additions, deletions, insertions, or substitutions to an amino acid sequence which alters a single amino acid or a small percentage of amino acids is a “conservative modification” wherein the alteration of a protein results in a protein with similar functions.

Examples of properties of amino acid side chains are hydrophobic amino acids (alanine, isoleucine, leucine, methionine, phenylalanine, proline, tryptophan, tyrosine, valine), hydrophilic amino acids (arginine, aspartic acid, aspargin, cystein, glutamic acid, glutamine, glycine, histitidine, lysine, serine, threonine), and side chains having the following functional groups or characteristics in common: an aliphatic side-chain (glycine, alanine, valine, leucine, isoleucine, proline); a hydroxyl group containing side-chain (serine, threonine, tyrosine); a sulfur atom containing side-chain (C, M); a carboxylic acid and amide containing side-chain (aspartic acid, aspargine, glutamic acid, glutamine); a base containing side-chain (arginine, lysine, histidine); and an aromatic containing side-chain (histidine, phenylalanine, tyrosine, tryptophan). Furthermore, conservative substitution tables providing functionally similar amino acids are well known in the art. For example, the following eight groups each contain amino acids that are conservative substitutions for one another:

(1) Alanine (A), Glycine (G);

(2) Aspartic acid (D), Glutamic acid (E);

(3) Aspargine (N), Glutamine (Q);

(4) Arginine (R), Lysine (K);

(5) Isoleucine (I), Leucine (L), Methionine (M), Valine (V);

(6) Phenylalanine (F), Tyrosine (Y), Tryptophan (W);

(7) Serine (S), Threonine (T); and

(8) Cystein (C), Methionine (M)

(see, e.g., Thomas E. Creighton, Proteins Publisher: New York: W.H. Freeman, c1984).

Such conservatively modified polypeptides are included in the CX protein. However, the present invention is not restricted thereto and the CX protein includes non-conservative modifications so long as they retain any one of the biological activity of the CX protein. The number of amino acids to be mutated in such a modified protein is generally 10 amino acids of less, for example, 6 amino acids of less, for example, 3 amino acids or less.

An example of a protein modified by addition of one or more amino acids residues is a fusion protein of the CX protein. Fusion proteins include fusions of the CX protein and other peptides or proteins, which also can be used in the present invention. Fusion proteins can be made by techniques well known to a person skilled in the art, for example, by linking the DNA encoding the CX gene with a DNA encoding other peptides or proteins, so that the frames match, inserting the fusion DNA into an expression vector and expressing it in a host. There is no restriction as to the peptides or proteins fused to the CX protein so long as the resulting fusion protein retains any one of the objective biological activity of the CX proteins.

Known peptides that can be used as peptides to be fused to the CX protein include, for example, FLAG (Hopp T P, et al., Biotechnology 6: 1204-10 (1988)), 6×His containing six His (histidine) residues, 10×His, Influenza agglutinin (HA), human c-myc fragment, VSP-GP fragment, p18HIV fragment, T7-tag, HSV-tag, E-tag, SV40T antigen fragment, lck tag, alpha-tubulin fragment, B-tag, Protein C fragment, and the like. Examples of proteins that can be fused to a protein of the invention include GST (glutathione-S-transferase), Influenza agglutinin (HA), immunoglobulin constant region, beta-galactosidase, MBP (maltose-binding protein), and such.

Furthermore, the modified proteins do not exclude polymorphic variants, interspecies homologues, and those encoded by alleles of these proteins.

Methods known in the art to isolate functional equivalent proteins include, for example, hybridization techniques (Sambrook and Russell, Molecular Cloning: A Laboratory Manual, 3rd ed., Cold Spring Harbor Lab. Press, 2001). One skilled in the art can readily isolate a DNA having high homology (i.e., sequence identity) with a whole or part of the human CX DNA sequences (e.g., SEQ ID NO: 1 for CDCA5, SEQ ID NO: 3 for EPHA7, SEQ ID NO: 5 for STK31, SEQ ID NO: 7 for WDHD1) encoding the human CX protein, and isolate functional equivalent proteins to the human CX protein from the isolated DNA. Thus, the proteins used for the present invention include those that are encoded by DNA that hybridize under stringent conditions with a whole or part of the DNA sequence encoding the human CX protein and are functional equivalent to the human CX protein. These proteins include mammal homologues corresponding to the protein derived from human or mouse (for example, a protein encoded by a monkey, rat, rabbit or bovine gene). In isolating a cDNA highly homologous to the DNA encoding the human CX gene from lung or esophagus cancer tissue or cell line, or tissues from testis (for CDCA5, STK31 or WDHD1) brain or kidney (for EPHA7) can be used.

The conditions of hybridization for isolating a DNA encoding a protein functional equivalent to the human CX gene can be routinely selected by a person skilled in the art. The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will differ under different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10 degree Centigrade lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions can also be achieved with the addition of destabilizing agents for example, formamide. For selective or specific hybridization, a positive signal is at least two times of background, for example, 10 times of background hybridization.

For example, hybridization can be performed by conducting prehybridization at 68° C. for 30 min or longer using “Rapid-hyb buffer” (Amersham LIFE SCIENCE), adding a labeled probe, and warming at 68 degrees C. for 1 h or longer. The following washing step can be conducted, for example, in a low stringent condition. A low stringent condition is, for example, 42° C., 2×SSC, 0.1% SDS, for example, 50° C., 2×SSC, 0.1% SDS. In some embodiments, high stringent condition is used. A high stringent condition is, for example, washing 3 times in 2×SSC, 0.01% SDS at room temperature for 20 min, then washing 3 times in 1×SSC, 0.1% SDS at 37 degrees C. for 20 min, and washing twice in 1×SSC, 0.1% SDS at 50 degrees C. for 20 min. However, several factors for example, temperature and salt concentration can influence the stringency of hybridization and one skilled in the art can suitably select the factors to achieve the requisite stringency.

In place of hybridization, a gene amplification method, for example, the polymerase chain reaction (PCR) method, can be utilized to isolate a DNA encoding a protein functional equivalent to the human CX gene, using a primer synthesized based on the sequence information of the DNA (SEQ ID NO: 1 for CDCA5; SEQ ID NO: 3 for EPHA7; SEQ ID NO: 5 for STK31; or SEQ ID NO: 7 for WDHD1;) encoding the human CX protein (SEQ ID NO: 2 for CDCA5; SEQ ID NO: 4 for EPHA7; SEQ ID NO: 6 for STK31; or SEQ ID NO: 8 for WDHD1), examples of primer sequences are pointed out in (3) Semi-quantitative RT-PCR in [EXAMPLE 1].

Proteins that are functional equivalent to the human CX protein encoded by the DNA isolated through the above hybridization techniques or gene amplification techniques, normally have a high homology (also referred to as sequence identity) to the amino acid sequence of the human CX protein. “High homology” (also referred to as “high sequence identity”) typically refers to the degree of identity between two optimally aligned sequences (either polypeptide or polynucleotide sequences). Typically, high homology or sequence identity refers to homology of 40% or higher, for example, 60% or higher, for example, 80% or higher, for example, 85%, 90%, 95%, 98%, 99%, or higher. The degree of homology or identity between two polypeptide or polynucleotide sequences can be determined by following the algorithm (Wilbur W J & Lipman D J. Proc Natl Acad Sci USA. 1983 February; 80 (3):726-30).

Additional examples of algorithms that are suitable for determining percent sequence identity and sequence similarity are the BLAST and BLAST 2.0 algorithms, which are described (Altschul S F, et al., J Mol Biol. 1990 Oct. 5; 215 (3):403-10; Nucleic Acids Res. 1997 Sep. 1; 25(17):3389-402). Software for performing BLAST analyses is publicly available through the National Center for Biotechnology Information (on the worldwide web at ncbi.nlm.nih.gov/). The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them.

The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached.

The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (Henikoff S & Henikoff J G. Proc Natl Acad Sci USA. 1992 Nov. 15; 89(22):10915-9).

A protein useful in the context of the present invention can have variations in amino acid sequence, molecular weight, isoelectric point, the presence or absence of sugar chains, or form, depending on the cell or host used to produce it or the purification method utilized. Nevertheless, so long as it has any one of the biological activity of the CX protein (SEQ ID NO: 2 for CDCA5, SEQ ID NO: 4 for EPHA7, SEQ ID NO: 6 for STK31, SEQ ID NO: 8 for WDHD1), it is useful in the present invention.

The present invention also encompasses the use of partial peptides of the CX protein. A partial peptide has an amino acid sequence specific to the protein of the CX protein and consists of less than about 400 amino acids, usually less than about 200 and often less than about 100 amino acids, and at least about 7 amino acids, for example, about 8 amino acids or more, for example, about 9 amino acids or more.

A partial peptide used for the screenings of the present invention suitably contains at least a cohesion binding domain and/or phosphorylation sites of CDCA5, Tyr kinase domain (633aa-890aa of SEQ ID NO: 4) and/or EGFR binding domain of EPHA7, Ser/Thr-kinase domain (745aa-972aa of SEQ ID NO: 6) of STK31, and/or phosphorylation sites of WDHD1. Furthermore, a partial CDCA5 peptide used for the screenings of the present invention suitably contains CDC2 binding region, ERK binding region and/or at least one of the phosphorylation motifs, e.g. consensus phosphorylation motif for CDC2 at amino acid residues 68-82 (S/T-P-x-R/K) of SEQ ID NO: 2, wherein phosphorylated site is Serine-21, Serine-75 and Threonine-159 of SEQ ID NO: 2, consensus phosphorylation motif for ERK (x-x-S/T-P) at amino acid residues 76-86 or 109-122, wherein phosphorylated site is Serine-21, Threonine-48, Serine-75, Serine-79, Threonine-111, Threonine-115, Threonine-159 and Serin-209 of SEQ ID NO: 2; a partial CDC2 peptide used for the screenings of the present invention suitably contains CDCA5 binding region and/or a Serine/Threonine protein kinases catalytic domain, e.g. amino acid residues 4-287 of SEQ ID NO: 48 (CDC2); and a partial ERK peptide used for the screenings of the present invention suitably contains CDCA5 binding region and/or a protein kinase domain, e.g. amino acid residues 72-369 of SEQ ID NO: 50 (ERK). Such partial peptides are also encompassed by the phrase “functional equivalent” of the CX protein.

The polypeptide or fragments used for the present method can be obtained from nature as naturally occurring proteins via conventional purification methods or through chemical synthesis based on the selected amino acid sequence. For example, conventional peptide synthesis methods that can be adopted for the synthesis include:
(1) Peptide Synthesis, Interscience, New York, 1966;
(2) The Proteins, Vol. 2, Academic Press, New York, 1976;
(3) Peptide Synthesis (in Japanese), Maruzen Co., 1975;
(4) Basics and Experiment of Peptide Synthesis (in Japanese), Maruzen Co., 1985;
(5) Development of Pharmaceuticals (second volume) (in Japanese), Vol. 14 (peptide synthesis), Hirokawa, 1991;
(6) WO99/67288; and
(7) Barany G. & Merrifield R. B., Peptides Vol. 2, “Solid Phase Peptide Synthesis”, Academic Press, New York, 1980, 100-118.

Alternatively, the protein can be obtained adopting any known genetic engineering methods for producing polypeptides (e.g., Morrison D A., et al., J Bacteriol. 1977 October; 132(1):349-51; Clark-Curtiss J E & Curtiss R 3rd. Methods Enzymol. 1983; 101:347-62). For example, first, a suitable vector comprising a polynucleotide encoding the objective protein in an expressible form (e.g., downstream of a regulatory sequence comprising a promoter) is prepared, transformed into a suitable host cell, and then the host cell is cultured to produce the protein. More specifically, a gene encoding the HJURP is expressed in host (e.g., animal) cells and such by inserting the gene into a vector for expressing foreign genes, for example, pSV2neo, pcDNA I, pcDNA3.1, pCAGGS, or pCD8.

A promoter can be used for the expression. Any commonly used promoters can be employed including, for example, the SV40 early promoter (Rigby in Williamson (ed.), Genetic engineering, vol. 3. Academic Press, London, 1982, 83-141), the EF-alpha promoter (Kim D W, et al. Gene. 1990 Jul. 16; 91(2):217-23), the CAG promoter (Niwa H, et al., Gene. 1991 Dec. 15; 108(2):193-9), the RSV LTR promoter (Cullen B R. Methods Enzymol. 1987; 152:684-704), the SR alpha promoter (Takebe Y, et al., Mol Cell Biol. 1988 January; 8(1):466-72), the CMV immediate early promoter (Seed B & Aruffo A. Proc Natl Acad Sci USA. 1987 May; 84(10):3365-9), the SV40 late promoter (Gheysen D & Fiers W. J Mol Appl Genet. 1982; 1(5):385-94), the Adenovirus late promoter (Kaufman R J, et al., Mol Cell Biol. 1989 March; 9(3):946-58), the HSV TK promoter, and such.

The introduction of the vector into host cells to express the CX gene can be performed according to any methods, for example, the electroporation method (Chu G, et al., Nucleic Acids Res. 1987 Feb. 11; 15(3):1311-26), the calcium phosphate method (Chen C & Okayama H. Mol Cell Biol. 1987 August; 7(8):2745-52), the DEAE dextran method (Lopata M A, et al., Nucleic Acids Res. 1984 Jul. 25; 12(14):5707-17; Sussman D J & Milman G. Mol Cell Biol. 1984 August; 4(8):1641-3), the Lipofectin method (Derijard B, et al., Cell. 1994 Mar. 25; 76(6):1025-37; Lamb B T, et al., Nat Genet. 1993 September; 5(1):22-30; Rabindran S K, et al., Science. 1993 Jan. 8; 259(5092):230-4), and such.

The CX proteins can also be produced in vitro adopting an in vitro translation system.

In the context of the present invention, the phrase “CX gene” encompasses polynucleotides that encode the human CX gene or any of the functional equivalents of the human CX gene.

The CX gene can be obtained from nature as naturally occurring proteins via conventional cloning methods or through chemical synthesis based on the selected nucleotide sequence. Methods for cloning genes using cDNA libraries and such are well known in the art.

### (2) Antibody

The terms “antibody” as used herein is intended to include immunoglobulins and fragments thereof which are specifically reactive to the designated protein or peptide thereof. An antibody can include human antibodies, primatized antibodies, chimeric antibodies, bispecific antibodies, humanized antibodies, antibodies fused to other proteins or radiolabels, and antibody fragments. Furthermore, an antibody herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity. An “antibody” indicates all classes (e.g. IgA, IgD, IgE, IgG and IgM).

The subject invention uses antibodies against CX proteins, including for example, antibodies against the N-terminal portion of EPHA7 (e.g., residues 526-580aa of SEQ ID NO: 4 of EPHA7). These antibodies can be useful for diagnosing lung cancer or esophageal cancer. The antibodies against CDCA5 polypeptide are also used, especially antibodies against at least one of phosphorylation regions of CDCA5 polypeptide, e.g. consensus phosphorylation motif for CDC2 at amino acid residues 68-82 (S/T-P-x-R/K) of SEQ ID NO: 2 (CDCA5), and amino acid residues 76-86 (x-x-S/T-P) of SEQ ID NO: 2 (CDCA5), and/or 109-122 (x-x-S/T-P) of SEQ ID NO: 2 (CDCA5). These antibodies can be useful for inhibiting and/or blocking CDC2-mediated phosphorylation of CDCA5 polypeptide or ERK-mediated phosphorylation of CDCA5 polypeptide and can be useful for treating and/or preventing cancers (over)expressing CDCA5, e.g. lung cancer or esophageal cancer. Furthermore, the subject invention uses antibodies against CDCA5 polypeptide or partial peptide of them, especially antibodies against CDC2 binding region of CDCA5 polypeptide or ERK binding region of CDCA5 polypeptide.

These antibodies can be useful for inhibiting and/or blocking an interaction, e.g. binding, between CDCA5 polypeptide and CDC2 polypeptide or an interaction, e.g. binding, between CDCA5 polypeptide and ERK polypeptide and can be useful for treating and/or preventing cancer (over)expressing CDCA5, e.g. lung cancer or esophageal cancer. Alternatively, the subject invention also uses antibodies against CDC2 polypeptide, ERK polypeptide or partial peptide of them, e.g. CDCA5 binding region of them. These antibodies will be provided by known methods. Exemplary techniques for the production of the antibodies used in accordance with the present invention are described.

### (i) Polyclonal Antibodies

Polyclonal antibodies can be raised in animals by multiple subcutaneous (sc) or intraperitoneal (ip) injections of the relevant antigen and an adjuvant. Conjugating the relevant antigen to a protein that is immunogenic in the species to be immunized finds use, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOC12, or R′N═C═NR, where R′ and R are different alkyl groups.

Animals are immunized against the antigen, immunogenic conjugates, or derivatives by combining, e.g. 100 micro g or 5 micro g of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. In some embodiments, the animal is boosted with the conjugate of the same antigen, but conjugated to a different protein and/or through a different cross-linking reagent.

Conjugates also can be made in recombinant cell culture as protein fusions. Also, aggregating agents for example, alum are suitably used to enhance the immune response.

### (ii) Monoclonal Antibodies

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

For example, the monoclonal antibodies can be made using the hybridoma method first described by Kohler G & Milstein C. Nature. 1975 Aug. 7; 256 (5517):495-7, or can be made by recombinant DNA methods (U.S. Pat. No. 4,816,567).

In the hybridoma method, a mouse or other appropriate host animal, for example, a hamster, is immunized as hereinabove described to elicit lymphocytes that produce or are capable of producing antibodies that will specifically bind to the protein used for immunization. Alternatively, lymphocytes can be immunized in vitro. Lymphocytes then are fused with myeloma cells using a suitable fusing agent, for example, polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that can contain one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

In some embodiments, myeloma cells are those that fuse efficiently, support stable high-level production of antibody by the selected antibody-producing cells, and are sensitive to a medium for example, HAT medium. Exemplary myeloma cell lines include murine myeloma lines, for example, those derived from MOPC-21 and MPC-11 mouse tumors available from the Salk Institute Cell Distribution Center, San Diego, Calif. USA, and SP-2 or X63-Ag8-653 cells available from the American Type Culture Collection, Manassas, Va., USA. Human myeloma and mouse-human heteromyeloma cell lines also have been described for the production of human monoclonal antibodies (Kozbor D, et al., J Immunol. 1984 December; 133(6):3001-5; Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. In some embodiments, the binding specificity of monoclonal antibodies produced by hybridoma cells is determined by immunoprecipitation or by an in vitro binding assay, for example, radioimmunoassay (RIA) or enzyme-linked immunoabsorbent assay (ELISA).

The binding affinity of the monoclonal antibody can, for example, be determined by the 30 Scatchard analysis of Munson P J & Rodbard D. Anal Biochem. 1980 Sep. 1; 107(1):220-39.

After hybridoma cells are identified that produce antibodies of the desired specificity, affinity, and/or activity, the clones can be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells can be grown in vivo as ascites tumors in an animal.

The monoclonal antibodies secreted by the subclones are suitably separated from the culture medium, ascites fluid, or serum by conventional immunoglobulin purification procedures for example, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a source of such DNA. Once isolated, the DNA can be placed into expression vectors, which are then transfected into host cells for example, E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra A. Curr Opin Immunol. 1993 April; 5 (2):256-62 and Plückthun A. Immunol Rev. 1992 December; 130:151-88.

Another method of generating specific antibodies, or antibody fragments, reactive against CX protein is to screen expression libraries encoding immunoglobulin genes, or portions thereof, expressed in bacteria with CX protein or peptide. For example, complete Fab fragments, VH regions and Fv regions can be expressed in bacteria using phage expression libraries. See for example, Ward E S, et al., Nature. 1989 Oct. 12; 341(6242):544-6; Huse W D, et al., Science. 1989 Dec. 8; 246(4935):1275-81; and McCafferty J, et al., Nature. 1990 Dec. 6; 348(6301):552-4. Screening such libraries with, CX protein, e.g. CX peptides, can identify immunoglobulin fragments reactive with the CX protein. Alternatively, the SCID-humouse (available from Genpharm) can be used to produce antibodies or fragments thereof.

In a further embodiment, antibodies or antibody fragments can be isolated from antibody phage libraries generated using the techniques described in McCafferty J, et al., Nature. 1990 Dec. 6; 348(6301):552-4; Clackson T, et al., Nature. 1991 Aug. 15; 352(6336):624-8; and Marks J D, et al., J MoL BioL, 222: 581-597 (1991) J Mol Biol. 1991 Dec. 5; 222(3):581-97 describe the isolation of murine and human antibodies, respectively, using phage libraries. Subsequent publications describe the production of high affinity (nM range) human antibodies by chain shuffling (Marks J D, et al., Biotechnology (N Y). 1992 July; 10(7):779-83), as well as combinatorial infection and in vivo recombination as a strategy for constructing very large phage libraries (Waterhouse P, et al., Nucleic Acids Res. 1993 May 11; 21(9):2265-6). Thus, these techniques are viable alternatives to traditional monoclonal antibody hybridoma techniques for isolation of monoclonal antibodies.

The DNA also can be modified, for example, by substituting the coding sequence for human heavy- and light-chain constant domains in place of the homologous murine sequences (U.S. Pat. No. 4,816,567; Morrison S L, et al., Proc Natl Acad Sci USA. 1984 November; 81(21):6851-5), or by covalently joining to the immunoglobulin coding sequence all or part of the coding sequence for a non-immunoglobulin polypeptide.

Typically, such non-immunoglobulin polypeptides are substituted for the constant domains of an antibody, or they are substituted for the variable domains of one antigen combining site of an antibody to create a chimeric bivalent antibody comprising one antigen-combining site having specificity for an antigen and another antigen-combining site having specificity for a different antigen.

(iii) Humanized Antibodies

Methods for humanizing non-human antibodies have been described in the art. In some embodiments, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones P T, et al., Nature. 1986 May 29-Jun. 4; 321(6069):522-5; Riechmann L, et al., Nature. 1988 Mar. 24; 332(6162):323-7; Verhoeyen M, et al., Science. 1988 Mar. 25; 239(4847):1534-6), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims M J, et al., J Immunol. 1993 Aug. 15; 151(4):2296-308; Chothia C & Lesk A M. J Mol Biol. 1987 Aug. 20; 196(4):901-17). Another method uses a particular framework region derived from the consensus sequence of all human antibodies of a particular subgroup of light or heavy chains. The same framework can be used for several different humanized antibodies (Carter P, et al., Proc Natl Acad Sci USA. 1992 May 15; 89(10):4285-9; Presta L G, et al., J Immunol. 1993 Sep. 1; 151(5):2623-32).

It is further important that antibodies be humanized with retention of high affinity for the antigen and other favorable biological properties. To achieve this goal, in some embodiments, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, for example, increased affinity for the target antigen, is achieved. In general, the hypervariable region residues are directly and most substantially involved in influencing antigen binding.

### (iv) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ line mutant mice will result in the production of human antibodies upon antigen challenge. See, e.g., Jakobovits A, et al., Proc Natl Acad Sci USA. 1993 Mar. 15; 90(6):2551-5; Nature. 1993 Mar. 18; 362(6417):255-8; Brüggemann M, et al., Year Immunol. 1993; 7:33-40; and U.S. Pat. Nos. 5,591,669; 5,589,369 and 5,545,807.

Alternatively, phage display technology (McCafferty J, et al., Nature. 1990 Dec. 6; 348(6301):552-4) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, for example, M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson K S & Chiswell D J. Curr Opin Struct Biol. 1993; 3:564-71. Several sources of V-gene segments can be used for phage display.

Clackson T, et al., Nature. 1991 Aug. 15; 352(6336):624-8 isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self antigens) can be isolated essentially following the techniques described by Marks J D, et al., J Mol Biol. 1991 Dec. 5; 222(3):581-97, or Griffiths A D, et al., EMBO J. 1993 February; 12(2):725-34. See, also, U.S. Pat. Nos. 5,565,332 and 5,573,905.

Human antibodies can also be generated by in vitro activated B cells (see U.S. Pat. Nos. 5,567,610 and 5,229,275).

### (v) Non-Antibody Binding Proteins

The present invention also contemplates non-antibody binding proteins against CX proteins, including against the N-terminal portion of EPHA7. The terms “non-antibody binding protein” or “non-antibody ligand” or “antigen binding protein” interchangeably refer to antibody mimics that use non-immunoglobulin protein scaffolds, including adnectins, avimers, single chain polypeptide binding molecules, and antibody-like binding peptidomimetics, as discussed in more detail below.

Other compounds have been developed that target and bind to targets in a manner similar to antibodies. Certain of these “antibody mimics” use non-immunoglobulin protein scaffolds as alternative protein frameworks for the variable regions of antibodies.

For example, Ladner et al. (U.S. Pat. No. 5,260,203) describe single polypeptide chain binding molecules with binding specificity similar to that of the aggregated, but molecularly separate, light and heavy chain variable region of antibodies. The single-chain binding molecule contains the antigen binding sites of both the heavy and light chain variable regions of an antibody connected by a peptide linker and will fold into a structure similar to that of the two peptide antibody. The single-chain binding molecule displays several advantages over conventional antibodies, including, smaller size, greater stability and are more easily modified.

Ku et al. (Proc Natl Acad Sci USA 92(14):6552-6556 (1995)) discloses an alternative to antibodies based on cytochrome b562. Ku et al. (1995) generated a library in which two of the loops of cytochrome b562 were randomized and selected for binding against bovine serum albumin. The individual mutants were found to bind selectively with BSA similarly with anti-BSA antibodies.

Lipovsek et al. (U.S. Pat. Nos. 6,818,418 and 7,115,396) discloses an antibody mimic featuring a fibronectin or fibronectin-like protein scaffold and at least one variable loop. Known as Adnectins, these fibronectin-based antibody mimics exhibit many of the same characteristics of natural or engineered antibodies, including high affinity and specificity for any targeted ligand. Any technique for evolving new or improved binding proteins can be used with these antibody mimics.

The structure of these fibronectin-based antibody mimics is similar to the structure of the variable region of the IgG heavy chain. Therefore, these mimics display antigen binding properties similar in nature and affinity to those of native antibodies. Further, these fibronectin-based antibody mimics exhibit certain benefits over antibodies and antibody fragments. For example, these antibody mimics do not rely on disulfide bonds for native fold stability, and are, therefore, stable under conditions which would normally break down antibodies. In addition, since the structure of these fibronectin-based antibody mimics is similar to that of the IgG heavy chain, the process for loop randomization and shuffling can be employed in vitro that is similar to the process of affinity maturation of antibodies in vivo.

Beste et al. (Proc Natl Acad Sci USA 96(5):1898-1903 (1999)) discloses an antibody mimic based on a lipocalin scaffold (Anticalin®). Lipocalins are composed of a beta-barrel with four hypervariable loops at the terminus of the protein. Beste (1999), subjected the loops to random mutagenesis and selected for binding with, for example, fluorescein. Three variants exhibited specific binding with fluorescein, with one variant showing binding similar to that of an anti-fluorescein antibody. Further analysis revealed that all of the randomized positions are variable, indicating that Anticalin® would be suitable to be used as an alternative to antibodies.

Anticalins® are small, single chain peptides, typically between 160 and 180 residues, which provides several advantages over antibodies, including decreased cost of production, increased stability in storage and decreased immunological reaction.

Hamilton et al. (U.S. Pat. No. 5,770,380) discloses a synthetic antibody mimic using the rigid, non-peptide organic scaffold of calixarene, attached with multiple variable peptide loops used as binding sites. The peptide loops all project from the same side geometrically from the calixarene, with respect to each other. Because of this geometric conformation, all of the loops are available for binding, increasing the binding affinity to a ligand. However, in comparison to other antibody mimics, the calixarene-based antibody mimic does not consist exclusively of a peptide, and therefore it is less vulnerable to attack by protease enzymes. Neither does the scaffold consist purely of a peptide, DNA or RNA, meaning this antibody mimic is relatively stable in extreme environmental conditions and has a long life span. Further, since the calixarene-based antibody mimic is relatively small, it is less likely to produce an immunogenic response.

Murali et al. (Cell Mol Biol. 49(2):209-216 (2003)) discusses a methodology for reducing antibodies into smaller peptidomimetics, they term “antibody like binding peptidomimetics” (ABiP) which can also be useful as an alternative to antibodies.

Silverman et al. (Nat Biotechnol. (2005), 23: 1556-1561) discloses fusion proteins that are single-chain polypeptides comprising multiple domains termed “avimers.” Developed from human extracellular receptor domains by in vitro exon shuffling and phage display the avimers are a class of binding proteins somewhat similar to antibodies in their affinities and specificities for various target molecules. The resulting multidomain proteins can comprise multiple independent binding domains that can exhibit improved affinity (in some cases sub-nanomolar) and specificity compared with single-epitope binding proteins. Additional details concerning methods of construction and use of avimers are disclosed, for example, in US Pat. App. Pub. Nos. 20040175756, 20050048512, 20050053973, 20050089932 and 20050221384.

In addition to non-immunoglobulin protein frameworks, antibody properties have also been mimicked in compounds comprising RNA molecules and unnatural oligomers (e.g., protease inhibitors, benzodiazepines, purine derivatives and beta-turn mimics) all of which are suitable for use with the present invention.

As known in the art, aptamers are macromolecules composed of nucleic acid that bind tightly to a specific molecular target. Tuerk and Gold (Science. 249:505-510 (1990)) discloses SELEX (Systematic Evolution of Ligands by Exponential Enrichment) method for selection of aptamers. In the SELEX method, a large library of nucleic acid molecules {e.g., 10¹⁵ different molecules) is produced and/or screened with the target molecule. Isolated aptamers can then be further refined to eliminate any nucleotides that do not contribute to target binding and/or aptamer structure (i.e., aptamers truncated to their core binding domain). See, e.g., Jayasena, 1999, Clin. Chem. 45:1628-1650 for review of aptamer technology.

Although the construction of test agent libraries is well known in the art, herein below, additional guidance in identifying test agents and construction libraries of such agents for the present screening methods are provided.

### (vi) Antibody Fragments

Various techniques have been developed for the production of antibody fragments. Traditionally, these fragments were derived via proteolytic digestion of intact antibodies (see, e.g., Morimoto K & Inouye K. J Biochem Biophys Methods. 1992 March; 24(1-2):107-17; Brennan M, et al., Science. 1985 Jul. 5; 229(4708):81-3). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F (ab′) 2 fragments (Carter P, et al., Biotechnology (N Y). 1992 February; 10(2):163-7). According to another approach, F (ab′) 2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. Nos. 5,571,894 and 5,587,458. The antibody fragment can also be a “linear antibody”, e.g., as described in U.S. Pat. No. 5,641,870 for example. Such linear antibody fragments can be monospecific or bispecific.

(vii) Selecting the Antibody or Antibody Fragment

The antibody or antibody fragment which prepared by aforementioned method is selected by detecting affinity of CX genes expressing cells like cancers cell. Unspecific binding to these cells is blocked by treatment with PBS containing 3% BSA for 30 min at room temperature. Cells are incubated for 60 min at room temperature with candidate antibody or antibody fragment. After washing with PBS, the cells are stained by FITC-conjugated secondary antibody for 60 min at room temperature and detected by using fluorometer. Alternatively, a biosensor using the surface plasmon resonance phenomenon can be used as a mean for detecting or quantifying the antibody or antibody fragment in the present invention. The antibody or antibody fragment which can detect the CX peptide on the cell surface is selected in the presence invention.

### (3) Double-Stranded Molecule

The term “polynucleotide” and “oligonucleotide” are used interchangeably herein unless otherwise specifically indicated and are referred to by their commonly accepted single-letter codes. The terms apply to nucleic acid (nucleotide) polymers in which one or more nucleic acids are linked by ester bonding. The polynucleotide or oligonucleotide can be composed of DNA, RNA or a combination thereof.

As use herein, the term “isolated double-stranded molecule” refers to a nucleic acid molecule that inhibits expression of a target gene including, for example, short interfering RNA (siRNA; e.g., double-stranded ribonucleic acid (dsRNA) or small hairpin RNA (shRNA)) and short interfering DNA/RNA (siD/R-NA; e.g. double-stranded chimera of DNA and RNA (dsD/R-NA) or small hairpin chimera of DNA and RNA (shD/R-NA)).

As use herein, the term “siRNA” refers to a double-stranded RNA molecule which prevents translation of a target mRNA. Standard techniques of introducing siRNA into the cell are used, including those in which DNA is a template from which RNA is transcribed. The siRNA includes a ribonucleotide corresponding to a sense nucleic acid sequence of CX gene (also referred to as “sense strand”), a ribonucleotide corresponding to an antisense nucleic acid sequence of CX gene (also referred to as “antisense strand”) or both. The siRNA can be constructed such that a single transcript has both the sense and complementary antisense nucleic acid sequences of the target gene, e.g., a hairpin. The siRNA can either be a dsRNA or shRNA.

As used herein, the term “dsRNA” refers to a construct of two RNA molecules comprising complementary sequences to one another and that have annealed together via the complementary sequences to form a double-stranded RNA molecule. The sequence of two strands can comprise not only the “sense” or “antisense” RNAs selected from a protein coding sequence of target gene sequence, but also RNA molecule having a nucleotide sequence selected from non-coding region of the target gene.

The term “shRNA”, as used herein, refers to an siRNA having a stem-loop structure, comprising a first and second regions complementary to one another, i.e., sense and antisense strands. The degree of complementarity and orientation of the region is sufficient such that base pairing occurs between the regions, the first and second regions being joined by a loop region, the loop resulting from a lack of base pairing between nucleotides (or nucleotide analogs) within the loop region. The loop region of an shRNA is a single-stranded region intervening between the sense and antisense strands and can also be referred to as “intervening single-strand”.

As use herein, the term “siD/R-NA” refers to a double-stranded molecule which is composed of both RNA and DNA, and includes hybrids and chimeras of RNA and DNA and prevents translation of a target mRNA. Herein, a hybrid indicates a molecule wherein an oligonucleotide composed of DNA and an oligonucleotide composed of RNA hybridize to each other to form the double-stranded molecule; whereas a chimera indicates that one or both of the strands composing the double stranded molecule can contain RNA and DNA. Standard techniques of introducing siD/R-NA into the cell are used. The siD/R-NA includes a sense nucleic acid sequence of CX gene (also referred to as “sense strand”), an antisense nucleic acid sequence of CX gene (also referred to as “antisense strand”) or both. The siD/R-NA can be constructed such that a single transcript has both the sense and complementary antisense nucleic acid sequences from the target gene, e.g., a hairpin. The siD/R-NA can either be a dsD/R-NA or shD/R-NA.

As used herein, the term “dsD/R-NA” refers to a construct of two molecules comprising complementary sequences to one another and that have annealed together via the complementary sequences to form a double-stranded polynucleotide molecule. The nucleotide sequence of two strands can comprise not only the “sense” or “antisense” polynucleotides sequence selected from a protein coding sequence of target gene sequence, but also polynucleotide having a nucleotide sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, comprising a first and second regions complementary to one another, i.e., sense and antisense strands. The degree of complementarity and orientation of the regions is sufficient such that base pairing occurs between the regions, the first and second regions being joined by a loop region, the loop resulting from a lack of base pairing between nucleotides (or nucleotide analogs) within the loop region. The loop region of an shD/R-NA is a single-stranded region intervening between the sense and antisense strands and can also be referred to as “intervening single-strand”.

### Overview

### (1) CDCA5

To identify biomarkers and/or therapeutic targets for cancer treatment, the present inventors analyzed the gene expression profiles of 120 cases of clinical lung and esophageal carcinomas using a cDNA microarray containing 27,648 genes. Among the genes that were up-regulated commonly in these tumors, a CDCA5 that encodes a substrate of the anaphase-promoting complex was identified. Northern-blot analysis identified a CDCA5 transcript only in testis among 23 normal tissues examined. Treatment of cancer cells with siRNAs against CDCA5 suppressed its expression and suppressed growth of the cells. On the other hand, induction of exogenous expression of CDCA5 conferred growth-promoting activity in mammalian cells. In vitro kinase assay detected the CDC2-mediated phosphorylation of CDCA5 polypeptide or ERK-mediated phosphorylation of CDCA5. Since CDCA5 can be categorized as cancer-testis antigen and is indispensable for cell growth and/or survival, targeting the CDCA5 and/or the enzymatic activity of CDC2 polypeptide or ERK polypeptide on CDCA5 polypeptide is a promising strategy for developing treatment of lung and esophageal carcinoma for example, molecular targeted drugs and cancer vaccines.

### (2) EPHA7

The present inventors investigated gene-expression profiles of lung and esophageal cancers, and identified elevated expression of ephrin receptor A7 (EPHA7) that belongs to the ephrin receptor subfamily of the protein-tyrosine kinase family, in the majority of lung cancers and esophageal squamous-cell carcinomas (ESCCs). Immunohistochemical staining using tumor tissue microarray consisting of 402 archived non-small cell lung cancers (NSCLCs) and 292 ESCC specimens demonstrated that a high level of EPHA7 expression was associated with poor prognosis for patients with NSCLC as well as ESCC, and multivariate analysis confirmed its independent prognostic value for NSCLC. The present inventors established an ELISA to measure serum EPHA7 and found that the proportion of serum EPHA7-positive cases was 149 (56.4%) of 264 non-small cell cancer (NSCLC), 35 (44.3%) of 79 SCLC, and 81 (84.4%) of 96 ESCC patients, while only 6 (4.7%) of 127 healthy volunteers were falsely diagnosed. A combined ELISA for both EPHA7 and CEA classified 77.2% of the NSCLC patients as positive, and the use of both EPHA7 and ProGRP increased sensitivity in the detection of SCLCs up to 77.5%, while the false positive rate was 7-8%. In addition, treatment of lung cancer cells with siRNAs for EPHA7 suppressed the growth of the cells, whereas induction of EPHA7 increased the cellular invasion and growth-promoting activity. To investigate its function, we screened for downstream targets for EPHA7 kinase using a panel of antibodies against phospho-proteins related to cancer-cell signaling, and identified EPHA7-induced phosphorylation of EGFR (Tyr-845), PLCgamma (Tyr-783) (GenBank Accession No.: NM_{—}002660, SEQ ID NO.: 52), CDC25 (Ser-216) (GenBank Accession No.: NM_{—}001790, SEQ ID NO.: 54), MET (Tyr-1230/1234/1235, Tyr-1313, Tyr-1349, Tyr-1365) (GenBank Accession No.: NM_{—}000245, SEQ ID NO.: 56), Shc (Tyr317, Tyr239/240) (GenBank Accession No.: NM_{—}001130041, SEQ ID NO.: 58), ERK (p44/42 MAPK) (Thr202/Tyr204) (GenBank Accession No.: NM_{—}001040056, SEQ ID NO.: 50), Akt (Ser473) (GenBank Accession No.: NM_{—}001014431 SEQ ID NO.: 60), and STAT3 (Tyr705) (GenBank Accession No.: NM_{—}139276). These data are consistent with the conclusion that EPHA7 plays a significant role in cancer cell growth and invasion and should be useful as an effective tumor biomarker and a therapeutic target.

### (3) STK31

Gene-expression profile analysis of 27,648 genes using 120 lung and esophageal cancers revealed that a gene encoding a serine/threonine kinase 31 (STK31), was frequently transactivated in these cancers. STK31 showed testis-specific expression in normal tissues. STK31 was localized in the cytoplasm and nucleus of cancer cells. Immunohistochemical staining of STK31 on tissue microarray containing 368 lung cancers indicated an association of STK31 expression with poor clinical outcome (P=0.0178 by log-rank test), demonstrating its usefulness as a prognostic biomarker. Treatment of lung cancer cells with siRNAs against STK31 suppressed its expression and resulted in growth suppression. On the other hand, induction of exogenous expression of STK31 conferred growth-promoting activity in mammalian cells. Phosphorylation assay using recombinant STK31 protein proved its kinase activity, and induction of STK31 expression caused the phosphorylation of EGFR (Ser1046/1047), ERK (p44/42 MAPK) (Thr202/Tyr204) (GenBank Accession No.: NM_{—}001040056, SEQ ID NO.: 50) and MEK (Ser217/Ser221) in mammalian cells. Our data are consistent with the conclusion that the selective inhibition of the enzymatic activity of STK31 is a promising therapeutic strategy for development of molecular targeted agents and cancer vaccines.

### (4) WDHD1

Through a cDNA microarray analysis of 32,000 genes, the present inventors found abundant expression of the WD Repeat and HMG-box DNA Binding Protein 1 (WDHD1) in the majority of lung cancers and esophageal squamous cell carcinomas (ESCC). Northern-blot analysis identified no WDHD1 expression in any normal tissues examined except the testis. WDHD1 was localized in the nucleus of cancer cells. Immunoprecipitation of WDHD1 with anti-WDHD1 antibody followed by immunoblotting with pan-phospho-specific antibodies indicated phosphorylation of WDHD1 at its serine and tyrosine residues. Tissue microarray analyses covering 297 ESCC and 264 lung cancers showed an association of a high level of WDHD1 expression with poor prognosis (P=0.0285 and 0.0208 respectively by log-rank test). Suppression of WDHD1 expression with siRNA effectively suppressed the growth of cancer cells.

Concordantly, induction of exogenous expression of WDHD1 in COS-7 cells revealed its growth-promoting activity. WDHD1 was phosphorylated at its serine and tyrosine residues. The level of WDHD1 was increased at a transition period from G1 to S phases, reaching the maximum level at S phase, while it was decreased by phosphatidylinositol-3 kinase (PI3K) inhibitor, LY294002. These data implied that WDHD1 should be categorized in a cancer-testis antigen and plays a significant role in cell cycle progression through PI3K/AKT pathway. Selective inhibition of the oncogenic WDHD1 activity is a promising approach for developing molecular targeted agents to treat esophageal and lung cancers.

### Double-Stranded Molecule for CX Gene(s)

### (i) Target Sequence

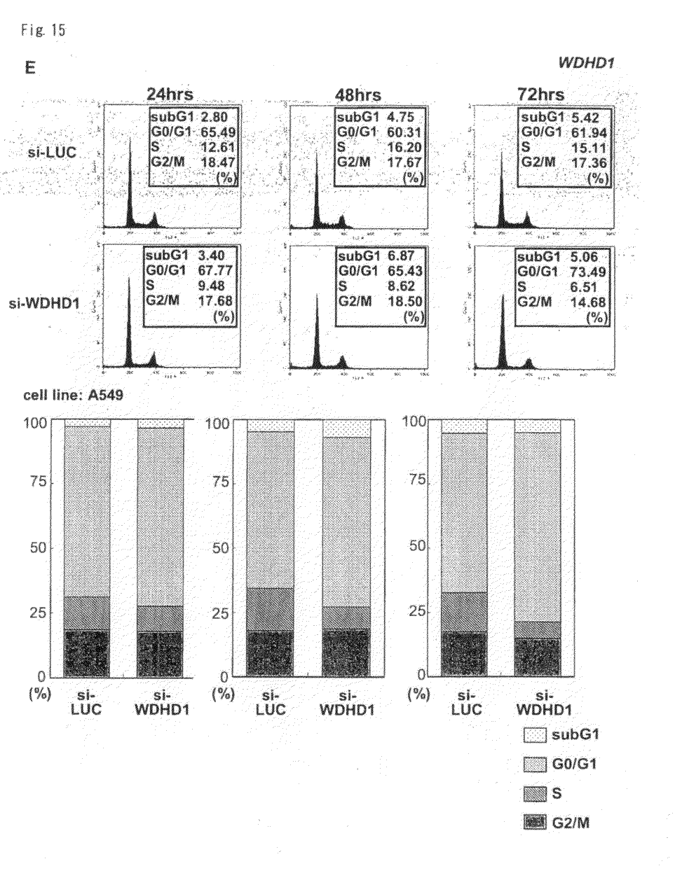

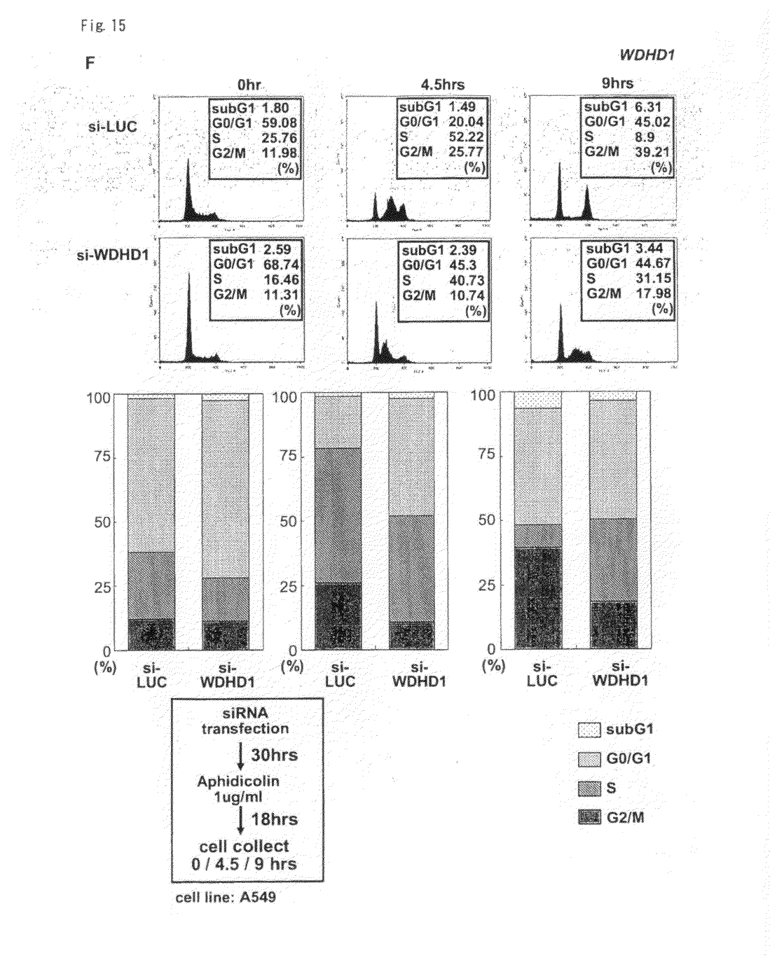

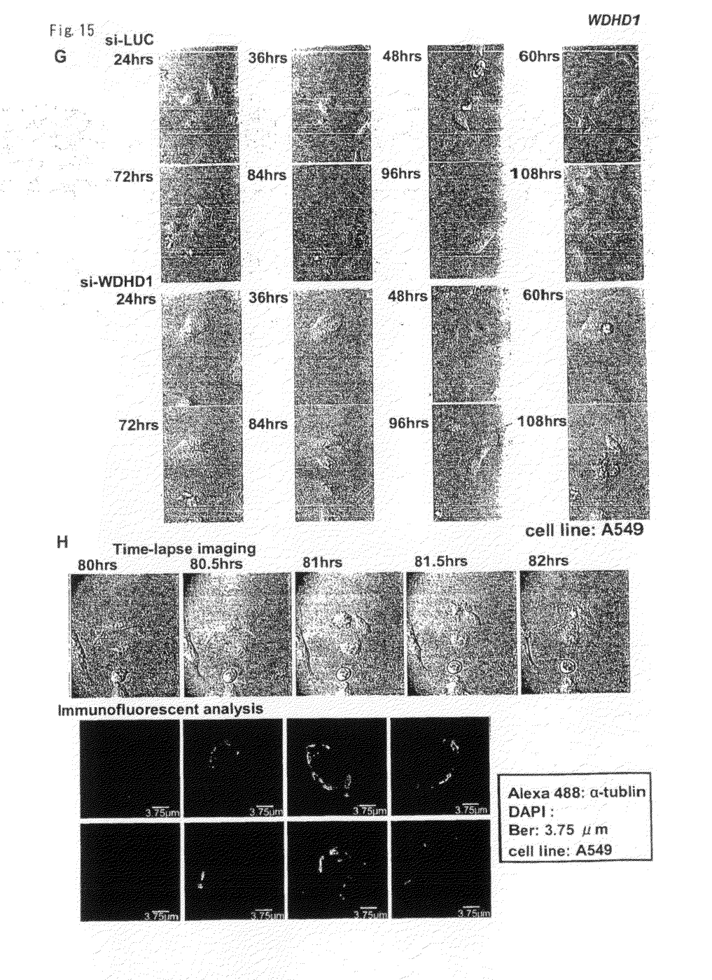

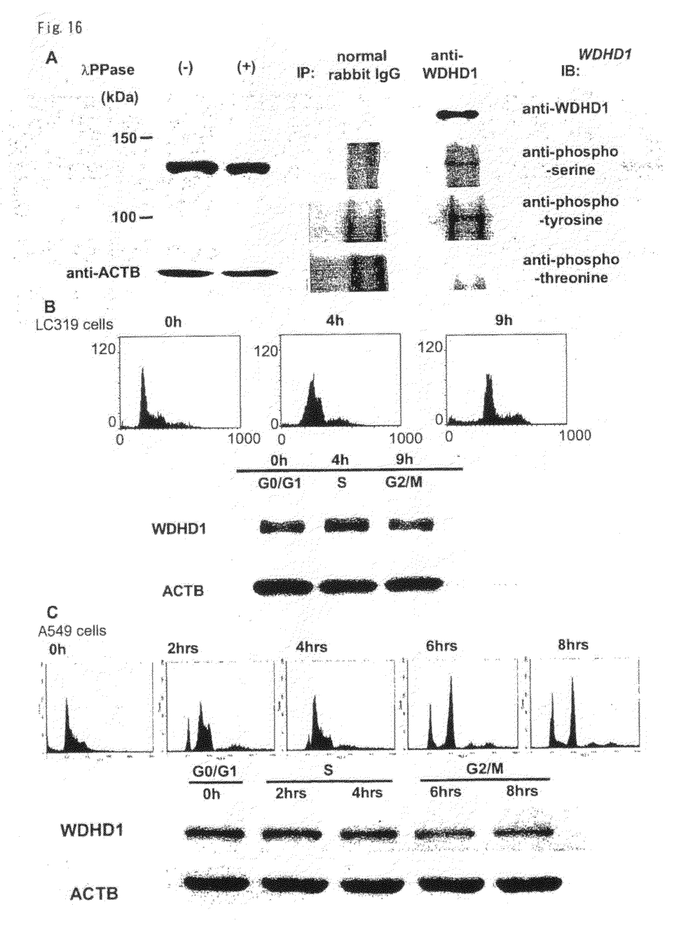

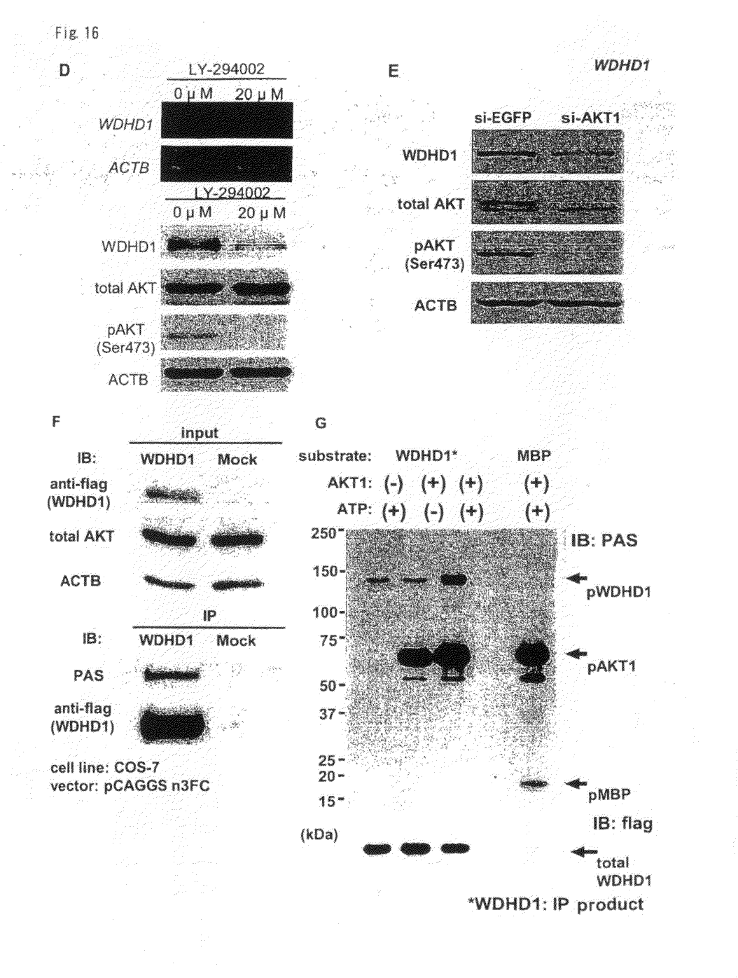

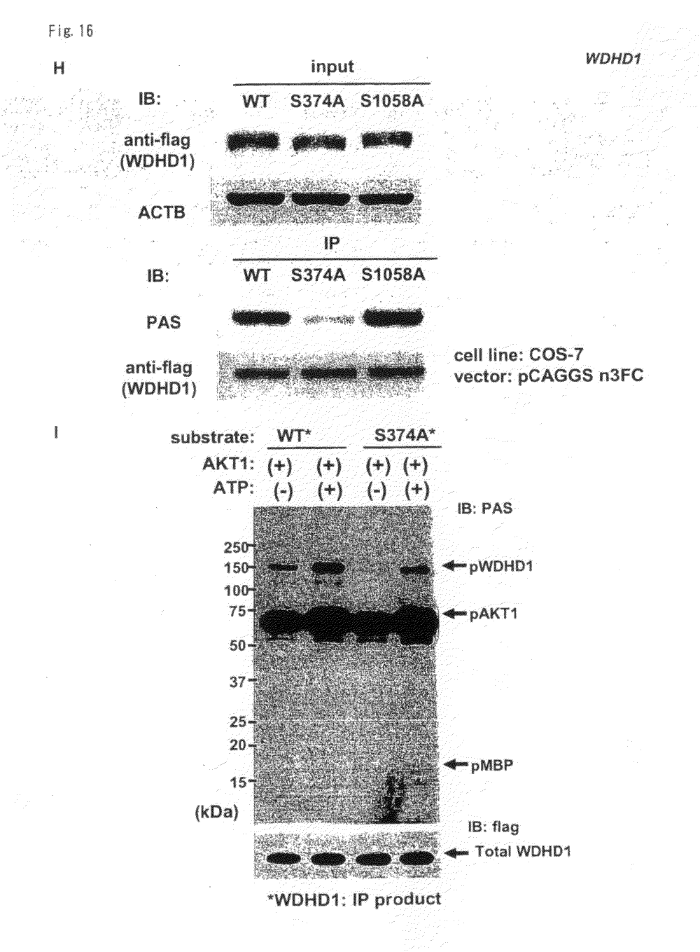

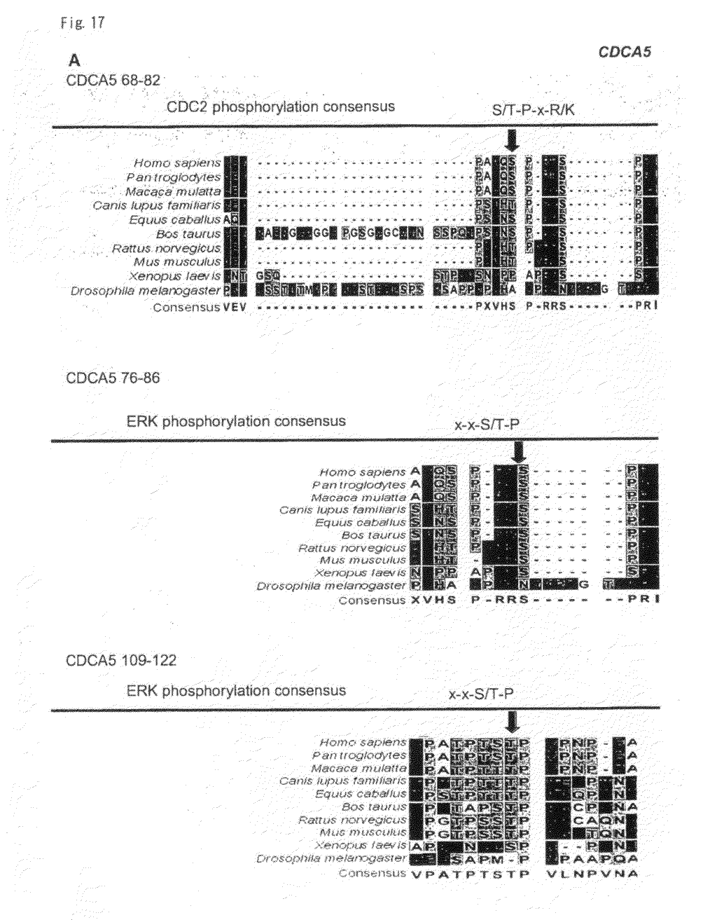

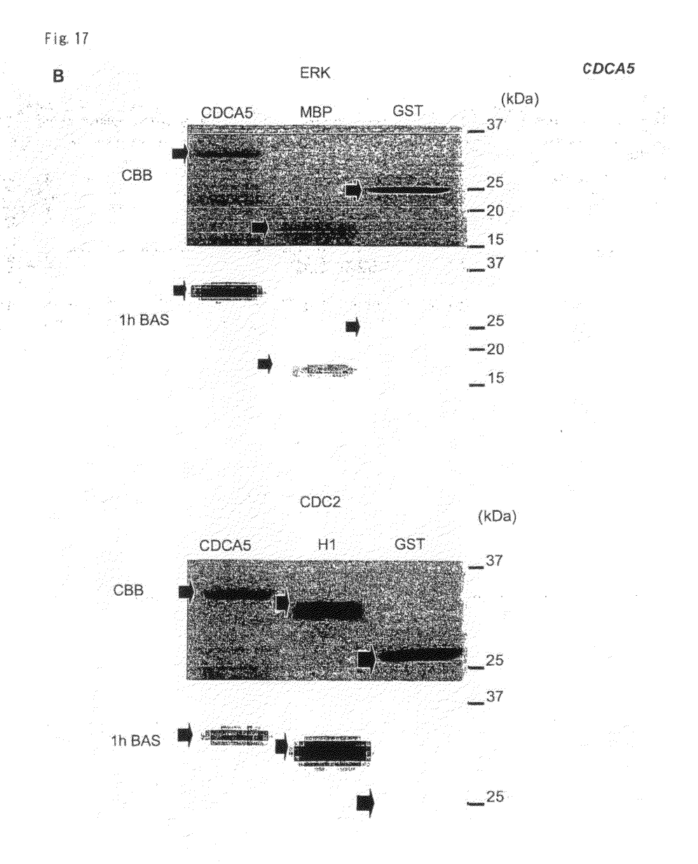

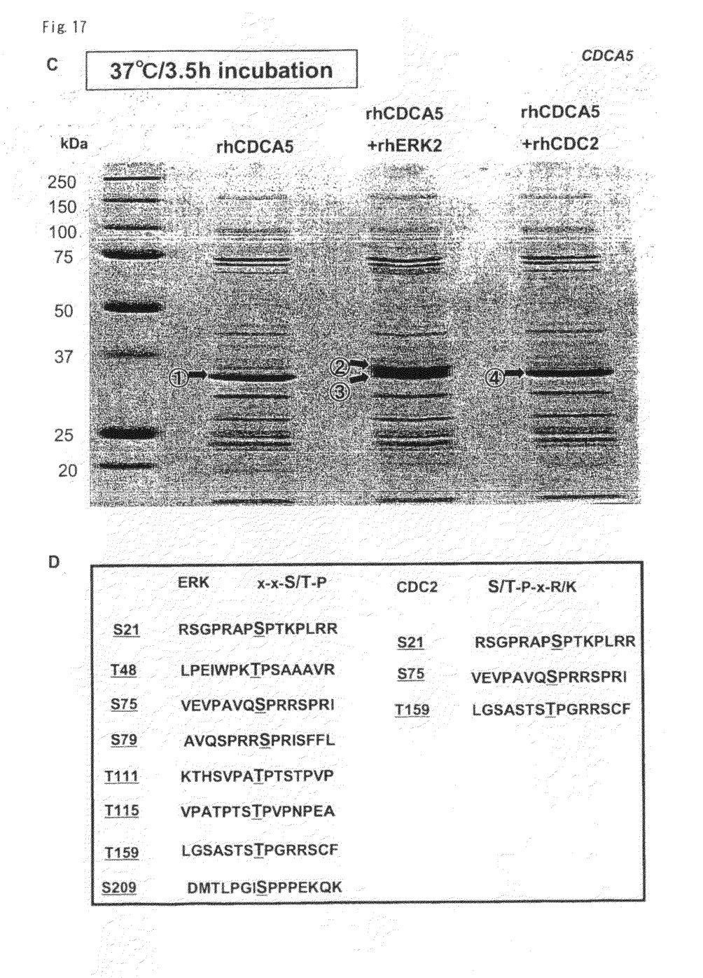

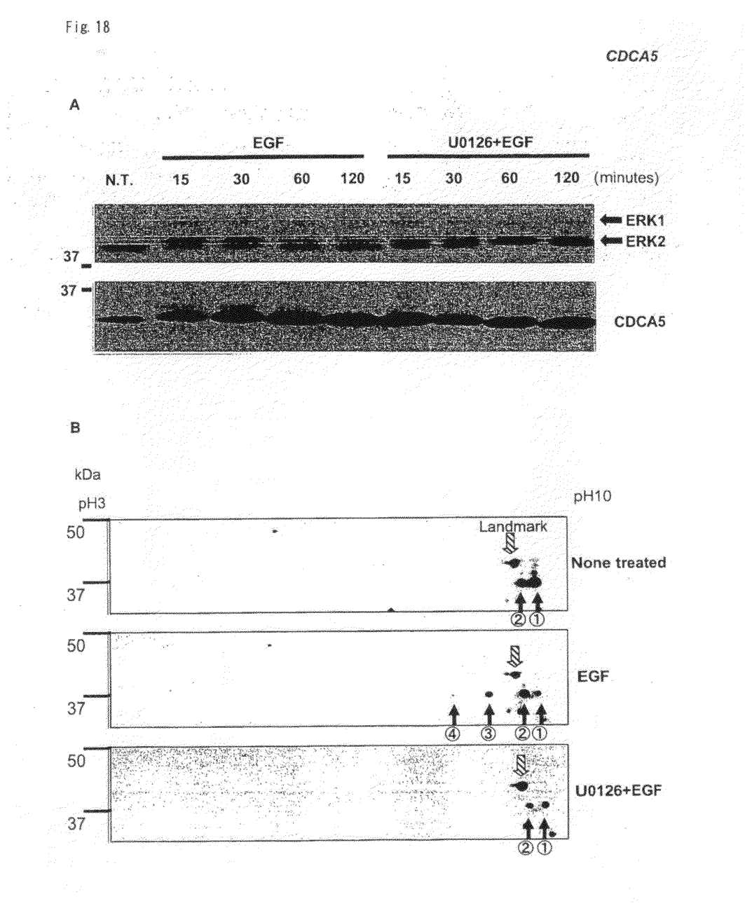

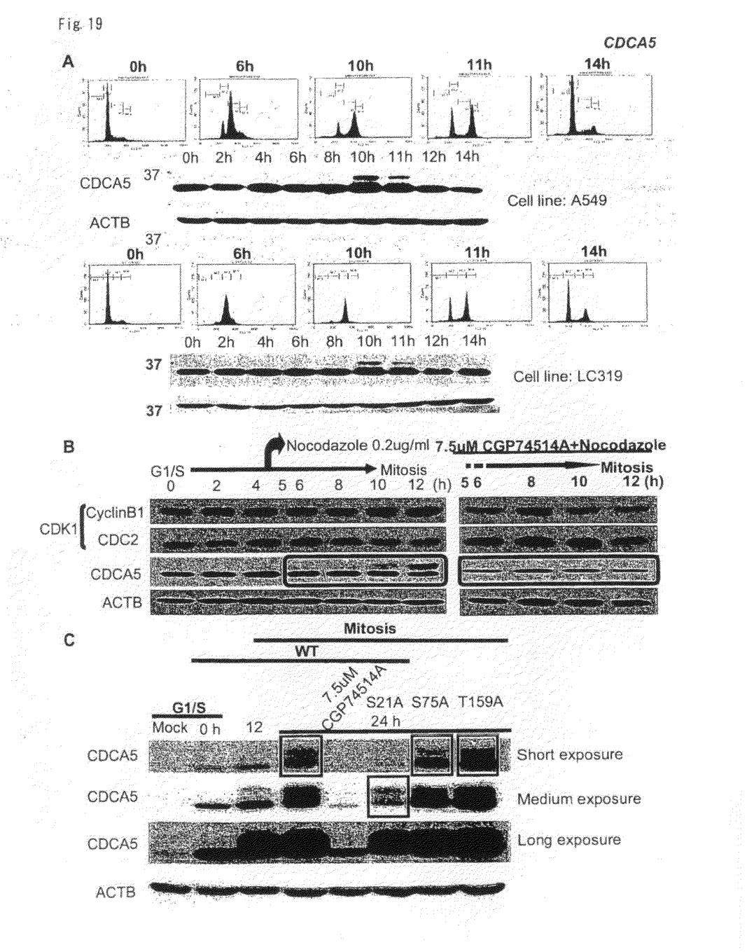

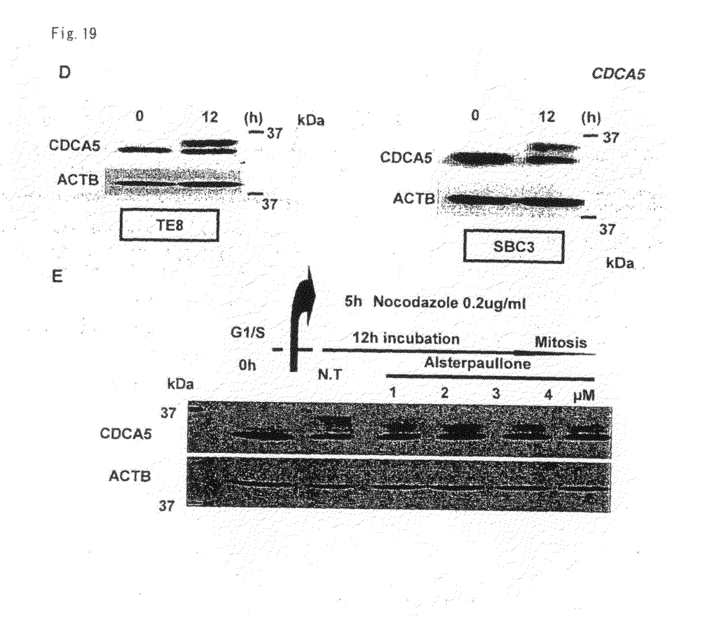

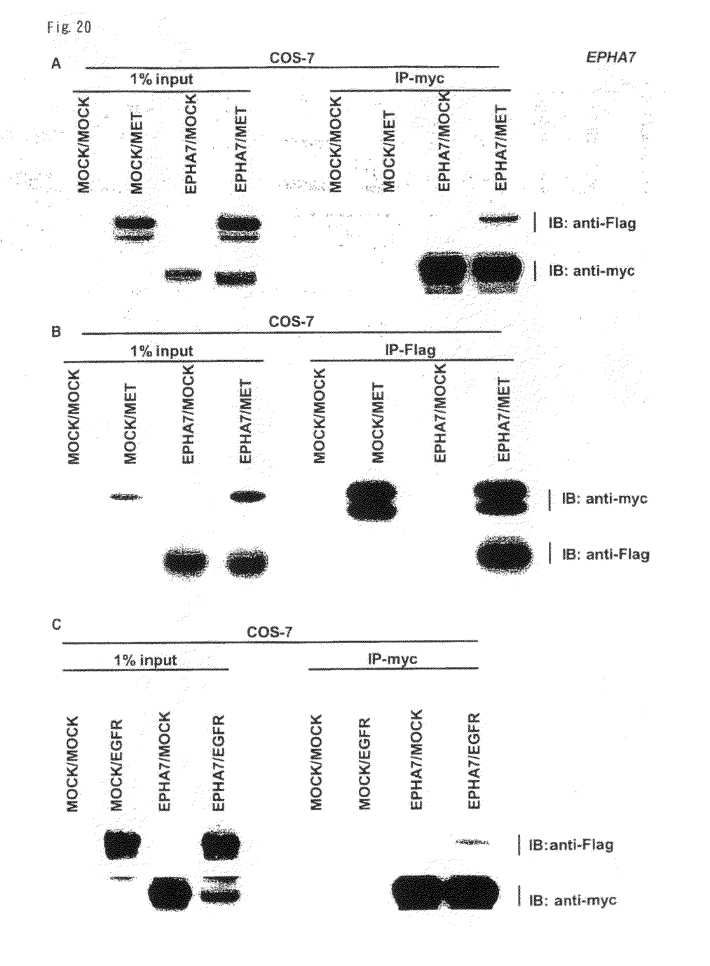

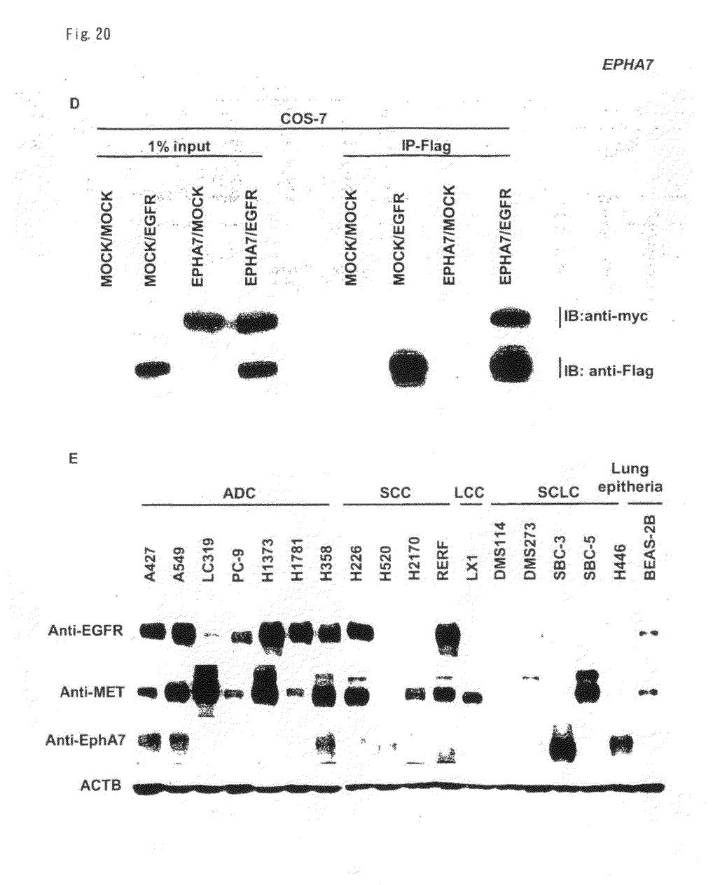

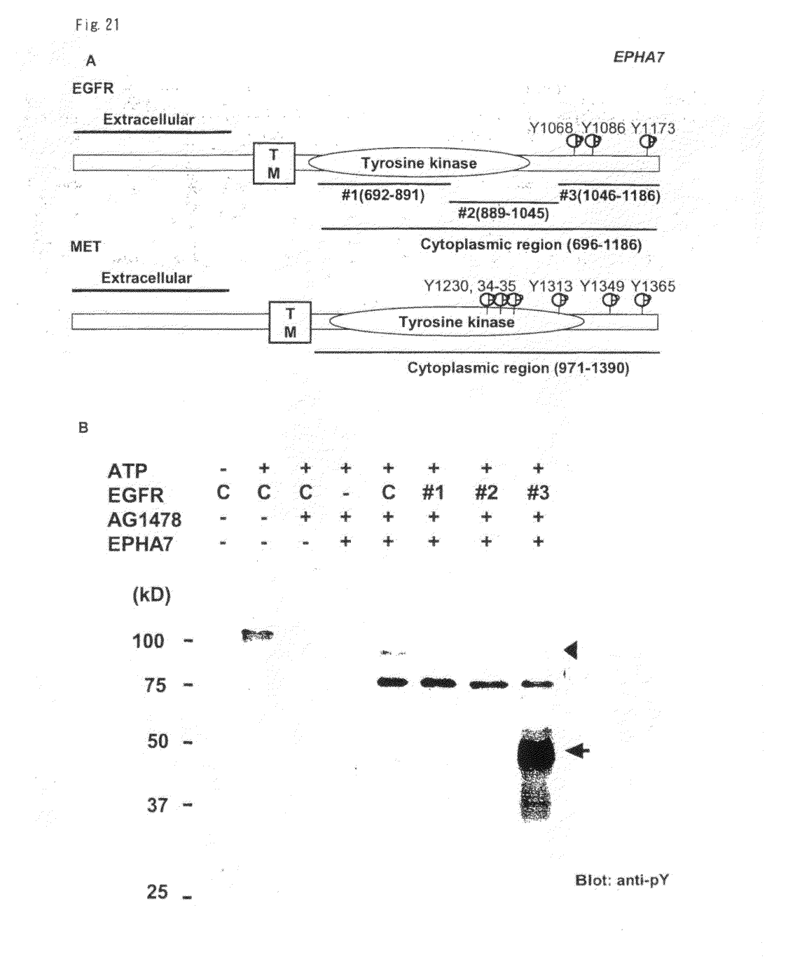

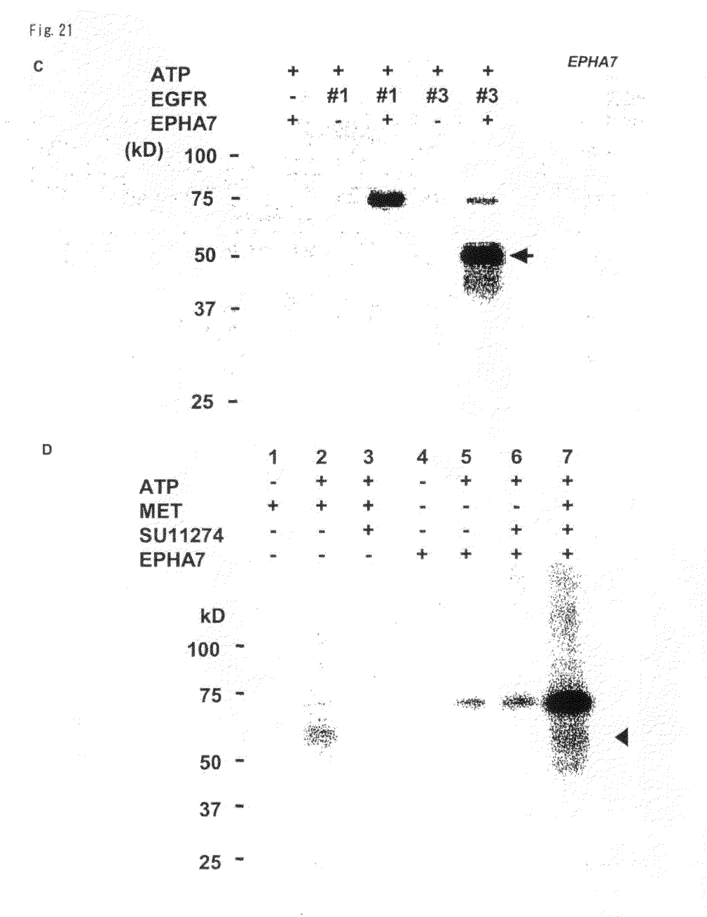

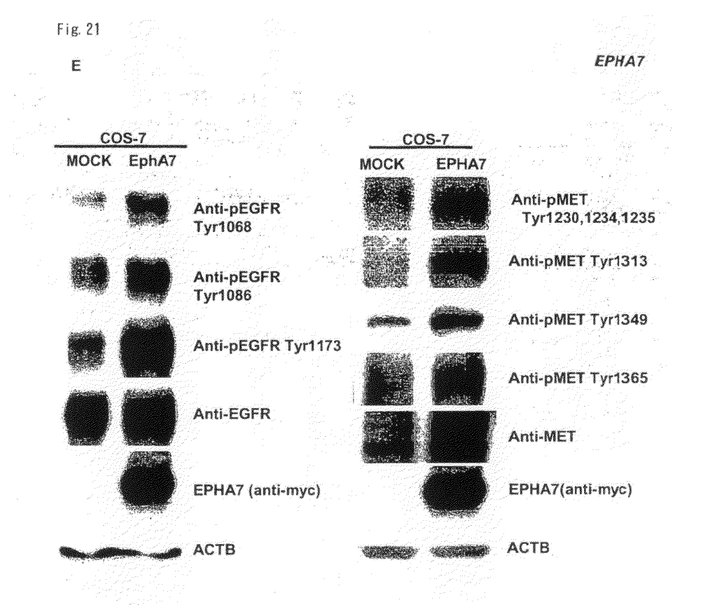

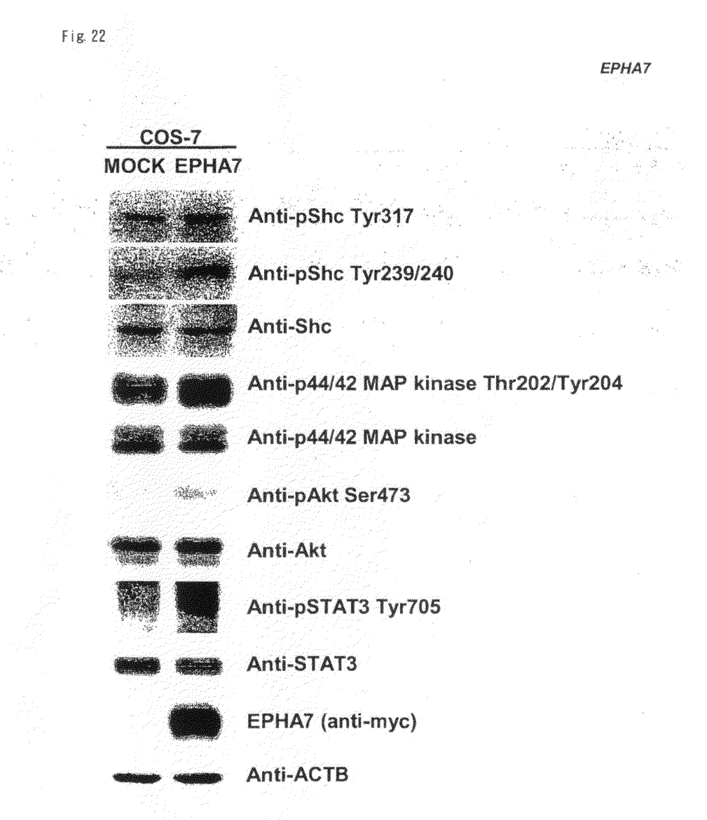

A double-stranded molecule against CX gene(s), which molecule hybridizes to target mRNA, inhibits or reduces production of CX protein(s) encoded by CX gene(s) by associating with the normally single-stranded mRNA transcript of the gene, thereby interfering with translation and thus, inhibiting expression of the protein encoded by target gene. The expression of CX gene(s) in cancer cell lines, was inhibited by double-stranded molecules of the present invention; the expression of CDCA5 in cancers cell lines was inhibited by two double-stranded molecules (FIGS. 2A and B, upper panels); the expression of EPHA7 in cancers cell lines was inhibited by two double-stranded molecules (FIG. 6A, upper panels); the expression of STK31 in cancers cell lines was inhibited by two double-stranded molecules (FIG. 11A); the expression of WDHD1 in cancers cell lines was inhibited by two double-stranded molecules (FIGS. 15 A and B, upper panels).

Therefore the present invention provides isolated double-stranded molecules having the property to inhibit or reduce the expression of CX gene in cancer cells when introduced into a cell. The target sequence of double-stranded molecule is designed by siRNA design algorithm mentioned below.

CDCA5 target sequence includes, for example, nucleotides

| | |
|---|---|
| 5′-GCAGTTTGATCTCCTGGT-3′ | (SEQ ID NO: 40) |

(at the position 808-827 nt of SEQ ID NO: 1)

| | |
|---|---|
| or | |
| 5′-GCCAGAGACTTGGAAATGT-3′ | (SEQ ID NO: 41) |

(at the position 470-488 nt of SEQ ID NO: 1)

EPHA7 target sequence includes, for example, nucleotides

| | |
|---|---|
| 5′-AAAAGAGATGTTGCAGTA-3′ | (SEQ ID NO: 42) |

(at the position 2182-2200 nt of SEQ ID NO: 3)

| | |
|---|---|
| or | |
| 5′-TAGCAAAGCTGACCAAGAA-3′ | (SEQ ID NO: 43) |

(at the position 1968-1987 nt of SEQ ID NO: 3)

STK31 target sequence includes, for example, nucleotides

| 5′-GGAGATAGCTCTGGTTGAT-3′ | (SEQ ID NO: 38) |
|---|---|
| (position at 1713-1732 nt of SEQ ID NO: 5) | |
| or | |
| 5′-GGGCTATTCTGTGGATGTTS-3′ | (SEQ ID NO: 39) |

(position at 2289-2308 nt of SEQ ID NO: 5)

WDHD1 target sequence includes, for example, nucleotides

| 5′-GATCAGACATGTGCTATTA-3′ | (SEQ ID NO: 44) |
|---|---|
| (at the position of SEQ ID NO: 7) | |
| or | |
| 5′-GGTAATACGTGGACTCCTA-3′ | (SEQ ID NO: 45) |

(at the position of SEQ ID NO: 7)

Specifically, the present invention provides the following double-stranded molecules [1] to [19]:

[1] An isolated double-stranded molecule, which,

(i) when introduced into a cell, inhibits in vivo expression of an CDCA5 gene and cell proliferation, wherein said double-stranded molecule acts at mRNA which matches a target sequence selected from the group SEQ ID NO: 40 (at the position 808-827 nt of SEQ ID NO: 1) and SEQ ID NO: 41 (at the position 470-488 nt of SEQ ID NO: 1);

(ii) when introduced into a cell, inhibits in vivo expression of an EPHA7 gene and cell proliferation, wherein said double-stranded molecule acts at mRNA which matches a target sequence selected from the group SEQ ID NO: 42 (at the position 2182-2200 nt of SEQ ID NO: 3) and SEQ ID NO: 43 (at the position 1968-1987 nt of SEQ ID NO: 3).

(iii) when introduced into a cell, inhibits in vivo expression of an STK31 gene and cell proliferation, wherein said double-stranded molecule acts at mRNA which matches a target sequence selected from the group SEQ ID NO: 38 (position at 1713-1732 nt of SEQ ID NO: 5) and SEQ ID NO: 39 (position at 2289-2308 nt of SEQ ID NO: 5).

(iv) when introduced into a cell, inhibits in vivo expression of an WDHD1 gene and cell proliferation, wherein said double-stranded molecule acts at mRNA which matches a target sequence selected from the group SEQ ID NO: 44 (at the position of SEQ ID NO: 7) and SEQ ID NO: 45 (at the position of SEQ ID NO: 7).

[2] The double-stranded molecule of [1], which comprises a sense strand and an antisense strand complementary thereto, hybridized to each other to form a double strand,

(i) wherein said sense strand comprises an oligonucleotide corresponding to a sequence selected from the group consisting of SEQ ID NO: 40 and SEQ ID NO: 41 for CDCA5;

(ii) wherein said sense strand comprises an oligonucleotide corresponding to a sequence selected from the group consisting of SEQ ID NO: 42 and SEQ ID NO: 43 for EPHA7;

(iii) wherein said sense strand comprises an oligonucleotide corresponding to a sequence selected from the group consisting of SEQ ID NO: 38 and SEQ ID NO: 39 for STK31;

(iv) wherein said sense strand comprises an oligonucleotide corresponding to a sequence selected from the group consisting of SEQ ID NO: 44 and SEQ ID NO: 45 for WDHD1.

[3] The double-stranded molecule of [1], wherein said target sequence comprises at least about 10 contiguous nucleotide from the nucleotide sequence selected from SEQ ID NO: 1 for CDCA5, SEQ ID NO: 3 for EPHA7, SEQ ID NO: 5 for STK31 or SEQ ID NO: 7 for WDHD1.

[4] The double-stranded molecule of [3], wherein said target sequence comprises from about 19 to about 25 contiguous nucleotide from the nucleotide sequence selected from SEQ ID NO: 1 for CDCA5, SEQ ID NO: 3 for EPHA7, SEQ ID NO: 5 for STK31 or SEQ ID NO: 7 for WDHD1.

[5] The double-stranded molecule of [2], which has a length of less than about 100 nucleotides.

[6] The double-stranded molecule of [5], which has a length of less than about 75 nucleotides.

[7] The double-stranded molecule of [6], which has a length of less than about 50 nucleotides.

[8] The double-stranded molecule of [7] which has a length of less than about 25 nucleotides.

[9] The double-stranded molecule of [8], which has a length of between about 19 and about 25 nucleotides.

[10] The double-stranded molecule of [1], which consists of a single oligonucleotide comprising both the sense and antisense strands linked by an intervening single-strand.

[11] The double-stranded molecule of [10], which has a general formula 5′-[A]-[B]-[A′]-3′, wherein

[A] is the sense strand comprising an oligonucleotide corresponding to a sequence selected from the group consisting of SEQ ID NO: 40 and SEQ ID NO: 41 for CDCA5, SEQ ID NO: 42 and SEQ ID NO: 43 for EPHA7, SEQ ID NO: 38 and SEQ ID NO: 39 for STK31, SEQ ID NO: 44 and SEQ ID NO: 45 for WDHD1;

[B] is the intervening single-strand; and

[A′] is the antisense strand comprising an oligonucleotide corresponding to a sequence complementary to the sequence selected in [A].

[12] The double-stranded molecule of [1], which comprises RNA.

[13] The double-stranded molecule of [1], which comprises both DNA and RNA.

[14] The double-stranded molecule of [13], which is a hybrid of a DNA polynucleotide and an RNA polynucleotide.

[15] The double-stranded molecule of [14] wherein the sense and the antisense strands are made of DNA and RNA, respectively.

[16] The double-stranded molecule of [13], which is a chimera of DNA and RNA.

[17] The double-stranded molecule of [16], wherein a 5′-end region of the target sequence in the sense strand, and/or a 3′-end region of the complementary sequence of the target sequence in the antisense strand consists of RNA.

[18] The double-stranded molecule of [17], wherein the RNA region consists of 9 to 13 nucleotides; and

[19] The double-stranded molecule of [2], which contains 3′ overhang.

The double-stranded molecule of the present invention will be described in more detail below.

Methods for designing double-stranded molecules having the ability to inhibit target gene expression in cells are known. (See, for example, U.S. Pat. No. 6,506,559, herein incorporated by reference in its entirety). For example, a computer program for designing siRNAs is available from the Ambion website (on the worldwide web at ambion.com/techlib/misc/siRNA_finder.html).

The computer program selects target nucleotide sequences for double-stranded molecules based on the following protocol.

### Design of Target Sites

1. Beginning with the AUG start codon of the transcript, scan downstream for AA di-nucleotide sequences. Record the occurrence of each AA and the 3′ adjacent 19 nucleotides as potential siRNA target sites. Tuschl et al. recommend to avoid designing siRNA to the 5′ and 3′ untranslated regions (UTRs) and regions near the start codon (within 75 bases) as these can be richer in regulatory protein binding sites, and UTR-binding proteins and/or translation initiation complexes can interfere with binding of the siRNA endonuclease complex.

2. Compare the potential target sites to the appropriate genome database (human, mouse, rat, etc.) and eliminate from consideration any target sequences with significant homology to other coding sequences. Basically, BLAST, which can be found on the NCBI server at: on the worldwide web at ncbi.nlm.nih.gov/BLAST/, is used (Altschul S F, et al., Nucleic Acids Res. 1997 Sep. 1; 25(17):3389-402).

3. Select qualifying target sequences for synthesis. Selecting several target sequences along the length of the gene to evaluate is typical.

By the protocol, the target sequence of the isolated double-stranded molecules of the present invention were designed as

CDCA5 target sequence includes, for example, nucleotides

| | |
|---|---|
| 5′-GCAGTTTGATCTCCTGGT-3′ | (SEQ ID NO: 40) |

(at the position 808-827 nt of SEQ ID NO: 1)

| | |
|---|---|
| or | |
| 5′-GCCAGAGACTTGGAAATGT-3′ | (SEQ ID NO: 41) |

(at the position 470-488 nt of SEQ ID NO: 1)

EPHA7 target sequence includes, for example, nucleotides

| | |
|---|---|
| 5′-AAAAGAGATGTTGCAGTA-3′ | (SEQ ID NO: 42) |

(at the position 2182-2200 nt of SEQ ID NO: 3)

| | |
|---|---|
| or | |
| 5′-TAGCAAAGCTGACCAAGAA-3′ | (SEQ ID NO: 43) |

(at the position 1968-1987 nt of SEQ ID NO: 3)

STK31 target sequence includes, for example, nucleotides

| 5′-GGAGATAGCTCTGGTTGAT-3′ | (SEQ ID NO: 38) |
|---|---|
| (position at 1713-1732 nt of SEQ ID NO: 5) | |
| or | |
| 5′-GGGCTATTCTGTGGATGTTS-3′ | (SEQ ID NO: 39) |

(position at 2289-2308 nt of SEQ ID NO: 5)

WDHD1 target sequence includes, for example, nucleotides

| 5′-GATCAGACATGTGCTATTA-3′ | (SEQ ID NO: 44) |
|---|---|
| (at the position of SEQ ID NO: 7) | |
| or | |
| 5′-GGTAATACGTGGACTCCTA-3′ | (SEQ ID NO: 45) |

(at the position of SEQ ID NO: 7)

Specifically, the present invention provides the following double-stranded molecules targeting the above-mentioned target sequences were respectively examined for their ability to inhibit or reduce the growth of cells expressing the target genes. The growth of cancer cell expressing CX gene(s), was inhibited or reduced by double-stranded molecules of the present invention; the growth of the CDCA5 expressing cells, e.g. lung cancer cell line A549 and LC319, was inhibited by two double stranded molecules (FIGS. 2A and B, middle and lower panels); the growth of the EPHA7 expressing cells, e.g. lung cancer cell line NCI-H520 and SBC-5, was inhibited by two double stranded molecules (FIG. 6A, middle and lower panels); the growth of the STK31 expressing cells, e.g. lung cancer cell line LC319 and NCI-H2170, was inhibited by two double stranded molecules (FIGS. 11B and C); the growth of the WDHD1 expressing cells, e.g. lung cancer cell line LC319 and TE9, was inhibited by two double stranded molecules (FIG. 15A middle and lower panels). Therefore, the present invention provides double-stranded molecules targeting any of the sequences selected from the group of

CDCA5 target sequence includes, for example, nucleotides

| | |
|---|---|
| 5′-GCAGTTTGATCTCCTGGT-3′ | (SEQ ID NO: 40) |

(at the position 808-827 nt of SEQ ID NO: 1)

| | |
|---|---|
| or | |
| 5′-GCCAGAGACTTGGAAATGT-3′ | (SEQ ID NO: 41) |

(at the position 470-488 nt of SEQ ID NO: 1)

EPHA7 target sequence includes, for example, nucleotides

| | |
|---|---|
| 5′-AAAAGAGATGTTGCAGTA-3′ | (SEQ ID NO: 42) |

(at the position 2182-2200 nt of SEQ ID NO: 3)

| | |
|---|---|
| or | |
| 5′-TAGCAAAGCTGACCAAGAA-3′ | (SEQ ID NO: 43) |

(at the position 1968-1987 nt of SEQ ID NO: 3)

STK31 target sequence includes, for example, nucleotides

| 5′-GGAGATAGCTCTGGTTGAT-3′ | (SEQ ID NO: 38) |
|---|---|
| (position at 1713-1732 nt of SEQ ID NO: 5) | |
| or | |
| 5′-GGGCTATTCTGTGGATGTTS-3′ | (SEQ ID NO: 39) |

(position at 2289-2308 nt of SEQ ID NO: 5)

WDHD1 target sequence includes, for example, nucleotides

| 5′-GATCAGACATGTGCTATTA-3′ | (SEQ ID NO: 44) |
|---|---|
| (at the position of SEQ ID NO: 7) | |
| or | |
| 5′-GGTAATACGTGGACTCCTA-3′ | (SEQ ID NO: 45) |

(at the position of SEQ ID NO: 7)

The double-stranded molecules of the present invention is directed to a single target CX gene sequence or can be directed to a plurality of target CX gene sequences.

A double-stranded molecule of the present invention targeting the above-mentioned targeting sequence of CX gene include isolated polynucleotide(s) that comprises any of the nucleic acid sequences of target sequences and/or complementary sequences to the target sequences. Examples of a double-stranded molecule targeting CDCA5 gene include an oligonucleotide comprising the sequence corresponding to SEQ ID NO: 40 or SEQ ID NO: 41, and complementary sequences thereto; a double-stranded molecule targeting EPHA7 gene include an oligonucleotide comprising the sequence corresponding to SEQ ID NO: 42 or SEQ ID NO: 43, and complementary sequences thereto; a double-strand molecule targeting STK31 gene include an oligonucleotide comprising the sequence corresponding to SEQ ID NO: 38 or SEQ ID NO: 39, and complementary sequences thereto; a double-stranded molecule targeting WDHD1 gene include an oligonucleotide comprising the sequence corresponding to SEQ ID NO: 44 or SEQ ID NO: 45, and complementary sequences thereto. However, the present invention is not limited to these examples, and minor modifications in the aforementioned nucleic acid sequences are acceptable so long as the modified molecule retains the ability to suppress the expression of CX gene. Herein, “minor modification” in a nucleic acid sequence indicates one, two or several substitution, deletion, addition or insertion of nucleic acids to the sequence.

According to the present invention, a double-stranded molecule of the present invention can be tested for its ability using the methods utilized in the Examples (see, (12) RNA interference assay in [EXAMPLE 1]). In the Examples, the double-stranded molecules comprising sense strands and antisense strands complementary thereto of various portions of mRNA of CX genes were tested in vitro for their ability to decrease production of CX gene product in cancers cell lines (e.g., using LC319 and A549 for CDCA5; NCI-H520 and SBC-5 for EPHA7; LC319 and NCI-H2170 for STK31; and LC319 for WDHD1) according to standard methods. Furthermore, for example, reduction in CX gene product in cells contacted with the candidate double-stranded molecule compared to cells cultured in the absence of the candidate molecule can be detected by, e.g. RT-PCR using primers for CX gene mRNA mentioned (see, (3) Semi-quantitative RT-PCR in [EXAMPLE 1]). Sequences which decrease the production of CX gene product in in vitro cell-based assays can then be tested for there inhibitory effects on cell growth. Sequences which inhibit cell growth in in vitro cell-based assay can then be tested for their in vivo ability using animals with cancer, e.g. nude mouse xenograft models, to confirm decreased production of CX gene product and decreased cancer cell growth.

When the isolated polynucleotide is RNA or derivatives thereof, base “t” should be replaced with “u” in the nucleotide sequences. As used herein, the term “complementary” refers to Watson-Crick or Hoogsteen base pairing between nucleotides units of a polynucleotide, and the term “binding” means the physical or chemical interaction between two polynucleotides. When the polynucleotide comprises modified nucleotides and/or non-phosphodiester linkages, these polynucleotides can also bind each other as same manner. Generally, complementary polynucleotide sequences hybridize under appropriate conditions to form stable duplexes containing few or no mismatches. Furthermore, the sense strand and antisense strand of the isolated polynucleotide of the present invention can form double-stranded molecule or hairpin loop structure by the hybridization. In one embodiment, such duplexes contain no more than 1 mismatch for every 10 matches. In some embodiments, where the strands of the duplex are fully complementary, such duplexes contain no mismatches.

The polynucleotide is less than 2507 nucleotides in length for CDCA5, less than 5229 nucleotides in length for EPHA7, less than 3244 nucleotides in length for STK31, and less than 1129 nucleotides in length for WDHD1. For example, the polynucleotide is less than 500, 200, 100, 75, 50, or 25 nucleotides in length for all of the genes. The isolated polynucleotides of the present invention are useful for forming double-stranded molecules against CX gene or preparing template DNAs encoding the double-stranded molecules. When the polynucleotides are used for forming double-stranded molecules, the polynucleotide can be longer than 19 nucleotides, for example, longer than 21 nucleotides, for example, between about 19 and 25 nucleotides.

The double-stranded molecules of the invention can contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which can be incorporated into the present molecules (WO03/070744; WO2005/045037). In one embodiment, modifications can be used to provide improved resistance to degradation or improved uptake. Examples of such modifications include phosphorothioate linkages, 2′-O-methyl ribonucleotides (especially on the sense strand of a double-stranded molecule), 2′-deoxy-fluoro ribonucleotides, 2′-deoxy ribonucleotides, “universal base” nucleotides, 5′-C-methyl nucleotides, and inverted deoxyabasic residue incorporation (US Pat Appl. No. 20060122137).

In another embodiment, modifications can be used to enhance the stability or to increase targeting efficiency of the double-stranded molecule. Modifications include chemical cross linking between the two complementary strands of a double-stranded molecule, chemical modification of a 3′ or 5′ terminus of a strand of a double-stranded molecule, sugar modifications, nucleobase modifications and/or backbone modifications, 2-fluoro modified ribonucleotides and 2′-deoxy ribonucleotides (WO2004/029212).

In another embodiment, modifications can be used to increased or decreased affinity for the complementary nucleotides in the target mRNA and/or in the complementary double-stranded molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deza, 7-alkyl, or 7-alkenyl purine. In another embodiment, when the double-stranded molecule is a double-stranded molecule with a 3′ overhang, the 3′-terminal nucleotide overhanging nucleotides can be replaced by deoxyribonucleotides (Elbashir S M et al., Genes Dev 2001 Jan. 15, 15(2): 188-200). For further details, published documents for example, US Pat Appl. No. 20060234970 are available. The present invention is not limited to these examples and any known chemical modifications can be employed for the double-stranded molecules of the present invention so long as the resulting molecule retains the ability to inhibit the expression of the target gene.

Furthermore, the double-stranded molecules of the invention can comprise both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded molecule made of a DNA strand (polynucleotide) and an RNA strand (polynucleotide), a chimera type double-stranded molecule comprising both DNA and RNA on any or both of the single strands (polynucleotides), or the like can be formed for enhancing stability of the double-stranded molecule. The hybrid of a DNA strand and an RNA strand can be either where the sense strand is DNA and the antisense strand is RNA, or the opposite so long as it has an activity to inhibit expression of the target gene when introduced into a cell expressing the gene.

In some embodiments, the sense strand polynucleotide is DNA and the antisense strand polynucleotide is RNA. Also, the chimera type double-stranded molecule can be either where both of the sense and antisense strands are composed of DNA and RNA, or where any one of the sense and antisense strands is composed of DNA and RNA so long as it has an activity to inhibit expression of the target gene when introduced into a cell expressing the gene. In order to enhance stability of the double-stranded molecule, in some embodiments, the molecule contains as much DNA as possible, whereas to induce inhibition of the target gene expression, the molecule is required to be RNA within a range to induce sufficient inhibition of the expression. In one example of the chimera type double-stranded molecule, an upstream partial region (i.e., a region flanking to the target sequence or complementary sequence thereof within the sense or antisense strands) of the double-stranded molecule is RNA.

In some embodiments, the upstream partial region indicates the 5′ side (5′-end) of the sense strand and the 3′ side (3′-end) of the antisense strand. That is, in some embodiments, a region flanking to the 3′-end of the antisense strand, or both of a region flanking to the 5′-end of sense strand and a region flanking to the 3′-end of antisense strand consists of RNA. For instance, the chimera or hybrid type double-stranded molecule of the present invention comprise following combinations.

| | |
|---|---|
| sense | |
| strand: | 5′-[DNA]-3′ |
| | 3′-(RNA)[DNA]-5′: antisense strand, |
| sense | |
| strand: | 5′-(RNA)-[DNA]-3′ |
| | 3′-(RNA)-[DNA]-5′: antisense strand, |
| | and |
| sense | |
| strand: | 5′-(RNA)-[DNA]-3′ |
| | 3′-(RNA)-5′: antisense strand. |

The upstream partial region can be a domain of about 9 to 13 nucleotides counted from the terminus of the target sequence or complementary sequence thereto within the sense or antisense strands of the double-stranded molecules. Moreover, examples of such chimera type double-stranded molecules include those having a strand length of 19 to 21 nucleotides in which at least the upstream half region (5′ side region for the sense strand and 3′ side region for the antisense strand) of the polynucleotide is RNA and the other half is DNA. In such a chimera type double-stranded molecule, the effect to inhibit expression of the target gene is much higher when the entire antisense strand is RNA (US Pat Appl. No. 20050004064).

In the present invention, the double-stranded molecule can form a hairpin, for example, a short hairpin RNA (shRNA) and short hairpin made of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA comprises the sense target sequence and the antisense target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

A loop sequence made of an arbitrary nucleotide sequence can be located between the sense and antisense sequence in order to form the hairpin loop structure. Thus, the present invention also provides a double-stranded molecule having the general formula 5′-[A]-[B]-[A′]-3′, wherein [A] is the sense strand comprising a target sequence, [B] is an intervening single-strand and [A′] is the antisense strand comprising a complementary sequence to [A]. The target sequence can be selected from the group consisting of, for example, nucleotides

SEQ ID NO: 40 or SEQ ID NO: 41 for CDCA5; nucleotides, or

SEQ ID NO: 42 or SEQ ID NO: 43 for EPHA7; nucleotides

SEQ ID NO: 38 or SEQ ID NO: 39 for STK1; nucleotides

SEQ ID NO: 44 or SEQ ID NO: 45 for WDHD1; nucleotides

The present invention is not limited to these examples, and the target sequence in [A] can be modified sequences from these examples so long as the double-stranded molecule retains the ability to suppress the expression of the targeted CDCA5, EPHA7, STK31 or WDHD1 gene and result in inhibits or reduces the cell expressing these genes. The region [A] hybridizes to [A′] to form a loop comprising the region [B]. The intervening single-stranded portion [B], i.e., the loop sequence can be 3 to 23 nucleotides in length. The loop sequence, for example, can be selected from group consisting of following sequences (on the worldwide web at ambion.com/techlib/tb/tb_{—}506.html). Furthermore, loop sequence consisting of 23 nucleotides also provides active siRNA (Jacque J M et al., Nature 2002 Jul. 25, 418(6896): 435-8, Epub 2002 Jun. 26):

CCC, CCACC, or CCACACC: Jacque J M et al., Nature 2002 Jul. 25, 418(6896): 435-8, Epub 2002 Jun. 26;

UUCG: Lee N S et al., Nat Biotechnol 2002 May, 20(5): 500-5; Fruscoloni P et al., Proc Natl Acad Sci USA 2003 Feb. 18, 100(4): 1639-44, Epub 2003 Feb. 10; and

UUCAAGAGA: Dykxhoorn D M et al., Nat Rev Mol Cell Biol 2003 Jun., 4(6): 457-67.

Exemplary double-stranded molecules having hairpin loop structure of the present invention are shown below. In the following structure, the loop sequence can be selected from group consisting of AUG, CCC, UUCG, CCACC, CTCGAG, AAGCUU, CCACACC, and UUCAAGAGA; however, the present invention is not limited thereto:

(for target sequence SEQ ID NO: 40)
GCAGTTTGATCTCCTGGT-[B]-ACCAGGAGATCAAACTGC;
and
(for target sequence SEQ ID NO: 41)
GCCAGAGACTTGGAAATGT-[B]-ACATTTCCAAGTCTCTGGC;
for CDCA5
(for target sequence SEQ ID NO: 42)
AAAAGAGATGTTGCAGTA-[B]-TACTGCAACATCTCTTTT;
and
(for target sequence SEQ ID NO: 43)
TAGCAAAGCTGACCAAGAA-[B]-TTCTTGGTCAGCTTTGCTA;
for EPHA7
(for target sequence SEQ ID NO: 38)
GGAGATAGCTCTGGTTGAT-[B]-ATCAACCAGAGCTATCTCC;
and
(for target sequence SEQ ID NO: 39)
GGGCTATTCTGTGGATGTT-[B]-AACATCCACAGAATAGCCC;
for STK31 and
(for target sequence SEQ ID NO: 44)
GATCAGACATGTGCTATTA-[B]-TAATAGCACATGTCTGATC;
and
(for target sequence SEQ ID NO: 45)
GGTAATACGTGGACTCCTA-[B]-TAGGAGTCCACGTATTACC.
for WDHD1

Furthermore, in order to enhance the inhibition activity of the double-stranded molecules, nucleotide “u” can be added to 3′ end of the antisense strand of the target sequence, as 3′ overhangs. The number of “u”s to be added is at least 2, generally 2 to 10, for example, 2 to 5. The added “u”s form single strand at the 3′ end of the antisense strand of the double-stranded molecule.

The method of preparing the double-stranded molecule can use any chemical synthetic method known in the art. According to the chemical synthesis method, sense and antisense single-stranded polynucleotides are separately synthesized and then annealed together via an appropriate method to obtain a double-stranded molecule. In one embodiment for the annealing, the synthesized single-stranded polynucleotides are mixed in a molar ratio of at least about 3:7, for example, about 4:6, for example, substantially equimolar amount (i.e., a molar ratio of about 5:5). Next, the mixture is heated to a temperature at which double-stranded molecules dissociate and then is gradually cooled down. The annealed double-stranded polynucleotide can be purified by usually employed methods known in the art. Example of purification methods include methods utilizing agarose gel electrophoresis or wherein remaining single-stranded polynucleotides are optionally removed by, e.g., degradation with appropriate enzyme.

The regulatory sequences flanking target sequences can be identical- or different, such that their expression can be modulated independently, or in a temporal or spatial manner. The double-stranded molecules can be transcribed intracellularly by cloning CX gene templates into a vector containing, e.g., a RNA pol III transcription unit from the small nuclear RNA (snRNA) U6 or the human H1 RNA promoter.

### (ii) Vector

Also included in the invention is a vector containing one or more of the double-stranded molecules described herein, and a cell containing the vector. A vector of the present invention encodes a double-stranded molecule of the present invention in an expressible form. Herein, the phrase “in an expressible form” indicates that the vector, when introduced into a cell, will express the molecule. In one embodiment, the vector includes regulatory elements necessary for expression of the double-stranded molecule. Such vectors of the present invention can be used for producing the present double-stranded molecules, or directly as an active ingredient for treating cancer.

Vectors of the present invention can be produced, for example, by cloning a sequence comprising target sequence into an expression vector so that regulatory sequences are operatively-linked to the sequence in a manner to allow expression (by transcription of the DNA molecule) of both strands (Lee N S et al., Nat Biotechnol 2002 May, 20(5): 500-5). For example, RNA molecule that is the antisense to mRNA is transcribed by a first promoter (e.g., a promoter sequence flanking to the 3′ end of the cloned DNA) and RNA molecule that is the sense strand to the mRNA is transcribed by a second promoter (e.g., a promoter sequence flanking to the 5′ end of the cloned DNA). The sense and antisense strands hybridize in vivo to generate a double-stranded molecule constructs for silencing of the gene. Alternatively, two vectors constructs respectively encoding the sense and antisense strands of the double-stranded molecule are utilized to respectively express the sense and anti-sense strands and then forming a double-stranded molecule construct. Furthermore, the cloned sequence can encode a construct having a secondary structure (e.g., hairpin); namely, a single transcript of a vector contains both the sense and complementary antisense sequences of the target gene.

The vectors of the present invention can also be equipped so to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687).

The vectors of the present invention can be, for example, viral or bacterial vectors. Examples of expression vectors include attenuated viral hosts, for example, vaccinia or fowlpox (see, e.g., U.S. Pat. No. 4,722,848). This approach involves the use of vaccinia virus, e.g., as a vector to express nucleotide sequences that encode the double-stranded molecule. Upon introduction into a cell expressing the target gene, the recombinant vaccinia virus expresses the molecule and thereby suppresses the proliferation of the cell. Another example of useable vector includes Bacille Calmette Guerin (BCG). BCG vectors are described in Stover et al., Nature 1991, 351: 456-60. A wide variety of other vectors are useful for therapeutic administration and production of the double-stranded molecules; examples include adeno and adeno-associated virus vectors, retroviral vectors, Salmonella typhi vectors, detoxified anthrax toxin vectors, and the like. See, e.g., Shata et al., Mol Med Today 2000, 6: 66-71; Shedlock et al., J Leukoc Biol 2000, 68: 793-806; and Hipp et al., In Vivo 2000, 14: 571-85.

(iii) Methods of Inhibiting or Reducing a Growth of Cancer Cells and Treating or Preventing Cancer Using Double-Stranded Molecules

In the present invention, double-stranded molecules targeting the above-mentioned target sequences were respectively examined for their ability to inhibit or reduce the growth of cells (over)expressing the target genes. The growth of cancer cells (over)expressing CX gene(s), was inhibited or reduced by double-stranded molecules of the present invention; the growth of the CDCA5 (over)expressing cells, e.g. lung cancer cell line A549 and LC319, was inhibited by two double stranded molecules (FIGS. 2A and B, middle and lower panels); the growth of the EPHA7 expressing cells, e.g. lung cancer cell line NCI-H520 and SBC-5, was inhibited by two double stranded molecules (FIG. 6A, middle and lower panels); the growth of the STK31 expressing cells, e.g. lung cancer cell line LC319 and NCI-H2170, was inhibited by two double stranded molecules (FIGS. 11B and C); the growth of the WDHD1 expressing cells, e.g. lung cancer cell line LC319 and TE9, was inhibited by two double stranded molecules (FIG. 15A middle and lower panels).

Therefore, the present invention provides methods for inhibiting cell growth, i.e., cancerous cell growth of a cell from a cancer resulting from overexpression of a CX gene, or that is mediated by a CX gene, by inhibiting the expression of the CX gene. CX gene expression can be inhibited by any of the aforementioned double-stranded molecules of the present invention which specifically target expression of a complementary CX gene or the vectors of the present invention that can express any of the double-stranded molecules.

Such ability of the present double-stranded molecules and vectors to inhibit cell growth of cancerous cells indicates that they can be used for methods for treating cancer, a cancer resulting from overexpression of a CX gene, or that is mediated by a CX gene. Thus, the present invention provides methods to treat patients with a cancer resulting from overexpression of a CX gene, or that is mediated by a CX gene by administering a double-stranded molecule, i.e., an inhibitory nucleic acid, against a CX gene or a vector expressing the molecule without adverse effect because those genes were hardly detected in normal organs.

Specifically, the present invention provides the following methods [1] to [22]:

[1] A method for inhibiting or reducing a growth of a cell (over)expressing a CX gene selected from the group consisting of CDCA5, EPHA7, STK31 and WDHD1, or a method for treating or preventing cancer (over)expressing a gene selected from the group consisting of CDCA5, EPHA7, STK31 and WDHD1, wherein said method comprising the step of giving at least one double-stranded molecule, wherein said double-stranded molecule is introduced into a cell, and inhibits or reduces in vivo expression of said CX gene.

[2] The method of [1], wherein said double-stranded molecule acts at mRNA which shares sequence identity with or is complementary to a target sequence selected from the group SEQ ID NO: 40 (at positions of 808-827 nt of SEQ ID NO: 1) and SEQ ID NO: 41 (at positions of 470-488 nt of SEQ ID NO: 1) for CDCA5, SEQ ID NO: 42 (at positions of 2182-2200 nt of SEQ ID NO: 3) and SEQ ID NO: 43 (at positions of 1968-1987 nt of SEQ ID NO: 3) for EPHA7, SEQ ID NO: 38 (at positions of 1713-1732 nt of SEQ ID NO: 5) and SEQ ID NO: 39 (at positions of 2289-2308 nt of SEQ ID NO: 5) for STK31, SEQ ID NO: 44 (at positions of 577-596 nt of SEQ ID NO: 7) and SEQ ID NO: 45 (at positions of 2041-2060 nt of SEQ ID NO: 7) for WDHD1.

[3] The method of [2], wherein said double-stranded molecule comprises a sense strand and an antisense strand complementary thereto, hybridized to each other to form a double strand, wherein said sense strand comprises an oligonucleotide corresponding to a sequence selected from the group consisting of SEQ ID NO: 40 and SEQ ID NO: 41 for CDCA5, SEQ ID NO: 42 and SEQ ID NO: 43 for EPHA7, SEQ ID NO: 38 and SEQ ID NO: 39 for STK31, SEQ ID NO: 44 and SEQ ID NO: 45 for WDHD1.

[4] The method of [1], wherein a plurality of double-stranded molecules are administered; In some embodiments, the double-stranded molecules comprise different nucleic acid sequences.

[5] The method of [4], wherein the plurality of double-stranded molecules target the same gene;

[6] The method of [1], wherein the double-stranded molecule has a length of less than about 100 nucleotides;

[7] The method of [6], wherein the double-stranded molecule has a length of less than about 75 nucleotides;

[8] The method of [7], wherein the double-stranded molecule has a length of less than about 50 nucleotides;

[9] The method of [8], wherein the double-stranded molecule has a length of less than about 25 nucleotides;

[10] The method of [9], wherein the double-stranded molecule has a length of between about 19 and about 25 nucleotides in length;

[11] The method of [1], wherein said double-stranded molecule consists of a single oligonucleotide comprising both the sense and antisense strands linked by an intervening single-strand.

[12] The method of [11], wherein said double-stranded molecule has a general formula 5′-[A]-[B]-[A′]-3′, wherein

[A] is the sense strand comprising an oligonucleotide corresponding to a sequence selected from the group consisting of SEQ ID NO: 40 and SEQ ID NO: 41 for CDCA5, SEQ ID NO: 42 and SEQ ID NO: 43 for EPHA7, SEQ ID NO: 38 and SEQ ID NO: 39 for STK31, SEQ ID NO: 44 and SEQ ID NO: 45 for WDHD1;

[B] is the intervening single-strand; and

[A′] is the antisense strand comprising an oligonucleotide corresponding to a sequence complementary to the sequence selected in [A].

[13] The method of [1], wherein the double-stranded molecule comprises RNA.

[14] The method of [1], wherein the double-stranded molecule comprises both DNA and RNA.

[15] The method of [14], wherein the double-stranded molecule is a hybrid of a DNA polynucleotide and an RNA polynucleotide.

[16] The method of [15] wherein the sense and antisense strand polynucleotides a made of DNA and RNA, respectively.

[17] The method of [14], wherein the double-stranded molecule is a chimera of DNA and RNA.

[18] The method of [17], wherein a region flanking to the 5′-end of one or both of the sense and antisense polynucleotides a made of RNA.

[19] The method of [18], wherein the flanking region consists of 9 to 13 nucleotides.

[20] The method of [1], wherein the double-stranded molecule contains 3′ overhangs.

[21] The method of [1], wherein the double-stranded molecule is encoded by a vector.

[22] The method of [21], wherein said double-stranded molecule has a general formula 5′-[A]-[B]-[A′]-3′, wherein

[A] is the sense strand comprising an oligonucleotide corresponding to a sequence selected from the group consisting of SEQ ID NO: 40 and SEQ ID NO: 41 for CDCA5, SEQ ID NO: 42 and SEQ ID NO: 43 for EPHA7, SEQ ID NO: 38 and SEQ ID NO: 39 for STK31, SEQ ID NO: 44 and SEQ ID NO: 45 for WDHD1;

[B] is the intervening single-strand; and

[A′] is the antisense strand comprising an oligonucleotide corresponding to a sequence complementary to the sequence selected in [A].

[23] The method of [1], wherein the double-stranded molecule is contained in a composition which comprises in addition to the molecule a transfection-enhancing agent and cell permeable agent.

The method of the present invention will be described in more detail below.

The growth of cells (over)expressing a CX gene is inhibited by contacting the cells with a double-stranded molecule against CX gene, a vector expressing the molecule or a composition comprising the same. The cell is further contacted with a transfection agent. Suitable transfection agents are known in the art. The phrase “inhibition of cell growth” indicates that the cell proliferates at a lower rate or has decreased viability compared to a cell not exposed to the molecule. Cell growth can be measured by methods known in the art, e.g., using the MTT cell proliferation assay.

The growth of any kind of cell can be suppressed according to the present method so long as the cell expresses or over-expresses the target gene of the double-stranded molecule of the present invention. Exemplary cells include cancers cells.

Thus, patients suffering from or at risk of developing disease related to CX gene can be treated by administering at least one of the present double-stranded molecules, at least one vector expressing at least one of the molecules or at least one composition comprising at least one of the molecules. For example, patients of cancers can be treated according to the present methods. The type of cancer can be identified by standard methods according to the particular type of tumor to be diagnosed. In some embodiments, patients treated by the methods of the present invention are selected by detecting the (over)expression of a CX gene in a biopsy from the patient by RT-PCR, hybridization or immunoassay. In some embodiments, before the treatment of the present invention, the biopsy specimen from the subject is confirmed for CX gene over-expression by methods known in the art, for example, immunohistochemical analysis, hybridization or RT-PCR (see, (3) Semi-quantitative RT-PCR, (4) Northern-blot analysis, (5) Western-blotting, (8) Immunohistochemistry or (10) ELISA in [EXAMPLE 1]).

According to the present method to inhibit or reduce cell growth and thereby treating cancer, when administering plural kinds of the double-stranded molecules (or vectors expressing or compositions containing the same), each of the molecules can direct to the different target sequence of same gene, or different target sequences of different gene. For example, the method can utilize different double-stranded molecules directing to same CX gene transcript. Alternatively, for example, the method can utilize double-stranded molecules directed to one, two or more target sequences selected from same CX gene.

For inhibiting cell growth, a double-stranded molecule of present invention can be directly introduced into the cells in a form to achieve binding of the molecule with corresponding mRNA transcripts. Alternatively, as described above, a DNA encoding the double-stranded molecule can be introduced into cells as a vector. For introducing the double-stranded molecules and vectors into the cells, transfection-enhancing agent, for example, FuGENE (Roche diagnostics), Lipofectamine 2000 (Invitrogen), Oligofectamine (Invitrogen), and Nucleofector (Wako pure Chemical), can be employed.

A treatment is determined efficacious if it leads to clinical benefit for example, reduction in expression of the CX gene, or a decrease in size, prevalence, or metastatic potential of the cancer in the subject. When the treatment is applied prophylactically, “efficacious” means that it retards or prevents cancers from forming or prevents or alleviates a clinical symptom of cancer. Efficaciousness is determined in association with any known method for diagnosing or treating the particular tumor type.

It is understood that the double-stranded molecule of the invention degrades the target mRNA (CX gene transcript) in substoichiometric amounts. Without wishing to be bound by any theory, it is believed that the double-stranded molecule of the invention causes degradation of the target mRNA in a catalytic manner. Thus, compared to standard cancer therapies, significantly less a double-stranded molecule needs to be delivered at or near the site of cancer to exert therapeutic effect.

One skilled in the art can readily determine an effective amount of the double-stranded molecule of the invention to be administered to a given subject, by taking into account factors for example, body weight, age, sex, type of disease, symptoms and other conditions of the subject; the route of administration; and whether the administration is regional or systemic. Generally, an effective amount of the double-stranded molecule of the invention comprises an intercellular concentration at or near the cancer site of from about 1 nanomolar (nM) to about 100 nM, for example, from about 2 nM to about 50 nM, for example, from about 2.5 nM to about 10 nM. It is contemplated that greater or smaller amounts of the double-stranded molecule can be administered.

The present methods can be used to inhibit the growth or metastasis of cancer; for example, a cancer resulting from overexpression of a CX gene or that is mediated by a CX gene, e.g., lung cancer or esophagus cancer. In particular, a double-stranded molecule directed to a target sequence selected from the group consisting of SEQ ID NO: 40 (at the position of 808-827 nt of SEQ ID NO: 1) and SEQ ID NO: 41 (at the position of 470-488 nt of SEQ ID NO: 1) for CDCA5, SEQ ID NO: 42 (at the position of 2182-2200 nt of SEQ ID NO: 3) and SEQ ID NO: 43 (at the position of 1968-1987 nt of SEQ ID NO: 3) for EPHA7, SEQ ID NO: 38 (at the position of 1713-1732 nt of SEQ ID NO: 5) and SEQ ID NO: 39 (at the position of 2289-2308 nt of SEQ ID NO: 5) for STK31, SEQ ID NO: 44 (at the position of 577-596 nt of SEQ ID NO: 7) and SEQ ID NO: 45 (at the position of 2041-2060 nt of SEQ ID NO: 7) for WDHD1 finds use for the treatment of cancers.

For treating cancer, e.g., a cancer promoted by a CX gene, the double-stranded molecule of the invention can also be administered to a subject in combination with a pharmaceutical agent different from the double-stranded molecule. Alternatively, the double-stranded molecule of the invention can be administered to a subject in combination with another therapeutic method designed to treat cancer. For example, the double-stranded molecule of the invention can be administered in combination with therapeutic methods currently employed for treating cancer or preventing cancer metastasis (e.g., radiation therapy, surgery and treatment using chemotherapeutic agents, for example, cisplatin, carboplatin, cyclophosphamide, 5-fluorouracil, adriamycin, daunorubicin or tamoxifen).

In the present methods, the double-stranded molecule can be administered to the subject either as a naked double-stranded molecule, in conjunction with a delivery reagent, or as a recombinant plasmid or viral vector which expresses the double-stranded molecule.

Suitable delivery reagents for administration in conjunction with the present a double-stranded molecule include the Mirus Transit TKO lipophilic reagent; lipofectin; lipofectamine; cellfectin; or polycations (e.g., polylysine), or liposomes. In one embodiment, the delivery reagent is a liposome.

Liposomes can aid in the delivery of the double-stranded molecule to a particular tissue, for example, retinal or tumor tissue, and can also increase the blood half-life of the double-stranded molecule. Liposomes suitable for use in the invention are formed from standard vesicle-forming lipids, which generally include neutral or negatively charged phospholipids and a sterol, for example, cholesterol. The selection of lipids is generally guided by consideration of factors for example, the desired liposome size and half-life of the liposomes in the blood stream. A variety of methods are known for preparing liposomes, for example as described in Szoka et al., Ann Rev Biophys Bioeng 1980, 9: 467; and U.S. Pat. Nos. 4,235,871; 4,501,728; 4,837,028; and 5,019,369, the entire disclosures of which are herein incorporated by reference.

In some embodiments, the liposomes encapsulating the present double-stranded molecule comprises a ligand molecule that can deliver the liposome to the cancer site. Ligands which bind to receptors prevalent in tumor or vascular endothelial cells, for example, monoclonal antibodies that bind to tumor antigens or endothelial cell surface antigens, find use.

In some embodiments, the liposomes encapsulating the present double-stranded molecule are modified so as to avoid clearance by the mononuclear macrophage and reticuloendothelial systems, for example, by having opsonization-inhibition moieties bound to the surface of the structure. In one embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example, solid tumors, will efficiently accumulate these liposomes; see Gabizon et al., Proc Natl Acad Sci USA 1988, 18: 6949-53. In addition, the reduced uptake by the RES lowers the toxicity of stealth liposomes by preventing significant accumulation in liver and spleen. Thus, liposomes of the invention that are modified with opsonization-inhibition moieties can deliver the present double-stranded molecule to tumor cells.

Opsonization inhibiting moieties suitable for modifying liposomes can be water-soluble polymers with a molecular weight from about 500 to about 40,000 daltons, for example, from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers for example, polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, for example, ganglioside GM₁. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

In some embodiments, the opsonization-inhibiting moiety is a PEG, PPG, or derivatives thereof. Liposomes modified with PEG or PEG-derivatives are sometimes called “PEGylated liposomes”.

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture for example, tetrahydrofuran and water in a 30:12 ratio at 60° C.

Vectors expressing a double-stranded molecule of the invention are discussed above. Such vectors expressing at least one double-stranded molecule of the invention can also be administered directly or in conjunction with a suitable delivery reagent, including the Mirus Transit LT1 lipophilic reagent; lipofectin; lipofectamine; cellfectin; polycations (e.g., polylysine) or liposomes. Methods for delivering recombinant viral vectors, which express a double-stranded molecule of the invention, to an area of cancer in a patient are within the skill of the art.

The double-stranded molecule of the invention can be administered to the subject by any means suitable for delivering the double-stranded molecule into cancer sites. For example, the double-stranded molecule can be administered by gene gun, electroporation, or by other suitable parenteral or enteral administration routes.

Suitable enteral administration routes include oral, rectal, or intranasal delivery.

Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection, intra-retinal injection, or subretinal injection); subcutaneous injection or deposition including subcutaneous infusion (for example, by osmotic pumps); direct application to the area at or near the site of cancer, for example by a catheter or other placement device (e.g., a retinal pellet or a suppository or an implant comprising a porous, non-porous, or gelatinous material); and inhalation. In some embodiments, injections or infusions of the double-stranded molecule or vector be given at or near the site of cancer.

The double-stranded molecule of the invention can be administered in a single dose or in multiple doses. Where the administration of the double-stranded molecule of the invention is by infusion, the infusion can be a single sustained dose or can be delivered by multiple infusions. Injection of the agent can be directly into the tissue or near the site of cancer. Multiple injections of the agent into the tissue at or near the site of cancer can be administered.

One skilled in the art can also readily determine an appropriate dosage regimen for administering the double-stranded molecule of the invention to a given subject. For example, the double-stranded molecule can be administered to the subject once, for example, as a single injection or deposition at or near the cancer site. Alternatively, the double-stranded molecule can be administered once or twice daily to a subject for a period of from about three to about twenty-eight days, for example, from about seven to about ten days. In one exemplary dosage regimen, the double-stranded molecule is injected at or near the site of cancer once a day for seven days. Where a dosage regimen comprises multiple administrations, it is understood that the effective amount of a double-stranded molecule administered to the subject can comprise the total amount of a double-stranded molecule administered over the entire dosage regimen.

### (iv) Compositions

Furthermore, the present invention provides pharmaceutical compositions comprising at least one of the present double-stranded molecules or the vectors coding for the molecules. Specifically, the present invention provides the following compositions [1] to [24]:

[1] A composition for inhibiting or reducing a growth of cell expressing a gene selected from the group consisting of CDCA5, EPHA7, STK31 and WDHD1, or a composition for treating or preventing a cancer expressing a CX gene selected from the group consisting of CDCA5, EPHA7, STK31 and WDHD1, which comprising at least one double-stranded molecule, wherein said double-stranded molecule is introduced into a cell, inhibits or reduces in vivo expression of said gene.

[2] The composition of [1], wherein said double-stranded molecule acts at mRNA which matched a target sequence selected from the group SEQ ID NO: 40 (at the position of 808-827 nt of SEQ ID NO: 1) and SEQ ID NO: 41 (at the position of 470-488 nt of SEQ ID NO: 1) for CDCA5, SEQ ID NO: 42 (at the position of 2182-2200 nt of SEQ ID NO: 3) and SEQ ID NO: 43 (at the position of 1968-1987 nt of SEQ ID NO: 3) for EPHA7, SEQ ID NO: 38 (at the position of 1713-1732 nt of SEQ ID NO: 5) and SEQ ID NO: 39 (at the position of 2289-2308 nt of SEQ ID NO: 5) for STK31, SEQ ID NO: 44 (at the position of 577-596 nt of SEQ ID NO: 7) and SEQ ID NO: 45 (at the position of 2041-2060 nt of SEQ ID NO: 7) for WDHD1.

[3] The composition of [2], wherein said double-stranded molecule comprises a sense strand and an antisense strand complementary thereto, hybridized to each other to form a double strand, wherein said sense strand comprises an oligonucleotide corresponding to a sequence selected from the group consisting of SEQ ID NO: 40 and SEQ ID NO: 41 for CDCA5, SEQ ID NO: 42 and SEQ ID NO: 43 for EPHA7, SEQ ID NO: 38 and SEQ ID NO: 39 for STK31, SEQ ID NO: 44 and SEQ ID NO: 45 for WDHD1.

The composition of [1], wherein the cancer to be treated is a cancer resulting from overexpression of a CX gene, or which is mediated by a CX gene.

[4] The composition of [1], wherein the cancer to be treated is lung cancer or esophageal cancer;

[5] The composition of [4], wherein the lung cancer is small cell lung cancer or non-small cell lung cancer;

[6] The composition of [1], wherein the composition contains plural kinds of the double-stranded molecules;

[7] The composition of [6], wherein the plural kinds of the double-stranded molecules target the same gene;

[8] The composition of [1], wherein the double-stranded molecule has a length of less than about 100 nucleotides;

[9] The composition of [8], wherein the double-stranded molecule has a length of less than about 75 nucleotides;

[10] The composition of [9], wherein the double-stranded molecule has a length of less than about 50 nucleotides;

[11] The composition of [10], wherein the double-stranded molecule has a length of less than about 25 nucleotides;

[12] The composition of [11], wherein the double-stranded molecule has a length of between about 19 and about 25 nucleotides;

[13] The composition of [1], wherein said double-stranded molecule consists of a single oligonucleotide comprising both the sense and antisense strands linked by an intervening single-strand.

[14] The composition of [13], wherein said double-stranded molecule has a general formula 5′-[A]-[B]-[A′]-3′, wherein

[A] is the sense strand comprising an oligonucleotide corresponding to a sequence selected from the group consisting of SEQ ID NO: 40 and SEQ ID NO: 41 for CDCA5, SEQ ID NO: 42 and SEQ ID NO: 43 for EPHA7, SEQ ID NO: 38 and SEQ ID NO: 39 for STK31, SEQ ID NO: 44 and SEQ ID NO: 45 for WDHD1;

[B] is the intervening single-strand; and

[A′] is the antisense strand comprising an oligonucleotide corresponding to a sequence complementary to the sequence selected in [A].

[15] The composition of [1], wherein the double-stranded molecule comprises RNA;

[16] The composition of [1], wherein the double-stranded molecule comprises DNA and RNA;

[17] The composition of [16], wherein the double-stranded molecule is a hybrid of a DNA polynucleotide and an RNA polynucleotide;

[18] The composition of [17], wherein the sense and antisense strand polynucleotides are made of DNA and RNA, respectively;

[19] The composition of [18], wherein the double-stranded molecule is a chimera of DNA and RNA;

[20] The composition of [19], wherein at least a region flanking to the 5′-end of one or both of the sense and antisense polynucleotides consists of RNA.

[21] The composition of [20], wherein the flanking region consists of 9 to 13 nucleotides;

[22] The composition of [1], wherein the double-stranded molecule contains 3′ overhangs;

[23] The composition of [1], wherein the double-stranded molecule is encoded by a vector and contained in the composition;

[24] The composition of [1], which further comprising a transfection-enhancing agent, cell permeable agent and pharmaceutically acceptable carrier.

The method of the present invention will be described in more detail below.

The double-stranded molecules of the invention can be formulated as pharmaceutical compositions prior to administering to a subject, according to techniques known in the art. Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is herein incorporated by reference.

The present pharmaceutical formulations comprise at least one of the double-stranded molecules or vectors encoding them of the present invention (e.g., 0.1 to 90% by weight), or a physiologically acceptable salt of the molecule, mixed with a physiologically acceptable carrier medium. Exemplary physiologically acceptable carrier media include, for example, water, buffered water, normal saline, 0.4% saline, 0.3% glycine, hyaluronic acid and the like.

According to the present invention, the composition can contain plural kinds of the double-stranded molecules, each of the molecules can be directed to the same target sequence, or different target sequences of CX gene. For example, the composition can contain double-stranded molecules directed to CX gene. Alternatively, for example, the composition can contain double-stranded molecules directed to one, two or more target sequences selected from CX genes.

Furthermore, the present composition can contain a vector coding for one or plural double-stranded molecules. For example, the vector can encode one, two or several kinds of the present double-stranded molecules. Alternatively, the present composition can contain plural kinds of vectors, each of the vectors coding for a different double-stranded molecule.

Moreover, the present double-stranded molecules can be contained as liposomes in the present composition. See under the item of “Methods of treating cancer” for details of liposomes.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (for example, for example, DTPA or DTPA-bisamide) or calcium chelate complexes (for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can comprise any of the carriers and excipients listed above and 10-95%, for example, 25-75%, of one or more double-stranded molecule of the invention. A pharmaceutical composition for aerosol (inhalational) administration can comprise 0.01-20% by weight, for example, 1-10% by weight, of one or more double-stranded molecule of the invention encapsulated in a liposome as described above, and propellant. A carrier can also be included as desired; e.g., lecithin for intranasal delivery.

In addition to the above, the present composition can contain other pharmaceutical active ingredients so long as they do not inhibit the in vivo function of the present double-stranded molecules. For example, the composition can contain chemotherapeutic agents conventionally used for treating cancers.

The present invention also provides the use of the double-stranded nucleic acid molecules of the present invention in manufacturing a pharmaceutical composition for treating a cancer (over)expressing the CX gene. For example, the present invention relates to the use of double-stranded nucleic acid molecule inhibiting the (over)expression of a CX gene in a cell, which over-expresses the gene, wherein the CX gene is selected from the group consisting of CDCA5, EPHA7, STK31 and WDHD1, which molecule comprises a sense strand and an antisense strand complementary thereto, hybridized to each other to form the double-stranded nucleic acid molecule and targets a sequence selected from the group consisting of SEQ ID NOs: 38 to 45, for manufacturing a pharmaceutical composition for treating a cancer (over)expressing the CX gene.

The present invention further provides a method or process for manufacturing a pharmaceutical composition for treating a cancer (over)expressing the CX gene, wherein the method or process comprises step for formulating a pharmaceutically or physiologically acceptable carrier with a double-stranded nucleic acid molecule inhibiting the (over)expression of a CX gene in a cell, which over-expresses the gene, wherein the CX gene is selected from the group consisting of CDCA5, EPHA7, STK31 and WDHD1, which molecule comprises a sense strand and an antisense strand complementary thereto, hybridized to each other to form the double-stranded nucleic acid molecule and targets a sequence selected from the group consisting of SEQ ID NOs: 38 to 45 as active ingredients.

The present invention also provides a method or process for manufacturing a pharmaceutical composition for treating a cancer (over)expressing the CX gene, wherein the method or process comprises step for admixing an active ingredient with a pharmaceutically or physiologically acceptable carrier, wherein the active ingredient is a double-stranded nucleic acid molecule inhibiting the expression of a CX gene in a cell, which over-expresses the gene, wherein the CX gene is selected from the group consisting of CDCA5, EPHA7, STK31 and WDHD1, which molecule comprises a sense strand and an antisense strand complementary thereto, hybridized to each other to form the double-stranded nucleic acid molecule and targets a target sequence selected from the group consisting of SEQ ID NOs: 38 to 45.

### Method for Diagnosing CX Gene-Mediated Cancers

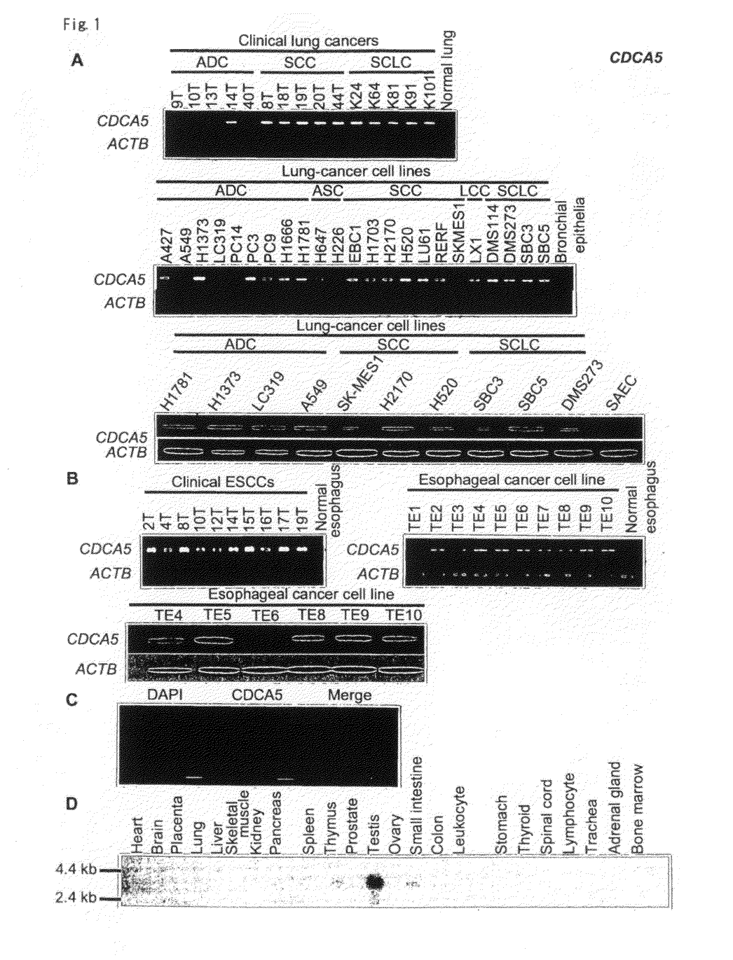

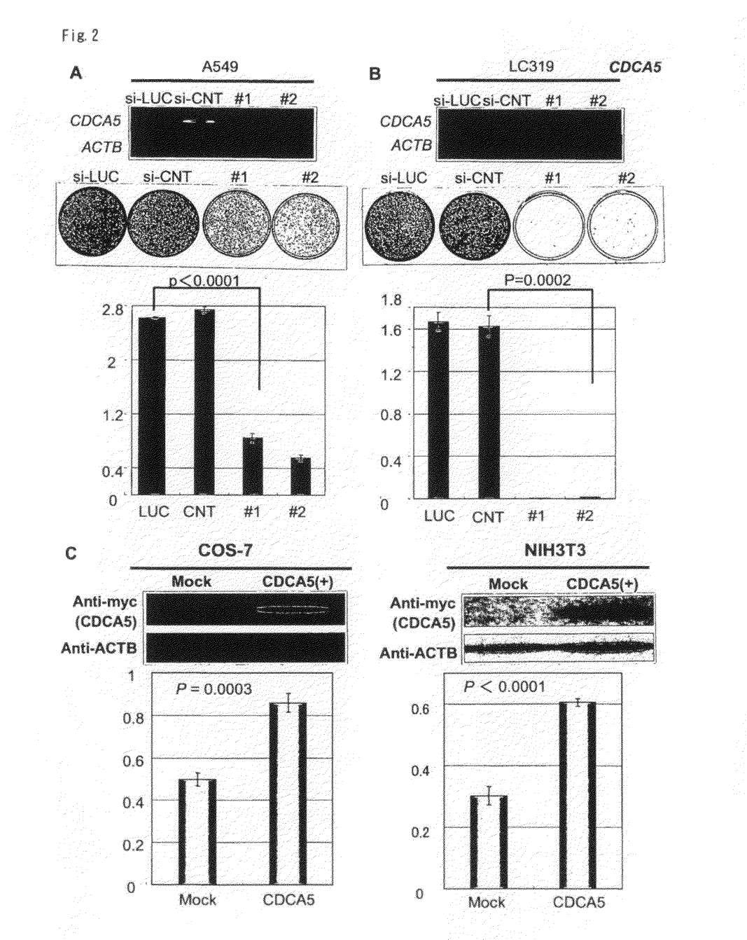

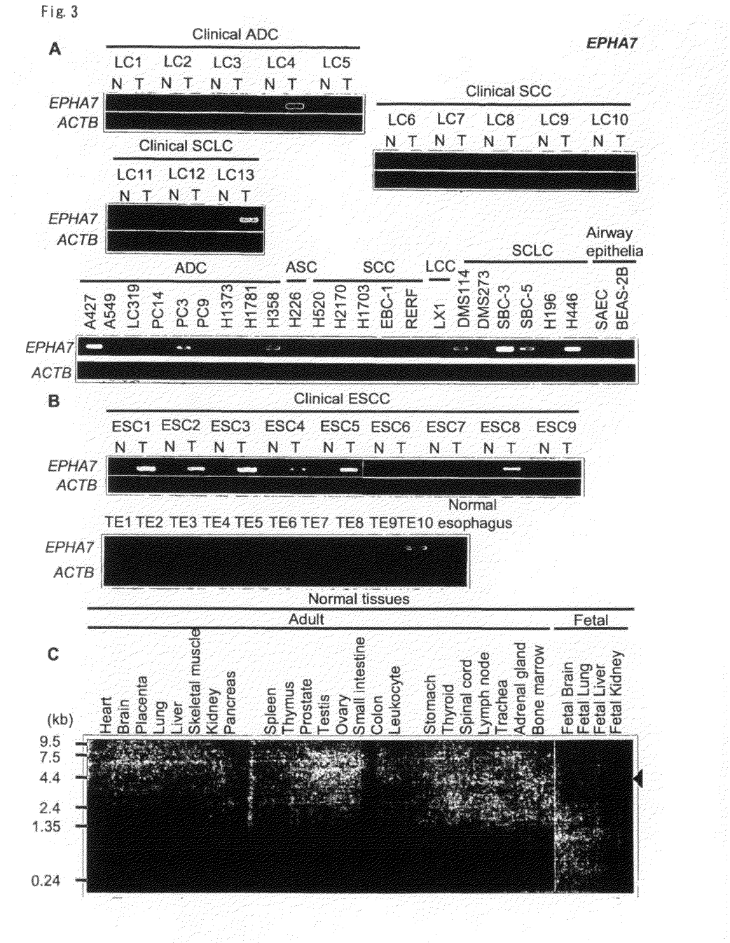

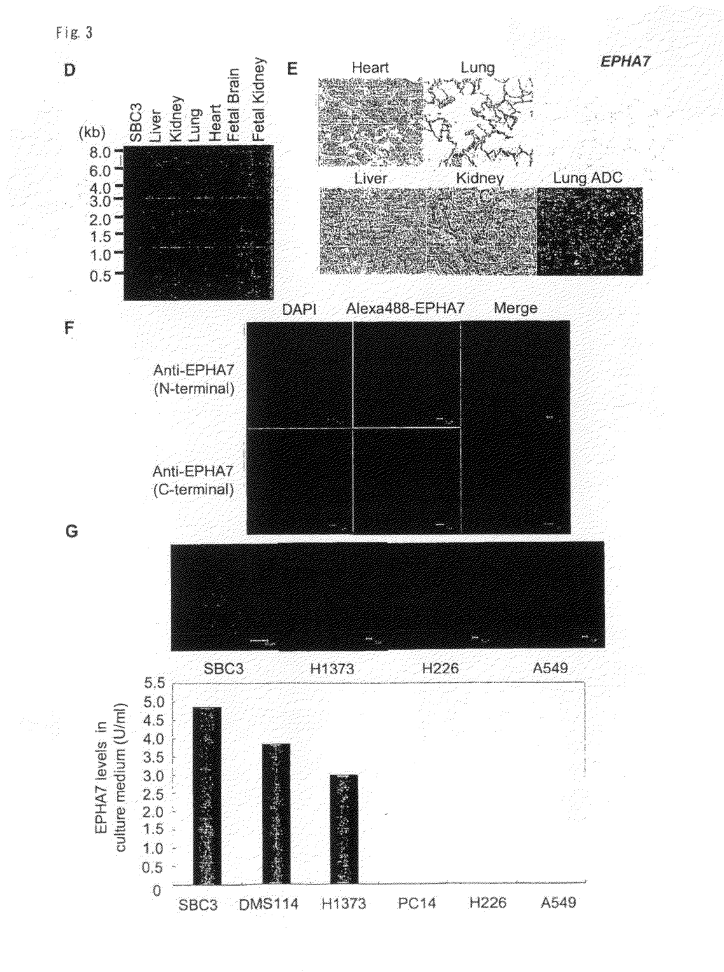

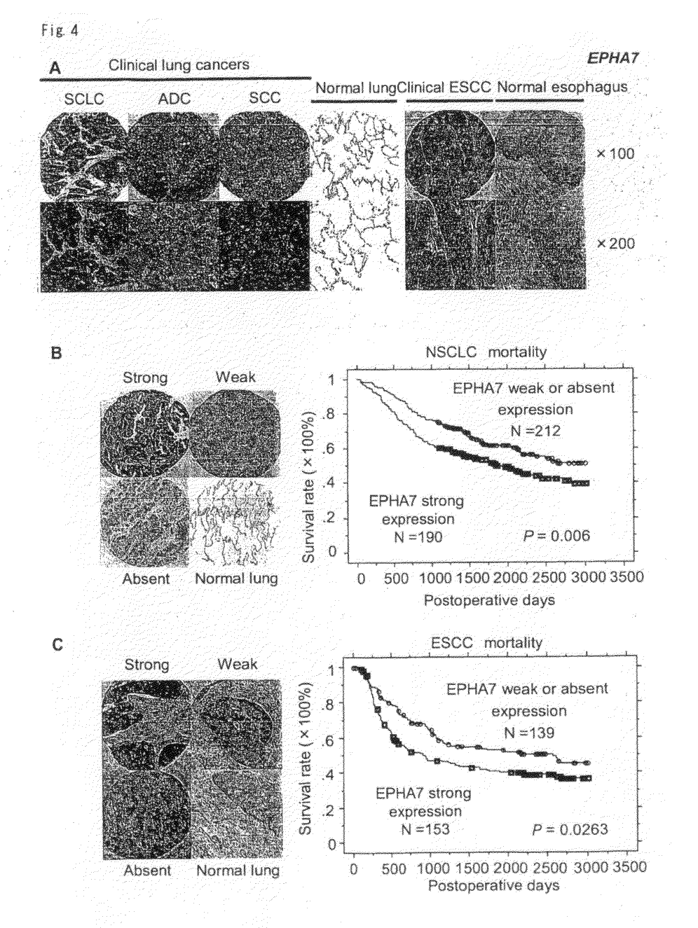

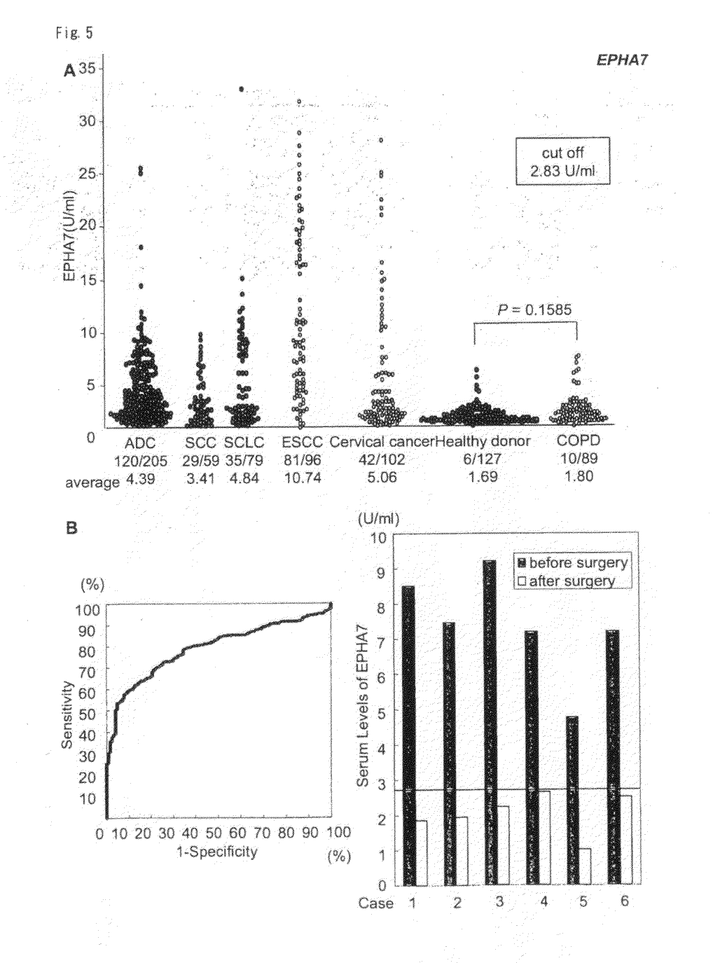

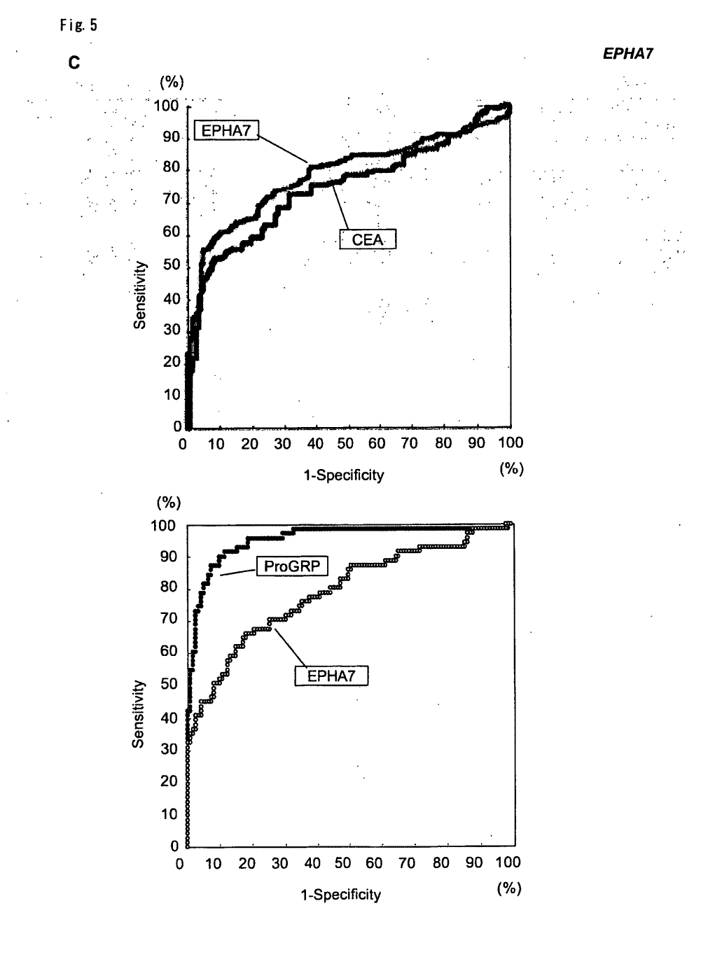

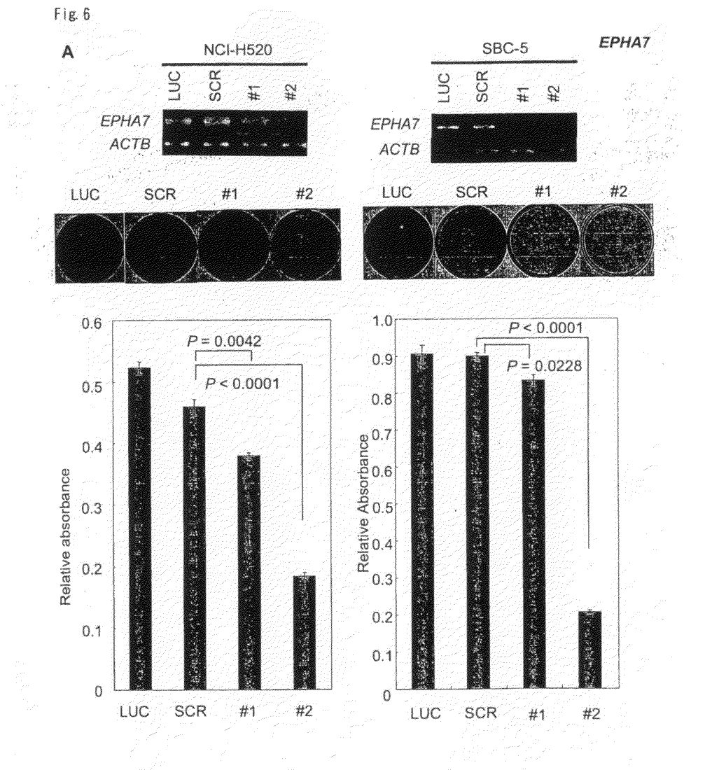

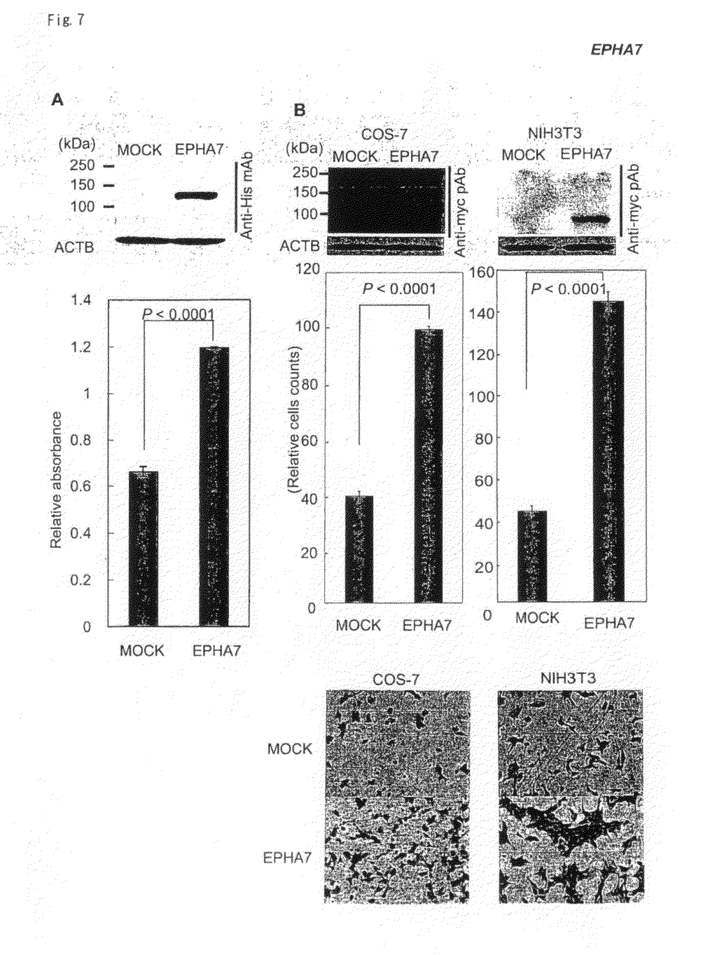

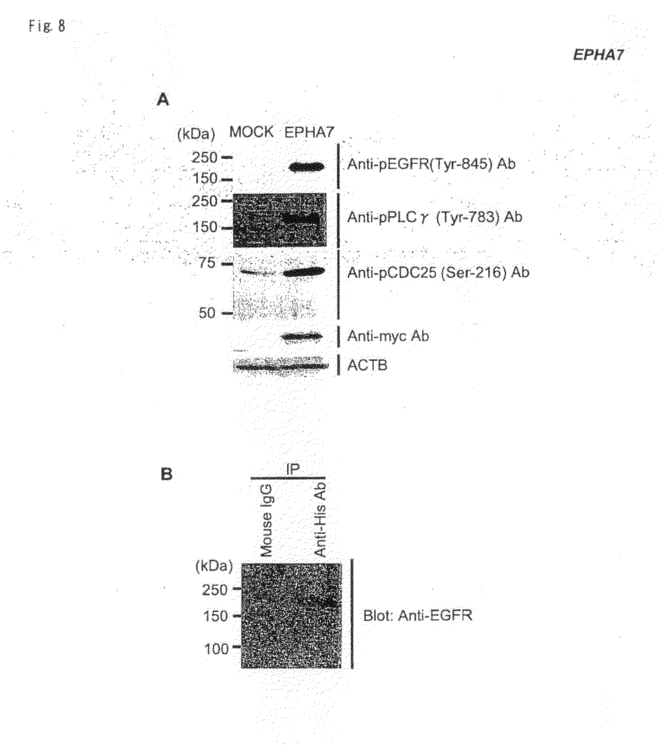

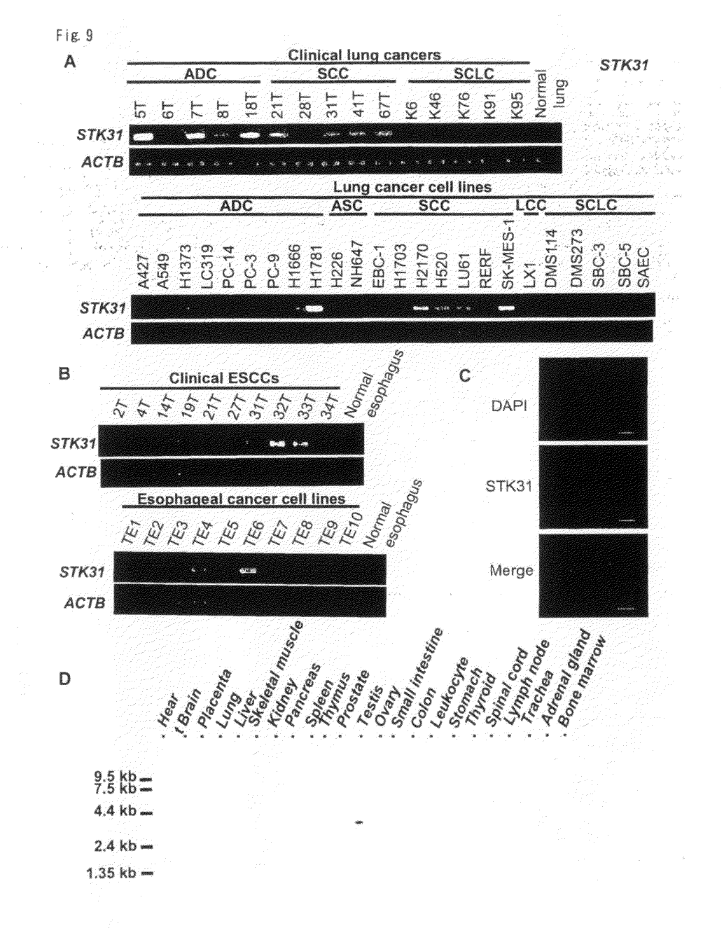

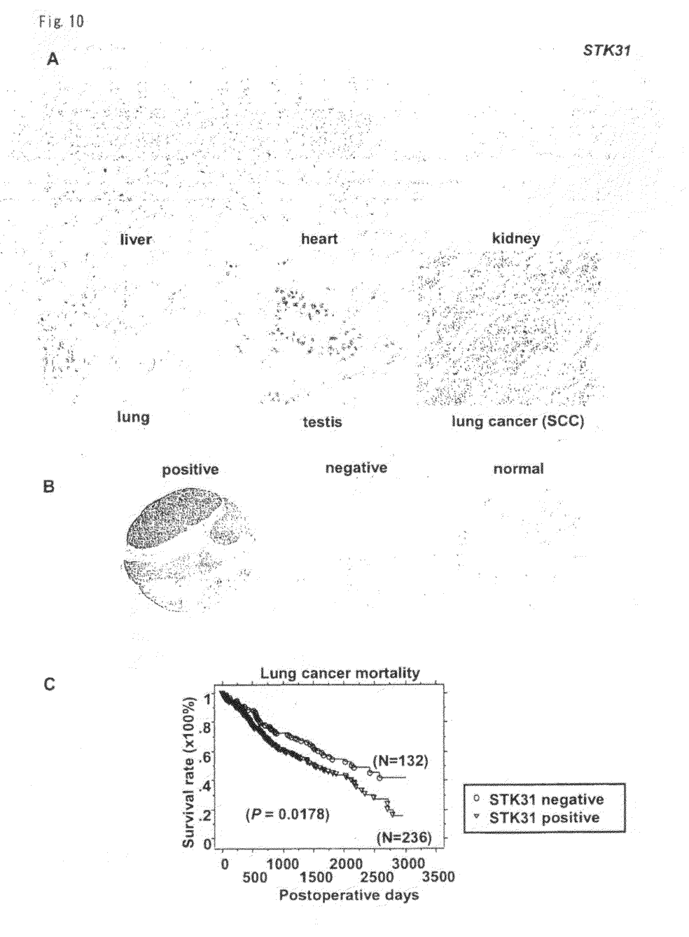

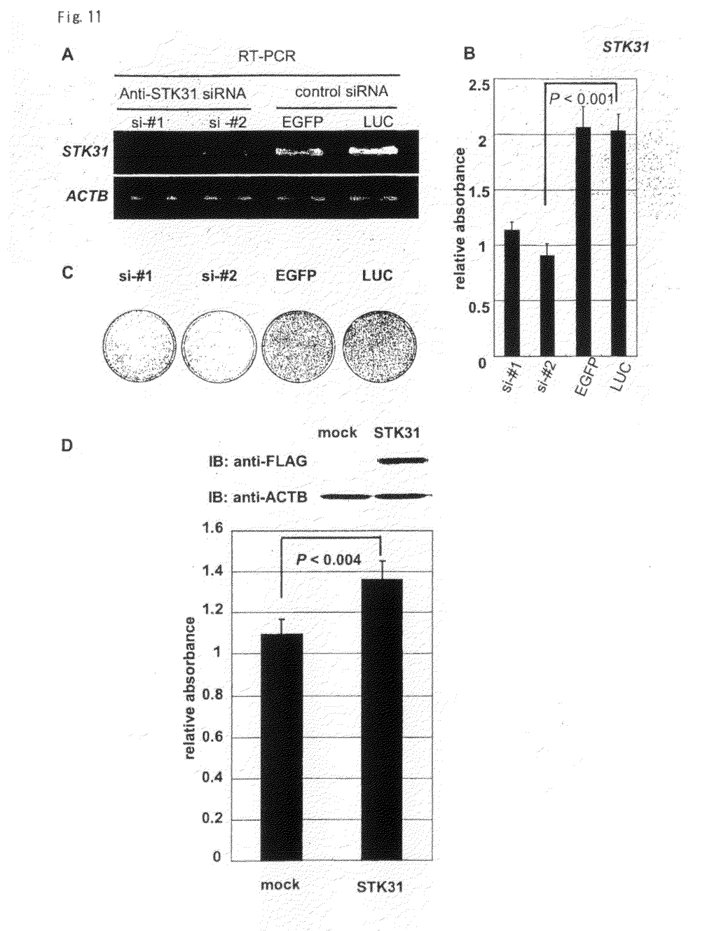

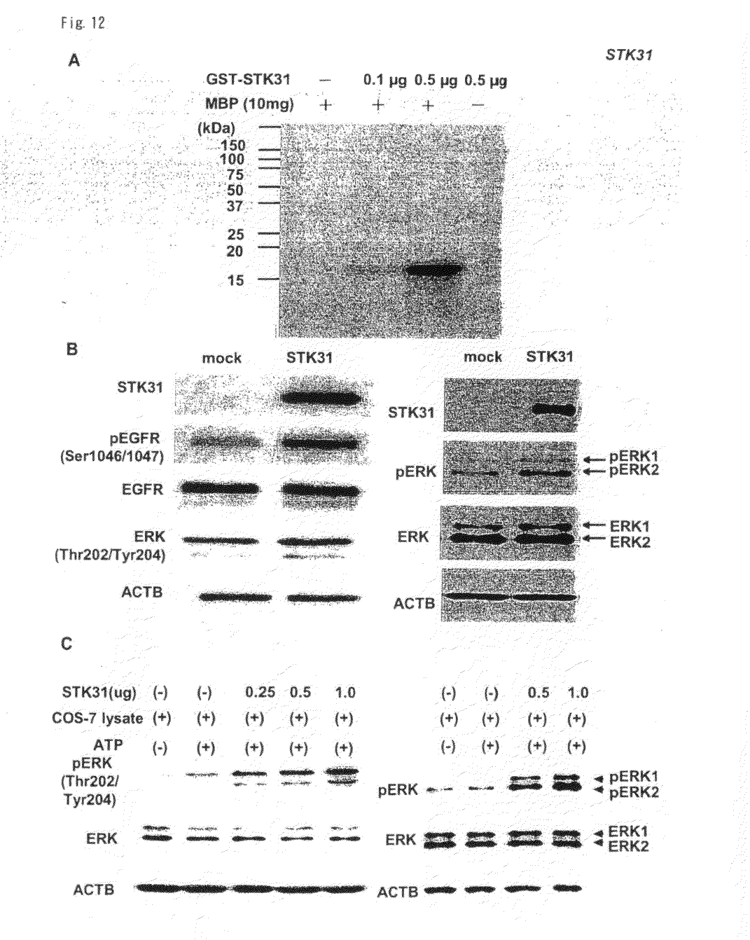

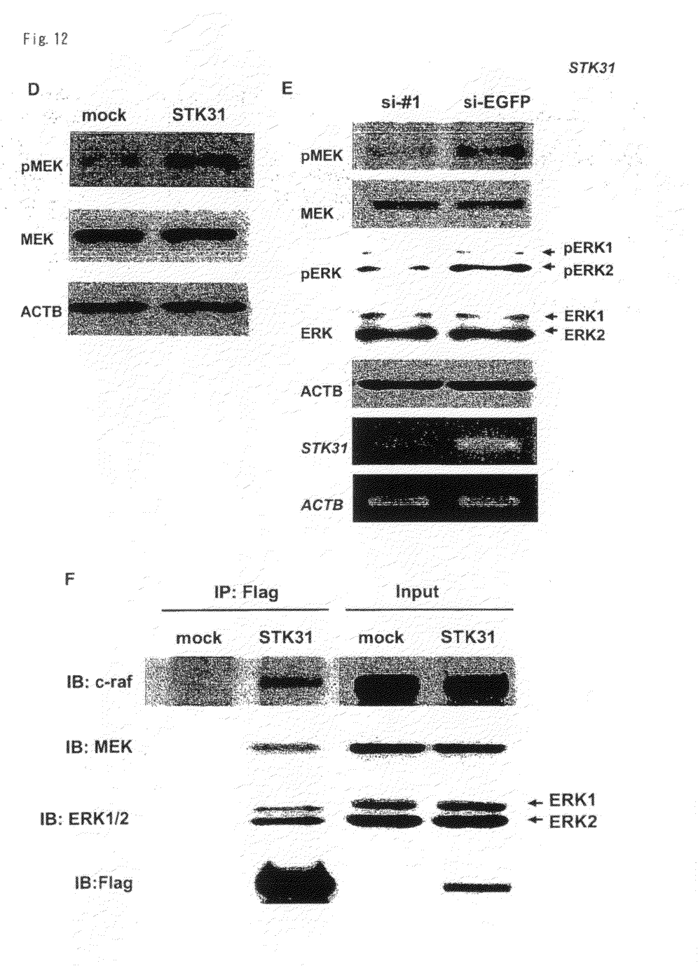

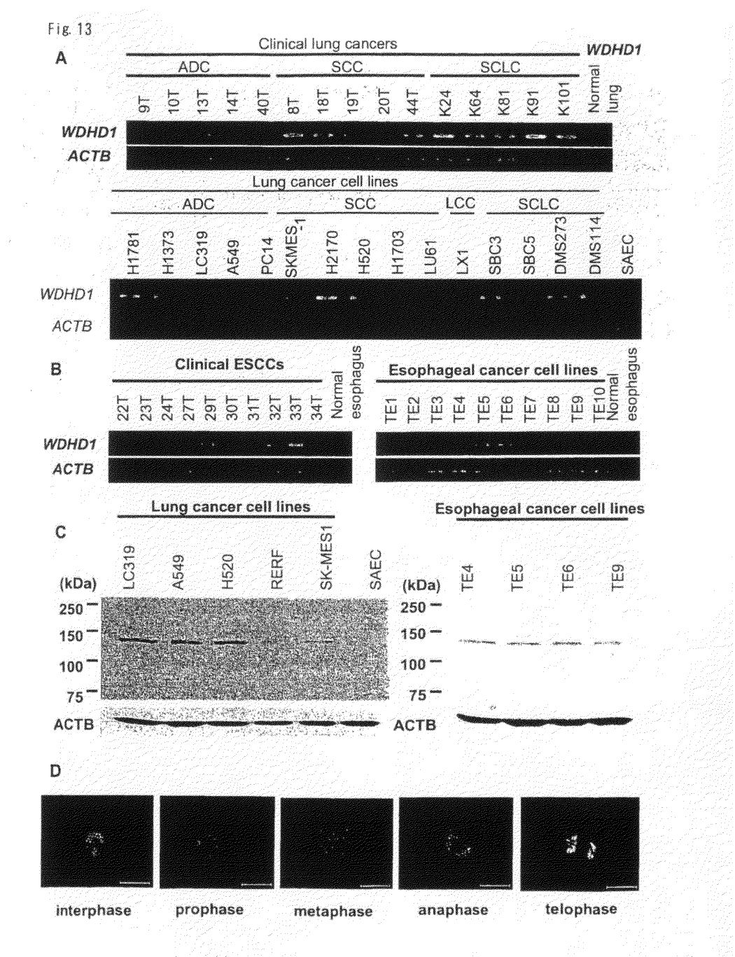

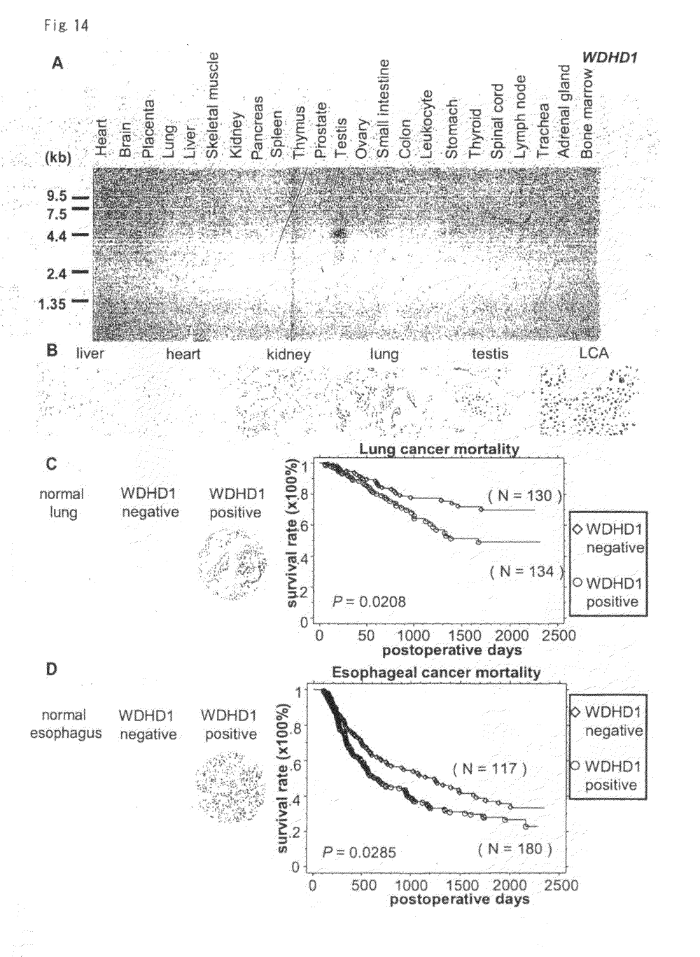

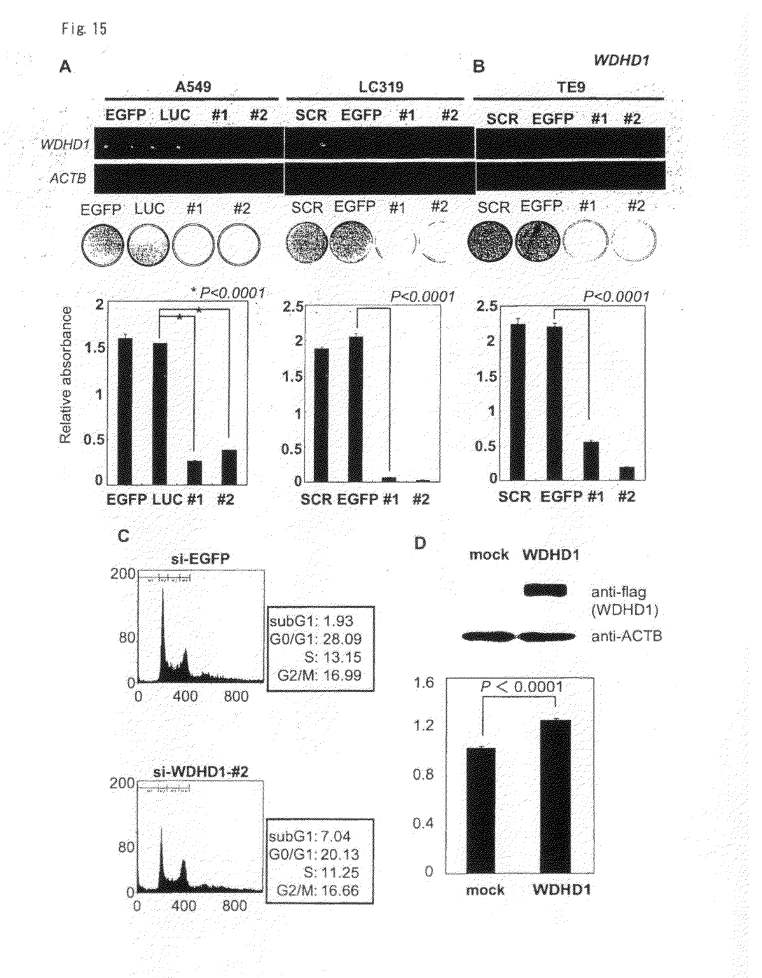

The expression of CX gene(s) were found to be specifically elevated in lung and esophageal cancers tissues compared with corresponding normal tissues (FIG. 1 for CDCA5; FIG. 3 for EPHA7; FIG. 9 for STK31; and FIG. 13 for WDHD1). Therefore, the genes identified herein as well as its transcription and translation products have diagnostic utility as markers for cancers mediated by one or more CX genes and by measuring the expression of the CX gene(s) in a sample derived from a patient suspected to be suffering from cancers, these cancers can be diagnosed. Specifically, the present invention provides a method for diagnosing cancers mediated by one or more CX genes by determining the expression level of the CX gene(s) in the subject. The CX gene-promoted cancers that can be diagnosed by the present method include lung and esophageal cancers. Lung cancers include non-small lung cancer and small lung cancer. The CX genes can be selected from the group consisting of CDCA5, EPHA7, STK31 and WDHD1.

According to the present invention, an intermediate result for examining the condition of a subject can be provided. Such intermediate result can be combined with additional information to assist a doctor, nurse, or other practitioner to diagnose that a subject suffers from the disease. Alternatively, the present invention can be used to detect cancerous cells in a subject-derived tissue, and provide a doctor with useful information to diagnose that the subject suffers from the disease.

Specifically, the present invention provides the following methods [1] to [10]:

[1] A method for diagnosing cancers, e.g., cancers mediated or promoted by a CX gene, wherein said method comprising the steps of:

(a) detecting the expression level of the gene selected from the group consisting of CDCA5, EPHA7, STK31 and WDHD1 in a biological sample; and

(b) relating an increase of the expression level compared to a normal control level of the gene to the disease.

[2] The method of [1], wherein the expression level is at least 10% greater than normal control level.

[3] The method of [2], wherein the expression level is detected by any one of the method select from the group consisting of:

(a) detecting the mRNA encoding the polypeptide selected from the group consisting of CDCA5, EPHA7, STK31 and WDHD1;

(b) detecting the polypeptide selected from the group consisting of CDCA5, EPHA7, STK31 and WDHD1; and

(c) detecting the biological activity of the polypeptide selected from the group consisting of CDCA5, EPHA7, STK31 and WDHD1.

The method of [1], wherein the cancer results from overexpression of a CX gene, or is mediated or promoted by a CX gene.

[4] The method of [1], wherein the cancers is lung cancer or esophageal cancer.

[5] The method of [4], wherein the lung cancer is non-small cell lung cancer or small cell lung cancer.

[6] The method of [3], wherein the expression level is determined by detecting a hybridization of probe to the gene transcript encoding the polypeptide selected from the group consisting of CDCA5, EPHA7, STK31 and WDHD1.

[7] The method of [3], wherein the expression level is determined by detecting a binding of an antibody against the polypeptide selected from the group consisting of CDCA5, EPHA7, STK31 and WDHD1.

[8] The method of [1], wherein the biological sample comprises biopsy, sputum or blood.

[9] The method of [1], wherein the subject-derived biological sample comprises an epithelial cell, serum, pleural effusion or esophageal mucosa.

[10] The method of [1], wherein the subject-derived biological sample comprises a cancer cell.

[11] The method of [1], wherein the subject-derived biological sample comprises a cancerous epithelial cell.

The method of diagnosing cancers will be described in more detail below.

A subject to be diagnosed by the present method is can be a mammal. Exemplary mammals include, but are not limited to, e.g., human, non-human primate, mouse, rat, dog, cat, horse, and cow.

In performing the present methods, a biological sample is collected from the subject to be diagnosed to perform the diagnosis. Any biological material can be used as the biological sample for the determination so long as it comprises the objective transcription or translation product of CX gene(s). The biological samples include, but are not limited to, bodily tissues and fluids, for example, blood, e.g. serum, sputum, urine and pleural effusion. In some embodiments, the biological sample contains a cell population comprising an epithelial cell, for example, a cancerous epithelial cell or an epithelial cell derived from tissue suspected to be cancerous. Further, if necessary, the cell can be purified from the obtained bodily tissues and fluids, and then used as the biological sample.

According to the present invention, the expression level of CX gene(s) in the subject-derived biological sample is determined. The expression level can be determined at the transcription (nucleic acid) product level, using methods known in the art. For example, the mRNA of CX gene(s) can be quantified using probes by hybridization methods (e.g. Northern blot analysis). The detection can be carried out on a chip or an array. The use of an array can be for detecting the expression level of a plurality of genes (e.g., various cancer specific genes) including CX genes. Those skilled in the art can prepare such probes utilizing the sequence information of the CDCA5 (SEQ ID NO: 1; GenBank Accession No. BC011000), EPHA7 (SEQ ID NO: 3; GenBank Accession No. NM_{—}004440), STK31 (SEQ ID NO: 5; GenBank Accession No. NM_{—}032944.1) or WDHD1 (SEQ ID NO: 7; GenBank Accession No. NM_{—}007086.2). For example, the cDNA of CX gene(s) can be used as the probes. If necessary, the probe can be labeled with a suitable label, for example, dyes, fluorescent and isotopes, and the expression level of the gene can be detected as the intensity of the hybridized labels (see, (4) Northern-blot analysis in [EXAMPLE 1]).

Furthermore, the transcription product of CX genes can be quantified using primers by amplification-based detection methods (e.g., RT-PCR). Such primers can also be prepared based on the available sequence information of the gene. For example, the primers (SEQ ID NO: 11 and 12 or SEQ ID NO: 19 and 20 for CDCA5, SEQ ID NO: 13 and 14 for EPHA7, SEQ ID NO: 15 and 16 or SEQ ID NO: 21 and 16 for STK31 and SEQ ID NO: 17 and 18 or SEQ ID NO: 22 and 18 for WDHD1) used in the Example can be employed for the detection by RT-PCR or Northern blot, but the present invention is not restricted thereto (see, (3) Semi-quantitative RT-PCR and (4) Northern-blot analysis in [EXAMPLE 1]).

Specifically, a probe or primer used for the present method hybridizes under stringent, moderately stringent, or low stringent conditions to the mRNA of CX genes.

Alternatively, the translation product can be detected for the diagnosis of the present invention. For example, the quantity of CX protein can be determined. A method for determining the quantity of the protein as the translation product includes immunoassay methods that use an antibody specifically recognizing the protein. The antibody can be monoclonal or polyclonal. Furthermore, any fragment or modification (e.g., chimeric antibody, scFv, Fab, F(ab′)2, Fv, etc.) of the antibody can be used for the detection, so long as the fragment retains the binding ability to CX protein. Methods to prepare these kinds of antibodies for the detection of proteins are well known in the art, and any method can be employed in the present invention to prepare such antibodies and equivalents thereof (see, (2) Antibody in Definition).

As another method to detect the expression level of CX gene based on its translation product, the intensity of staining can be observed via immunohistochemical analysis using an antibody against CX protein. Namely, the observation of strong staining indicates increased presence of the protein and at the same time high expression level of CX gene (see, (8) Immunohistochemistry and Tissue-microarray analysis in [EXAMPLE 1]).

Moreover, in addition to the expression level of CX gene, the expression level of other cancer-associated genes, for example, genes known to be differentially expressed in cancers can also be determined to improve the accuracy of the diagnosis.

The expression level of cancer marker gene including CX gene in a biological sample can be considered to be increased if it increases from the control level of the corresponding cancer marker gene (e.g., in a normal or non-cancerous cell) by, for example, 10%, 25%, or 50%; or increases to more than 1.1 fold, more than 1.5 fold, more than 2.0 fold, more than 5.0 fold, more than 10.0 fold, or more.

The control level can be determined at the same time with the test biological sample by using a sample(s) previously collected and stored from a subject/subjects whose disease state (cancerous or non-cancerous) is/are known. Alternatively, the control level can be determined by a statistical method based on the results obtained by analyzing previously determined expression level(s) of CX gene in samples from subjects whose disease state are known. Furthermore, the control level can be a database of expression patterns from previously tested cells. Moreover, according to an aspect of the present invention, the expression level of a CX gene in a biological sample can be compared to multiple control levels, which control levels are determined from multiple reference samples. In some embodiments, a control level determined from a reference sample derived from a tissue type similar to that of the patient-derived biological sample is used. In some embodiments, the standard value of the expression levels of CX gene in a population with a known disease state is used. The standard value can be obtained by any method known in the art. For example, a range of mean+/−2 S.D. or mean+/−3 S.D. can be used as standard value.

In the context of the present invention, a control level determined from a biological sample that is known not to be cancerous is called “normal control level”. On the other hand, if the control level is determined from a cancerous biological sample, it will be called “cancerous control level”.

When the expression level of CX gene is increased compared to the normal control level or is similar to the cancerous control level, the subject can be diagnosed to be suffering from or at a risk of developing cancer, e.g., a cancer that is mediated by or results from overexpression of a CX gene. Furthermore, in case where the expression levels of multiple CX genes are compared, a similarity in the gene expression pattern between the sample and the reference which is cancerous indicates that the subject is suffering from or at a risk of developing cancer, e.g., a cancer that is mediated by or results from overexpression of a CX gene.

Difference between the expression levels of a test biological sample and the control level can be normalized to the expression level of control nucleic acids, e.g., housekeeping genes, whose expression levels are known not to differ depending on the cancerous or non-cancerous state of the cell. Exemplary control genes include, but are not limited to, beta-actin, glyceraldehyde 3 phosphate dehydrogenase, and ribosomal protein P1.

### Method for Assessing the Prognosis of a CX Gene-Mediated Cancer

The present invention is based, in part, on the discovery that EPHA7, STK31 or WDHD1 (over)expression is significantly associated with poorer prognosis of patients with CX gene-mediated cancers, e.g., lung or esophageal cancers Thus, the present invention provides a method for determining or assessing the prognosis of a patient with cancer, e.g., a cancer mediated by or resulting from overexpression of a CX gene, e.g., lung cancer and/or esophageal cancer, by detecting the expression level of the EPHA7, STK31 or WDHD1 gene in a biological sample of the patient; comparing the detected expression level to a control level; and determining a increased expression level to the control level as indicative of poor prognosis (poor survival).

Herein, the term “prognosis” refers to a forecast as to the probable outcome of the disease as well as the prospect of recovery from the disease as indicated by the nature and symptoms of the case. Accordingly, a less favorable, negative or poor prognosis is defined by a lower post-treatment survival term or survival rate. Conversely, a positive, favorable, or good prognosis is defined by an elevated post-treatment survival term or survival rate.

The terms “assessing the prognosis” refer to the ability of predicting, forecasting or correlating a given detection or measurement with a future outcome of cancer of the patient (e.g., malignancy, likelihood of curing cancer, estimated time of survival, and the like). For example, a determination of the expression level of EPHA7, STK31 or WDHD1 over time enables a predicting of an outcome for the patient (e.g., increase or decrease in malignancy, increase or decrease in grade of a cancer, likelihood of curing cancer, survival, and the like).

In the context of the present invention, the phrase “assessing (or determining) the prognosis” is intended to encompass predictions and likelihood analysis of cancer, progression, particularly cancer recurrence, metastatic spread and disease relapse. The present method for assessing prognosis is intended to be used clinically in making decisions concerning treatment modalities, including therapeutic intervention, diagnostic criteria for example, disease staging, and disease monitoring and surveillance for metastasis or recurrence of neoplastic disease.

The patient-derived biological sample used for the method can be any sample derived from the subject to be assessed so long as the EPHA7, STK31 or WDHD1 gene can be detected in the sample. In some embodiments, the biological sample comprises a lung cell (a cell obtained from lung or esophageal). Furthermore, the biological sample includes bodily fluids for example, sputum, blood, serum, plasma, pleural effusion, esophageal mucosa, and so on. Moreover, the sample can be cells purified from a tissue. The biological samples can be obtained from a patient at various time points, including before, during, and/or after a treatment.

According to the present invention, it was shown that the higher the expression level of the EPHA7, STK31 or WDHD1 gene measured in the patient-derived biological sample, the poorer the prognosis for post-treatment remission, recovery, and/or survival and the higher the likelihood of poor clinical outcome. Thus, according to the present method, the “control level” used for comparison can be, for example, the expression level of the EPHA7, STK31 or WDHD1 gene detected before any kind of treatment in an individual or a population of individuals who showed good or positive prognosis of cancer, after the treatment, which herein will be referred to as “good prognosis control level”. Alternatively, the “control level” can be the expression level of the EPHA7, STK31 or WDHD1 gene detected before any kind of treatment in an individual or a population of individuals who showed poor or negative prognosis of cancer, after the treatment, which herein will be referred to as “poor prognosis control level”. The “control level” is a single expression pattern derived from a single reference population or from a plurality of expression patterns. Thus, the control level can be determined based on the expression level of the EPHA7, STK31 or WDHD1 gene detected before any kind of treatment in a patient of cancer, or a population of the patients whose disease state (good or poor prognosis) is known. In some embodiments, the cancer is lung cancer. In some embodiments, the standard value of the expression levels of the EPHA7, STK31 or WDHD1 gene in a patient group with a known disease state is used. The standard value can be obtained by any method known in the art. For example, a range of mean+/−2 S.D. or mean+/−3 S.D. can be used as standard value.

The control level can be determined at the same time with the test biological sample by using a sample(s) previously collected and stored before any kind of treatment from cancer patient(s) (control or control group) whose disease state (good prognosis or poor prognosis) are known.

Alternatively, the control level can be determined by a statistical method based on the results obtained by analyzing the expression level of the EPHA7, STK31 or WDHD1 gene in samples previously collected and stored from a control group. Furthermore, the control level can be a database of expression patterns from previously tested cells or patients. Moreover, according to an aspect of the present invention, the expression level of the EPHA7, STK31 or WDHD1 gene in a biological sample can be compared to multiple control levels, which control levels are determined from multiple reference samples. In some embodiments, a control level determined from a reference sample derived from a tissue type similar to that of the patient-derived biological sample is used.

According to the present invention, a similarity in the expression level of the EPHA7, STK31 or WDHD1 gene to the good prognosis control level indicates a more favorable prognosis of the patient and an increase in the expression level in comparison to the good prognosis control level indicates less favorable, poorer prognosis for post-treatment remission, recovery, survival, and/or clinical outcome. On the other hand, a decrease in the expression level of the EPHA7, STK31 or WDHD1 gene in comparison to the poor prognosis control level indicates a more favorable prognosis of the patient and a similarity in the expression level to the poor prognosis control level indicates less favorable, poorer prognosis for post-treatment remission, recovery, survival, and/or clinical outcome.

An expression level of the EPHA7, STK31 or WDHD1 gene in a biological sample can be considered altered (i.e., increased or decreased) when the expression level differs from the control level by more than 1.0, 1.5, 2.0, 5.0, 10.0, or more fold.

The difference in the expression level between the test biological sample and the control level can be normalized to a control, e.g., housekeeping gene. For example, polynucleotides whose expression levels are known not to differ between the cancerous and non-cancerous cells, including those coding for beta-actin, glyceraldehyde 3-phosphate dehydrogenase, and ribosomal protein P1, can be used to normalize the expression levels of the EPHA7, STK31 or WDHD1 gene.

The expression level can be determined by detecting the gene transcript in the patient-derived biological sample using techniques well known in the art. The gene transcripts detected by the present method include both the transcription and translation products, for example, mRNA and protein.

For instance, the transcription product of the EPHA7, STK31 or WDHD1 gene can be detected by hybridization, e.g., Northern blot hybridization analyses, that use an EPHA7, STK31 or WDHD1 gene probe to the gene transcript. The detection can be carried out on a chip or an array. An array can be used for detecting the expression level of a plurality of genes including the EPHA7, STK31 or WDHD1 gene. As another example, amplification-based detection methods, for example, reverse-transcription based polymerase chain reaction

(RT-PCR) which use primers specific to the EPHA7, STK31 or WDHD1 gene can be employed for the detection (see (3) Semi-quantitative RT-PCR in [EXAMPLE 1]). The EPHA7, STK31 or WDHD1 gene-specific probe or primers can be designed and prepared using conventional techniques by referring to the whole sequence of the EPHA7 (SEQ ID NO: 3), STK31 (SEQ ID NO: 5) and WDHD1 (SEQ ID NO: 7). For example, the primers (SEQ ID NOs: 13 and 14 (EPHA7), SEQ ID NOs: 15 and 16 (STK31), SEQ ID NOs: 17 and 18 (WDHD1)) used in the Example can be employed for the detection by RT-PCR, but the present invention is not restricted thereto.

Specifically, a probe or primer used for the present method hybridizes under stringent, moderately stringent, or low stringent conditions to the mRNA of the EPHA7, STK31 or WDHD1 gene. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degree Centigrade lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degree Centigrade for short probes or primers (e.g., 10 to 50 nucleotides) and at least about 60 degree Centigrade for longer probes or primers. Stringent conditions can also be achieved with the addition of destabilizing agents, for example, formamide.

Alternatively, the translation product can be detected for the assessment of the present invention. For example, the quantity of the EPHA7, STK31 or WDHD1 protein can be determined. A method for determining the quantity of the protein as the translation product includes immunoassay methods that use an antibody specifically recognizing the EPHA7, STK31 or WDHD1 protein. The antibody can be monoclonal or polyclonal. Furthermore, any fragment or modification (e.g., chimeric antibody, scFv, Fab, F(ab′)2, Fv, etc.) of the antibody can be used for the detection, so long as the fragment retains the binding ability to the EPHA7, STK31 or WDHD1 protein. Methods to prepare these kinds of antibodies for the detection of proteins are well known in the art, and any method can be employed in the present invention to prepare such antibodies and equivalents thereof.

As another method to detect the expression level of the EPHA7, STK31 or WDHD1 gene based on its translation product, the intensity of staining can be observed via immunohistochemical analysis using an antibody against EPHA7, STK31 or WDHD1 protein. Namely, the observation of strong staining indicates increased presence of the EPHA7, STK31 or WDHD1 protein and at the same time high expression level of the EPHA7, STK31 or WDHD1 gene.

Furthermore, the EPHA7, STK31 or WDHD1 protein is known to have a cell proliferating activity. Therefore, the expression level of the EPHA7, STK31 or WDHD1 gene can be determined using such cell proliferating activity as an index. For example, cells which express EPHA7, STK31 or WDHD1 are prepared and cultured in the presence of a biological sample, and then by detecting the speed of proliferation, or by measuring the cell cycle or the colony forming ability the cell proliferating activity of the biological sample can be determined.

Moreover, in addition to the expression level of the EPHA7, STK31 or WDHD1 gene, the expression level of other lung cell-associated genes, for example, genes known to be differentially expressed in lung cancer or esophageal cancer, can also be determined to improve the accuracy of the assessment. Such other lung cancer-associated genes include those described in WO 2004/031413 and WO 2005/090603; and such other esophageal cancer-associated genes in dude those described in WO 2007/013671.

The patient to be assessed for the prognosis of cancer according to the method can be a mammal and includes human, non-human primate, mouse, rat, dog, cat, horse, and cow.

Alternatively, according to the present invention, an intermediate result can also be provided in addition to other test results for assessing the prognosis of a subject. Such intermediate result can assist a doctor, nurse, or other practitioner to assess, determine, or estimate the prognosis of a subject. Additional information that can be considered, in combination with the intermediate result obtained by the present invention, to assess prognosis includes clinical symptoms and physical conditions of a subject.

### Kits for Diagnosing Cancer or Assessing the Prognosis of Cancer

The present invention provides a kit for diagnosing cancer or assessing the prognosis of cancer. In some embodiments, the cancer is mediated by a CX gene or resulting from overexpression of a CX gene, e.g., lung cancer and/or esophageal cancer. Specifically, the kit comprises at least one reagent for detecting the expression of the CDCA5, EPHA7, STK31 or WDHD1 gene in a patient-derived biological sample, which reagent can be selected from the group of:

(a) a reagent for detecting mRNA of the CDCA5, EPHA7, STK31 or WDHD1 gene;

(b) a reagent for detecting the CDCA5, EPHA7, STK31 or WDHD1 protein; and

(c) a reagent for detecting the biological activity of the CDCA5, EPHA7, STK31 or WDHD1 protein.

Suitable reagents for detecting mRNA of the CDCA5, EPHA7, STK31 or WDHD1 gene include nucleic acids that specifically bind to or identify the CDCA5, EPHA7, STK31 or WDHD1 mRNA, for example, oligonucleotides which have a complementary sequence to a part of the CDCA5, EPHA7, STK31 or WDHD1 mRNA. These kinds of oligonucleotides are exemplified by primers and probes that are specific to the CDCA5, EPHA7, STK31 or WDHD1 mRNA. These kinds of oligonucleotides can be prepared based on methods well known in the art. If needed, the reagent for detecting the CDCA5, EPHA7, STK31 and WDHD1 mRNA can be immobilized on a solid matrix. Moreover, more than one reagent for detecting the CDCA5, EPHA7, STK31 or WDHD1 mRNA can be included in the kit.

On the other hand, suitable reagents for detecting the CDCA5, EPHA7, STK31 or WDHD1 protein include antibodies to the CDCA5, EPHA7, STK31 or WDHD1 protein. The antibody can be monoclonal or polyclonal. Furthermore, any fragment or modification (e.g., chimeric antibody, scFv, Fab, F(ab′)2, Fv, etc.) of the antibody can be used as the reagent, so long as the fragment retains the binding ability to the CDCA5, EPHA7, STK31 or WDHD1 protein. Methods to prepare these kinds of antibodies for the detection of proteins are well known in the art, and any method can be employed in the present invention to prepare such antibodies and equivalents thereof. Furthermore, the antibody can be labeled with signal generating molecules via direct linkage or an indirect labeling technique. Labels and methods for labeling antibodies and detecting the binding of antibodies to their targets are well known in the art and any labels and methods can be employed for the present invention. Moreover, more than one reagent for detecting the CDCA5, EPHA7, STK31 or WDHD1 protein can be included in the kit.

Furthermore, the biological activity can be determined by, for example, measuring the cell proliferating activity due to the expressed CDCA5, EPHA7, STK31 or WDHD1 protein in the biological sample. For example, the cell is cultured in the presence of a patient-derived biological sample, and then by detecting the speed of proliferation, or by measuring the cell cycle or the colony forming ability the cell proliferating activity of the biological sample can be determined. If needed, the reagent for detecting the CDCA5, EPHA7, STK31 or WDHD1 mRNA can be immobilized on a solid matrix. Moreover, more than one reagent for detecting the biological activity of the CDCA5, EPHA7, STK31 or WDHD1 protein can be included in the kit.

The kit can comprise more than one of the aforementioned reagents. Furthermore, the kit can comprise a solid matrix and reagent for binding a probe against the CDCA5, EPHA7, STK31 or WDHD1 gene or antibody against the CDCA5, EPHA7, STK31 or WDHD1 protein, a medium and container for culturing cells, positive and negative control reagents, and a secondary antibody for detecting an antibody against the CDCA5, EPHA7, STK31 or WDHD1 protein. For example, tissue samples obtained from patient with good prognosis or poor prognosis can serve as useful control reagents. A kit of the present invention can further include other materials desirable from a commercial and user standpoint, including buffers, diluents, filters, needles, syringes, and package inserts (e.g., written, tape, CD-ROM, etc.) with instructions for use. These reagents and such can be comprised in a container with a label. Suitable containers include bottles, vials, and test tubes. The containers can be formed from a variety of materials, for example, glass or plastic.

As an embodiment of the present invention, when the reagent is a probe against the CDCA5, EPHA7, STK31 or WDHD1 mRNA, the reagent can be immobilized on a solid matrix, for example, a porous strip, to form at least one detection site. The measurement or detection region of the porous strip can include a plurality of sites, each containing a nucleic acid (probe). A test strip can also contain sites for negative and/or positive controls. Alternatively, control sites can be located on a strip separated from the test strip. Optionally, the different detection sites can contain different amounts of immobilized nucleic acids, i.e., a higher amount in the first detection site and lesser amounts in subsequent sites. Upon the addition of test sample, the number of sites displaying a detectable signal provides a quantitative indication of the amount of CDCA5, EPHA7, STK31 or WDHD1 mRNA present in the sample. The detection sites can be configured in any suitably detectable shape and are typically in the shape of a bar or dot spanning the width of a test strip.

The kit of the present invention can further comprise a positive control sample or CDCA5, EPHA7, STK31 or WDHD1 standard sample. The positive control sample of the present invention can be prepared by collecting CDCA5, EPHA7, STK31 or WDHD1 positive blood samples and then those CDCA5, EPHA7, STK31 or WDHD1 level are assayed. Alternatively, purified CDCA5, EPHA7, STK31 or WDHD1 protein or polynucleotide can be added to CDCA5, EPHA7, STK31 or WDHD1 free serum to form the positive sample or the CDCA5, EPHA7, STK31 or WDHD1 standard. In the present invention, purified CDCA5, EPHA7, STK31 or WDHD1 can be recombinant protein. The CDCA5, EPHA7, STK31 or WDHD1 level of the positive control sample is, for example more than cut off value.

Hereinafter, the present invention is described in more detail with reference to the Examples. However, the following materials, methods and examples only illustrate aspects of the invention and in no way are intended to limit the scope of the present invention. As such, methods and materials similar or equivalent to those described herein can be used in the practice or testing of the present invention.

### Methods for Diagnosing Cancers

In the present invention, it was confirmed that that the N-terminal domain of EPHA7 protein is cleaved and secreted into extracellular space (FIG. 3G). Therefore the agent recognizing specific for the N-terminal domain of EPHA7 protein (526-580aa of SEQ ID NO: 4), is useful for detection a secreted type EPHA7. For example, the agent can be an antibody against the N-terminal domain of EPHA7 protein, especially an antibody against 526-580aa of SEQ ID NO: 4, e.g. rabbit polyclonal antibodies (Catalog No. sc25459, Santa Cruz, Santa Cruz, Calif.) for epitope(s) from N-terminal portion of human EPHA7, which used in [EXAMPLE 3]. The biological sample, e.g. body fluid can be examined by the agent whether EPHA7 is contained. The body fluid can include whole blood, serum, plasma, sputum, pleural effusion, esophageal mucosa, and so on. The detecting system can an immunoassay, ELISA or Western-blot.

Furthermore, the present inventors established an ELISA to measure serum EPHA7 and found that the proportion of serum EPHA7-positive cases was 149 (56.4%) of 264 non-small cell cancer (NSCLC), 35 (44.3%) of 79 SCLC, and 81 (84.4%) of 96 ESCC patients, while only 6 (4.7%) of 127 healthy volunteers were falsely diagnosed (FIG. 5, upper panel). The concentration of serum EPHA7 was dramatically reduced after surgical resection of primary tumors (FIG. 5B, right panel).

By measuring the level of EPHA7 in a subject-derived biological sample, the occurrence of cancer or a predisposition to develop cancer in a subject can be determined. In some embodiments, the cancer is mediated by a CX gene or results from overexpression of a CX gene, e.g., lung cancer and/or esophageal cancer. Accordingly, the present invention involves determining (e.g., measuring) the level of EPHA7 in a biological sample. Alternatively, according to the present invention, an intermediate result for examining the condition of a subject can be provided. Such intermediate result can be combined with additional information to assist a doctor, nurse, or other practitioner to diagnose that a subject suffers from the disease. Alternatively, the present invention can be used to detect cancerous cells in a subject-derived tissue, and provide a doctor with useful information to diagnose that the subject suffers from the disease. Further, subjects with suspected lung cancer and/or esophageal cancer can be screened by the present invention. Specifically, the present invention provides the following double-stranded molecules [1] to [5]:

[1] A method for diagnosing cancers in a subject or assessing efficacy of therapy for cancers, comprising the steps of:

(a) collecting a body fluid from a subject to be diagnosed;

(b) determining a level of EPHA7 protein or fragment thereof in the body fluid by immunoassay;

(c) comparing the level determined in step (b) with that of a normal control; and

(d) judging that a high level in the blood sample, compared to the normal control, indicates that the subject suffers from cancers.

[2] The method of claim [1], wherein the body fluid is selected from the group consisting of whole blood, serum and plasma.

[3] The method of claim [1], wherein the immunoassay is an ELISA.

[4] The method of [1], the cancer is lung cancer and/or esophageal cancer.

[5] The method of [3], the method is combined with other serum biomarkers.

[6] The method of [5], the other serum biomarkers selected from the group consisting of CEA and ProGRP.

[7] The method of [1], the therapy is surgery.

Any biological materials can be used as the biological sample for determining the level of EPHA7 protein can be detected in the sample. In some embodiments, the biological sample comprises blood, serum or other bodily fluids for example, sputum, pleural effusion, esophageal mucosa, and so on. In some embodiments, the biological sample is blood or blood derived sample. The blood derived sample includes serum, plasma, or whole blood. The subject diagnosed for cancer according to the method can be a mammal and includes human, non-human primate, mouse, rat, dog, cat, horse and cow.

In the embodiment, the level of EPHA7 is determined by measuring the quantity of EPHA7 protein in a biological sample. A method for determining the quantity of the EPHA7 protein in a biological sample includes immunoassay methods. In one embodiment, the immunoassay comprises an ELISA.

The EPHA7 level in the biological sample is then compared with an EPHA7 level associated with a reference sample, for example, a normal control sample. The phrase “normal control level” refers to the level of EPHA7 typically found in a biological sample of a population not suffering from cancer. The reference sample can be of a similar nature to that of the test sample. For example, if the test sample comprises patient serum, the reference sample should also be serum. The EPHA7 level in the biological samples from control and test subjects can be determined at the same time or, alternatively, the normal control level can be determined by a statistical method based on the results obtained by analyzing the level of EPHA7 in samples previously collected from a control group.

The EPHA7 level can also be used to monitor the course of treatment of cancer. In this method, a test biological sample is provided from a subject undergoing treatment for cancer. In some embodiments, the cancer is lung cancer and/or esophageal cancer. In some embodiments, the multiple test biological samples are obtained from the subject at various time points before, during or after the treatment. The level of EPHA7 in the post-treatment sample can then be compared with the level of EPHA7 in the pre-treatment sample or, alternatively, with a reference sample (e.g., a normal control level). For example, if the post-treatment EPHA7 level is lower than the pre-treatment EPHA7 level, one can conclude that the treatment was efficacious. Likewise, if the post-treatment EPHA7 level is similar to the normal control EPHA7 level, one can also conclude that the treatment was efficacious.

An “efficacious” treatment is one that leads to a reduction in the level of EPHA7 or a decrease in size, prevalence or metastatic potential of cancer in a subject. When a treatment is applied prophylactically, “efficacious” means that the treatment retards or prevents occurrence of cancer or alleviates a clinical symptom of cancer. The assessment of cancer can be made using standard clinical protocols. Furthermore, the efficaciousness of a treatment can be determined in association with any known method for diagnosing or treating cancer. For example, cancer is routinely diagnosed histopathologically or by identifying symptomatic anomalies for example, chronic cough, hoarseness, coughing up blood, weight loss, loss of appetite, shortness of breath, wheezing, repeated bouts of bronchitis or pneumonia and chest pain.

Moreover, the present method for diagnosing cancer can also be applied for assessing the prognosis of a patient with the cancer by comparing the level of EPHA7 in a patient-derived biological sample with that of a reference sample. In some embodiments, the cancer is lung cancer. Alternatively, the level of EPHA7 in the biological sample can be measured over a spectrum of disease stages to assess the prognosis of the patient. An increase in the level of EPHA7 as compared to a normal control level indicates less favorable prognosis. A similarity in the level of EPHA7 as compared to a normal control level indicates a more favorable prognosis of the patient.

In the method of diagnosis of the present invention, the blood concentration of either CEA or proGRP, or both, can be referred to, in addition to the blood concentration of EPHA7, to detect lung cancer. Therefore, the present invention provides methods for diagnosing lung cancer, in which NSCLC is detected when the blood concentration of CEA, in addition to the blood concentration of EPHA7, is higher as compared with healthy individuals. Alternatively, the present invention provides methods for diagnosing lung cancer, in which SCLC is detected when the blood concentration of proGRP, in addition to the blood concentration of EPHA7, is higher as compared with healthy individuals.

The carcinoembryonic Antigen (CEA) was one of the oncofetal antigens to be applied clinically. It is a complex glycoprotein of molecular weight 20,000 that is associated with the plasma membrane of tumor cells, from which it can be released into the blood.

Although CEA was first identified in colon cancer, an abnormal CEA blood level is specific neither for colon cancer nor for malignancy in general. Elevated CEA levels are found in a variety of cancers other than colonic, including lung, pancreatic, gastric, and breast. As described above, CEA has already been used as serological marker for diagnosing or detecting lung cancer. However, the sensitivity of CEA as a marker for lung cancer, especially NSCLC is somewhat insufficient for detecting lung cancer, completely. Alternatively, it is also well known that gastrin-releasing peptide precursor (proGRP) is a serological tumor marker for SCLC. As described above, proGRP has already been used as serological marker for diagnosing or detecting SCLC. However, the sensitivity of proGRP as a marker for SCLC is somewhat insufficient for detecting SCLC, completely. Accordingly, it is required that the sensitivity of diagnosing lung cancer e.g. NSCLC and SCLC would be improved.

In the present invention, the serological marker for lung cancer EPHA7 is provided. Improvement in the sensitivity of diagnostic or detection method for lung cancer can be achieved by the present invention.

By the combination between EPHA7 and CEA and/or proGRP, the sensitivity for detection of lung i.e. NSCLC and/or SCLC can be significantly improved. For example, in the group analyzed in the working example mentioned later, CEA for NSCLC is a sensitivity of 37.9% (88/232) and a specificity of 89.8% (114/127); FIG. 5C, upper panel). In the meantime, the combination of EPHA7 and CEA improves overall sensitivity for detection of NSCLC to 76.7% (178 of 232). In the present invention, “combination of EPHA7 and CEA” refers either or both level of EPHA7 and CEA is used as marker. In some embodiments, patients testing positive for either of EPHA7 and CEA can be judged as suffering from NSCLC. The use of combination of EPHA7 and CEA as serological marker for NSCLC is not disclosed in the art.

Similarly, for example, in the group analyzed in the working example mentioned later, sensitivity of proGRP for SCLC is about 64.8% (46 of 71) and a specificity of 97.6% (120 of 123) (FIG. 5C, lower panel). In the meantime, that of combination between EPHA7 and proGRP improves overall sensitivity for detection of SCLC to 77.5% (55 of 71). In the present invention, “combination of EPHA7 and proGRP” refers either or both level of EPHA7 and proGRP is used as marker. In some embodiments, patients testing positive for either of EPHA7 and proGRP can be judged as suffering from SCLC. The use of combination of EPHA7 and proGRP as serological marker for SCLC is not disclosed in the art.

Therefore, the present invention can greatly improve the sensitivity for detecting NSCLC or SCLC patients, compared to determinations based on results of measuring CEA or proGRP alone. Behind this improvement is the fact that the group of CEA- or proGRP-positive patients and the group of EPHA7-positive patients do not match completely. This fact is further described specifically.

First, among patients who, as a result of CEA or proGRP measurements, were determined to have a lower value than a standard value (i.e. not to have lung cancer), there is actually a certain percentage of patients having lung cancer (i.e. NSCLC or SCLC). Such patients are referred to as CEA- or proGRP-false negative patients. By combining a determination based on CEA or proGRP with a determination based on EPHA7, patients whose EPHA7 value is above the standard value can be found from among the CEA- or proGRP-false-negative patients. That is, from among patients falsely determined to be “negative” due to a low blood concentration of CEA or proGRP, the present invention allows to find patients actually having lung cancer. The sensitivity for detecting lung cancer patients was thus improved by the present invention. Generally, simply combining the results from determinations using multiple markers can increase the detection sensitivity, but on the other hand, it often causes a decrease in specificity. However, by determining the best balance between sensitivity and specificity, the present invention has determined a characteristic combination that can increase the detection sensitivity without compromising the specificity.

In the present invention, in order to consider the results of CEA or proGRP measurements at the same time, for example, the blood concentration of CEA or proGRP can be measured and compared with standard values, in the same way as for the aforementioned comparison between the measured values and standard values of EPHA7. For example, how to measure the blood concentration of CEA or proGRP and compare it to standard values are already known. Moreover, ELISA kits for CEA or proGRP are also commercially available. These methods described in known reports can be used in the method of the present invention for diagnosing or detecting lung cancer.

In the present invention, the standard value of the blood concentration of EPHA7 can be determined statistically. For example, the blood concentration of EPHA7 in healthy individuals can be measured to determine the standard blood concentration of EPHA7 statistically. When a statistically sufficient population can be gathered, a value in the range of twice or three times the standard deviation (S.D.) from the mean value is often used as the standard value. Therefore, values corresponding to the mean value+2×S.D. or mean value+3×S.D. can be used as standard values. The standard values set as described theoretically comprise 90% and 99.7% of healthy individuals, respectively.

Alternatively, standard values can also be set based on the actual blood concentration of EPHA7 in lung cancer patients. Generally, standard values set this way minimize the percentage of false positives, and are selected from a range of values satisfying conditions that can maximize detection sensitivity. Herein, the percentage of false positives refers to a percentage, among healthy individuals, of patients whose blood concentration of EPHA7 is judged to be higher than a standard value. On the contrary, the percentage, among healthy individuals, of patients whose blood concentration of EPHA7 is judged to be lower than a standard value indicates specificity. That is, the sum of the false positive percentage and the specificity is always 1. The detection sensitivity refers to the percentage of patients whose blood concentration of EPHA7 is judged to be higher than a standard value, among all lung cancer patients within a population of individuals for whom the presence of lung cancer has been determined.

Furthermore, in the present invention, the percentage of lung cancer patients among patients whose EPHA7 concentration was judged to be higher than a standard value represents the positive predictive value. On the other hand, the percentage of healthy individuals among patients whose EPHA7 concentration was judged to be lower than a standard value represents the negative predictive value. The relationship between these values is summarized in Table 1. As the relationship shown below indicates, each of the values for sensitivity, specificity, positive predictive value, and negative predictive value, which are indexes for evaluating the diagnostic accuracy for lung cancer, varies depending on the standard value for judging the level of the blood concentration of EPHA7.

TABLE 1

| Blood | | | |
|---|---|---|---|
| concentration of | Lung cancer | Healthy | |
| EPHA7 | patients | individuals | |
| High | a: True positive | b: False | Positive |
| | | positive | predictive value |
| | | | a/(a + b) |
| Low | c: False negative | d: True | Negative |
| | | negative | predictive value |
| | | | d/(c + d) |
| | Sensitivity | Specificity | |
| | a/(a + c) | d/(b + d) | |

As already mentioned, a standard value is usually set such that the false positive ratio is low and the sensitivity is high. However, as also apparent from the relationship shown above, there is a trade-off between the false positive ratio and sensitivity. That is, if the standard value is decreased, the detection sensitivity increases. However, since the false positive ratio also increases, it is difficult to satisfy the conditions to have a “low false positive ratio”. Considering this situation, for example, values that give the following predicted results can be selected as representative standard values in the present invention. Standard values for which the false positive ratio is 50% or less (that is, standard values for which the specificity is not less than 50%).

Standard values for which the sensitivity is not less than 20%.

In the present invention, the standard values can be set using an ROC curve. A receiver operating characteristic (ROC) curve is a graph that shows the detection sensitivity on the vertical axis and the false positive ratio (that is, “1-specificity”) on the horizontal axis. In the present invention, an ROC curve can be obtained by plotting the changes in the sensitivity and the false positive ratio, which were obtained after continuously varying the standard value for determining the high/low degree of the blood concentration of EPHA7.

The “standard value” for obtaining the ROC curve is a value temporarily used for the statistical analyses. The “standard value” for obtaining the ROC curve can generally be continuously varied within a range that covers all selectable standard values. For example, the standard value can be varied between the smallest and largest measured EPHA7 values in an analyzed population.

Based on the obtained ROC curve, a representative standard value to be used in the present invention can be selected from a range that satisfies the above-mentioned conditions. Alternatively, a standard value can be selected based on an ROC curve produced by varying the standard values from a range that comprises most of the measured EPHA7 values.

EPHA7 in the blood can be measured by any method that can quantitate proteins. For example, immunoassay, liquid chromatography, surface plasmon resonance (SPR), mass spectrometry, or such can be applied as methods for quantitating proteins. In mass spectrometry, proteins can be quantitated by using a suitable internal standard. Isotope-labeled EPHA7 and such can be used as the internal standard. The concentration of EPHA7 in the blood can be determined from the peak intensity of EPHA7 in the blood and that of the internal standard. Generally, the matrix-assisted laser desorption/ionization (MALDI) method is used for mass spectrometry of proteins. With an analysis method that uses mass spectrometry or liquid chromatography, EPHA7 can also be analyzed simultaneously with other tumor markers (e.g. CEA and/or proGRP).

An exemplary method for measuring EPHA7 in the present invention is the immunoassay. The amino acid sequence of EPHA7 is known (GenBank Accession Number NP_{—}004431.1). The amino acid sequence of EPHA7 is shown in SEQ ID NO:, and the nucleotide sequence of the cDNA encoding it is shown in SEQ ID NO:. Therefore, those skilled in the art can prepare antibodies by synthesizing necessary immunogens based on the amino acid sequence of EPHA7. The peptide used as immunogen can be easily synthesized using a peptide synthesizer. The synthetic peptide can be used as an immunogen by linking it to a carrier protein. In some embodiments, the antigen peptide comprises the N-terminal region of EPHA7 or can be a fragment of the N-terminal region of EPHA7 (526-580aa of SEQ ID NO: 4).

Keyhole limpet hemocyanin, myoglobin, albumin, and such can be used as the carrier protein. Exemplary carrier proteins are KLH, bovine serum albumin, and such. The maleimidobenzoyl-N-hydroxysuccinimide ester method (hereinafter abbreviated as the MBS method) and such are generally used to link synthetic peptides to carrier proteins.

Specifically, a cysteine is introduced into the synthetic peptide and the peptide is crosslinked to KLH by MBS using the cysteine's SH group. The cysteine residue can be introduced at the N-terminus or C-terminus of the synthesized peptide.

Alternatively, EPHA7 can be obtained as a genetic recombinant based on the nucleotide sequence of EPHA7 (GenBank Accession Number NM_{—}004440). DNAs comprising the necessary nucleotide sequence can be cloned using mRNAs prepared from EPHA7-expressing tissues. Alternatively, commercially available cDNA libraries can be used as the cloning source. The obtained genetic recombinants of EPHA7, or fragments thereof, can also be used as the immunogen. EPHA7 recombinants expressed in this manner can be used as the immunogen for obtaining the antibodies used in the present invention. Commercially available EPHA7 recombinants can also be used as the immunogen. The antibody of the present invention can be prepared by conventional methods mentioned in (2) Antibody of Definition.

When antibodies against EPHA7 contact EPHA7, the antibodies bind to the antigenic determinant (epitope) that the antibodies recognize through an antigen-antibody reaction. The binding of antibodies to antigens can be detected by various immunoassay principles. Immunoassays can be broadly categorized into heterogeneous analysis methods and homogeneous analysis methods. To maintain the sensitivity and specificity of immunoassays to a high level, the use of monoclonal antibodies is desirable. Methods of the present invention for measuring EPHA7 by various immunoassay formats are specifically explained.

First, methods for measuring EPHA7 using a heterogeneous immunoassay are described. In heterogeneous immunoassays, a mechanism for detecting antibodies that bound to EPHA7 after separating them from those that did not bind to EPHA7 is required. To facilitate the separation, immobilized reagents are generally used. For example, a solid phase onto which antibodies recognizing EPHA7 have been immobilized is first prepared (immobilized antibodies). EPHA7 is made to bind to these, and secondary antibodies are further reacted thereto.

When the solid phase is separated from the liquid phase and further washed, as necessary, secondary antibodies remain on the solid phase in proportion to the concentration of EPHA7. By labeling the secondary antibodies, EPHA7 can be quantitated by measuring the signal derived from the label.

Any method can be used to bind the antibodies to the solid phase. For example, antibodies can be physically adsorbed to hydrophobic materials for example, polystyrene. Alternatively, antibodies can be chemically bound to a variety of materials having functional groups on their surfaces. Furthermore, antibodies labeled with a binding ligand can be bound to a solid phase by trapping them using a binding partner of the ligand. Combinations of a binding ligand and its binding partner include avidin-biotin and such. The solid phase and antibodies can be conjugated at the same time or before the reaction between the primary antibodies and EPHA7.

Similarly, the secondary antibodies do not need to be directly labeled. That is, they can be indirectly labeled using antibodies against antibodies or using binding reactions for example, that of avidin-biotin.

The concentration of EPHA7 in a sample is determined based on the signal intensities obtained using standard samples with known EPHA7 concentrations.

Any antibody can be used as the immobilized antibody and secondary antibody for the heterogeneous immunoassays mentioned above, so long as it is an antibody, or a fragment comprising an antigen-binding site thereof, that recognizes EPHA7. Therefore, it can be a monoclonal antibody, a polyclonal antibody, or a mixture or combination of both. For example, a combination of monoclonal antibodies and polyclonal antibodies is an exemplary combination in the present invention. Alternatively, when both antibodies are monoclonal antibodies, combining monoclonal antibodies recognizing different epitopes finds use.

Since the antigens to be measured are sandwiched by antibodies, such heterogenous immunoassays are called sandwich methods. Since sandwich methods excel in the measurement sensitivity and the reproducibility, they are a suitable measurement principle in the present invention.

The principle of competitive inhibition reactions can also be applied to the heterogeneous immunoassays. Specifically, they are immunoassays based on the phenomenon where EPHA7 in a sample competitively inhibits the binding between EPHA7 with a known concentration and an antibody. The concentration of EPHA7 in the sample can be determined by labeling EPHA7 with a known concentration and measuring the amount of EPHA7 that reacted (or did not react) with the antibody.

A competitive reaction system is established when antigens with a known concentration and antigens in a sample are simultaneously reacted to an antibody. Furthermore, analyses by an inhibitory reaction system are possible when antibodies are reacted with antigens in a sample, and antigens with a known concentration are reacted thereafter. In both types of reaction systems, reaction systems that excel in the operability can be constructed by setting either one of the antigens with a known concentration used as a reagent component or the antibody as the labeled component, and the other one as the immobilized reagent.

Radioisotopes, fluorescent substances, luminescent substances, substances having an enzymatic activity, macroscopically observable substances, magnetically observable substances, and such are used in these heterogeneous immunoassays. Specific examples of these labeling substances are shown below.

Substances having an enzymatic activity:
peroxidase,
alkaline phosphatase,
urease, catalase,
glucose oxidase,
lactate dehydrogenase, or
amylase, etc.

Fluorescent substances:
fluorescein isothiocyanate,
tetramethylrhodamine isothiocyanate,
substituted rhodamine isothiocyanate, or
dichlorotriazine isothiocyanate, etc.

Radioisotopes:
tritium,
¹²⁵I, or
¹³¹I, etc.

Among these, non-radioactive labels for example, enzymes are an advantageous label in terms of safety, operability, sensitivity, and such. Enzymatic labels can be linked to antibodies or to EPHA7 by known methods for example, the periodic acid method or maleimide method.

As the solid phase, beads, inner walls of a container, fine particles, porous carriers, magnetic particles, or such are used. Solid phases formed using materials for example, polystyrene, polycarbonate, polyvinyltoluene, polypropylene, polyethylene, polyvinyl chloride, nylon, polymethacrylate, latex, gelatin, agarose, glass, metal, ceramic, or such can be used. Solid materials in which functional groups to chemically bind antibodies and such have been introduced onto the surface of the above solid materials are also known. Known binding methods, including chemical binding for example, poly-L-lysine or glutaraldehyde treatment and physical adsorption, can be applied for solid phases and antibodies (or antigens).

Although the steps of separating the solid phase from the liquid phase and the washing steps are required in all heterogeneous immunoassays exemplified herein, these steps can easily be performed using the immunochromatography method, which is a variation of the sandwich method.

Specifically, antibodies to be immobilized are immobilized onto porous carriers capable of transporting a sample solution by the capillary phenomenon, then a mixture of a sample comprising EPHA7 and labeled antibodies is deployed therein by this capillary phenomenon. During deployment, EPHA7 reacts with the labeled antibodies, and when it further contacts the immobilized antibodies, it is trapped at that location. The labeled antibodies that did not react with EPHA7 pass through, without being trapped by the immobilized antibodies.

As a result, the presence of EPHA7 can be detected using, as an index, the signals of the labeled antibodies that remain at the location of the immobilized antibodies. If the labeled antibodies are maintained upstream in the porous carrier in advance, all reactions can be initiated and completed by just dripping in the sample solutions, and an extremely simple reaction system can be constructed. In the immunochromatography method, labeled components that can be distinguished macroscopically, for example, colored particles, can be combined to construct an analytical device that does not even require a special reader.

Furthermore, in the immunochromatography method, the detection sensitivity for EPHA7 can be adjusted. For example, by adjusting the detection sensitivity near the cutoff value described below, the aforementioned labeled components can be detected when the cutoff value is exceeded. By using such a device, whether a subject is positive or negative can be judged very simply. By adopting a constitution that allows a macroscopic distinction of the labels, necessary examination results can be obtained by simply applying blood samples to the device for immunochromatography.

Various methods for adjusting the detection sensitivity of the immunochromatography method are known. For example, a second immobilized antibody for adjusting the detection sensitivity can be placed between the position where samples are applied and the immobilized antibodies (Japanese Patent Application Kokai Publication No. (JP-A) H06-341989 (unexamined, published Japanese patent application)). EPHA7 in the sample is trapped by the second immobilized antibody while deploying from the position where the sample was applied to the position of the first immobilized antibody for label detection. After the second immobilized antibody is saturated, EPHA7 can reach the position of the first immobilized antibody located downstream. As a result, when the concentration of EPHA7 comprised in the sample exceeds a predetermined concentration, EPHA7 bound to the labeled antibody is detected at the position of the first immobilized antibody.

Next, homogeneous immunoassays are explained. As opposed to heterogeneous immunological assay methods that require a separation of the reaction solutions as described above, EPHA7 can also be measured using homogeneous analysis methods. Homogeneous analysis methods allow the detection of antigen-antibody reaction products without their separation from the reaction solutions.

A representative homogeneous analysis method is the immunoprecipitation reaction, in which antigenic substances are quantitatively analyzed by examining precipitates produced following an antigen-antibody reaction. Polyclonal antibodies are generally used for the immunoprecipitation reactions. When monoclonal antibodies are applied, multiple types of monoclonal antibodies that bind to different epitopes of EPHA7 can be used. The products of precipitation reactions that follow the immunological reactions can be macroscopically observed or can be optically measured for conversion into numerical data.

The immunological particle agglutination reaction, which uses as an index the agglutination by antigens of antibody-sensitized fine particles, is a common homogeneous analysis method. As in the aforementioned immunoprecipitation reaction, polyclonal antibodies or a combination of multiple types of monoclonal antibodies can be used in this method as well. Fine particles can be sensitized with antibodies through sensitization with a mixture of antibodies, or they can be prepared by mixing particles sensitized separately with each antibody. Fine particles obtained in this manner gives matrix-like reaction products upon contact with EPHA7. The reaction products can be detected as particle aggregation. Particle aggregation can be macroscopically observed or can be optically measured for conversion into numerical data.

Immunological analysis methods based on energy transfer and enzyme channeling are known as homogeneous immunoassays. In methods utilizing energy transfer, different optical labels having a donor/acceptor relationship are linked to multiple antibodies that recognize adjacent epitopes on an antigen. When an immunological reaction takes place, the two parts approach and an energy transfer phenomenon occurs, resulting in a signal for example, quenching or a change in the fluorescence wavelength. On the other hand, enzyme channeling utilizes labels for multiple antibodies that bind to adjacent epitopes, in which the labels are a combination of enzymes having a relationship such that the reaction product of one enzyme is the substrate of another. When the two parts approach due to an immunological reaction, the enzyme reactions are promoted; therefore, their binding can be detected as a change in the enzyme reaction rate.

In the present invention, blood for measuring EPHA7 can be prepared from blood drawn from patients. Exemplary blood samples include serum or plasma. Serum or plasma samples can be diluted before the measurements. Alternatively, the whole blood can be measured as a sample and the obtained measured value can be corrected to determine the serum concentration. For example, concentration in whole blood can be corrected to the serum concentration by determining the percentage of corpuscular volume in the same blood sample.

In one embodiment, the immunoassay comprises an ELISA. The present inventors established sandwich ELISA to detect serum EPHA7 in patients with respectable lung cancer.

The EPHA7 level in the blood samples is then compared with an EPHA7 level associated with a reference sample for example, a normal control sample. The phrase “normal control level” refers to the level of EPHA7 typically found in a blood sample of a population not suffering from lung cancer. The reference sample can be of a similar nature to that of the test sample. For example, if the test samples comprise patient serum, the reference sample should also be serum. The EPHA7 level in the blood samples from control and test subjects can be determined at the same time or, alternatively, the normal control level can be determined by a statistical method based on the results obtained by analyzing the level of EPHA7 in samples previously collected from a control group.

The EPHA7 level can also be used to monitor the course of treatment of lung cancer. In this method, a test blood sample is provided from a subject undergoing treatment for lung cancer. In some embodiments, multiple test blood samples are obtained from the subject at various time points before, during, or after the treatment. The level of EPHA7 in the post-treatment sample can then be compared with the level of EPHA7 in the pre-treatment sample or, alternatively, with a reference sample (e.g., a normal control level). For example, if the post-treatment EPHA7 level is lower than the pre-treatment EPHA7 level, one can conclude that the treatment was efficacious. Likewise, if the post-treatment EPHA7 level is similar to the normal control EPHA7 level, one can also conclude that the treatment was efficacious.

An “efficacious” treatment is one that leads to a reduction in the level of EPHA7 or a decrease in size, prevalence, or metastatic potential of lung cancer in a subject. When a treatment is applied prophylactically, “efficacious” means that the treatment retards or prevents occurrence of lung cancer or alleviates a clinical symptom of lung cancer. The assessment of lung cancer can be made using standard clinical protocols. Furthermore, the efficaciousness of a treatment can be determined in association with any known method for diagnosing or treating lung cancer. For example, lung cancer is routinely diagnosed histopathologically or by identifying symptomatic anomalies.

The diagnosis and detection of lung cancers have been encountering high difficulties. The present invention provides an ELISA for serum EPHA7 is a promising tool to screen lung cancer by combining with other serum makers, e.g. CEA and/or proGRP.

Components used to carry out the diagnosis of lung cancer according to the present invention can be combined in advance and supplied as a testing kit. Accordingly, the present invention provides a kit for detecting a lung cancer, comprising:

(i) an immunoassay reagent for determining a level of EPHA7 in a blood sample; and

(ii) a positive control sample for EPHA7.

In some embodiments, the kit of the present invention can further comprise:

(iii) an immunoassay reagent for determining a level of either of CEA and proGRP or both in a blood sample; and

(iv) a positive control sample for CEA and/or proGRP.

The reagents for the immunoassays which constitute a kit of the present invention can comprise reagents necessary for the various immunoassays described above. Specifically, the reagents for the immunoassays comprise an antibody that recognizes the substance to be measured. The antibody can be modified depending on the assay format of the immunoassay. ELISA can be used as an exemplary assay format of the present invention. In ELISA, for example, a first antibody immobilized onto a solid phase and a second antibody having a label are generally used.

Therefore, the immunoassay reagents for ELISA can comprise a first antibody immobilized onto a solid phase carrier. Fine particles or the inner walls of a reaction container can be used as the solid phase carrier. Magnetic particles can be used as the fine particles. Alternatively, multi-well plates for example, 96-well microplates are often used as the reaction containers. Containers for processing a large number of samples, which are equipped with wells having a smaller volume than in 96-well microplates at a high density, are also known. In the present invention, the inner walls of these reaction containers can be used as the solid phase carriers.

The immunoassay reagents for ELISA can further comprise a second antibody having a label. The second antibody for ELISA can be an antibody onto which an enzyme is directly or indirectly linked. Methods for chemically linking an enzyme to an antibody are known. For example, immunoglobulins can be enzymatically cleaved to obtain fragments comprising the variable regions. By reducing the —SS— bonds comprised in these fragments to —SH groups, bifunctional linkers can be attached. By linking an enzyme to the bifunctional linkers in advance, enzymes can be linked to the antibody fragments.

Alternatively, to indirectly link an enzyme, for example, the avidin-biotin binding can be used. That is, an enzyme can be indirectly linked to an antibody by contacting a biotinylated antibody with an enzyme to which avidin has been attached. In addition, an enzyme can be indirectly linked to a second antibody using a third antibody which is an enzyme-labeled antibody recognizing the second antibody. For example, enzymes for example, those exemplified above can be used as the enzymes to label the antibodies.

Kits of the present invention comprise a positive control for EPHA7. A positive control for EPHA7 comprises EPHA7 whose concentration has been determined in advance. Exemplary concentrations include, for example, a concentration set as the standard value in a testing method of the present invention. Alternatively, a positive control having a higher concentration can also be combined. The positive control for EPHA7 in the present invention can additionally comprise CEA and/or proGRP whose concentration has been determined in advance. A positive control comprising either CEA or proGRP, or both, and EPHA7 finds use as the positive control of the present invention.

Therefore, the present invention provides a positive control for detecting lung cancer, which comprises either CEA or proGRP, or both, in addition to EPHA7 at concentrations above a normal value. Alternatively, the present invention relates to the use of a blood sample comprising CEA and/or proGRP and EPHA7 at concentrations above a normal value in the production of a positive control for the detection of lung cancer. It has been known that CEA and proGRP can serve as an index for lung cancer. However, the use of EPHA7 as an index for lung cancer has not been described. Therefore, positive controls comprising EPHA7 in addition to CEA or proGRP were not known before the present invention. The positive controls of the present invention can be prepared by adding CEA and/or proGRP and EPHA7 at concentrations above a standard value to blood samples. For example, sera comprising CEA and/or proGRP and EPHA7 at concentrations above a standard value can be used as the positive controls of the present invention.

In some embodiments, the positive controls in the present invention are in a liquid form. In the present invention, blood samples are used as samples. Therefore, samples used as controls also need to be in a liquid form. Alternatively, by dissolving a dried positive control with a predefined amount of liquid at the time of use, a control that gives the tested concentration can be prepared. By packaging, together with a dried positive control, an amount of liquid necessary to dissolve it, the user can obtain the necessary positive control by just mixing them. EPHA7 used as the positive control can be a naturally-derived protein or it can be a recombinant protein. Similarly, for CEA, a naturally-derived protein can be used. Not only positive controls, but also negative controls can be combined in the kits of the present invention. The positive controls or negative controls are used to verify that the results indicated by the immunoassays are correct.

### Screening Methods

### (1) Test Compounds for Screening

In the context of the present invention, agents to be identified through the present screening methods can be any compound or composition including several compounds. Furthermore, the test agent exposed to a cell or protein according to the screening methods of the present invention can be a single compound or a combination of compounds. When a combination of compounds is used in the methods, the compounds can be contacted sequentially or simultaneously.

Any test agent, for example, cell extracts, cell culture supernatant, products of fermenting microorganism, extracts from marine organism, plant extracts, purified or crude proteins, peptides, non-peptide compounds, synthetic micro-molecular compounds (including nucleic acid constructs, for example, antisense RNA, siRNA, ribozymes, etc.) and natural compounds can be used in the screening methods of the present invention. The test agent of the present invention can be also obtained using any of the numerous approaches in combinatorial library methods known in the art, including

(1) biological libraries,

(2) spatially addressable parallel solid phase or solution phase libraries,

(3) synthetic library methods requiring deconvolution,

(4) the “one-bead one-compound” library method and

(5) synthetic library methods using affinity chromatography selection.

The biological library methods using affinity chromatography selection is limited to peptide libraries, while the other four approaches are applicable to peptide, non-peptide oligomer or small molecule libraries of compounds (Lam, Anticancer Drug Des 1997, 12: 145-67). Examples of methods for the synthesis of molecular libraries can be found in the art (DeWitt et al., Proc Natl Acad Sci USA 1993, 90: 6909-13; Erb et al., Proc Natl Acad Sci USA 1994, 91: 11422-6; Zuckermann et al., J Med Chem 37: 2678-85, 1994; Cho et al., Science 1993, 261: 1303-5; Carell et al., Angew Chem Int Ed Engl 1994, 33: 2059; Carell et al., Angew Chem Int Ed Engl 1994, 33: 2061; Gallop et al., J Med Chem 1994, 37: 1233-51). Libraries of compounds can be presented in solution (see Houghten, Bio/Techniques 1992, 13: 412-21) or on beads (Lam, Nature 1991, 354: 82-4), chips (Fodor, Nature 1993, 364: 555-6), bacteria (U.S. Pat. No. 5,223,409), spores (U.S. Pat. Nos. 5,571,698; 5,403,484 and 5,223,409), plasmids (Cull et al., Proc Natl Acad Sci USA 1992, 89: 1865-9) or phage (Scott and Smith, Science 1990, 249: 386-90; Devlin, Science 1990, 249: 404-6; Cwirla et al., Proc Natl Acad Sci USA 1990, 87: 6378-82; Felici, J Mol Biol 1991, 222: 301-10; US Pat. Application 2002-103360).

A compound in which a part of the structure of the compound screened by any of the present screening methods is converted by addition, deletion and/or replacement, is included in the agents obtained by the screening methods of the present invention.

Furthermore, when the screened test agent is a protein, for obtaining a DNA encoding the protein, either the whole amino acid sequence of the protein can be determined to deduce the nucleic acid sequence coding for the protein, or partial amino acid sequence of the obtained protein can be analyzed to prepare an oligo DNA as a probe based on the sequence, and screen cDNA libraries with the probe to obtain a DNA encoding the protein. The obtained DNA finds use in preparing the test agent which is a candidate for treating or preventing cancer.

Test agents useful in the screening described herein can also be antibodies or non-antibody binding proteins that specifically bind to the CX protein or partial CX peptides that lack the activity to binding for partner or the activity to phosphorylate a substrate or phosphorylated by kinases in vivo. Such partial protein or antibody can be prepared by the methods described herein (see (1) Cancer-related genes and cancer-related protein, and functional equivalent thereof in Definition or Antibodies) and can be tested for their ability to block phosphorylation of the CX protein or binding of the protein (e.g., EPHA7/EGFR, STK31 or WDHD1) with its binding partners.

### (i) Molecular Modeling

Construction of test agent libraries is facilitated by knowledge of the molecular structure of compounds known to have the properties sought, and/or the molecular structure of the target molecules to be inhibited, i.e., CDCA5, EPHA7, STK31 or WDHD1. One approach to preliminary screening of test agents suitable for further evaluation is computer modeling of the interaction between the test agent and its target.

Computer modeling technology allows the visualization of the three-dimensional atomic structure of a selected molecule and the rational design of new compounds that will interact with the molecule. The three-dimensional construct typically depends on data from x-ray crystallographic analysis or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

An example of the molecular modeling system described generally above includes the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, for example, Rotivinen et al. Acta Pharmaceutica Fennica 1988, 97: 159-66; Ripka, New Scientist 1988, 54-8; McKinlay & Rossmann, Annu Rev Pharmacol Toxiciol 1989, 29: 111-22; Perry & Davies, Prog Clin Biol Res 1989, 291: 189-93; Lewis & Dean, Proc R Soc Lond 1989, 236: 125-40, 141-62; and, with respect to a model receptor for nucleic acid components, Askew et al., J Am Chem Soc 1989, 111: 1082-90.

Other computer programs that screen and graphically depict chemicals are available from companies for example, BioDesign, Inc., Pasadena, Calif., Allelix, Inc, Mississauga, Ontario, Canada, and Hypercube, Inc., Cambridge, Ontario. See, e.g., DesJarlais et al., J Med Chem 1988, 31: 722-9; Meng et al., J Computer Chem 1992, 13: 505-24; Meng et al., Proteins 1993, 17: 266-78; Shoichet et al., Science 1993, 259: 1445-50.

Once an inhibitor of the CX activity has been identified, combinatorial chemistry techniques can be employed to construct any number of variants based on the chemical structure of the identified inhibitor, as detailed below. The resulting library of candidate inhibitors, or “test agents” can be screened using the methods of the present invention to identify test agents of the library that disrupt the CDCA5, EPHA7, STK31 or WDHD1 activity.

### (ii) Combinatorial Chemical Synthesis

Combinatorial libraries of test agents can be produced as part of a rational drug design program involving knowledge of core structures existing in known inhibitors of the CDCA5, EPHA7, STK31 or WDHD1 activity. This approach allows the library to be maintained at a reasonable size, facilitating high throughput screening. Alternatively, simple, particularly short, polymeric molecular libraries can be constructed by simply synthesizing all permutations of the molecular family making up the library. An example of this latter approach would be a library of all peptides six amino acids in length. Such a peptide library could include every 6 amino acid sequence permutation. This type of library is termed a linear combinatorial chemical library.

Preparation of combinatorial chemical libraries is well known to those of skill in the art, and can be generated by either chemical or biological synthesis. Combinatorial chemical libraries include, but are not limited to, peptide libraries (see, e.g., U.S. Pat. No. 5,010,175; Furka, Int J Pept Prot Res 1991, 37: 487-93; Houghten et al., Nature 1991, 354: 84-6). Other chemistries for generating chemical diversity libraries can also be used. Such chemistries include, but are not limited to: peptides (e.g., PCT Publication No. WO 91/19735), encoded peptides (e.g., WO 93/20242), random bio-oligomers (e.g., WO 92/00091), benzodiazepines (e.g., U.S. Pat. No. 5,288,514), diversomers for example, hydantoins, benzodiazepines and dipeptides (DeWitt et al., Proc Natl Acad Sci USA 1993, 90:6909-13), vinylogous polypeptides (Hagihara et al., J Amer Chem Soc 1992, 114: 6568), nonpeptidal peptidomimetics with glucose scaffolding (Hirschmann et al., J Amer Chem Soc 1992, 114: 9217-8), analogous organic syntheses of small compound libraries (Chen et al., J. Amer Chem Soc 1994, 116: 2661), oligocarbamates (Cho et al., Science 1993, 261: 1303), and/or peptidylphosphonates (Campbell et al., J Org Chem 1994, 59: 658), nucleic acid libraries (see Ausubel, Current Protocols in Molecular Biology, 1990-2008, John Wiley Interscience; Sambrook and Russell, Molecular Cloning: A Laboratory Manual, 3^{rd} Ed., 2001, Cold Spring Harbor Laboratory, New York, USA), peptide nucleic acid libraries (see, e.g., U.S. Pat. No. 5,539,083), antibody libraries (see, e.g., Vaughan et al., Nature Biotechnology 1996, 14(3):309-14 and PCT/US96/10287), carbohydrate libraries (see, e.g., Liang et al., Science 1996, 274: 1520-22; U.S. Pat. No. 5,593,853), and small organic molecule libraries (see, e.g., benzodiazepines, Gordon E M. Curr Opin Biotechnol. 1995 Dec. 1; 6(6):624-31; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

(iii) Phage Display

Another approach uses recombinant bacteriophage to produce libraries. Using the “phage method” (Scott & Smith, Science 1990, 249: 386-90; Cwirla et al., Proc Natl Acad Sci USA 1990, 87: 6378-82; Devlin et al., Science 1990, 249: 404-6), very large libraries can be constructed (e.g., 106-108 chemical entities). A second approach uses primarily chemical methods, of which the Geysen method (Geysen et al., Molecular Immunology 1986, 23: 709-15; Geysen et al., J Immunologic Method 1987, 102: 259-74); and the method of Fodor et al. (Science 1991, 251: 767-73) are examples. Furka et al. (14th International Congress of Biochemistry 1988, Volume #5, Abstract FR:013; Furka, Int J Peptide Protein Res 1991, 37: 487-93), Houghten (U.S. Pat. No. 4,631,211) and Rutter et al. (U.S. Pat. No. 5,010,175) describe methods to produce a mixture of peptides that can be tested as agonists or antagonists.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

### (2) Screening Methods

### (i) General Screening Method

For screening of compounds that bind to a CX protein, in immunoprecipitation, an immune complex is formed by adding these antibodies or non-antibody binding proteins to a cell lysate prepared using an appropriate detergent. The immune complex consists of a polypeptide, a polypeptide having a binding affinity for the polypeptide, and an antibody or non-antibody binding protein. Immunoprecipitation can be also conducted using antibodies against a polypeptide, in addition to using antibodies against the above epitopes, which antibodies can be prepared as described above (see Antibodies).

An immune complex can be precipitated, for example, by Protein A sepharose or Protein G sepharose when the antibody is a mouse IgG antibody. If the polypeptide of the present invention is prepared as a fusion protein with an epitope, for example GST, an immune complex can be formed in the same manner as in the use of the antibody against the polypeptide, using a substance specifically binding to these epitopes, for example glutathione-Sepharose 4B.

Immunoprecipitation can be performed by following or according to, for example, the methods in the literature (Harlow and Lane, Antibodies, 511-52, Cold Spring Harbor Laboratory publications, New York (1988)).

SDS-PAGE is commonly used for analysis of immunoprecipitated proteins and the bound protein can be analyzed by the molecular weight of the protein using gels with an appropriate concentration. Since the protein bound to the polypeptide is difficult to detect by a common staining method, for example Coomassie staining or silver staining, the detection sensitivity for the protein can be improved by culturing cells in culture medium containing radioactive isotope, ³⁵S-methionine or ³⁵S-cysteine, labeling proteins in the cells, and detecting the proteins. The target protein can be purified directly from the SDS-polyacrylamide gel and its sequence can be determined, when the molecular weight of a protein has been revealed.

As a method for screening for proteins that bind to the CX polypeptide using the polypeptide, for example, West-Western blotting analysis (Skolnik et al., Cell 65: 83-90 (1991)) can be used. Specifically, a protein binding to the CX polypeptide can be obtained by preparing a cDNA library from cells, tissues, organs (see (1) Cancer-related genes and cancer-related protein, and functional equivalent thereof in Definition), or cultured cells expected to express a protein binding to the CX polypeptide using a phage vector (e.g., ZAP), expressing the protein on LB-agarose, fixing the protein expressed on a filter, reacting the purified and labeled CX polypeptide with the above filter, and detecting the plaques expressing proteins bound to the CX polypeptide according to the label. The CX polypeptide can be labeled by utilizing the binding between biotin and avidin, or by utilizing an antibody that specifically binds to the CX polypeptide, or a peptide or polypeptide (for example, GST) that is fused to the CX polypeptide. Methods using radioisotope or fluorescence and such can be also used.

The terms “label” and “detectable label” are used herein to refer to any composition detectable by spectroscopic, photochemical, biochemical, immunochemical, electrical, optical or chemical means. Such labels include biotin for staining with labeled streptavidin conjugate, magnetic beads (e.g., DYNABEADS™), fluorescent dyes (e.g., fluorescein, Texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels for example colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,275,149; and 4,366,241. Means of detecting such labels are well known to those of skill in the art. Thus, for example, radiolabels can be detected using photographic film or scintillation counters, fluorescent markers can be detected using a photodetector to detect emitted light. Enzymatic labels are typically detected by providing the enzyme with a substrate and detecting, the reaction product produced by the action of the enzyme on the substrate, and calorimetric labels are detected by simply visualizing the colored label.

Alternatively, in another embodiment of the screening method of the present invention, a two-hybrid system utilizing cells can be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet 10: 286-92 (1994)”).

In the two-hybrid system, the polypeptide of the invention is fused to the SRF-binding region or GAL4-binding region and expressed in yeast cells. A cDNA library is prepared from cells expected to express a protein binding to the polypeptide of the invention, such that the library, when expressed, is fused to the VP16 or GAL4 transcriptional activation region. The cDNA library is then introduced into the above yeast cells and the cDNA derived from the library is isolated from the positive clones detected (when a protein binding to the polypeptide of the invention is expressed in yeast cells, the binding of the two activates a reporter gene, making positive clones detectable). A protein encoded by the cDNA can be prepared by introducing the cDNA isolated above to E. coli and expressing the protein.

As a reporter gene, for example, Ade2 gene, lacZ gene, CAT gene, luciferase gene and such can be used in addition to the HIS3 gene.

A compound binding to CX polypeptide can also be screened using affinity chromatography. For example, the CX polypeptide can be immobilized on a carrier of an affinity column, and a test compound, containing a protein capable of binding to the CX polypeptide, is applied to the column. A test compound herein can be, for example, cell extracts, cell lysates, etc. After loading the test compound, the column is washed, and compounds bound to the CX polypeptide can be prepared.

When the test compound is a protein, the amino acid sequence of the obtained protein is analyzed, an oligo DNA is synthesized based on the sequence, and cDNA libraries are screened using the oligo DNA as a probe to obtain a DNA encoding the protein.

A biosensor using the surface plasmon resonance phenomenon can be used as a means for detecting or quantifying the bound compound in the present invention. When such a biosensor is used, the interaction between the CX polypeptide and a test compound can be observed real-time as a surface plasmon resonance signal, using only a minute amount of polypeptide and without labeling (for example, BIAcore, Pharmacia). Therefore, it is possible to evaluate the binding between the CX polypeptide and a test compound using a biosensor, for example, BIAcore.

As a method of screening for compounds that inhibit the binding between a CXprotein and a binding partner thereof (e.g., EPHA7/EGFR, CDCA5/CDC2, CDCA5/ERK, STK31/c-raf, STK31/MEK and STK31/ERK), many methods well known by one skilled in the art can be used. For example, screening can be carried out as an in vitro assay system, for example, a cellular system. More specifically, first, either the CX protein or the binding partner thereof is bound to a support, and the other protein is added together with a test compound thereto. For instance, either the CDCA5 polypeptide, CDC2 polypeptide or ERK polypeptide is bound to a support, and the binding partner polypeptide is added together with a test compound thereto. Next, the mixture is incubated, washed and the other protein bound to the support is detected and/or measured.

In the context of the present invention, “inhibition of binding” between two proteins refers to at least reducing binding between the proteins. Thus, in some cases, the percentage of binding pairs in a sample in the presence of a test agent will be decreased compared to an appropriate (e.g., not treated with test compound or from a non-cancer sample, or from a cancer sample) control. The reduction in the amount of proteins bound can be, e.g., less than 90%, 80%, 70%, 60%, 50%, 40%, 25%, 10%, 5%, 1% or less (e.g., 0%), than the pairs bound in a control sample.

Examples of supports that can be used for binding proteins include, for example, insoluble polysaccharides, for example, agarose, cellulose and dextran; and synthetic resins, for example, polyacrylamide, polystyrene and silicon; for example, commercial available beads and plates (e.g., multi-well plates, biosensor chip, etc.) prepared from the above materials can be used. When using beads, they can be filled into a column. Alternatively, the use of magnetic beads is also known in the art, and enables one to readily isolate proteins bound on the beads via magnetism.

The binding of a protein to a support can be conducted according to routine methods, for example, chemical bonding and physical adsorption, for example. Alternatively, a protein can be bound to a support via antibodies that specifically recognize the protein. Moreover, binding of a protein to a support can be also conducted by means of avidin and biotin.

The methods of screening for molecules that bind when the immobilized polypeptide is exposed to synthetic chemical compounds, or natural substance banks, or a random phage peptide display library, and the methods of screening using high-throughput based on combinatorial chemistry techniques (Wrighton et al., Science 273: 458-63 (1996); Verdine, Nature 384: 11-3 (1996)) to isolate not only proteins but chemical compounds that bind to the protein (including agonist and antagonist) are well known to one skilled in the art.

Furthermore, the phosphorylation level of a polypeptide or functional equivalent thereof can be detected according to any method known in the art. For example, a test compound is contacted with the polypeptide expressing cell, the cell is incubated for a sufficient time to allow phosphorylation of the polypeptide, and then, the amount of phosphorylated polypeptide can be detected. Alternatively, a test compound is contacted with the polypeptide in vitro, the polypeptide is incubated under condition that allows phosphorylation of the polypeptide, and then, the amount of phosphorylated polypeptide can be detected (see (14) In vitro and in vivo kinase assay).

In the present invention, the conditions suitable for the phosphorylation can be provided with an incubation of substrate and enzyme protein in the presence of phosphate donor, e.g. ATP. The conditions suitable for the phosphorylation also include conditions in culturing cells expressing the polypeptides. For example, the cell is a transformant cell harboring an expression vector comprising a polynucleotide encoding the CX polypeptide (see (1) Cancer-related genes and cancer-related protein, and functional equivalent thereof in Definition). After the incubation, the phosphorylation level of the substrate can be detected, for example, with an antibody recognizing phosphorylated substrate or by detecting labeled gamma-phosphate transferred by the ATP phosphate donor. Prior to the detection of phosphorylated substrate, substrate can be separated from other elements, or cell lysate of transformant cells. For instance, gel electrophoresis can be used for separation of substrate. Alternatively, substrate can be captured by contacting with a carrier having an antibody against substrate.

For detection of phosphorylated protein, SDS-PAGE or immunoprecipitation can be used. Furthermore, an antibody that recognizes a phosphorylated residue or transferred labeled phosphate can be used for detecting phosphorylated protein level. Any immunological techniques using an antibody recognizing the phosphorylated polypeptide can be used for the detection. ELISA or immunoblotting with antibodies recognizing phosphorylated polypeptide can be used for the present invention. When a labeled phosphate donor is used, the phosphorylation level of the substrate can be detected via tracing the label. For example, radio-labeled ATP (e.g. ³²P-ATP) can be used as phosphate donor, wherein radioactivity of the separated substrate correlates with the phosphorylation level of the substrate. Alternatively, an antibody specifically recognizing a phosphorylated substrate from un-phosphorylated substrate can be used for detection phosphorylated substrate.

If the detected amount of phosphorylated CX polypeptide contacted with a test compound is decreased to the amount detected in not contacted with the test compound, the test compound is deemed to inhibit polypeptide phosphorylation of a CX protein and thus have lung cancer and/or esophageal cancer suppressing ability. Herein, a phosphorylation level can be deemed to be “decreased” when it decreases by, for example, 10%, 25%, or 50% from, or at least 0.1 fold, at least 0.2 fold, at least 1 fold, at least 2 fold, at least 5 fold, or at least 10 fold or more compared to that detected for cells not contacted with the test agent. For example, Student's t-test, the Mann-Whitney U-test, or ANOVA can be used for statistical analysis.

Furthermore, the expression level of a polypeptide or functional equivalent thereof can be detected according to any method known in the art. For example, a reporter assay can be used. Suitable reporter genes and host cells are well known in the art. The reporter construct required for the screening can be prepared by using the transcriptional regulatory region of CX gene or downstream gene thereof. When the transcriptional regulatory region of the gene has been known to those skilled in the art, a reporter construct can be prepared by using the previous sequence information. When the transcriptional regulatory region remains unidentified, a nucleotide segment containing the transcriptional regulatory region can be isolated from a genome library based on the nucleotide sequence information of the gene. Specifically, the reporter construct required for the screening can be prepared by connecting reporter gene sequence to the transcriptional regulatory region of a CX gene of interest. The transcriptional regulatory region of a CX gene is the region from a start codon to at least 500 bp upstream, for example, 1000 bp, for example, 5000 or 10000 bp upstream. A nucleotide segment containing the transcriptional regulatory region can be isolated from a genome library or can be propagated by PCR. Methods for identifying a transcriptional regulatory region, and also assay protocol are well known (Sambrook and Russell, Molecular Cloning: A Laboratory Manual, 3rd Ed., Chapter 17, 2001, Cold Springs Harbor Laboratory Press).

Various low-throughput and high-throughput enzyme assay formats are known in the art and can be readily adapted for detection or measuring of the phosphorylation level of the CX polypeptide. For high-throughput assays, the substrate can conveniently be immobilized on a solid support. Following the reaction, the phosphorylated substrate can be detected on the solid support by the methods described above. Alternatively, the contact step can be performed in solution, after which the substrate can be immobilized on a solid support, and the phosphorylated substrate detected. To facilitate such assays, the solid support can be coated with streptavidin and the substrate labeled with biotin, or the solid support can be coated with antibodies against the substrate. The skilled person can determine suitable assay formats depending on the desired throughput capacity of the screen.

The assays of the invention are also suitable for automated procedures which facilitate high-throughput screening. A number of well-known robotic systems have been developed for solution phase chemistries. These systems include automated workstations like the automated synthesis apparatus developed by Takeda Chemical Industries, Ltd. (Osaka, Japan) and many robotic systems utilizing robotic arms (Zymate II, Zymark Corporation, Hopkinton, Mass.; Orca, Hewlett Packard, Palo Alto, Calif.), which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

(ii) Screening for Compounds that Bind to CX Protein(s)

In present invention, over-expression of CDCA5 in lung cancer and esophageal cancer was detected in spite of no expression in normal organ except testis (FIG. 1); over-expression of EPHA7 in lung cancer and esophageal cancer was detected in spite of no expression in normal organ except fetal brain and fetal kidney (FIG. 3); over-expression of STK31 in lung cancer and esophageal cancer was detected in spite of no expression in normal organ except testis (FIG. 9); over-expression of WDHD1 in lung cancer and esophageal cancer was detected in spite of no expression in normal organ except testis (FIGS. 13, 14A and B). Therefore, using the CDCA5, EPHA7, STK31 or WDHD1 gene, proteins encoded by the gene or transcriptional regulatory region of the gene, compounds can be screened that alter the expression of the gene or the biological activity of a polypeptide encoded by the gene. Such compounds are used as pharmaceuticals for treating or preventing lung cancer and esophageal cancer or detecting agents for diagnosing lung cancer and esophageal cancer and assessing a prognosis of lung cancer and/or esophageal cancer patient.

Specifically, the present invention provides the method of screening for an agent useful in diagnosing, treating or preventing cancers using the CDCA5, EPHA7, STK31 or WDHD1 polypeptide. An embodiment of this screening method comprises the steps of:

(a) contacting a test agent with a polypeptide selected from the group consisting of CDCA5, EPHA7, STK31 and WDHD1 protein, or fragment thereof;

(b) detecting binding between the polypeptide and said test agent;

(c) selecting the test agent that binds to said polypeptides of step (a).

The method of the present invention will be described in more detail below.

The CDCA5, EPHA7, STK31 and WDHD1 polypeptide to be used for screening can be a recombinant polypeptide or a protein derived from the nature or a partial peptide thereof. The polypeptide to be contacted with a test compound can be, for example, a purified polypeptide, a soluble protein, a form bound to a carrier or a fusion protein fused with other polypeptides.

As a method of screening for proteins, for example, that bind to the CDCA5, EPHA7, STK31 and WDHD1 polypeptide using the CDCA5, EPHA7, STK31 and WDHD1 polypeptide, many methods well known by a person skilled in the art can be used. Such a screening can be conducted by, for example, immunoprecipitation method. The gene encoding the CDCA5, EPHA7, STK31 and WDHD1 polypeptide is expressed in host (e.g., animal) cells and so on by inserting the gene to an expression vector for foreign genes, for example, pSV2neo, pcDNA I, pcDNA3.1, pCAGGS and pCD8.

The promoter to be used for the expression can be any promoter that can be used commonly and include, for example, the SV40 early promoter (Rigby in Williamson (ed.), Genetic Engineering, vol. 3. Academic Press, London, 83-141 (1982)), the EF-alpha promoter (Kim et al., Gene 91: 217-23 (1990)), the CAG promoter (Niwa et al., Gene 108: 193 (1991)), the RSV LTR promoter (Cullen, Methods in Enzymology 152: 684-704 (1987)) the SR alpha promoter (Takebe et al., Mol Cell Biol 8: 466 (1988)), the CMV immediate early promoter (Seed and Aruffo, Proc Natl Acad Sci USA 84: 3365-9 (1987)), the SV40 late promoter (Gheysen and Fiers, J Mol Appl Genet 1: 385-94 (1982)), the Adenovirus late promoter (Kaufman et al., Mol Cell Biol 9: 946 (1989)), the HSV TK promoter and so on.

The introduction of the gene into host cells to express a foreign gene can be performed according to any methods, for example, the electroporation method (Chu et al., Nucleic Acids Res 15: 1311-26 (1987)), the calcium phosphate method (Chen and Okayama, Mol Cell Biol 7: 2745-52 (1987)), the DEAE dextran method (Lopata et al., Nucleic Acids Res 12: 5707-17 (1984); Sussman and Milman, Mol Cell Biol 4: 1641-3 (1984)), the Lipofectin method (Derijard B., Cell 76: 1025-37 (1994); Lamb et al., Nature Genetics 5: 22-30 (1993): Rabindran et al., Science 259: 230-4 (1993)) and so on.

The polypeptide encoded by CDCA5, EPHA7, STK31 and WDHD1 gene can be expressed as a fusion protein comprising a recognition site (epitope) of a monoclonal antibody by introducing the epitope of the monoclonal antibody, whose specificity has been revealed, to the N- or C-terminus of the polypeptide. A commercially available epitope-antibody system can be used (Experimental Medicine 13: 85-90 (1995)). Vectors which can express a fusion protein with, for example, b-galactosidase, maltose binding protein, glutathione S-transferase, green florescence protein (GFP) and so on by the use of its multiple cloning sites are commercially available. Also, a fusion protein prepared by introducing only small epitopes consisting of several to a dozen amino acids so as not to change the property of the CX polypeptide by the fusion is also reported. Epitopes, for example, polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage) and such, and monoclonal antibodies recognizing them can be used as the epitope-antibody system for screening proteins binding to the CX polypeptide (Experimental Medicine 13: 85-90 (1995)).

In immunoprecipitation, an immune complex is formed by adding these antibodies to cell lysate prepared using an appropriate detergent. The immune complex consists of the CX polypeptide, a polypeptide comprising the binding ability with the polypeptide, and an antibody. Immunoprecipitation can be also conducted using antibodies against the CX polypeptide, besides using antibodies against the above epitopes, which antibodies can be prepared as described above. An immune complex can be precipitated, for example by Protein A sepharose or Protein G sepharose when the antibody is a mouse IgG antibody. If the polypeptide encoded by CX gene is prepared as a fusion protein with an epitope, for example, GST, an immune complex can be formed in the same manner as in the use of the antibody against the CX polypeptide, using a substance specifically binding to these epitopes, for example, glutathione-Sepharose 4B.

Immunoprecipitation can be performed by following or according to, for example, the methods in the literature (Harlow and Lane, Antibodies, 511-52, Cold Spring Harbor Laboratory publications, New York (1988)).

SDS-PAGE is commonly used for analysis of immunoprecipitated proteins and the bound protein can be analyzed by the molecular weight of the protein using gels with an appropriate concentration. Since the protein bound to the CDCA5, EPHA7, STK31 and WDHD1 polypeptide is difficult to detect by a common staining method, for example, Coomassie staining or silver staining, the detection sensitivity for the protein can be improved by culturing cells in culture medium containing radioactive isotope, ³⁵S-methionine or ³⁵S-cystein, labeling proteins in the cells, and detecting the proteins. The target protein can be purified directly from the SDS-polyacrylamide gel and its sequence can be determined, when the molecular weight of a protein has been revealed.

As a method of screening for proteins binding to the CDCA5, EPHA7, STK31 and WDHD1 polypeptide using the polypeptide, for example, West-Western blotting analysis (Skolnik et al., Cell 65: 83-90 (1991)) can be used. Specifically, a protein binding to the CX polypeptide can be obtained by preparing a cDNA library from cultured cells (e.g., lung cancer cell line or esophageal cancer cell line) expected to express a protein binding to the CX polypeptide using a phage vector (e.g., ZAP), expressing the protein on LB-agarose, fixing the protein expressed on a filter, reacting the purified and labeled CX polypeptide with the above filter, and detecting the plaques expressing proteins bound to the CDCA5, EPHA7, STK31 and WDHD1 polypeptide according to the label. The polypeptide of the invention can be labeled by utilizing the binding between biotin and avidin, or by utilizing an antibody that specifically binds to the CDCA5, EPHA7, STK31 and WDHD1 polypeptide, or a peptide or polypeptide (for example, GST) that is fused to the CDCA5, EPHA7, STK31 and WDHD1 polypeptide. Methods using radioisotope or fluorescence and such can be also used.

Alternatively, in another embodiment of the screening method of the present invention, a two-hybrid system utilizing cells can be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet 10: 286-92 (1994)”).

In the two-hybrid system, the polypeptide of the invention is fused to the SRF-binding region or GAL4-binding region and expressed in yeast cells. A cDNA library is prepared from cells expected to express a protein binding to the polypeptide of the invention, such that the library, when expressed, is fused to the VP16 or GAL4 transcriptional activation region. The cDNA library is then introduced into the above yeast cells and the cDNA derived from the library is isolated from the positive clones detected (when a protein binding to the polypeptide of the invention is expressed in yeast cells, the binding of the two activates a reporter gene, making positive clones detectable). A protein encoded by the cDNA can be prepared by introducing the cDNA isolated above to E. coli and expressing the protein. As a reporter gene, for example, Ade2 gene, lacZ gene, CAT gene, luciferase gene and such can be used in addition to the HIS3 gene.

A compound binding to the polypeptide encoded by CX gene can also be screened using affinity chromatography. For example, the polypeptide of the invention can be immobilized on a carrier of an affinity column, and a test compound, containing a protein capable of binding to the polypeptide of the invention, is applied to the column. A test compound herein can be, for example, cell extracts, cell lysates, etc. After loading the test compound, the column is washed, and compounds bound to the polypeptide of the invention can be prepared. When the test compound is a protein, the amino acid sequence of the obtained protein is analyzed, an oligo DNA is synthesized based on the sequence, and cDNA libraries are screened using the oligo DNA as a probe to obtain a DNA encoding the protein.

A biosensor using the surface plasmon resonance phenomenon can be used as a mean for detecting or quantifying the bound compound in the present invention. When such a biosensor is used, the interaction between the polypeptide of the invention and a test compound can be observed real-time as a surface plasmon resonance signal, using only a minute amount of polypeptide and without labeling (for example, BIAcore, Pharmacia). Therefore, it is possible to evaluate the binding between the polypeptide of the invention and a test compound using a biosensor for example, BIAcore.

The methods of screening for molecules that bind when the immobilized CX polypeptide is exposed to synthetic chemical compounds, or natural substance banks or a random phage peptide display library, and the methods of screening using high-throughput based on combinatorial chemistry techniques (Wrighton et al., Science 273: 458-64 (1996); Verdine, Nature 384: 11-13 (1996); Hogan, Nature 384: 17-9 (1996)) to isolate not only proteins but chemical compounds that bind to the CX protein (including agonist and antagonist) are well known to one skilled in the art.

(iii) Screening for Compound that Suppress the Biological Activity of CX Gene(s)

In the present invention, the CDCA5 protein has the activity of promoting cell proliferation of cancer cells (FIG. 2) and phosphorylation activity (FIG. 17C); EPHA7 protein has the activity of promoting cell proliferation of cells (FIG. 6), the activity of promoting cell invasion (FIG. 7), the binding activity to EGFR (FIG. 8B), the kinase activity to EGFR (Tyr-845, Tyr-1068, Tyr-1086, Tyr-1173) (FIG. 8A, 20E, 21) and the activity of promoting phosphorylation of PLCgamma (Tyr783), CDC25 (Ser-216), MET (Tyr-1230/1234/1235, Tyr-1313, Tyr-1349, Tyr-1365) (GenBank Accession No.: NM_{—}000245, SEQ ID NO.: 56) (FIG. 8A, FIG. 21); STK31 protein has the activity of promoting cell proliferation of cancer cells (FIG. 11), the kinase activity (FIG. 12A) and the activity of promoting phosphorylation of EGFR(Ser1046/1047), ERK (ERK1/2, P44/42 MAPK) (Thr202/Thr204) and MEK (FIG. 12B, D); WDHD1 protein has the activity of promoting cell proliferation of cancer cells (FIG. 15A), the promoting activity of cell viability (FIG. 15C) and phosphorylation activity (FIG. 16A). Using this biological activity, a compound which inhibits this activity of this protein can be screened. Therefore, the present invention provides a method of screening for a compound for treating or preventing cancers expressing CDCA5, EPHA7, STK31 or WDHD1 gene, e.g. lung cancers (non-small cell lung cancer or small cell lung cancer) or esophageal cancer, using the polypeptide encoded by CDCA5, EPHA7, STK31 or WDHD1 gene.

Specifically, the present invention provides the following methods of [1] to [19]:

[1] A method of screening for an agent useful in treating or preventing cancers expressing at least one gene elected from the group consisting of CDCA5, EPHA7, STK31 and WDHD1, said method comprising the steps of:

(a) contacting a test agent with a cell expressing a polynucleotide encoding a polypeptide encoded by the gene expressing in cancer, or functional equivalent thereof;

(b) detecting a level of said polynucleotide or polypeptide of step (a);

(c) comparing said level detected in the step (b) with those detected in the absence of the test agent; and

(d) selecting the test agent that reduce or inhibit said level of (c).

[2] The method of [1], wherein said level is detected by any one of the method select from the group consisting of:

(a) detecting the amount of the mRNA encoding the polypeptide selected from the group consisting of CDCA5, EPHA7, STK31 and WDHD1 polypeptide, or functional equivalent thereof;

(b) detecting the amount of the polypeptide selected from the group consisting of CDCA5, EPHA7, STK31 and WDHD1 polypeptide, or functional equivalent thereof; and

(c) detecting the biological activity of the polypeptide selected from the group consisting of CDCA5, EPHA7, STK31 and WDHD1 polypeptide, or functional equivalent thereof.

[3] The method of [2], wherein the biological activity is any one of the activity select from the group consisting of:

(a) a proliferation activity of the cell expressing a polypeptide selected from the group consisting of CDCA5, EPHA7, STK31 and WDHD1 polypeptide, or functional equivalent thereof;

(b) an invasion activity of the cell expressing an EPHA7 polypeptide or functional equivalent thereof; and

(c) a kinase activity of a polypeptide selected from the group consisting of EPHA7 and STK31 polypeptide, or functional equivalent thereof.

The method of the present invention will be described in more detail below.

Any polypeptides can be used for screening so long as they comprise the biological activity of the CDCA5, EPHA7, STK31 or WDHD1 protein. Such biological activity includes the cell-proliferating activity for CDCA5, EPHA7, STK31 or WDHD1; the activity of promoting cell invasion for EPHA7; the EGFR-binding activity for EPHA7; or extracellular secretion activity for the EPHA7 protein; the kinase activity for EPHA7 or STK31; the phosphorylation activity for WDHD1 or the promoting activity of cell viability for WDHD1. For example, CDCA5, EPHA7, STK31 or WDHD1 protein can be used and polypeptides functionally equivalent to these proteins can also be used. Such polypeptides can be expressed endogenously or exogenously by cells.

The compound isolated by this screening is a candidate for antagonists of the polypeptide encoded by CDCA5, EPHA7, STK31 or WDHD1 gene. The term “antagonist” refers to molecules that inhibit the function of the polypeptide by binding thereto. Said term also refers to molecules that reduce or inhibit expression of the gene encoding CDCA5, EPHA7, STK31 or WDHD1. Moreover, a compound isolated by this screening is a candidate for compounds which inhibit the in vivo interaction of the CDCA5, EPHA7, STK31 or WDHD1 polypeptide with molecules (including DNAs and proteins).

When the biological activity to be detected in the present method is cell proliferation, it can be detected, for example, by preparing cells which express the polypeptide selected from the group consisting of CDCA5, EPHA7, STK31 or WDHD1, culturing the cells in the presence of a test compound, and determining the speed of cell proliferation, measuring the cell cycle and such, as well as by measuring the colony formation activity, e.g. MTT assay, colony formation assay or FACS shown in [EXAMPLE 2-5].

When the biological activity to be detected in the present method is extracellular secretion of EPHA7, it can be detected, for example, by amount of the EPHA7 protein in the culture medium, culturing the cells which express the EPHA7 polypeptide in the presence of a test compound, for example, shown in FIG. 2G, lower panel.

The term of “suppress the biological activity” as defined herein refers to at least 10% suppression of the biological activity of CDCA5, EPHA7, STK31 or WDHD1 in comparison with in absence of the compound, for example, at least 25%, 50% or 75% suppression, for example, at least 90% suppression.

(iv) Screening for Compounds that Alter the Expression of CX Gene(s)

In the present invention, the decrease of the expression of CX gene(s) by a double-stranded molecule specific for CX gene(s) causes inhibiting cancer cell proliferation (FIG. 2 for CDCA5; FIG. 6 for EPHA7; FIG. 11 for STK31; and FIG. 15 for WDHD1). Therefore, compounds that can be used in the treatment or prevention of bladder cancer can be identified through screenings that use the expression levels of CX gene(s) as indices. In the context of the present invention, such screening can comprise, for example, the following steps:

(a) contacting a candidate compound with a cell expressing CDCA5, EPHA7, STK31 or WDHD; and

(b) selecting the candidate compound that reduces the expression level of CDCA5, EPHA7, STK31 or WDHD as compared to a control.

The method of the present invention will be described in more detail below.

Cells expressing the CDCA5, EPHA7, STK31 or WDHD include, for example, cell lines established from lung cancer or esophageal cancer; such cells can be used for the above screening of the present invention (e.g., A549 and LC319 for CDCA5; NCI-H520 and SBC-5 for EPHA7; LC319 and NCI-H2170 for STK31; and LC319 and TE9 for WDHD1). The expression level can be estimated by methods well known to one skilled in the art, for example, RT-PCR, Northern bolt assay, Western bolt assay, immunostaining, ELISA or flow cytometry analysis. The term of “reduce the expression level” as defined herein refers to at least 10% reduction of expression level of CDCA5, EPHA7, STK31 or WDHD in comparison to the expression level in absence of the compound, for example, at least 25%, 50% or 75% reduced level, for example, at least 95% reduced level. The compound herein includes chemical compound, double-strand nucleotide, and so on. The preparation of the double-strand nucleotide is in aforementioned description. In the method of screening, a compound that reduces the expression level of CDCA5, EPHA7, STK31 or WDHD can be selected as candidate agents to be used for the treatment or prevention of cancers, e.g. lung cancer and/or esophageal cancer.

Alternatively, the screening method of the present invention can comprise the following steps:

(a) contacting a candidate compound with a cell into which a vector, comprising the transcriptional regulatory region of CDCA5, EPHA7, STK31 or WDHD and a reporter gene that is expressed under the control of the transcriptional regulatory region, has been introduced;

(b) measuring the expression or activity of said reporter gene; and

(c) selecting the candidate compound that reduces the expression or activity of said reporter gene.

Suitable reporter genes and host cells are well known in the art. For example, reporter genes are luciferase, green florescence protein (GFP), Discosoma sp. Red Fluorescent Protein (DsRed), Chrolamphenicol Acetyltransferase (CAT), lacZ and beta-glucuronidase (GUS), and host cell is COST, HEK293, HeLa and so on. The reporter construct required for the screening can be prepared by connecting reporter gene sequence to the transcriptional regulatory region of CX. The transcriptional regulatory region of CX herein is the region from start codon to at least 500 bp upstream, for example, 1000 bp, for example, 5000 or 10000 bp upstream, but not restricted. A nucleotide segment containing the transcriptional regulatory region can be isolated from a genome library or can be propagated by PCR. Methods for identifying a transcriptional regulatory region, and also assay protocol are well known (Molecular Cloning third edition chapter 17, 2001, Cold Springs Harbor Laboratory Press).

The vector containing the said reporter construct is infected to host cells and the expression or activity of the reporter gene is detected by method well known in the art (e.g., using luminometer, absorption spectrometer, flow cytometer and so on). “Reduces the expression or activity” as defined herein refers to at least 10% reduction of the expression or activity of the reporter gene in comparison with in absence of the compound, for example, at least 25%, 50% or 75% reduction, for example, at least 95% reduction.

Aspects of the present invention are described in the following examples, which are not intended to limit the scope of the invention described in the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

The invention will be further described in the following examples, which do not limit the scope of the invention described in the claims.

### (v) Screening Using the Binding of EPHA7 and EGFR as an Index

In the present invention, it was confirmed that the EPHA7 protein interacts with EGFR protein (FIG. 8B), and phosphorylates at Tyr-845 of the EGFR protein (FIG. 8A). In addition, promotion of a phosphorylation of PLCgamma (Tyr-783), CDC25 (Ser-216), MET (Tyr-1230/1234/1235, Tyr-1313, Tyr-1349, Tyr-1365), Shc (Tyr317, Tyr239/240) (GenBank Accession No.: NM_{—}001130041, SEQ ID NO.:58), ERK (p44/42 MAPK) (Thr202/Tyr204), Akt (Ser473) (GenBank Accession No.: NM_{—}001014431, SEQ ID NO.:60) and STATS (Tyr705) (GenBank Accession No.: NM_{—}139276, SEQ ID NO.:62) (FIG. 8A, FIG. 21, FIG. 22) in the presence of EPHA7 protein was also confirmed. EPHA7 is known to have a consensus sequence of a protein kinase domain in 633-890aa. Hence, the present inventors identified EGFR as a substrate of EPHA7, whose pathway was well known to be involved in cellular proliferation and invasion. Thus, a compound that inhibits the binding between EPHA7 protein and EGFR protein can be screened using such a binding of EPHA7 protein and EGFR protein or phosphorylation level of EGFR protein (Tyr-845) as an index. Furthermore, the present inventors identified the interaction of MET with EPHA7. Therefore, the present invention also provides a method for screening a compound for inhibiting the binding between EPHA7 protein and EGFR or MET protein can be screened using such a binding of EPHA7 protein and EGFR or MET protein or phosphorylation level of EGFR protein (Tyr-845) as an index. Furthermore, the present invention also provides a method for screening a compound for inhibiting or reducing a growth of cancer cells expressing EPHA7, e.g. lung cancer cell and/or esophageal cancer cell, and a compound for treating or preventing cancers, e.g. lung cancer and/or esophageal cancer.

Specifically, the present invention provides the following methods of [1] to [5]:

[1] A method of screening for an agent interrupts a binding between an EPHA7 polypeptide and an EGFR or MET polypeptide, said method comprising the steps of:

(a) contacting an EPHA7 polypeptide or functional equivalent thereof with an EGFR or MET polypeptide or functional equivalent thereof in the presence of a test agent;

(b) detecting a binding between the polypeptides;

(c) comparing the binding level detected in the step (b) with those detected in the absence of the test agent; and

(d) selecting the test agent that reduce or inhibits the binding level.

[2] A method of screening for an agent useful in treating or preventing cancers, said method comprising the steps of:

(a) contacting an EPHA7 polypeptide or functional equivalent thereof with an EGFR or MET polypeptide or functional equivalent thereof in the presence of a test agent;

(b) detecting a binding between the polypeptides;

(c) comparing the binding level detected in the step (b) with those detected in the absence of the test agent; and

(d) selecting the test agent that reduce or inhibits the binding level.

[3] The method of [1] or [2], wherein the functional equivalent of EPHA7 comprising the EGFR-binding domain.

[4] The method of [1] or [2], wherein the functional equivalent of EGFR or MET comprising the EPHA7-binding domain.

[5] The method of [1], wherein the cancer is selected from the group consisting of lung cancers and esophageal cancer.

In the context of the present invention, a functional equivalent of an EPHA7, EGFR or MET polypeptide is a polypeptide that has a biological activity equivalent to an EPHA7 polypeptide (SEQ ID NO: 4), EGFR or MET polypeptide, respectively (see, (1) Cancer-related genes and cancer-related protein, and functional equivalent thereof in Definition or (6) Expression vector in [EXAMPLE 1]). More specifically, the functional equivalent of EGFR is a polypeptide fragment comprising amino acid sequence of SEQ ID NO: 75 and of MET is a polypeptide fragment comprising amino acid sequence of SEQ ID NO: 76 comprising the EPHA7-binding domain.

As a method of screening for compounds that modulates, e.g. inhibits, the binding of EPHA7 to EGFR, many methods well known by one skilled in the art can be used.

A polypeptide to be used for screening can be a recombinant polypeptide or a protein derived from natural sources, or a partial peptide thereof. Any test compound aforementioned can used for screening.

As a method of screening for proteins, for example, that bind to a polypeptide using EPHA7 or EGFR polypeptide or functionally equivalent thereof (see, (1) Cancer-related genes and cancer-related protein, and functional equivalent thereof in Definition), many methods well known by a person skilled in the art can be used. Such a screening can be conducted using, for example, an immunoprecipitation, West-Western blotting analysis (Skolnik et al., Cell 65: 83-90 (1991)), a two-hybrid system utilizing cells (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet 10: 286-92 (1994)”), affinity chromatography and A biosensor using the surface plasmon resonance phenomenon (see (i) General screening Method).

Any aforementioned test compound can be used (see (1) Test compounds for screening).

In some embodiments, this method further comprises the step of detecting the binding of the candidate compound to EPHA7 protein or EGFR, or detecting the level of binding EPHA7 protein to EGFR protein. Cells expressing EPHA7 protein and EGFR proteins include, for example, cell lines established from cancer, e.g. lung cancer and/or esophageal cancer, such cells can be used for the above screening of the present invention so long as the cells express these two genes. Alternatively cells can be transfected both or either of expression vectors of EPHA7 and EGFR, so as to express these two genes. The binding of EPHA7 protein to EGFR protein can be detected by immunoprecipitation assay using an anti-EPHA7 antibody and anti-EGFR antibody (FIG. 8B).

### (vi) Screening Using EPHA7-Mediated Phosphorylation as an Index

According to another aspect of the invention, agents that inhibits or reduces an EPHA7-mediated phosphorylation of EGFR, PLC-gamma (SEQ ID NO.: 52, GenBank Accession No.: NM_{—}002660), CDC25 (SEQ ID NO.: 54, GenBank Accession No.:NM_{—}001790), MET (SEQ ID NO.: 56, GenBank Accession No.: NM_{—}000245), Shc (SEQ ID NO.: 58, GenBank Accession No.: NM_{—}001130041), ERK (p44/42 MAPK) (SEQ ID NO.: 50, GenBank Accession No.: NM_{—}001040056), Akt (SEQ ID NO.: 60, GenBank Accession No.: NM_{—}001014431) or STAT3 (SEQ ID NO.: 62, GenBank Accession No.: NM_{—}139276) can be used for inhibiting or reducing a growth of cancer cells expressing EPHA7, e.g. lung cancer cell or esophageal cancer cell, and can be used for treating or preventing cancer expressing EPHA7, e.g. lung cancer or esophageal cancer, are screened using the EPHA7-mediated phosphorylation level as an index.

Specifically, the present invention provides the following methods of [1] to [5]:

[1] A method of screening for an agent that modulate an EPHA7-mediated phosphorylation or the agent for preventing or treating cancer expressing EPHA7 gene, the methods comprising the steps of:

(a) contacting a test agent with

(i) an EPHA7 polypeptide or functional equivalent thereof and

(ii) an EGFR, PLC-gamma, CDC25, MET, Shc, ERK (p44/42 MAPK), Akt or STAT3 polypeptide or functional equivalent thereof as a substrate;

under a condition that allows phosphorylation of the substrate;

(b) detecting the phosphorylation level of the substrate;

(c) comparing the phosphorylation level detected in the step (b) with those detected in the absence of the test agent; and

(d) selecting the test agent that inhibits or reduces the phosphorylation level as an inhibitor, or selecting the test agent that promotes or enhances the phosphorylation level as an enhancer.

[2] A method of screening for an agent for preventing or treating cancers, said method comprising the steps of:

(a) contacting a test agent with

(i) an EPHA7 polypeptide or functional equivalent thereof and

(ii) an EGFR, PLC-gamma, CDC25, MET, Shc, ERK (p44/42 MAPK), Akt or STAT3 polypeptide or functional equivalent thereof as a substrate;

under a condition that allows phosphorylation of the substrate;

(b) detecting the phosphorylation level of the substrate;

(c) comparing the phosphorylation level detected in the step (b) with those detected in the absence of the test agent; and

(d) selecting the test agent that inhibits or reduces the phosphorylation level.

[3] The method of [1] or [2], wherein the functional equivalent of EGFR, PLC-gamma, CDC25, MET, Shc, ERK (p44/42 MAPK), Akt or STAT3 polypeptide comprises at least one EPHA7-mediated phosphorylation site of the polypeptide.

[4] The method of [3], wherein the EPHA7-mediated phosphorylation site is Tyr845, Tyr-1068, Tyr-1086, or Tyr-1173 of EGFR, Tyr-783 of PLCgamma, Ser-216 of CDC25, Tyr-1230/1234/1235, Tyr-1313, Tyr-1349 or Tyr-1365 of MET, Tyr317 or Tyr239/240 of Shc, Thr202/Tyr204 of ERK (p44/42 MAPK), or Ser473 of Akt polypeptide.

[5] The method of [2], wherein the cancer is selected from the group consisting of lung cancers and esophageal cancer.

The EPHA7 polypeptide or functional equivalents thereof used in the screening can be prepared as a recombinant protein or natural protein, by methods well known to those skilled in the art. The polypeptides can be obtained adopting any known genetic engineering methods for producing polypeptides (e.g., Morrison J., J Bacteriology 1977, 132: 349-51; Clark-Curtiss & Curtiss, Methods in Enzymology (eds. Wu et al.) 1983, 101: 347-62) as mentioned above (see (1) Cancer-related genes and cancer-related protein, and functional equivalent thereof in Definition).

Further, a partial peptide of the EPHA7 protein can also be used for the invention so long as it retains the kinase activity of the protein. Such partial peptides can be produced by genetic engineering, by known methods of peptide synthesis, or by digesting the natural EPHA7 protein with an appropriate peptidase (see (1) Cancer-related genes and cancer-related protein, and functional equivalent thereof in Definition).

The EPHA7 polypeptide or functional equivalent thereof to be contacted with a test agent and EGFR protein can be, for example, a purified polypeptide, a soluble protein, or a fusion protein fused with other polypeptides.

Similarly to the EPHA7 polypeptide, EGFR polypeptide for the present screening can be prepared as a recombinant protein or natural protein. Furthermore, EGFR polypeptide can be prepared as a fusion protein so long as the resulting fusion protein can be phosphorylated by the EPHA7 polypeptide. The nucleotide sequence of EGFR is well known in the art. Further, EGFR is also commercially available.

In these embodiments, a condition that allows phosphorylation of EGFR polypeptide can be provided by incubating the EGFR polypeptide with EPHA7 polypeptide to be phosphorylated the EGFR polypeptide and ATP (see, (14) in vitro kinase assay in [EXAMPLE 1]). Further, in the present invention, a substance enhancing kinase activity of the EPHA7 polypeptide can be added to the reaction mixture of screening. When phosphorylation of the substrate is enhanced by the addition of the substance, phosphorylation level of a substrate can be determined with higher sensitivity.

The contact of the EPHA7 polypeptide or functional equivalent thereof, its substrate, and a test agent can be conducted in vivo or in vitro. The screening in vitro can be carried out in buffer, for example, but are not limited to, phosphate buffer and Tris buffer, so long as the buffer does not inhibit the phosphorylation of the substrate by the EPHA7 polypeptide or functional equivalent thereof.

In the present invention, the phosphorylation level of a substrate can be determined by methods known in the art (see (2) General screening Method).

(vii) Screening Using STK31 Kinase Activity as an Index

In the present invention, it was confirmed that a promotion of a phosphorylation of EGFR(Ser1046/1047), ERK (P44/42 MAPK)(Thr202/Tyr204) and MEK (S217/221) (FIG. 12B, C, D) in the presence of STK31 protein was also confirmed. STK31 protein is known to have a consensus sequence of a STYKc domain in 745-972aa. Hence, the present inventors identified EGFR, ERK (P44/42 MAPK), and MEK as the downstream targets of STK31. It was shown that Ser1046/1047 of EGFR was phosphorylated by Ca²⁺/calmodulin-dependant kinase II (CaM kinase II) and its phosphorylation attenuated EGFR kinase activity. CaM kinase II was also reported to cause ERK (P44/42 MAPK) activation that regulated cell growth. Thus, a compound inhibiting or reducing a STK31 kinase activity can be useful for inhibiting or reducing cancer cells expressing STK31, e.g. lung cancer cells and/or esophageal cancer cell, and can be useful for treating or preventing cancers expressing STK31, e.g. lung cancer and/or esophageal cancer. Furthermore, the present inventors confirmed the STK31 kinase activity using MBP as a substrate. Thus, a compound that inhibits the STK31 kinase activity can be screened using a phosphorylation level of MBP. Therefore, the present invention also provides a method for screening a compound for inhibiting or reducing cancer cell growth using such a STK31 kinase activity, as an index. Furthermore, the present invention also provides a method for screening a compound for inhibiting or reducing cancer cells expressing EPHA7, e.g. lung cancer cell and/or esophageal cancer cell. The method is particularly suited for screening agents that can be used in cancer expressing EPHA7, e.g. lung cancer and/or esophageal cancer.

Specifically, the present invention provides the following methods of [1] to [3]:

[1] A method of screening for an agent for preventing or treating cancers, wherein said method comprising the steps of:

(a) contacting a test agent with

(i) an STK31 polypeptide or functional equivalent thereof and

(ii) a substrate;

under a condition that allows phosphorylation of the substrate;

(b) detecting the phosphorylation level of the substrate;

(c) comparing the phosphorylation level detected in the step (b) with those detected in the absence of the test agent; and

(d) selecting the test agent that inhibits or reduces the phosphorylation level.

[2] The method of [1], wherein the substrate is MBP, EGFR, ERK (P44/42 MAPK), or MEK.

[3] The method of [1], wherein the cancer is selected from the group consisting of lung cancers and esophageal cancer.

The STK31 polypeptide or functional equivalents thereof used in the screening can be prepared as a recombinant protein or natural protein, by methods well known to those skilled in the art. The polypeptides can be obtained adopting any known genetic engineering methods for producing polypeptides (e.g., Morrison J., J Bacteriology 1977, 132: 349-51; Clark-Curtiss & Curtiss, Methods in Enzymology (eds. Wu et al.) 1983, 101: 347-62) as mentioned above (see (1) Cancer-related genes and cancer-related protein, and functional equivalent thereof in Definition).

Further, a partial peptide of the STK31 protein can also be used for the invention so long as it retains the kinase activity of the protein. Such partial peptides can be produced by genetic engineering, by known methods of peptide synthesis, or by digesting the natural STK31 protein with an appropriate peptidase (see (1) Cancer-related genes and cancer-related protein, and functional equivalent thereof in Definition).

The STK31 polypeptide or functional equivalent thereof to be contacted with a test agent and a substrate, e.g. MBP, EGFR, ERK (P44/42 MAPK), or MEK, can be, for example, a purified polypeptide, a soluble protein, or a fusion protein fused with other polypeptides.

In these embodiments, a condition that allows phosphorylation of a substrate can be provided by incubating the substrate with STK31 polypeptide to be phosphorylated the substrate and ATP (see, (14) in vitro kinase assay in [EXAMPLE 1]). Further, in the present invention, a substance enhancing kinase activity of the STK31 polypeptide can be added to the reaction mixture of screening. When phosphorylation of the substrate is enhanced by the addition of the substance, phosphorylation level of a substrate can be determined with higher sensitivity.

The contact of the STK31 polypeptide or functional equivalent thereof, its substrate, and a test agent can be conducted in vivo or in vitro. The screening in vitro can be carried out in buffer, for example, but are not limited to, phosphate buffer and Tris buffer, so long as the buffer does not inhibit the phosphorylation of the substrate by the STK31 polypeptide or functional equivalent thereof.

In the present invention, the phosphorylation level of a substrate can be determined by methods known in the art (see (2) General screening Method).

(viii) Screening Using the Binding of STK31 and c-raf, MEK or ERK (p44/42 MAPK) as an Index

In the present invention, it was confirmed that the STK31 protein interacts with c-raf (GenBank Accession No.: NM_{—}002880, SEQ ID NO.: 64), MEK or ERK protein (FIG. 12F), and phosphorylates at Ser-1046/1047 of the EGFR protein, Thr202/Tyr204 of ERK (p44/42 MAPK) and MEK (FIG. 12B, D). A compound that inhibits the binding between STK31 protein and c-raf, MEK or ERK (p44/42 MAPK) protein can be screened using such a binding of STK31 protein and c-raf, MEK or ERK (p44/42 MAPK) protein as an index. Therefore, the present invention also provides a method for screening a compound for inhibiting the binding between STK31 protein and c-raf, MEK or ERK (p44/42 MAPK) can be screened using such a binding of STK31 protein and c-raf, MEK or ERK (p44/42 MAPK). Furthermore, the present invention also provides a method for screening a compound for inhibiting or reducing a growth of cancer cells expressing STK31, e.g. lung cancer cell and/or esophageal cancer cell, and a compound for treating or preventing cancers, e.g. lung cancer and/or esophageal cancer.

Specifically, the present invention provides the following methods of [1] to [5]:

[1] A method of screening for an agent interrupts a binding between an STK31 polypeptide and a c-raf, MEK or ERK (p44/42 MAPK), said method comprising the steps of:

(a) contacting an STK31 polypeptide or functional equivalent thereof with an c-raf, MEK or ERK (p44/42 MAPK) polypeptide or functional equivalent thereof in the presence of a test agent;

(b) detecting a binding between the polypeptides;

(c) comparing the binding level detected in the step (b) with those detected in the absence of the test agent; and

(d) selecting the test agent that reduce or inhibits the binding level.

[2] A method of screening for an agent useful in treating or preventing cancers, said method comprising the steps of:

(a) contacting an STK31 polypeptide or functional equivalent thereof with an c-raf, MEK or ERK (p44/42 MAPK) polypeptide or functional equivalent thereof in the presence of a test agent;

(b) detecting a binding between the polypeptides;

(c) comparing the binding level detected in the step (b) with those detected in the absence of the test agent; and

(d) selecting the test agent that reduce or inhibits the binding level.

[3] The method of [1] or [2], wherein the functional equivalent of STK31 comprising the c-raf, MEK or ERK (p44/42 MAPK)-binding domain.

[4] The method of [1] or [2], wherein the functional equivalent of c-raf, MEK or ERK (p44/42 MAPK) comprising the STK31-binding domain.

[5] The method of [1], wherein the cancer is selected from the group consisting of lung cancers and esophageal cancer.

In the context of the present invention, a functional equivalent of an STK31, c-raf (SEQ ID NO.: 64), MEK or ERK (p44/42 MAPK) polypeptide is a polypeptide that has a biological activity equivalent to an STK31 polypeptide (SEQ ID NO: 6) or c-raf, MEK or ERK (p44/42 MAPK), respectively (see, (1) Cancer-related genes and cancer-related protein, and functional equivalent thereof in Definition or (6) Expression vector in [EXAMPLE 1]).

As a method of screening for compounds that modulates, e.g. inhibits, the binding of EPHA7 to EGFR, many methods well known by one skilled in the art can be used.

A polypeptide to be used for screening can be a recombinant polypeptide or a protein derived from natural sources, or a partial peptide thereof. Any test compound aforementioned can used for screening.

As a method of screening for proteins, for example, that bind to a polypeptide using STK31, c-raf, MEK or ERK (p44/42 MAPK) polypeptide or functionally equivalent thereof (see, (1) Cancer-related genes and cancer-related protein, and functional equivalent thereof in Definition), many methods well known by a person skilled in the art can be used. Such a screening can be conducted using, for example, an immunoprecipitation, West-Western blotting analysis (Skolnik et al., Cell 65: 83-90 (1991)), a two-hybrid system utilizing cells (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet 10: 286-92 (1994)”), affinity chromatography and A biosensor using the surface plasmon resonance phenomenon (see (i) General screening Method).

Any aforementioned test compound can be used (see (1) Test compounds for screening).

In some embodiments, this method further comprises the step of detecting the binding of the candidate compound to STK31 protein, c-raf, MEK or ERK (p44/42 MAPK), or detecting the level of binding STK31 protein to c-raf, MEK or ERK (p44/42 MAPK) protein. Cells expressing STK31 protein and c-raf, MEK or ERK (p44/42 MAPK) proteins include, for example, cell lines established from cancer, e.g. lung cancer and/or esophageal cancer, such cells can be used for the above screening of the present invention so long as the cells express these two genes. Alternatively cells can be transfected both or either of expression vectors of STK31 and c-raf, MEK or ERK (p44/42 MAPK), so as to express these two genes. The binding of STK31 protein to c-raf, MEK or ERK (p44/42 MAPK) protein can be detected by immunoprecipitation assay using an anti-STK31 antibody and anti-c-raf, MEK or ERK (p44/42 MAPK) antibody (FIG. 12).

### (ix) Screening Using the Phosphorylation Level of WDHD1 as an Index

Furthermore, in the present invention, it was confirmed that the WDHD1 proteins were modified by phosphorylation. And one of the phosphorylated regions of WDHD1 has consensus phosphorylation site for AKT kinase (GenBank Accession No.: NM_{—}001014431) (R—X—R—X—X—S374; ref. 33). PI3K/AKT signaling is important for cell proliferation and survival. And, inhibition of PI3K activity using LY294002 decreased the expression level of total and phosphorylated WDHD1 (FIG. 16C). This result indicates that WDHD1 is one of the components of the PI3K/AKT pathway and is stabilized by phosphorylation. Furthermore, a inhibition of WDHD1 expression involved in inhibition of cell growth and resulted in inducing apoptosis (FIG. 15C). Thus, a compound that inhibits the phosphorylation of WDHD1 protein can be useful for inhibiting or reducing a growth of cancer cells expressing WDHD1, can be useful for inducing apoptosis to cancer cells, or can be useful for treating or preventing cancers expressing WDHD1, screened using such modification as an index. The cancers can be lung cancer, e.g. non-small cell lung cancer or small cell lung cancer, and/or esophageal cancer. Therefore, the present invention also provides a method for screening a compound for inhibits the phosphorylation of WDHD1 protein. Furthermore, the present invention also provides a method for screening a compound for inhibiting or reducing a growth of cancer cells expressing WDHD1, and a compound for inducing apoptosis for cancer cells expressing WDHD1. The method is particularly suited for screening agents that can be used in treating or preventing cancer expressing WDHD1. The cancer is lung cancer, e.g. non-small cell lung cancer or small cell lung cancer, or esophageal cancer.

Specifically, the present invention provides the following methods of [1] to [5]:

[1] A method of screening for an agent for preventing or treating cancers, wherein said method comprising the steps of:

(a) contacting a test agent with a cell expressing a gene encoding WDHD1 polypeptide or functional equivalent thereof;

(b) culture under a condition that allows phosphorylation of said polypeptide of step (a);

(c) detecting phospho-serine or phospho-tyrosine level of said polypeptide of step (a);

(d) comparing the phosphorylation level detected in the step (c) with those detected in the absence of the test agent; and

(e) selecting the test agent that inhibits or reduces the phosphorylation level.

[2] The method of [1], wherein cancer is selected from the group consisting of lung cancers and esophageal cancer.

[3] The method of [1], wherein phospho-serine of WDHD1 is S374.

[4] The method of [1], wherein the test agent binds to WDHD1 polypeptide or functional equivalent thereof.

[5] The method of [1], wherein the agent phosphorylation activity of AKT at the site of WDHD1.

Herein, any cell can be used so long as it expresses the WDHD1 polypeptide or functional equivalents thereof (see, (1) Cancer-related genes and cancer-related protein, and functional equivalent thereof in Definition). The cell used in the present screening can be a cell naturally expressing the WDHD1 polypeptide including, for example, cells derived from and cell-lines established from lung cancer, esophageal cancer and testis. Cell-lines of lung cancer cell and/or esophageal cancer cell, for example, LC319, TE9 and so on, can be employed.

Alternatively, the cell used in the screening can be a cell that naturally does not express the WDHD1 polypeptide and which is transfected with an WDHD1 polypeptide- or an WDHD1 functional equivalent-expressing vector. Such recombinant cells can be obtained through known genetic engineering methods (e.g., Morrison D A., J Bacteriology 1977, 132: 349-51; Clark-Curtiss & Curtiss, Methods in Enzymology (eds. Wu et al.) 1983, 101: 347-62) as mentioned above (see (1) Cancer-related genes and cancer-related protein, and functional equivalent thereof in Definition).

Any of the aforementioned test compounds can be used for the present screening. In some embodiments, compounds that can permeate into a cell are selected. Alternatively, when the test compound is a polypeptide, the contact of a cell and the test agent in the present screening can be performed by transforming the cell with a vector that comprises the nucleotide sequence coding for the test agent and expressing the test agent in the cell.

In the present invention, as mentioned above, the biological activity of the WDHD1 protein includes phosphorylation activity. The skilled artisan can estimate phosphorylation level as mentioned above (see (2) General Screening Method).

When the biological activity to be detected in the present method is cell proliferation, it can be detected, for example, by preparing cells which express the polypeptide of the present invention, culturing the cells in the presence of a test compound, and determining the speed of cell proliferation, measuring the cell cycle and such, as well as by measuring the colony forming activity as described in the Examples.

### (x) Screening Using an Interaction Between CDCA5 and CDC2, or CDCA5 and ERK as an Index

In the present invention, it was confirmed that the CDCA5 polypeptide interacts with CDC2 polypeptide and ERK polypeptide, and CDCA5 polypeptide is phosphorylated by CDC2 polypeptide and ERK polypeptide (FIG. 2). Furthermore, CDCA5 polypeptide has a consensus phosphorylation motif for CDC2 at amino acid residues 68-82 (S/T-P-x-R/K), wherein Serine-75 of SEQ ID NO: 2 is the phosphorylated region or site (FIG. 1). CDCA5 polypeptide has a consensus phosphorylation motif for ERK at amino acid residues 76-86 and 109-122 (x-x-S/T-P), wherein Serine-79 and Threonine-115 of SEQ ID NO: 2 are the phosphorylated regions or sites (FIG. 1). These data are consistent with the conclusion that the CDCA5 polypeptide was phosphorylated by ERK polypeptide and CDC2 polypeptide.

The protein encoded by ERK gene is a member of the MAP kinase family proteins that function as an integration point for multiple biochemical signals, and are involved in a wide variety of cellular processes for example, proliferation, differentiation, transcription regulation and development. The MAPK cascade integrates and processes various extracellular signals by phosphorylating substrates, which alters their catalytic activities and conformation or creates binding site for protein-protein interactions.

On the other hand, cyclin-dependent kinases (CDKs) are heterodimeric complexes composed of a catalytic kinase subunit and a regulatory cyclin subunit, and comprise a family divided into two groups based on their roles in cell progression and transcriptional regulation. CDC2/CDK1 (CDC2-cyclin B complex) is a member of the first group, which are required for orderly G2 to M phase transition. Recently, CDC2 was implicated in cell survival during mitotic checkpoint activation (O'Connor D S, et al. Cancer Cell. 2002 July; 2(1):43-54).

Therefore these data showed that the phosphorylation of CDCA5 by ERK and CDC2 promoted cancer cell cycle progression that increases the malignant potential of tumors. In summary, these data demonstrate that CDCA5 promotes the growth of lung and esophagus cancers through its phosphorylation by MAPK or CDK pathway.

Specifically, the present invention provides the following methods of [1] to [14]:

[1] A method of screening for an agent interrupts an interaction or binding between a CDCA5 polypeptide and a CDC2 polypeptide, said method comprising the steps of:

(a) contacting polypeptide of (i) and (ii) in the presence of a test agent

(i) a CDCA5 polypeptide or functional equivalent thereof; and

(ii) a CDC2 polypeptide or functional equivalent thereof

(b) detecting a level of the interaction or binding between the polypeptides;

(c) comparing the level detected in the step (b) with those detected in the absence of the test agent; and

(d) selecting the test agent that reduce or inhibits the level.

[2] A method of [1], wherein the agent is useful in treating or preventing cancer expressing CDCA5.

[3] The method of [2], wherein the cancer is selected from the group consisting of lung cancers and esophageal cancer.

[4] The method of [3], wherein the lung cancer is non-small cell lung cancer or small cell lung cancer.

[5] The method of [1], wherein the test agent binds to CDCA5 polypeptide or functional equivalent thereof.

[6] The method of [1], wherein the functional equivalent of CDCA5 comprising the CDC2-interaction domain.

[7] The method of [1], wherein the functional equivalent of CDC2 comprising the CDCA5-interaction domain.

[8] A method of screening for an agent interrupts an interaction or binding between a CDCA5 polypeptide and a ERK polypeptide, said method comprising the steps of:

(a) contacting polypeptide of (i) and (ii) in the presence of a test agent

(i) a CDCA5 polypeptide or functional equivalent thereof; and

(ii) a ERK polypeptide or functional equivalent thereof

(b) detecting a level of the interaction or binding between the polypeptides;

(c) comparing the level detected in the step (b) with those detected in the absence of the test agent; and

(d) selecting the test agent that reduce or inhibits the level.

[9] A method of [8], wherein the agent is useful in treating or preventing cancer expressing CDCA5.

[10] The method of [9], wherein the cancer is selected from the group consisting of lung cancers and esophageal cancer.

[11] The method of [10], wherein the lung cancer is non-small cell lung cancer or small cell lung cancer.

[12] The method of [8], wherein the test agent binds to CDCA5 polypeptide or functional equivalent thereof.

[13] The method of [8], wherein the functional equivalent of CDCA5 comprising the CDC2-interaction domain.

[14] The method of [8], wherein the functional equivalent of CDC2 comprising the CDCA5-interaction domain.

In the context of the present invention, a functional equivalent of a CDCA5 polypeptide, a CDC2 polypeptide or an ERK polypeptide is a polypeptide that has a biological activity equivalent to a CDCA5 polypeptide (SEQ ID NO: 2), a CDC2 polypeptide (SEQ ID NO: 48) or an ERK polypeptide (SEQ ID NO: 50). (see, (1) Cancer-related genes and cancer-related protein, and functional equivalent thereof in Definition).

As a method of screening for compounds that modulates, e.g. inhibits, the binding between CDCA5 polypeptide and CDC2 polypeptide, or the binding between CDCA5 polypeptide and ERK polypeptide, the functional equivalent remains the binding activity. The functional equivalent of CDCA5 polypeptide can contain a CDCA2 binding region of CDCA5 polypeptide or an ERK binding region of CDCA5 polypeptide; the functional equivalent of CDC2 polypeptide can contain a CDCA5 binding region of CDC2 polypeptide; and the functional equivalent of ERK polypeptide can contain a CDCA5 binding region of ERK polypeptide.

Many methods of detecting a level of an interaction or binding between the polypeptides well known by one skilled in the art can be used. A polypeptide to be used for screening can be a recombinant polypeptide or a protein derived from natural sources, or a partial peptide thereof.

Any test compound aforementioned can be used for screening (see (1) Test compound for screening in Definition). For example, the test agent can be an antibody against CDCA5 polypeptide, an antibody against a CDC2 binding region of CDCA5 polypeptide or an antibody against an ERK binding region of CDCA5 polypeptide, or the test agent can be a partial peptide of CDCA5 polypeptide, CDC2 polypeptide or ERK polypeptide which effect as a dominant negative, e.g. a CDC2 binding region of CDCA5 polypeptide, an ERK binding region of CDCA5 polypeptide, CDCA5 binding region of CDC2 polypeptide or CDCA5 binding region of ERK polypeptide.

As a method of screening for proteins, for example, that bind to a polypeptide using CDCA5 polypeptide, CDC2 polypeptide, ERK polypeptide or functionally equivalent thereof (see, (1) Cancer-related genes and cancer-related protein, and functional equivalent thereof in Definition), many methods well known by a person skilled in the art can be used. Such a screening can be conducted using, for example, an immunoprecipitation, West-Western blotting analysis (Skolnik et al., Cell 65: 83-90 (1991)), a two-hybrid system utilizing cells (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet 10: 286-92 (1994)”), affinity chromatography and A biosensor using the surface plasmon resonance phenomenon (see (i) General screening Method).

Any aforementioned test compound can used (see (1) Test compounds for screening).

In some embodiments, this method further comprises the step of detecting the binding of the candidate compound to CDCA5 polypeptide, CDC2 polypeptide or ERK polypeptide, or detecting the level of binding between CDCA5 polypeptide and CDC2 polypeptide, or CDCA5 polypeptide and ERK polypeptide in the cell expressing these genes. Cells expressing these genes include, for example, cell lines established from cancer, e.g. a cancer resulting from overexpression of a CX gene or mediated by a CX gene, e.g., lung cancer and/or esophageal cancer, such cells can be used for the above screening of the present invention so long as the cells express these genes. Alternatively cells can be transfected both or either of expression vectors of CDCA5 and CDC2, or CDCA5 and ERK, so as to express these genes. The binding between CDCA5 and CDC2 or the binding between CDCA5 and ERK can be detected by immunoprecipitation assay using an anti-CDCA5 antibody, anti-CDC2 antibody and anti-ERK antibody.

### (xi) Screening Using the Phosphorylation of CDCA5 as an Index

According to another aspect of the invention, agents that inhibits or reduces a CDC2-mediated phosphorylation of CDCA5 or an ERK-mediated phosphorylation of CDCA5 can be used for inhibiting or reducing a cycle progression of cancer cells expressing CDCA5, e.g., cell from a cancer resulting from overexpression of a CX gene or mediated by a CX gene, e.g., lung cancer cell or esophageal cancer cell, and can be used for treating or preventing cancer expressing CDCA5, e.g. lung cancer or esophageal cancer, are screened using the CDC2-mediated phosphorylation level of a CDCA5 or an ERK-mediated phosphorylation level of CDCA5 as an index.

Specifically, the present invention provides the following methods of [1] to [14]:

[1] A method of screening for an agent that modulate a CDC2-mediated phosphorylation of CDCA5, the methods comprising the steps of:

(a) contacting polypeptide of (i) and (ii) in the presence of a test agent

(i) a CDCA5 polypeptide or functional equivalent thereof; and

(ii) a CDC2 polypeptide or functional equivalent thereof

(b) detecting a phosphorylation level of the polypeptides of (a)(i);

(c) comparing the phosphorylation level detected in the step (b) with those detected in the absence of the test agent; and

(d) selecting the test agent that inhibits or reduces the phosphorylation level as an inhibitor, or selecting the test agent that promotes or enhances the phosphorylation level as an enhancer.

[2] A method of [1], wherein the agent is useful for preventing or treating cancers expressing CDCA5.

[3] The method of [2], wherein the cancer is selected from the group consisting of lung cancers and esophageal cancer.

[4] The method of [3], wherein the lung cancer is non-small cell lung cancer or small cell lung cancer.

[5] The method of [1], wherein the test agent binds to CDCA5 polypeptide or functional equivalent thereof.

[6] The method of [1], wherein the functional equivalent of CDCA5 polypeptide comprises at least one CDC2-mediated phosphorylation site of the CDCA5 polypeptide.

[7] The method of [6], wherein the CDC2-mediated phosphorylation site is Serine-21, Serine-75 or Threonine-159 of SEQ ID NO: 2 (CDCA5).

[8] A method of screening for an agent that modulate an ERK-mediated phosphorylation of CDCA5, the methods comprising the steps of:

(a) contacting polypeptide of (i) and (ii) in the presence of a test agent

(i) a CDCA5 polypeptide or functional equivalent thereof; and

(ii) an ERK polypeptide or functional equivalent thereof

(b) detecting a phosphorylation level of the polypeptides of (a)(i);

(c) comparing the phosphorylation level detected in the step (b) with those detected in the absence of the test agent; and

(d) selecting the test agent that inhibits or reduces the phosphorylation level as an inhibitor, or selecting the test agent that promotes or enhances the phosphorylation level as an enhancer.

[9] A method of [8], wherein the agent is useful for preventing or treating cancers expressing CDCA5.

[10] The method of [9], wherein the cancer is selected from the group consisting of lung cancers and esophageal cancer.

[11] The method of [10], wherein the lung cancer is non-small cell lung cancer or small cell lung cancer.

[12] The method of [8], wherein the test agent binds to CDCA5 polypeptide or functional equivalent thereof.

[13] The method of [8], wherein the functional equivalent of CDCA5 polypeptide comprises at least one ERK-mediated phosphorylation site of the CDCA5 polypeptide.

[14] The method of [13], wherein the ERK-mediated phosphorylation site is Serine-21, Threonine-48, Serine-75, Serine-79, Threonine-111, Threonine-115, Threonine-158 or Serine-209 of SEQ ID NO: 2 (CDCA5).

In another embodiment, the present invention provides the following methods of [1] to [9]:

[1] A method of screening for an agent useful in preventing or treating cancers, wherein said method comprising the steps of:

(a) contacting a test agent with a cell expressing a gene encoding CDCA5 polypeptide or functional equivalent thereof;

(b) culturing under a condition that allows phosphorylation of said polypeptide of step (a);

(c) detecting phosphorylation level of said polypeptide of step (a);

(d) comparing the phosphorylation level detected in the step (c) with those detected in the absence of the test agent; and

(e) selecting the test agent that inhibits or reduces the phosphorylation level.

[2] A method of [1], wherein the agent is useful for preventing or treating cancers expressing CDCA5.

[3] The method of [2], wherein the cancer is selected from the group consisting of lung cancers and esophageal cancer.

[4] The method of [3], wherein the lung cancer is non-small cell lung cancer or small cell lung cancer.

[5] The method of [1], wherein the agent inhibits or reduces CDC2-mediated phosphorylation activity of CDCA5.

[6] The method of [1], wherein the agent inhibits or reduces ERK-mediated phosphorylation of CDCA5.

[7] The method of [1], wherein the phosphorylation level is phospho-serine or phospho-threonine level.

[8] The method of [6], wherein phospho-serine of CDCA5 is Serine-21, Serine-75, Serine-79 or Serine-209 of SEQ ID NO: 2 (CDCA5).

[9] The method of [5], wherein phospho-threonine of CDCA5 is Threonine-48, Threonine-111, Threonine-115 or Threonine-159 of SEQ ID NO: 2 (CDCA5).

In the context of the present invention, a functional equivalent of a CDCA5 polypeptide, CDC2 polypeptide or an ERK polypeptide is a polypeptide that has a biological activity equivalent to a CDCA5 polypeptide, CDC2 polypeptide or an ERK polypeptide. (see, (1) Cancer-related genes and cancer-related protein, and functional equivalent thereof in Definition). In the method mentioned above, a biological activity is interaction, e.g. a CDC2-mediated phosphorylation of CDCA5 polypeptide or an ERK-mediated phosphorylation of CDCA5 polypeptide.

A functional equivalent of CDCA5 polypeptide used for the screenings of the present invention suitably contains CDCA2 binding region, ERK binding region and/or at least one of the phosphorylation site, e.g. a consensus phosphorylation motif for CDC2 at amino acid residues 68-82 (S/T-P-x-R/K), in which Serine-75 of SEQ ID NO: 2 is phosphorylated, a consensus phosphorylation motif for ERK at amino acid residues 76-86 (x-x-S/T-P), in which Serine-79 of SEQ ID NO: 2 is phosphorylated and/or a consensus phosphorylation motif for ERK at amino acid residues 109-122 (x-x-S/T-P), in which Threonine-115 of SEQ ID NO: 2 is phosphorylated; a functional equivalent of CDC2 peptide used for the screenings of the present invention suitably contains CDCA5 binding region and/or a Serine/Threonine protein kinases catalytic domain, e.g. amino acid residues 4-287 of SEQ ID NO: 48 (CDC2); and a functional equivalent of ERK peptide used for the screenings of the present invention suitably contains CDCA5 binding region and/or a protein kinase domain, e.g. amino acid residues 72-369 of SEQ ID NO: 50 (ERK). (see, (1) Cancer-related genes and cancer-related protein, and functional equivalent thereof in Definition)

Herein, any cell can be used so long as it expresses the CDCA5 polypeptide or functional equivalents thereof (see, (1) Cancer-related genes and cancer-related protein, and functional equivalent thereof in Definition). The cell used in the present screening can be a cell naturally expressing the CDCA5 polypeptide including, for example, cells derived from and cell-lines established from lung cancer, esophageal cancer and testis. Cell-lines of lung cancer cell and/or esophageal cancer cell, for example, A549, LC319 and so on, can be employed.

Alternatively, the cell used in the screening can be a cell that naturally does not express the CDCA5 polypeptide and which is transfected with a CDCA5 polypeptide- or a CDCA5 functional equivalent-expressing vector. Such recombinant cells can be obtained through known genetic engineering methods (e.g., Morrison D A., J Bacteriology 1977, 132: 349-51; Clark-Curtiss & Curtiss, Methods in Enzymology (eds. Wu et al.) 1983, 101: 347-62) as mentioned above (see (1) Cancer-related genes and cancer-related protein, and functional equivalent thereof in Definition).

Any of the aforementioned test compounds can be used for the present screening. In some embodiments, compounds that can permeate into a cell is selected. Alternatively, when the test compound is a polypeptide, the contact of a cell and the test agent in the present screening can be performed by transforming the cell with a vector that comprises the nucleotide sequence coding for the test agent and expressing the test agent in the cell.

In the present invention, as mentioned above, the biological activity of the CDCA5 protein includes phosphorylation activity. The skilled artisan can estimate phosphorylation level as mentioned above (see (i) General Screening Method).

When the biological activity to be detected in the present method is cell cycle promotion, it can be detected, for example, by preparing cells which express the polypeptide of the present invention, culturing the cells in the presence of a test compound, and determining the speed of cell proliferation, measuring the cell cycle and such, as well as by measuring the colony forming activity or FACS analysis as described in the Examples.

Unless otherwise defined, all technical and scientific terms used herein have the same meaning as commonly understood by one of ordinary skill in the art to which this invention belongs. In case of conflict, the present specification, including definitions, will control.

In these embodiments, a condition that allows phosphorylation of CDCA5 polypeptide can be provided by incubating the CDCA5 polypeptide with CDC2 polypeptide or ERK polypeptide to be phosphorylated the CDCA5 polypeptide and ATP (see, (14) in vitro kinase assay in [EXAMPLE 1]). Further, in the present invention, a substance enhancing phosphorylation activity of the CDCA5 polypeptide can be added to the reaction mixture of screening. When phosphorylation of the CDCA5 polypeptide is enhanced by the addition of the substance, the phosphorylation level can be determined with higher sensitivity.

The contact of the CDCA5 polypeptide or functional equivalent thereof, CDC2 polypeptide, ERK polypeptide, functional equivalent thereof, and a test agent can be conducted in vivo or in vitro. The screening in vitro can be carried out in buffer, for example, but are not limited to, phosphate buffer and Tris buffer, so long as the buffer does not inhibit the phosphorylation of CDCA5 polypeptide or functional equivalent thereof.

In the present invention, the phosphorylation level of a substrate can be determined by methods known in the art (see (2) General screening Method). Unless otherwise defined, all technical and scientific terms used herein have the same meaning as commonly understood by one of ordinary skill in the art to which this invention belongs. In case of conflict, the present specification, including definitions, will control.

### Isolated Compounds and Pharmaceutical Compositions

A compound isolated by the above screenings is a candidate for drugs which inhibit the activity of the CX polypeptides of the present invention and finds use in the treatment of cancers resulting from overexpression of a CX gene or mediated by a CX gene, e.g. lung cancer and/or esophageal cancer. More particularly, when the biological activity of the CX proteins is used as the index, compounds screened by the present method serve as a candidate for drugs for the treatment of cancers expressing CX gene, e.g. lung cancer and/or esophageal cancer. For instance, the present invention provides a composition for inhibiting or reducing a growth of cancer cells, a compound for inducing apoptosis for cancer cells, and a compounds for treating or preventing cancers, said composition comprising a pharmaceutically effective amount of an inhibitor having at least one function selected from the group consisting of:

(a) inhibiting an expression level of a polypeptide selected from the group consisting of CDCA5, EPHA7, STK31 and WDHD1 polypeptide, or functional equivalent thereof

(b) inhibiting a proliferation activity of the cell expressing a polypeptide selected from the group consisting of CDCA5, EPHA7, STK31 and WDHD1 polypeptide, or functional equivalent thereof;

(c) inducing an apoptosis to the cell expressing a WDHD1 polypeptide or functional equivalent thereof;

(d) inhibiting an invasive activity of the cell expressing an EPHA7 polypeptide or functional equivalent thereof;

(e) inhibiting a binding activity between EPHA7 polypeptide and EGFR polypeptide, or functional equivalent thereof;

(f) inhibiting a kinase activity of a polypeptide selected from the group consisting of EPHA7 and STK31 polypeptide, or functional equivalent thereof; and

(g) inhibiting a phosphorylation level of a WDHD1 protein, or functional equivalent thereof.

(h) inhibiting a cell cycle of the cell expressing a CDCA5 polypeptide or functional equivalent thereof; and

(i) inhibiting a interaction or binding between a CDCA5 polypeptide and CDC2 polypeptide, or functional equivalent thereof.

(j) inhibiting a interaction or binding between a CDCA5 polypeptide and ERK polypeptide, or functional equivalent thereof.

(k) inhibiting a phosphorylation level of a CDCA5 polypeptide, or functional equivalent thereof.

Efficacy of the candidate compounds for treating or preventing cancer can be evaluated by second and/or further screening to identify a therapeutic agent for cancer. For example, when a compound inhibiting the expression of the CDCA5 polypeptide inhibits the activity of cancer, for example, cell growth or invasion, it can be concluded that such a compound has a CDCA5-specific therapeutic effect.

A “pharmaceutically effective amount” of a compound is a quantity that is sufficient to treat and/or ameliorate cancer in an individual. An example of a pharmaceutically effective amount includes an amount needed to decrease the expression or biological activity of CDCA5, EPHA7, STK31 or WDHD1, when administered to an animal. The decrease can be, e.g., at least a 5%, 10%, 20%, 30%, 40%, 50%, 75%, 80%, 90%, 95%, 99%, or 100% change in expression.

Such active ingredient inhibiting an expression of any one gene selected from the group consisting of CDCA5, EPHA7, STK31 and WDHD1 genes (a)-(k) can also be an inhibitory oligonucleotide (e.g., antisense-oligonucleotide, double-stranded molecule, or ribozyme) against the gene, or derivatives, for example, expression vector, of the antisense-oligonucleotide, double-stranded molecule or ribozyme, as described above (see (3) Double-stranded molecule). Alternatively, an active ingredient (e)-(f) can be, for example, a dominant negative mutant of CDCA5, EPHA7, EGFR, STK31 or WDHD1. Further, an antagonist of EPHA7 can be used as an active ingredient inhibiting binding between EPHA7 and EGFR. Furthermore, an antagonist of CDCA5 can be used as an active ingredient inhibiting binding between CDCA5 polypeptide and CDC2 polypeptide, or binding between CDCA5 polypeptide and ERK polypeptide. Alternatively, such active ingredient can be selected by the screening method as described above (see Screening Method).

Moreover, compounds in which a part of the structure of the compound inhibiting the activity of one of the CX proteins is converted by addition, deletion and/or replacement are also included in the compounds obtainable by the screening method of the present invention.

An agent isolated by any of the methods of the invention can be administered as a pharmaceutical or can be used for the manufacture of pharmaceutical (therapeutic or prophylactic) compositions for humans and other mammals, for example, mice, rats, guinea-pigs, rabbits, cats, dogs, sheep, pigs, cattle, monkeys, baboons, and chimpanzees for treating or preventing cancers expressing CX gene, e.g. lung cancer and/or esophageal cancer. Exemplary cancers to be treated or prevented by the agents screened through the present methods include cancers over-expressing CX gene(s) or mediated by the uncontrolled function of CX gene(s), for example, lung cancers, e.g. non-small cell lung cancer or small-cell lung cancer, esophageal cancer, and such.

The isolated agents can be directly administered or can be formulated into dosage form using known pharmaceutical preparation methods. Pharmaceutical formulations can include those suitable for oral, rectal, nasal, topical (including buccal and sub-lingual), vaginal or parenteral (including intramuscular, sub-cutaneous and intravenous) administration, or for administration by inhalation or insufflation. For example, according to the need, the agents can be taken orally, as sugar-coated tablets, capsules, elixirs and microcapsules; or non-orally, in the form of injections of sterile solutions or suspensions with water or any other pharmaceutically acceptable liquid. For example, the agents can be mixed with pharmaceutically acceptable carriers or media, specifically, sterilized water, physiological saline, plant-oils, emulsifiers, suspending agents, surfactants, stabilizers, flavoring agents, excipients, vehicles, preservatives, binders, and such, in a unit dose form required for generally accepted drug implementation. The amount of active ingredients in these preparations makes a suitable dosage within the indicated range acquirable.

The phrase “pharmaceutically acceptable carrier” refers to an inert substance used as a diluent or vehicle for a drug.

Examples of additives that can be mixed to tablets and capsules are, binders for example, gelatin, corn starch, tragacanth gum and Arabic gum; excipients for example, crystalline cellulose; swelling agents for example, corn starch, gelatin and alginic acid; lubricants for example, magnesium stearate; sweeteners for example, sucrose, lactose or saccharin; flavoring agents for example, peppermint, Gaultheria adenothrix oil and cherry. When the unit dosage form is a capsule, a liquid carrier, for example, oil, can also be further included in the above ingredients. Sterile composites for injections can be formulated following normal drug implementations using vehicles for example, distilled water used for injections.

Physiological saline, glucose, and other isotonic liquids including adjuvants, for example, D-sorbitol, D-mannose, D-mannitol, and sodium chloride, can be used as aqueous solutions for injections. These can be used in conjunction with suitable solubilizers, for example, alcohol, specifically ethanol, polyalcohols for example, propylene glycol and polyethylene glycol, non-ionic surfactants, for example, Polysorbate 80 (TM) and HCO-50.

Sesame oil or Soy-bean oil can be used as a oleaginous liquid and can be used in conjunction with benzyl benzoate or benzyl alcohol as a solubilizers and can be formulated with a buffer, for example, phosphate buffer and sodium acetate buffer; a pain-killer, for example, procaine hydrochloride; a stabilizer, for example, benzyl alcohol, phenol; and an anti-oxidant. The prepared injection can be filled into a suitable ample.

Pharmaceutical formulations suitable for oral administration can conveniently be presented as discrete units, for example, capsules, cachets or tablets, each containing a predetermined amount of the active ingredient; as a powder or granules; or as a solution, a suspension or as an emulsion. The active ingredient can also be presented as a bolus electuary or paste, and be in a pure form, i.e., without a carrier. Tablets and capsules for oral administration can contain conventional excipients for example, binding agents, fillers, lubricants, disintegrant or wetting agents. A tablet can be made by compression or molding, optionally with one or more formulational ingredients. Compressed tablets can be prepared by compressing in a suitable machine the active ingredients in a free-flowing form for example, a powder or granules, optionally mixed with a binder, lubricant, inert diluent, lubricating, surface active or dispersing agent. Molded tablets can be made by molding in a suitable machine a mixture of the powdered compound moistened with an inert liquid diluent. The tablets can be coated according to methods well known in the art. Oral fluid preparations can be in the form of, for example, aqueous or oily suspensions, solutions, emulsions, syrups or elixirs, or can be presented as a dry product for constitution with water or other suitable vehicle before use. Such liquid preparations can contain conventional additives for example, suspending agents, emulsifying agents, non-aqueous vehicles (which can include edible oils), or preservatives. The tablets can optionally be formulated so as to provide slow or controlled release of the active ingredient therein.

Formulations for parenteral administration include aqueous and non-aqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents. The formulations can be presented in unit dose or multi-dose containers, for example sealed ampoules and vials, and can be stored in a freeze-dried (lyophilized) condition requiring only the addition of the sterile liquid carrier, for example, saline, water-for-injection, immediately prior to use. Alternatively, the formulations can be presented for continuous infusion. Extemporaneous injection solutions and suspensions can be prepared from sterile powders, granules and tablets of the kind previously described.

Formulations for rectal administration can be presented as a suppository with the usual carriers for example, cocoa butter or polyethylene glycol. Formulations for topical administration in the mouth, for example buccally or sublingually, include lozenges, comprising the active ingredient in a flavored base for example, sucrose and acacia or tragacanth, and pastilles comprising the active ingredient in a base for example, gelatin and glycerin or sucrose and acacia. For intra-nasal administration the compounds obtained by the invention can be used as a liquid spray or dispersible powder or in the form of drops. Drops can be formulated with an aqueous or non-aqueous base also comprising one or more dispersing agents, solubilizing agents or suspending agents. Liquid sprays are conveniently delivered from pressurized packs.

For administration by inhalation the compounds are conveniently delivered from an insufflator, nebulizer, pressurized packs or other convenient means of delivering an aerosol spray. Pressurized packs can comprise a suitable propellant for example, dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount.

Alternatively, for administration by inhalation or insufflation, the compounds can take the form of a dry powder composition, for example a powder mix of the compound and a suitable powder base for example, lactose or starch. The powder composition can be presented in unit dosage form, in for example, capsules, cartridges, gelatin or blister packs from which the powder can be administered with the aid of an inhalator or insufflators.

When desired, the above described formulations, adapted to give sustained release of the active ingredient, can be employed. The pharmaceutical compositions can also contain other active ingredients for example, antimicrobial agents, immunosuppressants or preservatives.

Exemplary unit dosage formulations are those containing an effective dose, as recited below, or an appropriate fraction of the active ingredient.

Methods well known to one skilled in the art can be used to administer the inventive pharmaceutical compound to patients, for example as intra-arterial, intravenous, percutaneous injections and also as intranasal, transbronchial, intramuscular or oral administrations. The dosage and method of administration vary according to the body-weight and age of a patient and the administration method; however, one skilled in the art can routinely select them. If said compound is encodable by a DNA, the DNA can be inserted into a vector for gene therapy and the vector administered to perform the therapy. The dosage and method of administration vary according to the body-weight, age, and symptoms of a patient but one skilled in the art can select them suitably.

For example, although there are some differences according to the symptoms, the dose of a compound that binds with the polypeptide of the present invention and regulates its activity is about 0.1 mg to about 100 mg per day, for example, about 1.0 mg to about 50 mg per day, for example, about 1.0 mg to about 20 mg per day, when administered orally to a normal adult (weight 60 kg).

When administering parenterally, in the form of an injection to a normal adult (weight 60 kg), although there are some differences according to the patient, target organ, symptoms and method of administration, it is convenient to intravenously inject a dose of about 0.01 mg to about 30 mg per day, for example, about 0.1 to about 20 mg per day, for example, about 0.1 to about 10 mg per day. Also, in the case of other animals too, it is possible to administer an amount converted to 60 kgs of body-weight.

The agents can be administered orally or by injection (intravenous or subcutaneous), and the precise amount administered to a subject will be determined under the responsibility of the attendant physician, considering a number of factors, including the age and sex of the subject, the precise disorder being treated, and its severity. Also the route of administration can vary depending upon the condition and its severity.

Moreover, the present invention provides a method for treating or preventing cancer expressing CX gene, e.g. lung cancer and/or esophageal cancer, using an antibody against a polypeptide of the present invention. According to the method, a pharmaceutically effective amount of an antibody against the polypeptide of the present invention is administered. Since the expression of the CX protein is up-regulated in cancer cells, and the suppression of the expression of these proteins leads to the decrease in cell proliferating activity, it is expected that lung cancer and/or esophageal cancer can be treated or prevented by binding the antibody and these proteins. Thus, an antibody against a polypeptide of the present invention can be administered at a dosage sufficient to reduce the activity of the protein of the present invention, which is in the range of 0.1 to about 250 mg/kg per day. The dose range for adult humans is generally from about 5 mg to about 17.5 g/day, for example, about 5 mg to about 10 g/day, for example, about 100 mg to about 3 g/day.

Generally, an efficacious or effective amount of one or more CX protein inhibitors is determined by first administering a low dose or small amount of a CX protein inhibitor and then incrementally increasing the administered dose or dosages, and/or adding a second CX protein inhibitor as needed, until a desired effect of inhibiting or preventing lung cancer and/or esophageal cancer is observed in the treated subject, with minimal or no toxic side effects. Applicable methods for determining an appropriate dose and dosing schedule for administration of a pharmaceutical composition of the present invention is described, for example, in Goodman and Gilman's The Pharmacological Basis of Therapeutics, 11th Ed., Brunton, et al., Eds., McGraw-Hill (2006), and in Remington: The Science and Practice of Pharmacy, 21st Ed., University of the Sciences in Philadelphia (USIP), Lippincott Williams & Wilkins (2005), both of which are hereby incorporated herein by reference.

The agents screened by the present methods further can be used for treating or preventing cancers expressing CX gene, e.g. lung cancer and/or esophageal cancer, in a subject. Administration can be prophylactic or therapeutic to a subject at risk of (or susceptible to) a disorder or having a disorder associated with aberrant phosphorylation activity of the CX protein. The method includes decreasing the function of CX protein in lung cancer cell and/or esophageal cancer cells. The function can be inhibited through the administration of an agent obtained by the screening method of the present invention.

Herein, the term “preventing” means that the agent is administered prophylactically to retard or suppress the forming of tumor or retards, suppresses, or alleviates at least one clinical symptom of cancer. Assessment of the state of tumor in a subject can be made using standard clinical protocols.

Alternatively, an antibody binding to a cell surface marker specific for tumor cells can be used as a tool for drug delivery. For example, the antibody conjugated with a cytotoxic agent is administered at a dosage sufficient to injure tumor cells.

### Screening Kits:

The present invention also provides an article of manufacture or kit containing materials for screening for an agent useful in treating or preventing cancer, particularly breast, bladder, or lung cancer. Such an article of manufacture can comprise one or more labeled containers of materials described herein along with instructions for use. Suitable containers include, for example, bottles, vials, and test tubes. The containers can be formed from a variety of materials for example, glass or plastic.

[1] A kit for screening for an agent interrupts a binding between an EPHA7 polypeptide and an EGFR polypeptide, wherein the kit comprises:

(a) a polypeptide comprising an EGFR-binding domain of an EPHA7 polypeptide;

(b) a polypeptide comprising an EPHA7-binding domain of an EGFR polypeptide; and

(c) means to detect the interaction or binding between the polypeptides.

In some embodiments, the polypeptide of (a), i.e., the polypeptide comprising the EGFR-binding domain, comprises an EPHA7 polypeptide. Similarly, in other embodiments, the polypeptide of (b), i.e., the polypeptide comprising the EPHA7-binding domain comprises an EGFR polypeptide.

[2] A kit for screening for an agent that modulate an EPHA7-mediated phosphorylation of EGFR, wherein the kit comprises:

(a) a polypeptide comprising an protein kinase domain of an EPHA7 polypeptide, or functional equivalent thereof;

(b) a polypeptide comprising an EPHA7-mediated phosphorylation site of an EGFR polypeptide, or functional equivalent thereof; and

(c) means to detect the phosphorylation level of the polypeptide of (b).

In some embodiments, the polypeptide of (a), i.e., the functional equivalent of EGFR polypeptide comprises at least one EPHA7-mediated phosphorylation site of the polypeptide. And the EPHA7-mediated phosphorylation site is Tyr845 of EGFR polypeptide

[3] A kit for screening for an agent for preventing or treating cancers, wherein the kit comprises:

(a) a polypeptide comprising an protein kinase domain of an STK31 polypeptide;

(b) a substrate; and

(c) means to detect the phosphorylation level of the substrate of (b).

In some embodiments, the substrate is BMP.

[4] A kit for screening for an agent for preventing or treating cancers, wherein the kit comprises:

(a) a cell expressing a gene encoding WDHD1 polypeptide or functional equivalent thereof; and

(b) means to detect the phosphorylation level of the polypeptide of (a).

In some embodiments, the polypeptide for the screening of the present invention is expressed in a living cell.

[5] A kit for screening for an agent interrupts an interaction or binding between a CDCA5 polypeptide and a CDC2 polypeptide, wherein the kit comprises:

(a) a polypeptide comprising a CDC2-interacting domain of a CDCA5 polypeptide;

(b) a polypeptide comprising a CDCA5-interacting domain of an CDC2 polypeptide; and

(c) means to detect the interaction or binding between the polypeptides.

[6] A kit for screening for an agent that modulate a CDC2-mediated phosphorylation of CDCA5, wherein the kit comprises:

(a) a polypeptide comprising a protein kinase domain of a CDC2 polypeptide;

(b) a polypeptide comprising a CDC2-mediated phosphorylation site of a CDCA5 polypeptide, or functional equivalent thereof; and

(c) means to detect the phosphorylation level of the polypeptide of (b).

[7] A kit for screening for an agent for preventing or treating cancers expressing CDCA5, wherein the kit comprises:

(a) a polypeptide comprising a protein kinase domain of a CDC2 polypeptide, or functional equivalent thereof;

(b) a polypeptide comprising a CDC2-mediated phosphorylation site of a CDCA5 polypeptide, or functional equivalent thereof; and

(c) means to detect the phosphorylation level of the polypeptide of (b).

[8] A kit for screening for an agent for preventing or treating cancers, wherein the kit comprises:

(a) a cell expressing a gene encoding CDCA5 polypeptide or functional equivalent thereof; and

(b) means to detect the phosphorylation level of the polypeptide of (a).

[9] A kit for screening for an agent interrupts an interaction or binding between a CDCA5 polypeptide and an ERK polypeptide, wherein the kit comprises:

(a) a polypeptide comprising an ERK-interacting domain of a CDCA5 polypeptide;

(b) a polypeptide comprising a CDCA5-interacting domain of an ERK polypeptide; and

(c) means to detect the interaction or binding between the polypeptides.

[10] A kit for screening for an agent that modulate an ERK-mediated phosphorylation of CDCA5, wherein the kit comprises:

(a) a polypeptide comprising a protein kinase domain of ERK polypeptide;

(b) a polypeptide comprising an ERK-mediated phosphorylation site of a CDCA5 polypeptide, or functional equivalent thereof; and

(c) means to detect the phosphorylation level of the polypeptide of (b).

[11] A kit for screening for an agent for preventing or treating cancers expressing CDCA5, wherein the kit comprises:

(a) a polypeptide comprising a protein kinase domain of an ERK polypeptide, or functional equivalent thereof;

(b) a polypeptide comprising an ERK-mediated phosphorylation site of a CDCA5 polypeptide, or functional equivalent thereof; and

(c) means to detect the phosphorylation level of the polypeptide of (b).

[12] A kit for screening for an agent for preventing or treating cancers, wherein the kit comprises:

(a) a cell expressing a gene encoding CDCA5 polypeptide or functional equivalent thereof; and

(b) means to detect the phosphorylation level of the polypeptide of (a).

The present invention further provides articles of manufacture and kits containing materials useful for treating the pathological conditions described herein are provided. Such an article of manufacture can comprise a container of a medicament as described herein with a label. As noted above, suitable containers include, for example, bottles, vials, and test tubes. The containers can be formed from a variety of materials for example, glass or plastic. In the context of the present invention, the container holds a composition having an active agent which is effective for treating a cell proliferative disease, for example, lung cancer or esophageal cancer. The active agent in the composition can be an identified test compound (e.g., antibody, small molecule, etc.) capable of disrupting the EPHA7/EGFR, CDCA5/CDC2 or CDCA5/ERK association in vivo, inhibiting an EPHA7-mediated phosphorylation of EGFR, inhibiting an STK31 kinase activity, or inhibiting a phosphorylation of WDHD1 or CDCA5. The label on the container can indicate that the composition is used for treating one or more conditions characterized by abnormal cell proliferation. The label can also indicate directions for administration and monitoring techniques, for example, those described herein.

In addition to the container described above, a kit of the present invention can optionally comprise a second container housing a pharmaceutically-acceptable diluent. It can further include other materials desirable from a commercial end-user standpoint, including other buffers, diluents, filters, needles, syringes, and package inserts with instructions for use.

The compositions can, if desired, be presented in a pack or dispenser device which can contain one or more unit dosage forms containing the active ingredient. The pack can, for example, comprise metal or plastic foil, for example, a blister pack. The pack or dispenser device can be accompanied by instructions for administration. Compositions comprising an agent of the invention formulated in a compatible pharmaceutical carrier can also be prepared, placed in an appropriate container, and labeled for treatment of an indicated condition.

Hereinafter, the present invention is described in more detail by reference to the Examples. However, the following materials, methods and examples only illustrate aspects of the invention and in no way are intended to limit the scope of the present invention. As such, methods and materials similar or equivalent to those described herein can be used in the practice or testing of the present invention.

### EXAMPLE

The invention will be further described in the following examples, which do not limit the scope of the invention described in the claims.

### Example 1

### (1) Cell Lines and Clinical Samples

The 23 human lung cancer cell lines used in this study included nine adenocarcinomas (ADCs; A427, A549, LC319, NCI-H1373, PC-3, PC-9, PC-14, NCI-H1666, and NCI-H1781), nine squamous cell carcinomas (SCCs; EBC-1, LU61, NCI-H520, NCI-H1703, NCI-H2170, RERF-LC-AI, and SK-MES-1, NCI-H226, and NCI-H647), one large-cell carcinoma (LCC; LX1), and four small-cell lung cancers (SCLCs; DMS114, DMS273, SBC-3, and SBC-5). The human esophageal carcinoma cell lines used in this study were as follows: nine SCC cell lines (TE1, TE2, TE3, TE4, TE5, TE6, TE8, TE9, and TE10) and one adenocarcinoma (ADC) cell line (TE7) (Nishihira T, et al., J Cancer Res Clin Oncol 1993; 119: 441-49).

All cells were grown in monolayers in appropriate media supplemented with 10% fetal calf serum (FCS) and were maintained at 37 degrees C. in an atmosphere of humidified air with 5% CO₂. Human small airway epithelial cells (SAEC) were grown in optimized medium (SAGM) purchased from Cambrex Bio Science Inc. (Walkersville, Md.). Primary lung cancer and ESCC samples had been obtained earlier with informed consent (Kikuchi T, et al., Oncogene 2003; 22: 2192-205; Taniwaki M, et al., Int J Oncol 2006; 29: 567-75; Yamabuki T, et al., Int J Oncol 2006; 28: 1375-84).

Clinical stage was judged according to the International Union Against Cancer TNM classification (Sobin L & Wittekind Ch. TNM Classification of Malignant Tumours, 6th edition. New York: Wiley-Liss; 2002). Formalin-fixed primary NSCLCs (total 402 cases for EPHA7; total 368 cases for STK31; total 264 cases for WDHD1) and adjacent normal lung-tissue samples for immunostaining on tissue microarray were also obtained from patients who underwent surgery. Formalin-fixed primary ESCCs (total 292 cases for EPHA7; total 297 cases for WDHD1) and adjacent normal esophageal tissue samples had also been obtained from patients undergoing curative surgery. 27 SCLC samples obtained from patients undergoing curative surgery for EPHA7. This study and the use of all clinical materials were approved by individual institutional ethical committees.

### (2) Serum Samples

Serum samples were obtained with written informed consent from 127 healthy control individuals (100 males and 27 females; median age of 53 with a range of 31-61 years), and from 89 non-neoplastic lung disease patients with chronic obstructive pulmonary disease (COPD) enrolled as a part of the Japanese Project for Personalized Medicine (BioBank Japan) or admitted to Hiroshima University Hospital (78 males and 11 females; median age of 68 with a range of 54-84 years). All of these patients were current and/or former smokers (The mean [+/−1 SD] of pack-year index (PYI) was 71.9+/−45.4; PYI was defined as the number of cigarette packs [20 cigarette per pack] consumed a day multiplied by years).

Serum samples were also obtained with informed consent from 214 lung cancer patients admitted to Hiroshima University Hospital, as well as Kanagawa Cancer Center Hospital, and from 129 patients with lung cancer who were registered in the BioBank Japan (229 males and 114 females; median age, 68+/−10.8 SD; range, 30-89 years). These 343 cases included 205 lung ADCs, 59 SCCs, and 79 SCLCs. Serum samples were also obtained with informed consent from 96 ESCC patients who were admitted to Keiyukai Sapporo Hospital or who were registered in the BioBank Japan (79 males and 17 females; median age of 63 with a range of 37-74 years), as well as from 102 cervical cancer patients who were registered in the BioBank Japan (102 females; median age of 46 with a range of 40-55 years).

Samples were selected for the study on the basis of the following criteria: (a) patients were newly diagnosed and previously untreated and (b) their tumors were pathologically diagnosed as lung cancers (stages I-IV). Serum was obtained at the time of diagnosis and stored at −150 degree Centigrade.

### (3) Semi-Quantitative RT-PCR

Total RNA was extracted from cultured cells using Trizol reagent (Life Technologies, Inc. Gaithersburg, Md.) according to the manufacturer's protocol. Extracted RNAs were treated with DNase I (Nippon Gene, Tokyo, Japan) and reversely-transcribed using oligo (dT) primer and SuperScript II. The primer sets for amplification were as follows:

(SEQ ID NO: 9)
ACTB-F: 5′-GAGGTGATAGCATTGCTTTCG-3′
and
(SEQ ID NO: 10)
ACTB-R: 5′-CAAGTCAGTGTACAGGTAAGC-3′,
for ACTB
(SEQ ID NO: 11)
CDCA5-F: 5′-CGCCAGAGACTTGGAAATGT-3′
and
(SEQ ID NO: 12)
CDCA5-R: 5′-GTTTCTGTTTCTCGGGTGGT-3′,
for CDCA5
(SEQ ID NO: 13)
EPHA7-F: 5′-GCAGGTAGTCAAGAAAATGCAAG-3′
and
(SEQ ID NO: 14)
EPHA7-R: 5′-CAGATCCTTCACCTCTTCCTTCT-3′,
for EPHA7
(SEQ ID NO: 15)
STK31-F: 5′-AAGCCAAAGAAGGAGCAAAT-3′
and
(SEQ ID NO: 16)
STK31-R: 5′-CAATGAGCCTTTCCTCTGAA-3′,
for STK31
(SEQ ID NO: 17)
WDHD1-F: 5′-AGTGAAGGAACTGAAGCAAAGAAG-3′
and
(SEQ ID NO: 18)
WDHD1-R: 5′-ATCCATTACTTCCCTAGGGTCAC-3′.
for WDHD1

PCR reactions were optimized for the number of cycles to ensure product intensity within the logarithmic phase of amplification.

### (4) Northern-Blot Analysis

Human multiple-tissue blots (23 normal tissues including heart, brain, placenta, lung, liver, skeletal muscle, kidney, pancreas, spleen, thymus, prostate, testis, ovary, small intestine, colon, leukocyte, stomach, thyroid, spinal cord, lymph node, trachea, adrenal gland, bone marrow; BD Biosciences Clontech, Palo Alto, Calif.) were hybridized with an [alpha-³²P]-dCTP-labeled PCR product of CDCA5, EPHA7, STK31. The partial-length cDNAs were prepared by RT-PCR using primers as follows:

CDCA5-F:

| | |
|---|---|
| 5′-GCTTGTAAAGTCCTCGGAAAGTT-3′ | (SEQ ID NO: 19) |
| and | |
| CDCA5-R: | |
| 5′-ATCTCAACTCTGCATCATCTGGT-3′ | (SEQ ID NO: 20) |
| for CDCA5, | |
| EPHA7-F: | |
| 5′-GCAGGTAGTCAAGAAAATGCAAG-3′ | (SEQ ID NO: 13) |
| and | |
| EPHA7-R: | |
| 5′-CAGATCCTTCACCTCTTCCTTCT-3′ | (SEQ ID NO: 14) |
| for EPHA7, | |
| STK31-F: | |
| 5′-GAAAATGGGAAAACCTGCTT-3′ | (SEQ ID NO: 21) |
| and | |
| STK31-R: | |
| 5′-CAATGAGCCTTTCCTCTGAA-3′ | (SEQ ID NO: 16) |
| for STK31 (516-bp) | |
| WDHD1-F: | |
| 5′-CTCTGATTCCAAAGCCGAAG-3′ | (SEQ ID NO: 22) |
| and | |
| WDHD1-R: | |
| 5′-ATCCATTACTTCCCTAGGGTCAC-3′ | (SEQ ID NO: 18) |
| for WDHD1 (535-bp). | |

Pre-hybridization, hybridization, and washing were performed according to the supplier's recommendations. The blots were autoradiographed with intensifying BAS screens (Bio-Rad Laboratories, Hercules, Calif.) at −80 degrees C. for 7 days. for CDCA5, at −80 degree Centigrade for 2 weeks for EPHA7, at room temperature for 30 h for STK31 or at −80 degree Centigrade for 7 days for WDHD1.

### (5) Western-Blotting

Tumor tissues or cells were lysed in lysis buffer; 50 mM Tris-HCl (pH 8.0), 150 mM NaCl, 0.5% NP-40, 0.5% deoxycholate-Na, 0.1% SDS, and Protease Inhibitor Cocktail Set III (EMD Biosciences, Inc., San Diego, Calif.). The protein content of each lysate was determined by a Bio-Rad protein assay (Hercules, Calif.) with bovine serum albumin (BSA) as a standard. Ten micrograms of each lysate were resolved on 10-12% denaturing polyacrylamide gels (with 3% polyacrylamide stacking gel) and transferred electrophoretically to a nitrocellulose membrane (GE Healthcare Bio-sciences, Piscataway, N.J.). For STK31, after blocking with 5% non-fat dry milk in TBST, the membrane was incubated with primary antibodies for 1 h at room temperature. For WDHD1, after blocking with Block Ace (Dainippon Seiyaku, Osaka, Japan) in TBS-Tween 20 (TBST), the membrane was incubated with primary antibodies for overnight at −4 degree Centigrade. Immunoreactive proteins were incubated with horseradish peroxidase-conjugated secondary antibodies (GE Healthcare Bio-sciences) for 1 h at room temperature. After washing with TBST, the reactants were developed using the enhanced chemiluminescence kit (GE Healthcare Bio-sciences).

Commercially available antibodies used in this studies were as follows:

Rabbit polyclonal antibodies (Catalog No. sc25459, Santa Cruz, Santa Cruz, Calif.) for epitope(s) from N-terminal portion of human EPHA7;

Rabbit polyclonal antibodies (Catalog No. ab5411, Abcam) for epitope(s) from C-terminal portion of human EPHA7;

Rabbit polyclonal antibody to human STK31 (ABGENT, San Diego, Calif.); and

Rabbit polyclonal antibody to human WDHD1 (ATLAS Antibodies AB (Stockholm, Sweden)).

To identify substrate and/or downstream target proteins that would be phosphorylated through EPHA7 signaling and activate cell-proliferation signaling. The present inventors performed immunoblot-screening of kinase substrates for EPHA7 using cell lysates of COS-7 cells transfected with EPHA7-expression vector and a series of antibodies specific for phospho-proteins related to cancer-cell signaling (see Table 2).

TABLE 2
The list of a series of antibodies specific for phospho-proteins
related to cancer-cell signaling

| | | Catalog | | |
|---|---|---|---|---|
| antibody | company | No. | EPHA7 | STK31 |
| pEGFR(Tyr845) | Cell | #2231L | ∘ | ∘ |
| | signaling | | | |
| pEGFR(Tyr1068) | Cell | #2234 | ∘ | |
| | Signaling | | | |
| pEGFR(Tyr992) | Cell | #2235L | ∘ | ∘ |
| | signaling | | | |
| pEGFR(Tyr1068) | Cell | #2236L | ∘ | ∘ |
| (1H12) | signaling | | | |
| pEGFR(Tyr1045) | Cell | #2237L | ∘ | ∘ |
| | signaling | | | |
| pEGFR(Ser1046/1047)) | Cell | #2238S | | ∘ |
| | signaling | | | |
| Phospho-Shc (Tyr317) | Cell | #2431 | ∘ | |
| | Signaling | | | |
| Phospho-Shc | Cell | #2434 | ∘ | |
| (Tyr239/240) | Signaling | | | |
| phospho-Chk2 (Thr68) | Cell | #2661 | | ∘ |
| | signaling | | | |
| Phospho-PLCgamma1 | Cell | #2821 | ∘ | |
| (Tyr783) | Signaling | | | |
| Phospho-PLCgamma1 | Cell | #2824 | ∘ | |
| (Tyr771) | Signaling | | | |
| phospho-nucleophosmin | Cell | #3541 | ∘ | |
| (Thr199) | Signaling | | | |
| Phospho-Gab2 (Tyr452) | Cell | #3881 | ∘ | |
| | Signaling | | | |
| pAKT(Ser473)(587F11) | Cell | #4051L | | ∘ |
| | signaling | | | |
| Phospho-EGF Receptor | Cell | #4404 | ∘ | ∘ |
| (Tyr1148) | Signaling | | | |
| phospho-ATM | Cell | #4526 | ∘ | |
| (Ser1981)(10H11.E12) | Signaling | | | |
| phospho-p38 MAPK | Cell | #4631 | ∘ | ∘ |
| (Thr180/Tyr182)(12F8) | Signaling | | | |
| Rabbit mAb | | | | |
| phospho-p44/42 Map | Cell | #9101 | ∘ | ∘ |
| Kinase | Signaling | | | |
| (Thr202/Tyr204) | | | | |
| Antibody | | | | |
| pSTAT3(Tyr705) | Cell | #9131 | ∘ | |
| | Signaling | | | |
| pSTAT3(Ser727) | Cell | #9134L | | ∘ |
| | signaling | | | |
| pSTAT3(Ser727)(6E4) | Cell signaling | #9136L | | ∘ |
| pSTAT3(Tyr705)(3E2) | Cell Signaling | #9138 | ∘ | |
| pSTAT1(Tyr701) | Cell Signaling | #9171 | ∘ | |
| Phospho-SAPK/JNK | Cell Signaling | #9251 | ∘ | ∘ |
| (Thr183/Tyr185) | | | | |
| pAKT(Ser473) | Cell signaling | #9271L | | ∘ |
| pAKT(Thr308) | Cell signaling | #9275L | | ∘ |
| phospho-p53 (ser20) | Cell signaling | #9287S | | ∘ |
| pSTAT5(Tyr694) | Cell Signaling | #9351 | ∘ | |
| phospho-cdc25 (ser216) | Cell Signaling | #9528 | ∘ | |
| pEGFR(Tyr1173)(9H2) | Upatate | 05-483 | ∘ | |
| phospho-nucleophosmin | Cell | 3541S | | ∘ |
| (Thr199) | signaling | | | |
| phosph-ser46-p53/rabbit | CALBIOCHEM | DR1024 | | ∘ |
| phosph-ser15-p53/rabbit | CALBIOCHEM | PC386 | | ∘ |
| anti-p-SMAD2/3 | Santa Cruz | sc-11769 | | ∘ |
| (Ser433/435)-R | | | | |
| anti-p-SMAD1 | Santa Cruz | sc-12353 | ∘ | ∘ |
| (Ser463/Ser465)-R | | | | |
| p-Bcl-2 Ab | Santa Cruz | sc-16323-R | ∘ | ∘ |
| (Rabbit: ser87) | | | | |
| anti-p-IKK | Santa Cruz | sc-21660 | ∘ | ∘ |
| alpha/beta(Thr23) | | | | |
| p-p38(D-8), human | Santa Cruz | sc-7973 | ∘ | ∘ |
| p-Akt1/2/3(Ser473) | Santa Cruz | sc-7985-R | | ∘ |
| p-Bad (Ser136) | Santa Cruz | sc-7999 | | ∘ |
| anti-p-IkB-alpha(B-9) | Santa Cruz | sc-8404 | ∘ | |

### (6) Expression Vector

The entire coding sequence of CDCA5 (74-829 nt of SEQ ID NO: 1) or EPHA7 (214-3210 nt of SEQ ID NO: 3) or WDHD1 (79-3468 nt of SEQ ID NO: 5) was cloned into the appropriate site of pcDNA3.1 myc-His plasmid vector (invitrogen). The entire coding sequence of STK31 (467-3457 nt of SEQ ID NO: 7) was cloned into the appropriate site of pCAGGSn3FC vector.

c-Myc-tagged CDCA5 (pcDNA3.1/myc-His-CDCA5), c-Myc-tagged EPHA7 (pcDNA3.1/myc-His-EPHA7), c-Myc-tagged WDHD1 (pcDNA3.1/myc-His-WDHD1) or FLAG-tagged STK31 (pCAGGSn3FC-STK31) or mock (pcDNA3.1/myc-His or pCAGGSn3FC) was transfected into COS-7 cells using FuGENE6 transfection reagent (Roche).

### (7) Immunocytochemical Analysis

Cultured cells were washed twice with PBS(−), fixed in 4% formaldehyde solution for 30 min at room temperature and then rendered permeable with PBS(−) containing 0.1% Triton X-100 for 3 min at room temperature. Nonspecific binding was blocked by Casblock (ZYMED, San Francisco, Calif.) for 10 min at room temperature for CDCA5 and WDHD1, by Casblock (ZYMED, San Francisco, Calif.) for 7 min at room temperature for EPHA7, 3% bovine serum albumin in PBS(−) for 7 min at room temperature for STK31. Cells were then incubated for 60 min (for CDCA5, EPHA7 or STK31) or 10 min (for WDHD1) at room temperature with primary antibodies diluted in PBS containing 3% BSA. After being washed with PBS(−), the cells were stained by a donkey anti-rabbit secondary antibody conjugated to Alexa488 (Molecular Probes) (for CDCA5 and EPHA7) or FITC-conjugated secondary antibody (Santa Cruz Biotechnology, Santa Cruz, Calif.) (for STK31 and WDHD1) at 1:1,000 dilutions for 60 min at room temperature. After another wash with PBS(−), each specimen was mounted with Vectashield (Vector Laboratories, Inc., Burlingame, Calif.) containing 4′,6-diamidino-2-phenylindole and visualized with Spectral Confocal Scanning Systems (TSC SP2 AOBS; Leica Microsystems, Wetzlar, Germany).

Commercially available antibodies used as primary antibodies in this studies were as follows:

Rabbit polyclonal anti-c-Myc antibody (Santa Cruz Biotechnology, Santa Cruz, Calif.) for exogenous CDCA5;

Rabbit polyclonal antibodies (Catalog No. sc25459, Santa Cruz, Santa Cruz, Calif.) for epitope(s) from N-terminal portion of human EPHA7;

Rabbit polyclonal antibodies (Catalog No. ab5411, Abcam) for epitope(s) from C-terminal portion of human EPHA7;

Rabbit polyclonal antibody against human STK31 (ABGENT, San Diego, Calif.) for STK31; and

Rabbit polyclonal anti-WDHD1 antibody (ATLAS Antibodies AB) for WDHD1.

### (8) Immunohistochemistry and Tissue-Microarray Analysis

The tissue sections were stained tissue sections using ENVISION+ Kit/HRP (DakoCytomation, Glostrup, Denmark). The primary antibody was added after blocking of endogenous peroxidase and proteins, and each section was incubated with HRP-labeled anti-rabbit IgG (Histofine Simple Stain MAX PO (G), Nichirei, Tokyo, Japan) as the secondary antibody. Substrate-chromogen was added and the specimens were counterstained with hematoxylin. Tumor-tissue microarrays were constructed as published previously, using formalin-fixed NSCLCs (Chin S F, et al., Mol Pathol. 2003 October; 56(5): 275-9; Callagy G, et al., Diagn Mol Pathol. 2003 March; 12(1): 27-34; J Pathol. 2005 February; 205(3):388-96). Tissue areas for sampling were selected based on visual alignment with the corresponding HE-stained sections on slides. Three, four, or five tissue cores (diameter 0.6 mm; height 3-4 mm) taken from donor-tumor blocks were placed into recipient paraffin blocks using a tissue microarrayer (Beecher Instruments, Sun Prairie, Wis.). A core of normal tissue was punched from each case, and 5-1 μm sections of the resulting microarray block were used for immunohistochemical analysis. Positivity of staining was assessed semi-quantitatively by three independent investigators without prior knowledge of the clinicopathological data and clinical follow-up data. The intensity of staining was evaluated using following criteria:

positive (1+), brown staining appreciable in the nucleus and cytoplasm of tumor cells;

negative (0), no appreciable staining in tumor cells.

Cases were accepted only as strong positive if reviewers independently defined them as such.

Commercially available antibodies used as primary antibodies in these studies were as follows:

Rabbit polyclonal antibodies (Catalog No. sc25459, Santa Cruz, Santa Cruz, Calif.) for epitope(s) from N-terminal portion of human EPHA7;

Rabbit polyclonal antibody against human STK31 (ABGENT, San Diego, Calif.) for STK31; and

Rabbit polyclonal anti-WDHD1 antibody (ATLAS Antibodies AB) for WDHD1.

### (9) Statistical Analysis

Statistical analyses were performed using the StatView statistical program (SaS, Cary, N.C., USA). We used contingency tables to analyze the relationship between CX gene expression and clinicopathological variables in NSCLC or ESCC patients. Tumor-specific survival curves were calculated from the date of surgery to the time of death related to NSCLC or ESCC, or to the last follow-up observation. Kaplan-Meier curves were calculated for each relevant variable and for CX gene expression; differences in survival times among patient subgroups were analyzed using the log-rank test. Univariate and multivariate analyses were performed with the Cox proportional-hazard regression model to determine associations between clinicopathological variables and CX mortality. First, we analyzed associations between death and prognostic factors including age, gender, smoking history, histological type, pT-classification, and pN-classification, taking into consideration one factor at a time. Second, multivariate Cox analysis was applied on backward (stepwise) procedures that always forced CX gene expression into the model, along with any and all variables that satisfied an entry level of a P-value less than 0.05. As the model continued to add factors, independent factors did not exceed an exit level of P<0.05.

### (10) ELISA

Serum levels of EPHA7 were measured by ELISA system which had been originally constructed. First of all, a rabbit polyclonal antibody specific to N-terminal portion of human EPHA7 (Catalog No. sc25459, Santa Cruz, Santa Cruz, Calif.) was added to a 96-well microplate (Apogent, Denmark) as a capture antibody and incubated for 2 hours at room temperature. After washing away any unbound antibody, 5% BSA was added to the wells and incubated for 16 hours at 4 degree Centigrade for blocking. After a wash, 3-fold diluted sera were added to a 96-well microplate precoated with capture antibody and incubated for 2 hours at room temperature. After washing away any unbound substances, a biotinylated polyclonal antibody specific for EPHA7 using Biotin Labeling Kit-NH2 (Dojindo Molecular Technologies, Inc., Kumamoto, Japan) was added to the wells and incubated for 2 hours at room temperature. After a wash to remove any unbound antibody-enzyme reagent, HRP-streptavisin was added to the wells and incubated for 20 minutes. After a wash, a substrate solution (R&D Systems, Inc., Minneapolis, Minn.) was added to the wells and allowed to react for 30 minutes. The reaction was stopped by adding 100 μl of 2N sulfuric acid. Color intensity was determined by a photometer at a wavelength of 450 nm, with a reference wavelength of 570 nm. Levels of CEA in serum were measured by ELISA with a commercially available enzyme test kit (HOPE Laboratories, Belmont, Calif.), according to the supplier's recommendations. Levels of ProGRP in serum were measured by ELISA with a commercially available enzyme test kit (TFB, Tokyo, Japan), according to the manufacturer's protocol. Differences in the levels of EPHA7, CEA, and ProGRP between tumor groups and a healthy control group were analyzed by Mann-Whitney U tests. The levels of EPHA7, CEA, and ProGRP were evaluated by receiver-operating characteristic (ROC) curve analysis to determine cutoff levels with optimal diagnostic accuracy and likelihood ratios. The correlation coefficients between EPHA7 and CEA/ProGRP were calculated with Spearman rank correlation. Significance was defined as P<0.05.

### (11) RNA Interference Assay

### (i) Oligo Based Assay

Small interfering RNA (siRNA) duplexes (Dharmacon, Inc., Lafayette, Colo.) (600 pM) were transfected into lung-cancer cell lines LC319 and A549 for CDCA5; NCI-H520 and SBC-5 for EPHA7; LC319 for WDHD1, and esophageal cancer cell line TE9 for WDHD1 using 30 μl of Lipofectamine 2000 (Invitrogen, Carlsbad, Calif.) following the manufacturer's protocol. The transfected cells were cultured for 7 days, and the number of colonies was counted by Giemsa staining, and viability of cells was evaluated by 3-(4,5-dimethylthiazol-2-yl)-2,5-diphenyltetrazolium bromide (MTT) assay (cell counting kit-8 solution; Dojindo Laboratories, Kumanoto, Japan), at 7 days after transfection. To confirm suppression of gene expression, semiquantitative RT-PCR was carried out with synthesized primers described above. The siRNA sequences used were as follows:

control-1
(si-LUC: luciferase gene from Photinus pyralis):

| | |
|---|---|
| 5′-NNCGUACGCGGAAUACUUCGA-3′; | (SEQ ID NO: 23) |
| control-2 | |

(CNT: ON-TARGETplus siCONTROL Non-targeting

| | |
|---|---|
| siRNAs pool): | |
| mixture of | |
| 5′-UGGUUUACAUGUCGACUAA-3′, | (SEQ ID NO: 24) |
| 5′-UGGUUUACAUGUUUUCUGA-3′, | (SEQ ID NO: 25) |
| 5′-UGGUUUACAUGUUUUCCUA-3′ | (SEQ ID NO: 26) |
| and | |
| 5′-UGGUUUACAUGUUGUGUGA-3′; | (SEQ ID NO: 27) |
| control-3 | |

(Scramble/SCR: chloroplast Euglena gracilis gene

| | |
|---|---|
| coding for 5S and 16S rRNAs): | |
| 5′-NNGCGCGCUUUGUAGGAUUCG-3′; | (SEQ ID NO: 28) |
| control-4 | |

(EGFP: enhanced green fluorescent protein (GFP)
gene, a mutant of Aequorea victoria GFP),

| | |
|---|---|
| 5′-NNGAAGCAGCACGACUUCUUC-3′ | (SEQ ID NO: 29) |
| si-CDCA5-#1: | |
| 5′-GCAGUUUGAUCUCCUGGUUU-3′; | (SEQ ID NO: 30) |
| si-CDCA5-#2: | |
| 5′-GCCAGAGACUUGGAAAUGU UU-3′; | (SEQ ID NO: 31) |
| si-EPHA7-#1 (D-003119-05): | |
| 5′-AAAAGAGAUGUUGCAGUA-3′; | (SEQ ID NO: 32) |
| si-EPHA7-#2 (D-003119-08): | |
| 5′-UAGCAAAGCUGACCAAGAA-3′; | (SEQ ID NO: 33) |
| si-WDHD1-#1 (D-019780-01): | |
| 5′-GAUCAGACAUGUGCUAUUA UU-3′; | (SEQ ID NO: 34) |
| and | |
| si-WDHD1-#2 (D-019780-02): | |
| 5′-GGUAAUACGUGGACUCCUA UU-3′. | (SEQ ID NO: 35) |

### (ii) Vector Based Assay

The present inventors had established previously a vector-based RNAi system, psiH1BX3.0, which was designed to synthesize small interfering RNAs (siRNA) in mammalian cells (Suzuki C, et al., Cancer Res. 2003 Nov. 1; 63(21): 7038-41). Ten micrograms of siRNA expression vector were transfected using 30 μL Lipofectamine 2000 (Invitrogen) into lung cancer cell lines, LC319 and NCI-H2170. The transfected cells were cultured for 7 days in the presence of appropriate concentrations of geneticin (G418), and the number of colonies was counted by Giemsa staining, and viability of cells was evaluated by 3-(4,5-dimethylthiazol-2-yl)-2,5-diphenyltetrazolium bromide (MTT) assay (cell counting kit-8 solution; Dojindo, Kumamoto, Japan), at 7 days after the G418 treatment. To confirm suppression of STK31 protein expression, Western blotting was carried out with affinity-purified polyclonal antibody to STK31 according to the standard protocol. The target sequences of the synthetic oligonucleotides for RNAi were as follows:

control 1
(enhanced green fluorescent protein (EGFP) gene,
a mutant of Aequorea victoria GFP),

| | |
|---|---|
| 5′-GAAGCAGCACGACTTCTTC-3′; | (SEQ ID NO: 36) |
| control 2 | |

(Luciferase/LUC: Photinus pyralis luciferase

| | |
|---|---|
| gene), | |
| 5′-CGTACGCGGAATACTTCGA-3′; | (SEQ ID NO: 37) |
| si-STK31-#1, | |
| 5′-GGAGATAGCTCTGGTTGAT-3′; | (SEQ ID NO: 38) |
| and | |
| si-STK31-#2, | |
| 5′-GGGCTATTCTGTGGATGTT-3′. | (SEQ ID NO: 49) |

### (12) Cell-Growth Assay

COS-7 cells transfected either with plasmids expressing myc-His-tagged EPHA7, FLAG-tagged STK31 or with mock plasmids were grown for eight days in DMEM containing 10% FCS in the presence of appropriate concentrations of geneticin (G418). Viability of cells was evaluated by MTT assay; briefly, cell-counting kit-8 solution (DOJINDO) was added to each dish at a concentration of 1/10 volume, and the plates were incubated at 37 degree Centigrade for additional 2 hours. Absorbance was then measured at 490 nm, and at 630 nm as a reference, with a Microplate Reader 550 (BIO-RAD, Hercules, Calif.).

c-Myc/His-tagged CDCA5 expression vector (pcDNA3.1-c-Myc/His-CDCA5) or mock vector (pcDNA3.1-c-Myc/His) was transfected into COS-7 or NIH3T3 cells using FuGENE6 transfection reagent (Roche). Transfected cells were incubated in the culture medium containing 0.4 mg/ml, neomycin (Geneticin, Invitrogen). 7 days later, viability of cells was evaluated by MTT assay.

The entire coding sequence of EPHA7 was cloned, which was amplified by RT-PCR using the primer sets (5′-CGCGGATCCCACCATGGTTTTTCAAACTCG-3′ (SEQ ID NO: 65) and 5′-CCGCTCGAGCACTTGAATGCCAGTTCCATGTAA-3′ (SEQ ID NO: 66), into the appropriate site of pcDNA3.1 myc-His plasmid vector (invitrogen). COS-7 cells transfected either with plasmids expressing myc-His-tagged EPHA7 or with mock plasmids were grown for eight days in DMEM containing 10% FCS in the presence of appropriate concentrations of geneticin (G418). Viability of cells was evaluated by MTT assay; briefly, cell-counting kit-8 solution (DOJINDO) was added to each dish at a concentration of 1/10 volume, and the plates were incubated at 37 degrees C. for additional 2 hours. Absorbance was then measured at 490 nm, and at 630 nm as a reference, with a Microplate Reader 550 (BIO-RAD, Hercules, Calif.).

### (13) Matrigel Invasion Assay

COS-7 and NIH3T3 cells transfected either with plasmids expressing EPHA7 or with mock plasmids were grown to near confluence in DMEM containing 10% FCS. The cells were harvested by trypsinization, washed in DMEM without addition of serum or proteinase inhibitor, and suspended in DMEM at 5×10⁵ cells/ml. Before preparing the cell suspension, the dried layer of Matrigel matrix (Becton Dickinson Labware) was rehydrated with DMEM for 2 hours at room temperature. DMEM (0.75 ml) containing 10% FCS was added to each lower chamber in 24-well Matrigel invasion chambers, and 0.5 ml (2.5×10⁵ cells) of cell suspension were added to each insert of the upper chamber. The plates of inserts were incubated for 22 hours at 37 degree Centigrade. After incubation, the chambers were processed; cells invading through the Matrigel were fixed and stained by Giemsa as directed by the supplier (Becton Dickinson Labware).

### (14) In Vitro Kinase Assay

The present inventors did in vitro kinase assay using full-length recombinant STK31 protein (Invitrogen). Briefly, 0.5 μg STK31 protein was incubated in 30 μl kinase buffer {250 mmol/L Tris-HCl (pH 7.4)/50 μmol/L MgCl2/5 mmol/L NaF/10 mmol/L DTT/20 μmol/L ATP} and then supplemented with 5 μCi of [gamma-³²P]-ATP (GE Healthcare). For the substrates, we added 10 μg MBP in the reaction solutions. After 30-minute incubation at 30° C., the reactions were terminated by addition of SDS sample buffer. After boiling the protein samples were electrophoresed on 15% gel (Bio-Rad Laboratories), and then autoradiographed. Recombinant STK31 was also incubated with whole extracts prepared from COS-7 cells in the reaction solutions for 30-minute incubation at 30° C., reaction were stopped by addition of SDS sample buffer. After boiling, the protein sample was resolved by SDS-PAGE and then western-blot.

In vitro kinase assay was also performed using full-length recombinant GST-CDCA5 (pGEX-6p-1/CDCA5 cleaved with Precision Protease). Briefly, 1.0 μg each of GST-CDCA5, Histone H1 (Upstate), MBP, or GST was incubated in 200 of kinase buffer (50 mM Tris-HCl, 10 mM MgCl₂, 1 mM EGTA, 2 mM DTT, 0.01% Briji 35, 1 mMATP, pH7.5 25° C.) supplemented with 1 μCi of [gamma-³²P]-ATP (GE Healthcare) and 2 unit of CDC2 (BioLabs) or 50 ng of ERK2 (Upstate) for 20 min at 30° C. The reactions were terminated with Laemmli SDS sample buffer to a final volume of 30 μl, and half of samples were subjected to 5-15% gradient gel (Bio-Rad Laboratories), and phosphorylation were visualized by autoradiography. MBP was used as ERK substrate, and H1 as CDC2 substrate (positive control). GST was served as a negative control substrate.

In vitro kinase assay was further performed using immunoprecipitant of wild type or mutated WDHD1 proteins. Immunoprecipitant of wild type or mutated WDHD1 proteins were incubated with recombinant AKT1 (AKT1; Invitrogen, Carlsbad, Calif.) (GenBank Accession No.: NM_{—}001014431, SEQ ID NO.: 60) in kinase buffer [20 mmol/L Tris (pH 7.5), 10 mmol/L MgCl₂, 2 mmol/L MnCl2, 1 mmol/L phenylmethylsulfonyl fluoride, 1 mmol/L DTT] supplemented with a mixture of protease inhibitors, 10 mmol/L NaF, 5 nmol/L microcystin LR, and 50 μmol/L ATP. The reaction was terminated by the addition of a 0.2 volume of 5× protein sample buffer and the proteins were analyzed by SDS-PAGE.

### (15) Flow Cytometry

Cells were collected in PBS, and fixed in 70% cold ethanol for 30 minutes. After treatment with 100 μg/mL RNase (Sigma/Aldrich, St. Louis, Mo.), the cells were stained with 50 μg/mL propidium iodide (Sigma/Aldrich, St.) in PBS. Flow cytometry was done on a Becton Dickinson FACScan and analyzed by ModFit software (Verity Software House, Inc., Topsham, Me.). The cells selected from at least 20,000 ungated cells were analyzed for DNA content.

### (16) Analysis of WDHD1 Expression During Cell Cycle Progression

LC319 cells at densities of 5×10⁵ cells/100 mm dish were synchronized at G0/G1 with RPMI1640 containing 1% FBS and 4 μg/ml of aphidicolin (Sigma/Aldrich, St. Louis, Mo.) for 24 hours and released from G1 arrest by the removal of aphidicolin. Then the cells were trypsinized at 0, 4, and 9 hours after removal of aphidicolins and were harvested for flow cytometric and western-blot analyses. A549 cells at densities of 5×10⁵ cells/100 mm dish were synchronized at G0/G1 with RPMI1640 containing 1% FBS and 1 μg/ml of aphidicolin (Sigma/Aldrich, St. Louis, Mo.) for 18 hours and released from G1 arrest by the removal of aphidicolin. Then the cells were trypsinized at 0, 2, 4, 6, 8, and 10 hours after removal of aphidicolins and were harvested for flow cytometric and western-blot analyses.

### (17) Live Cell Imaging

Cells were grown on a 35 mm glass-bottom dish in phenol red-free Dulbecco's modified Eagle's medium containing 10% fetal bovine serum (FBS). Cells were transfected with siRNA and subjected to time-lapse imaging using a computer-assisted fluorescence microscope (Olympus, LCV100) equipped with an objective lens (Olympus, UAPO 40×/340 N.A.=0.90), a halogen lamp, a red LED (620 nm), a CCD camera (Olympus, DP30), differential interference contrast (DIC) optical components, and interference filters. For DIC imaging, the red LED was used with a filter cube containing an analyzer. Image acquisition and analysis were performed by using MetaMorph 6.13 software (Universal Imaging, Media, Pa.).

### (18) MALDI-TOF Mass Spectrometry Analysis

CDCA5 recombinant protein was incubated with ERK or CDC2 for 3.5 hours at 37° C. Samples ware separated on SDS-PAGE gel. After electrophoresis, the gels were stained by R-250 (Bio-Rad). Specific bands corresponding to CDCA5 were digested with tripsin as previously described (Kato T., et al. Clin Cancer Res 2008; 14:2363-70) and served for analysis by matrix-assisted laser desorption/ionization mass spectrometry analysis (MALDI-QIT-TOF; Shimadzu Biotech, Kyoto, Japan). The mass spectral data was evaluated using the Mascot search engine (http://www.matrixscience.com) to identify proteins from primary sequence databases.

### (19) Cell Synchronization at Mitosis and EGF Stimulation Assay

Cultured A549 and LC319 lung cancer cells as well as cervical squamous cell carcinoma Hela cells were synchronized in G1/S phase by 2 μg/ml aphidilcoline for 16 hours incubation. For mitosis synchronization, the cells were released at 0 hour from G1/S phase. Nocodazole was added at 5 hours to prevent mitotic exit. At the point, CDC2 inhibitors or PBS were added to the cell cultures. For the EGF stimulation assay, Hela cells were cultured in FBS free medium for 20 hours. Then, the cells were stimulated by 50 μg/ml EGF for 30 min with or without 10 μM MEK inhibitor U0126 (Promega)

### (20) Identification of EPHA7 Associated Protein

COS-7 cells (5×10⁶), transfected with plasmids expressing EPHA7 (pcDNA3.1/myc-His-EPHA7), or the empty vector (pcDNA3.1/myc-His as control), were incubated in 1 mL lysis buffer (0.5% NP40, 50 mmol/L Tris-HCl, 150 mmol/L NaCl) in the presence of inhibitors against proteinase (EMD, San Diego, Calif.) and phosphatase (EMD). Cell extracts were precleared by incubation at 4 degrees C. for 1 hour with 60 μL protein G-Agarose beads (Invitrogen), in final volumes of 1.2 mL of immunoprecipitation buffer (0.5% NP40, 50 mmol/L Tris-HCl, 150 mmol/L NaCl) in the presence of proteinase inhibitor. After centrifugation at 1,500 rpm for 1 minute at 4° C., the supernatants were incubated at 4 degrees C. with anti-c-myc agarose (Sigma) for 2 hours. After the beads were collected from each sample by centrifugation at 3,000 rpm for 1 minutes and washed six times with 1 mL of immunoprecipitation buffer, beads were resuspended in 30 μL of Laemmli sample buffer and boiled for 5 minutes before the proteins were separated on 5% to 10% SDS-PAGE gels (Bio-Rad). After electrophoresis, the gels were stained with silver. Protein bands found specifically in EPHA7-transfected extracts were excised to serve for analysis by matrix-assisted laser desorption/ionization time of flight mass spectrometry (MALDI-TOF-MS; AXIMA-CFR plus, SHIMADZU BIOTECH, Kyoto, Japan). To confirm the interaction between EPHA7 and MET (GenBank Accession No.: NM_{—}000245), we carried out the immunoprecipitation experiment. To achieve FLAG-tagged MET, we cloned the entire coding sequence, which was amplified by RT-PCR using the primer sets (5′-TTGCGGCCGCAAATGAAGGCCCCCGCTGTGCTTG-3′ (SEQ ID NO: 67) and 5′-CCGCTCGAGCGGTGATGTCTCCCAGAAGGAGGCTG-3′ (SEQ ID NO: 68), into the appropriate site of pCAGGSn-3Fc plasmid vector. The extracts from COS-7 cells transfected with pCCAGGSn-3Fc-MET and pcDNA3.1/myc-His-EphA7 were immunoprecipitated with anti-c-Myc-agarose. Immunoblot was done using anti-FLAG M2 monoclonal antibody (Sigma-Aldrich). For further confirmation we also performed immunoblot using anti-c-myc polyclonal antibody (Santa-Cruz) followed by immunoprecipitation of the same extracts using anti-Flag agarose. To confirm interaction between EPHA7 and EGFR we cloned the entire coding sequence into the appropriate site of pCAGGSn-3Fc plasmid vector. The extracts from COS-7 cells transfected with pCCAGGSn-3Fc-EGFR and pcDNA3.1/myc-His-EphA7 were immunoprecipitated and immunoblot was done by the same method as MET.

### In Vitro EPHA7 Kinase Assay.

Active recombinant EPHA7 (Carnabioscience, Kobe, Japan), EGFR (Millipore, Billerica, Mass.), MET (Millipore), EGFR inhibitor AG1478 (EMD), and MET inhibitor SU11274 were commercially purchased. We constructed plasmids expressing partial fragments of EGFR (#1: codons 692-891, #2: codons 889-1045, #3: codons 1046-1186) that contained GST-tagged epitopes at their N-terminals were prepared using pGEX vector (GE Healthcare Bio-sciences). The recombinant peptides were expressed in Escherichia coli, BL21 codon-plus strain (Stratagene, La Jolla, Calif.), and purified using TALON resin (BD Biosciences Clontech) according to the supplier's protocol. The purified proteins were extracted on an SDS-PAGE gel. To avoid EGFR or MET autophosphorylation we preliminarily determined minimum inhibitory concentration of AG1478 or SU11274, and confirmed that these inhibitors did not inhibit EPHA7 autophosphorylation at such concentration. EPHA7 kinase assay using EGFR as a substrate comprised a following reaction mixture: 20 ng of EPHA7 protein, 50 ng of EGFR protein (active recombinant protein with 1 mM AG1478 [EGFR inhibitor; see above] or partial inactive EGFR fragments without inhibitor), 50 mM tris-HCl, 10 mM MgCl₂, 2 mM DTT, 1 mM NaF, and 0.1 μL protease inhibitor, followed by addition of 1 mM ATP containing 3 μCi [gamma-³²P] ATP (GE Healthcare Bio-sciences). After incubation at 30 degrees C. for 30 minutes the reactions were terminated by addition of SDS sample buffer. After boiling, the protein samples were electrophoresed on 5% to 15% gradient gel (Bio-Rad), and then signals were visualized by Molecular imager FX (Bio-Rad). In EPHA7 kinase assay using MET as substrate, we adopted the same protocol as above mentioned EPHA7-EGFR kinase reaction, using 50 ng of MET and 12.5 μM of SU11274 (MET inhibitor; see above), instead of EGFR and AG1478. To determine the presence of tyrosine phosphorylated proteins in kinase reaction, we performed the in vitro kinase assay using 1 mM ATP that did not contain [gamma-³²P] ATP, and detected phosphorylated proteins using anti-pan phospho-tyrosine antibody (Invitrogen).

Identification of Downstream Signaling Pathways of EPHA7.

For identification of activated signaling pathway related to EGFR/MET, we performed immunoblot screening using extract of COS-7 cells exogenously expressing EPHA7. Briefly, COS-7 cells were seeded dishes at a number of 1×10⁶, and 24 hours later the cells were transfected with plasmids expressing EPHA7 (pcDNA3.1/myc-His-EPHA7), or the empty vector (pcDNA3.1/myc-His as control) and incubated for 48 hours. The cells were washed with cold PBS twice and immediately applied 0.5 mL of lysis buffer in the presence of proteinase inhibitor and phosphatase inhibitor. Extracts were then sonicated and centrifuged at 15,000 rpm for 15 minutes, and supernatants were gathered as samples. Specific antibodies used for immunoblotting were anti-EGFR, anti-phospho-EGFR (Tyr1068, Tyr1086, and Tyr1173), anti-phospho-MET (Tyr1349) anti-p44/42 MAP kinase (ERK), anti-phospho-p44/42 MAP kinase (ERK) (Thr202/Tyr204), anti-Akt, anti-phospho-Akt (Ser473), anti-Shc, anti-phospho-Shc (Tyr317), anti-phospho-Shc (Tyr239/240), anti-STAT1, anti-phospho-STAT1 (Tyr701), anti-STAT3, anti-phospho-STAT3 (Tyr705), anti-STATS, and anti-phospho-STAT5 (Tyr694) which were purchased from Cell Signaling technology (Danvers, Mass.), anti-MET and anti-phospho-MET (Tyr1313) antibodies that were from Santa-Cruz. Anti-phospho-MET (Tyr1230/1234/1235, Tyr1365) antibodies were from Invitrogen.

### Example 2

### CDCA5

### (1) Expression of CDCA5 in Lung and Esophageal Cancers and Normal Tissues.

The present inventors previously screened 27,648 genes on a cDNA microarray to detect transcripts indicating 3-fold or higher expression in cancer cells than in normal control cells in more than 40% of clinical samples analyzed (WO2004/031413, WO2007/013665, WO2007/013671). Among the up-regulated genes, the present inventors identified the CDCA5 transcript and confirmed its increased expression in 9 of 10 representative NSCLC cases, all of 5 SCLC cases, and in all of the 23 lung-cancer cell lines by semiquantitative RT-PCR experiments (FIG. 1A, top and middle panels). It was also observed high levels of CDCA5 expression in all of 10 ESCC cases and in all of the 10 esophageal cancer cell lines, whereas PCR product was hardly detected in cells derived from normal small airway epithelia (SAEC) and normal esophagus sample (FIG. 1B, top and middle panels). Furthermore, the strong expression of endogenous CDCA5 protein was confirmed in lung cancer and esophageal cancer cell lines using anti-CDCA5 antibody (FIG. 1A, B, bottom panels).

To examine the subcellular localization of exogenous CDCA5 in COS-7 cell line immunofluorescence analysis was performed and it was found that CDCA5 was located at nucleus of interphase cells (FIG. 1C), but was observed diffusely within M-phase cells (data not shown). Northern blot analysis using a CDCA5 cDNA fragment as a probe identified a 2.8-kb transcript to be highly expressed in testis, but its transcript was hardly detectable in any other normal tissues (FIG. 1D).

### (2) Growth Promotive Activity of CDCA5.

We knocked down the expression of endogenous CDCA5 in lung cancer cell lines A549 and LC319, which showed high level of CDCA5 expression, by means of siRNA oligonucleotide for CDCA5. We examined the expression levels of CDCA5 by semiquantitative RT-PCR and found that two CDCA5-specific siRNAs (si-CDCA5-#1 and si-CDCA5-#2) significantly suppressed expression of CDCA5 as compared with a control siRNA construct (si-LUC and si-CNT) (FIGS. 2A and 2B, upper panels). Colony formation and MTT assays revealed that introduction of si-CDCA5s significantly suppressed the growth of both A549 and LC319 cells, in accordance with its knockdown effect on CDCA5 expression (FIGS. 2A and 2B, middle and lower panels). We next examined a role of CDCA5 in promoting cell growth. We prepared plasmids designed to express CDCA5 (pcDNA3.1-CDCA5-c-Myc/His) and transfected them into COS-7 or NIH3T3 cells. As shown in FIG. 2C, transfection of CDCA5 cDNA into COS-7 or NIH3T3 cells significantly enhanced the cell growth, compared with that of mock vector.

### (3) Phosphorylation of CDCA5 by ERK and CDC2 Protein Kinases In Vitro.

To analyze the function of CDCA5 in carcinogenesis, we focused on the phosphorylation sites on CDCA5 protein. According to previous report using proteomic phospho-peptides screening, CDCA5 was supposed to be phosphorylated at Serine-75, Serine-79, and Threonine-115 (Olsen J V, Blagoev B, Gnad F. Global, In vivo and Site-Specific Phosphorylation Dynamics in Signaling Networks. Cell 2006; 127(3):635-648). To identify the cognate kinase for CDCA5 phosphorylation, we compared the peptide sequence of CDCA5 including Serine-75, Serine-79, and Threonine-115 with phosphorylation sites, and found that Serine-75 of CDCA5 completely matched the consensus CDC2 protein kinase phosphorylation site [S/T-P-x-R/K], while Serine-79 and Threonine-115 concordantly matched the ERK phosphorylation site [x-x-S/T-P] (FIG. 17A). These consensus sequences were highly conserved in many species (FIG. 17A). We subsequently performed in vitro kinase assay by incubating recombinant CDC2 or ERK with CDCA5, and found that CDCA5 was directly phosphorylated by both ERK and CDC2 (FIG. 17B). The results are consistent with the conclusion that CDCA5 is involved in the CDC2 and/or ERK pathway.

To determine the direct phosphorylation sites on CDCA5 by these kinases, we performed in vitro kinase assay coupled with subsequent MALDI-QIT-TOF analysis. Recombinant CDCA5 protein was incubated with the ERK or CDC2 protein kinases for 3.5 hours at 37° C. On the gels, CDCA5 protein which was incubated with ERK comprised two bands after kinase assay, although CDCA5 incubated with CDC2 appeared to be a single band. We cut 4 bands for MS analysis (FIG. 17C), and identified 8 ERK-dependent and 3 CDC2-dependent phosphorylation sites (FIG. 17D). Serine-21, Serine-75, and Threonine-159 were phosphorylated by both ERK and CDC2.

### (4) Identification of ERK-Dependent In Vivo Phosphorylation of CDCA5.

To prove that endogenous CDCA5 was phosphorylated by ERK in mammalian cells, serum-starved Hela cells were stimulated with EGF in the presence or absence of MEK inhibitor U0126. Western blotting using anti-ERK antibody indicated that ERK was highly activated at 15 and 30 minutes after EGF stimulation, but the level was decreased at 60 and 120 minutes (FIG. 18 A, left panels). In accordance with the increased levels of ERK phosphorylation, a CDCA5 band detected by anti-CDCA5 antibody was shifted to higher molecular weight. In contrast, treatment of the cells with both EGF and MEK inhibitor U0126 reduced the levels of ERK phosphorylation and completely inhibited the upper shift of CDCA5 band (FIG. 18 A, right panels). These results demonstrate the possible phosphorylation of endogenous CDCA5 protein by ERK pathway.

To confirm MAP kinase pathway-dependent phosphorylation of CDCA5 and identify the phosphorylation sites in cultured cell, Hela cells transfected with plasmids designed to express myc-tagged CDCA5 were stimulated with EGF in the presence or absence of MEK inhibitor U0126, and their cell extracts were served for 2D-western-blotting using anti-myc antibody. In Hela cells without treatment of EGF and U0126, 2 spots were detected (spots no. 1 and 2), however treatment with EGF resulted in relatively remarkable increase in the signal of one of the spots (spot no. 2), while it induced two new spot signals (spots no. 3 and 4) with more acidic pI values. These shifted spots with more acidic pI were significantly reduced by pre-incubation of the cells with MEK inhibitor U0126 (FIG. 18B). In addition, the signal of spot no. 2 that had been increased by EGF stimulation was also reduced by U0126 treatment. These results suggest that CDCA5 was specifically phosphorylated by MAPK cascade in response to EGF ligand stimulation.

### (5) Identification of CDK1/CDC2-Dependent In Vivo Phosphorylation of CDCA5.

CDK1/CDC2 and its binding protein cyclin B1 are known to be required for M phase entry and maintenance of mitotic state in mammalian cells, suggesting the possible enhanced phosphorylation of the substrate protein(s) of CDC2 kinase in mitosis (Minshull L, et al. Cell 1989; 56: 947-956., Nurse P, et al. Nature 1990; 344: 503-508). Based on this hypothesis, lung cancer cell lines A549 and LC319 were synchronized at G1/S phase with aphidicolin treatment. After release from G1/S phase, the phosphorylation status of endogenous CDCA5 protein throughout the cell cycle was detected by western-blotting. Interestingly, an upper-sifted band was observed during M phase (mainly at 10˜11 hours), suggesting that CDCA5 might be phosphorylated by CDC2 pathway (FIG. 19A). The shifted band was also observed in esophageal cancer cell line TE8 and small cell lung cancer cell line SBC-3 that were synchronized at M phase by treatment with nocodazole (FIG. 19D).

To determine whether endogenous CDCA5 phosphorylation in mitosis was CDC2-dependent, we further treated the lung cancer cells at 5 hours after release from G1/S phase with nocodazole alone or both nocodazole and CDC2 inhibitor CGP74514A, and measured the status of CDCA5 phosphorylation by western blotting. Mitotic cells treated with nocodazole alone gradually expressed phosphorylated CDCA5 (shifted bands) (FIG. 19B). However, the cells treated with both nocodazole and CGP74514A showed no upper shifted bands indicating that CDCA5 phosphorylation in mitosis was significantly inhibited (FIG. 19B). These results indicate that phosphorylation of endogenous CDCA5 in mitosis was dependent on CDC2 activity. We also examined this experiment using other CDC2 inhibitor alsterpaullone, 4 μM alsterpaullone could strictly inhibit CDCA5 phosphorylation, although its CDC2-inhibitory activity appeared to be lower compared with the other CDC2 inhibitor CGP74514A (FIG. 19E).

In vitro kinase assay identified 3 phosphorylation sites (Serine-21, Serine-75 and Threonine-159) on CDCA5. To determine CDC2-dependent phosphorylation sites on CDCA5 in cultured cells, we constructed mutant CDCA5 expressing plasmids with the amino acid substitution; serine/threonine to alanine at codon 21, 75, or 159 (S21A, S75A or T159A, respectively), and transfected non-tagged wild type CDCA5-expressing plasmids or either of the three mutant CDCA5 constructs to Hela cells. We then synchronized the cells at G1/S phase with aphidicolin treatment. 24 hours after release from G1/S phase, and subsequent synchronization at M phase with nocodazole, 3 different bands corresponding to wild type CDCA5 were detected in cells transfected with wild type CDCA5 expression vector, however, cells transfected with alanine substitutent at Serine-21, Serine-75 or Threonine-159 showed the shifted band patterns of CDCA5 that were different from wild type CDCA5 (FIG. 19C). The result indicates that CDCA5 was phosphorylated in mammalian cells. Furthermore, CDCA5 protein seems to be unstable when the cells were treated with CDC2 inhibitor CGP74514A or its serine residue at codon 21 was not phosphorylated (FIG. 19C).

These data are consistent with the conclusion that the CDC5 is phosphorylated by ERK and CDC2. The protein encoded by ERK gene is a member of the MAP kinase family proteins that function as an integration point for multiple biochemical signals, and are involved in a wide variety of cellular processes for example, proliferation, differentiation, transcription regulation, and development. The MAPK cascade integrates and processes various extracellular signals by phosphorylating substrates, which alters their catalytic activities and conformation or creates binding site for protein-protein interactions. On the other hand, cyclin-dependent kinases (CDKs) are heterodimeric complexes composed of a catalytic kinase subunit and a regulatory cyclin subunit, and comprise a family divided into two groups based on their roles in cell progression and transcriptional regulation. CDC2/CDK1 (CDC2-cyclin B complex) is a member of the first group, which are required for orderly G2 to M phase transition. Recently, CDC2 was implicated in cell survival during mitotic checkpoint activation (O'Connor D S, Wall N R, Porter A C G. A p34cdc2 survival checkpoint in cancer. Cancer cell 2002; 2:43-54). Therefore our data showed that the phosphorylation of CDC5 by ERK and CDC2 promotes cancer cell cycle progression that increase the malignant potential of tumors.

### (6) Discussion

Molecular-targeted drugs are expected to be highly specific to malignant cells, and have minimal adverse effects due to their well-defined mechanisms of action. In spite of improvement of model surgical techniques and adjuvant chemo-radiotherapy, lung cancer and ESCC are known to reveal the worst prognosis among malignant tumors. Therefore, it is now urgently required to develop effective diagnostic biomarkers for early detection of cancer and for the better choice of adjuvant treatment modalities to individual patients, as well as new types of anti-cancer drugs and/or cancer vaccines. To identify appropriate diagnostic and therapeutic target molecules, we combined genome-wide expression analysis (Kikuchi T, et al., Oncogene. 2003 Apr. 10; 22(14): 2192-205; Kakiuchi S, et al., Mol Cancer Res. 2003 May; 1(7): 485-99; Kakiuchi S, et al., Hum Mol Genet. 2004 Dec. 15; 13(24): 3029-43. Epub 2004 Oct. 20; Kikuchi T, et al. Int J Oncol. 2006 April; 28(4): 799-805; Taniwaki M, et al, Int J Oncol. 2006 September; 29(3): 567-75; Yamabuki T, et al., Int J Oncol. 2006 June; 28(6):1375-84) for selecting genes that were overexpressed in lung and esophagus-cancer cells with high-throughput screening of loss-of-function effects by means of the RNAi technique (Suzuki C, et al., Cancer Res. 2003 Nov. 1; 63(21): 7038-41; Ishikawa N, et al., Clin Cancer Res. 2004 Dec. 15; 10(24): 8363-70; Kato T, et al., Cancer Res. 2005 Jul. 1; 65(13): 5638-46; Furukawa C, et al., Cancer Res. 2005 Aug. 15; 65(16): 7102-10; Ishikawa N, et al., Cancer Res. 2005 Oct. 15; 65(20): 9176-84; Suzuki C, et al., Cancer Res. 2005 Dec. 15; 65(24): 11314-25; Ishikawa N, et al., Cancer Sci. 2006 August; 97(8): 737-45; Takahashi K, et al., Cancer Res. 2006 Oct. 1; 66(19): 9408-19; Hayama S, et al., Cancer Res. 2006 Nov. 1; 66(21): 10339-48; Kato T, et al., Clin Cancer Res. 2007 Jan. 15; 13(2 Pt 1): 434-42; Suzuki C, et al., Mol Cancer Ther. 2007 February; 6(2):542-51; Yamabuki T, et al., Cancer Res. 2007 Mar. 15; 67(6): 2517-25; Hayama S, et al., Cancer Res. 2007 May 1; 67(9): 4113-22). Using this systematic approach we found CDCA5 to be frequently overexpressed in clinical lung cancer and ESCC samples, and showed that overexpression of this gene product plays an indispensable role in the growth of lung-cancer cells.

Previous studies have demonstrated that CDCA5 interacts with cohesion on chromatin and functions there during interphase to support sister chromatid cohesion, and sister chromatids are further separated than normally in most G2 cells, consistent with the conclusion that CDCA5 is already required for establishment of cohesion during S phase (Schmitz J, et al., Curr Biol. 2007 Apr. 3; 17(7): 630-6. Epub 2007 Mar. 8). So far only one other protein is known to be specifically required for cohesion establishment: the budding yeast acetyltransferase Eco1/Ctf7 (Skibbens R V, et al., Genes Dev. 1999 Feb. 1; 13(3): 307-19; Tóth A, et al., Genes Dev. 1999 Feb. 1; 13(3): 320-33; Ivanov D, et al., Curr Biol. 2002 Feb. 19; 12(4): 323-8). Homologs of this enzyme are also required for cohesion in Drosophila and human cells (Williams B C, et al., Curr Biol. 2003 Dec. 2; 13(23): 2025-36; Hou F & Zou H. Mol Biol Cell. 2005 August; 16(8):3908-18. Epub 2005 Jun. 15), although it is not yet known whether these proteins also function in S phase. It will therefore be interesting to address whether CDCA5 and Eco1/Ctf7 homologs collaborate to establish cohesion in cancer cells.

Sister chromatid cohesion must be established and dismantled at the appropriate times in the cell cycle to effectively ensure accurate chromosome segregation. It has previously been shown that the activation of APCCdc20 controls the dissolution of cohesion by targeting the anaphase inhibitor securin for degradation. This allows the separase-dependent cleavage of Scc1/Rad21, triggering anaphase. The degradation of most cell cycle substrates of the APC is logical in terms of their function; degradation prevents the untimely presence of activity and in a ratchet-like way promotes cell cycle progression. The function of CDCA5 may also be redundant with that of other factors that regulate cohesion, with their combined activities ensuring the fidelity of chromosome replication and segregation (Rankin S, et al., Mol Cell. 2005 Apr. 15; 18(2): 185-200) According to our microarray data, APC; CDC20 also expressed highly in lung and esophageal cancers; although their expressions in normal tissues are low. Furthermore, CDC20 was confirmed with high expression in clinical small cell lung cancer using semi-quantitative RT-PCR and immunohistochemical analysis (Taniwaki M, et al., Int J Oncol. 2006 September; 29(3): 567-75). These data are consistent with the conclusion that CDCA5 in collaboration with CDC20 enhances the growth of cancer cells, by promoting cell cycle progression, although, no evidence shows that these molecules could interact directly with CDCA5.

CDCA5 was previously reported to be located in the nucleus at interphase, cytosolic in Mitosis (Rankin S, et al., Mol Cell. 2005 Apr. 15; 18(2): 185-200). However, its physiological function remains unclear. It was confirmed that CDCA5 localized at nucleus. The nucleus contains genetic material and its main function is to maintain the integrity of the genes and regulate gene expression. The nucleus is a dynamic structure that changes according to the cells requirements. In order to control the nuclear functions, the processes of entry and exit from the nucleus are regulated. The localization of CDCA5 in nucleus indicates that this molecule may play roles as an essential factor to control cell cycle (Kho C J, et al., Cell Growth Differ. 1996 September; 7(9):1157-66; Bader N, et al., Exp Gerontol. 2007 Apr. 10; [Epub ahead of print]). Although, CDCA5 was known to play important roles in cell cycle control, no studies proved that CDCA5 have any relationship with carcinogenesis process. The present inventors confirmed that introduction of si-CDCA5 significantly suppressed growth of lung cancer cells, whereas CDCA5 has a growth promoting effect on mammalian cells, demonstrating that CDCA5 plays an important role on cancer cell growth/survival. Furthermore, CDCA5 expression was observed only in testis, meaning this gene should be a promising target molecule for cancer immunotherapy for example, cancer vaccine with minimal side effect.

These data are consistent with the conclusion that CDCA5 is phosphorylated by ERK and CDC2. The protein encoded by ERK gene is a member of the MAP kinase family proteins that function as an integration point for multiple biochemical signals, and are involved in a wide variety of cellular processes for example, proliferation, differentiation, transcription regulation, and development. The MAPK cascade integrates and processes various extracellular signals by phosphorylating substrates, which alters their catalytic activities and conformation or creates binding site for protein-protein interactions. On the other hand, cyclin-dependent kinases (CDKs) are heterodimeric complexes composed of a catalytic kinase subunit and a regulatory cyclin subunit, and comprise a family divided into two groups based on their roles in cell progression and transcriptional regulation. CDC2/CDK1 (CDC2-cyclin B complex) is a member of the first group, which are required for orderly G2 to M phase transition. Recently, CDC2 was implicated in cell survival during mitotic checkpoint activation (O'Connor D S, Wall N R, Porter A C G. A p34cdc2 survival checkpoint in cancer. Cancer cell 2002; 2:43-54). Therefore our data showed that the phosphorylation of CDC5 by ERK and CDC2 promotes cancer cell cycle progression that increase the malignant potential of tumors.

In summary, these data demonstrated that CDCA5 promotes growth of lung and esophagus cancers, and indicating its use as an effective therapeutic target for development of anti-cancer drugs.

### Example 3

### EPHA7

### (1) Expression and Cellular Localization of EPHA7 in Lung Cancers and Normal Tissues.

Using a cDNA microarray to screen for elements that were highly transactivated in a large proportion of lung cancer (WO2007/013665) and/or esophageal cancers, the present inventors identified EPHA7 gene as a good candidate. This gene showed a 3-fold or higher level of expression in the majority of lung and esophageal cancers. Subsequently we confirmed its transactivation by semiquantitative RT-PCR experiments in 7 of 10 NSCLC cases (3 of 5 ADCs and 4 of 5 SCCs) and in all of 3 SCLC cases (FIG. 3A, upper panels) as well as in 9 of 19 NSCLC cell lines and 3 of 4 SCLC cell lines (FIG. 3A, lower panels). Up-regulation of EPHA7 was also detected in 7 of 9 ESCC cases and 2 of 10 esophageal cancer cell lines (FIG. 3B, upper and lower panels). To determine the subcellular localization of endogenous EPHA7 in cancer cells, immunocytochemical analysis was performed using anti-EPHA7 polyclonal antibodies; N-terminal portion of human EPHA7 was localized in the cytoplasmic membrane and cytoplasm of lung cancer derived SBC-3 cells, when using antibodies to extracellular portion of EPHA7 (FIG. 3F, upper panel). On the other hand, C-terminal portion of human EPHA7 was also detected at nucleus and cytoplasm of the SBC-3 cells, when using antibodies to intracellular portion of EPHA7 (FIG. 3F, lower panel). As EPHA7 was a type I membrane protein, the present inventors hypothesized that the N-terminal domain of EPHA7 protein is cleaved and secreted into extracellular space like other receptor tyrosine kinase proteins including ERBB family (McKay M M & Morrison D K. Oncogene. 2007 May 14; 26(22): 3113-21; Reinmuth N, et al., Int J Cancer. 2006 Aug. 15; 119(4): 727-34; Lemmon M A. Breast Dis. 2003; 18: 33-43). Therefore the present inventors applied ELISA method using a rabbit polyclonal antibody specific to N-terminal portion of human EPHA7 (extracellular portion of EPHA7) (Catalog No. sc25459, Santa Cruz, Santa Cruz, Calif.) to examine its presence in the culture media of lung cancer cell lines. High levels of EPHA7 protein were detected in media of SBC-3, DMS114 and NCI-H1373 cultures but not in the medium of PC-14, NCI-H226, and A549 cells (FIG. 3G). The amounts of detectable EPHA7 in the culture media accorded well with the expression levels of EPHA7 detected with semiquantitative RT-PCR and immunocytochemistry.

Northern blot analysis using EPHA7 cDNA as a probe identified a very low level of 6.8-kb transcript only in fetal brain and fetal kidney among 27 adult and fetal human tissues (FIG. 3C). Additional northern blotting using the same probe detected only the EPHA7 transcript in lung-cancer cell line SBC-3, much more abundantly than fetal brain and fetal kidney (FIG. 3D). Furthermore, we compared EPHA7 protein expressions in 5 normal tissues (heart, lung, liver, kidney, and testis) with those in lung cancers using anti-EPHA7 polyclonal antibodies by immunohistochemistry. EPHA7 expressed abundantly in mainly in cytoplasm and/or cytoplasmic membrane of lung cancer cells, but its expression was hardly detectable in the remaining four normal tissues (FIG. 3E).

(2) Association of EPHA7 Overexpression with Poor Prognosis.

Using tissue microarrays prepared from 402 NSCLCs and 27 SCLCs, the present inventors performed immunohistochemical analysis with anti-EPHA7 polyclonal antibodies. Positive staining of EPHA7 was observed in 74.6% of NSCLCs (300/402) and 85.2% of SCLCs (23/27), while no staining was observed in any of normal lung tissues examined (FIG. 4A, left panels). Of these EPHA7 positive NSCLC cases, 189 were ADCs (74.7% of 253); 78 were SCCs (71.6% of 109 cases); 23 were LCCs (85.2% of 27 cases); 10 were adenosqamous cell carcinomas (ASC; 76.9% of 13).

A pattern of EPHA7 expression on the tissue array was classified ranging from absent (scored as 0) to weak/strong positive (scored as 1+˜2+). Of the 402 NSCLCs, EPHA7 was strongly stained in 190 cases (47.3%; score 2+), weakly stained in 110 cases (27.3%; score 1+), and not stained in 102 cases (25.4%: score 0) (details are shown in Table 3A). The present inventors then tried to correlate expression of this protein in NSCLCs who had undergone curative surgery with various clinicopathologic variables. The sample size of SCLCs treated with identical protocol was too small to be evaluated further. Statistical analysis revealed that tumor size (higher in pT1-4; P=0.0256 by Fisher's exact test) were significantly associated with the strong EPHA7 positivity (the details are shown in Table 3A). NSCLC patients whose tumors showed strong EPHA7 expression revealed shorter tumor-specific survival periods compared to those with absent/weak EPHA7 expression (P=0.006 by the Log-rank test; FIG. 2B).

By univariate analysis, age (≧65 versus <65), gender (Male versus Female), pT stage (T2+T3 versus T1), pN stage (N1, N2 versus N0), non-ADC histology (non-ADC versus ADC), and strong EPHA7 expression were significantly related to poor tumor-specific survival among NSCLC patients (Table 3B). Furthermore, multivariate analysis using the Cox proportional-hazard model indicated that elderly, larger tumor size, lymph node metastasis, and strong EPHA7 staining were independent prognostic factors for NSCLC (Table 3B).

Positive staining of EPHA7 was observed by immunohistochemical analysis of 292 ESCCs in 88.3% of ESCCs (258/292), while no staining was observed in any of normal esophageal tissues examined (FIG. 4A, right panels). Of the 292 ESCC cases examined, EPHA7 was strongly stained in 153 cases (52.4%; score 2+), weakly stained in 105 cases (36.0%; score 1+) and not stained in 34 cases (11.6%; score 0) (details are shown in Table 4A). Statistical analysis revealed that tumor size (higher in pT2-4; P<0.0001 by Fisher's exact test) and lymph-node metastasis (higher in pN1-2; P=0.0006 by Fisher's exact test) were significantly associated with the strong positivity of EPHA7 (Table 4A).

The median survival time was significantly shorter in patients with EPHA7-strong positive ESCCs, than in those with EPHA7-weak positive/negative tumors (P=0.0263 by log-rank test; FIG. 4C). In univariate analysis to evaluate associations between ESCC patient prognosis and several factors, gender (Male versus Female), pT stage (T2+T3 versus T1), pN stage (N1, N2 versus N0), and EPHA7 status (score 2+ versus 0, 1+) were significantly associated with poor prognosis. In multivariate analysis, EPHA7 status did not reach the statistically significant level as independent prognostic factor for surgically treated ESCC patients enrolled in this study (P=0.5586), while pT and pN stages as well as gender did so, demonstrating the relevance of EPHA7 expression to these clinicopathological factors in esophageal cancer (Table 4B).

TABLE 3A
Association between EPHA7-strong positivity in NSCLC tissues and
patients' characteristics (n = 402)

| | | | | | | P-value |
|---|---|---|---|---|---|---|
| | | EPHA7 | EPHA7 | | | strong |
| | | strong | weak | EPHA7 | | vs weak |
| | Total | positive | positive | absent | Chi- | positive or |
| | n = 402 | n = 190 | n = 110 | n = 102 | square | absent |
| Gender | | | | | | |
| Female | 123 | 51 | 37 | 35 | 1.948 | NS |
| Male | 279 | 139 | 73 | 67 | | |
| Age (years) | | | | | | |
| <65 | 207 | 91 | 61 | 55 | 1.611 | NS |
| ≧65 | 195 | 99 | 49 | 47 | | |
| Histological | | | | | | |
| type | | | | | | |
| ADC | 253 | 121 | 68 | 64 | 0.138** | NS |
| SCC | 109 | 47 | 31 | 31 | | |
| Others | 40 | 22 | 11 | 7 | | |
| pT factor | | | | | | |
| T1 | 132 | 51 | 35 | 46 | 5.194 | 0.0256* |
| T2 + T3 + | 270 | 139 | 75 | 56 | | |
| T4 | | | | | | |
| pN factor | | | | | | |
| N0 | 244 | 110 | 66 | 68 | 1.016 | NS |
| N1 + N2 | 158 | 80 | 44 | 34 | | |
| Smoking | | | | | | |
| history | | | | | | |
| Never | 119 | 52 | 32 | 35 | 0.600 | NS |
| smoker | | | | | | |
| Smoker | 283 | 138 | 78 | 67 | | |

ADC, adenocarcinoma
non-ADC, squamous-cell carcinoma plus large-cell carcinoma and adenosquamous-cell carcinoma
NS, no significance
*P < 0.05 (Fisher's exact test)
**ADC versus other histology

TABLE 3B
Cox's proportional hazards model analysis of prognostic
factors in patients with NSCLCs

| | Hazards | | Unfavorable/ | |
|---|---|---|---|---|
| Variables | ratio | 95% CI | Favorable | P-value |
| Univariate | | | | |
| analysis | | | | |
| EPHA7 | 1.498 | 1.121-2.002 | Strong | 0.0064* |
| | | | Positive/Weak | |
| | | | Positive | |
| | | | or Negative | |
| Age (years) | 1.452 | 1.085-1.944 | >=65/>65 | 0.0121* |
| Gender | 1.743 | 1.239-2.53 | Male/Female | 0.0014* |
| pT factor | 2.669 | 1.838-3.875 | T2 + T3 + T4/T1 | <0.0001* |
| pN factor | 2.391 | 1.788-3.197 | N1 + N2/N0 | <0.0001* |
| Histological | 1.368 | 1.021-1.832 | non-ADC/ADC | 0.0355* |
| type | | | | |
| smoking | 1.201 | 0.868-1.661 | smoker/ | NS |
| | | | non-smoker | |
| Multivariate | | | | |
| analysis | | | | |
| EPHA7 | 1.412 | 1.052-1.896 | Strong | 0.0216* |
| | | | Positive/Weak | |
| | | | Positive | |
| | | | or Negative | |
| Age (years) | 1.624 | 1.202-2.194 | >=65/>65 | 0.0016* |
| Gender | 1.445 | 0.991-2.107 | Male/Female | NS |
| pT factor | 1.981 | 1.342-2.924 | T2 + T3 + T4/T1 | 0.0006* |
| pN factor | 2.361 | 1.742-3.201 | N1+ N2/N0 | <0.0001* |
| Histological | 0.973 | 0.704-1.345 | non-ADC/ADC | NS |
| type | | | | |

ADC, adenocarcinoma
non-ADC, squamous-cell carcinoma plus large-cell carcinoma and adenosquamous-cell carcinoma
NS, no significance
*P < 0.05

TABLE 4A
Association between EPHA7-strong positivity in ESCC tissues and
patients' characteristics (n = 292)

| | | EPHA7 | EPHA7 | | | P-value |
|---|---|---|---|---|---|---|
| | Total | strong | weak | EPHA7 | | strong vs |
| | n = | positive | positive | absent | Chi- | weak positive |
| | 292 | n = 153 | n = 105 | n = 34 | square | or absent |
| Gender | | | | | | |
| Female | 34 | 16 | 15 | 3 | 0.44 | NS |
| Male | 258 | 137 | 90 | 31 | | |
| Age | | | | | | |
| (years) | | | | | | |
| <65 | 180 | 95 | 68 | 17 | 0.027 | NS |
| >=65 | 112 | 58 | 37 | 17 | | |
| pT factor | | | | | | |
| T1 | 96 | 32 | 45 | 19 | 20.839 | <0.0001* |
| T2 + T3 | 196 | 121 | 60 | 15 | | |
| pN factor | | | | | | |
| N0 | 111 | 44 | 48 | 19 | 11.645 | 0.0006* |
| N1 + N2 | 181 | 109 | 57 | 15 | | |

ESCC, Esophageal sqamous-cell carcinoma
NS, no significance
*P < 0.05 (Fisher's exact test)

TABLE 4B
Cox's proportional hazards model analysis of prognostic factors
in patients with ESCC

| | Hazards | | Unfavorable/ | |
|---|---|---|---|---|
| Variables | ratio | 95% CI | Favorable | P-value |
| Univariate | | | | |
| analysis | | | | |
| EPHA7 | 1.429 | 1.041-1.962 | Strong Positive/Weak | 0.0271* |
| | | | Positive or Negative | |
| Age (years) | 1.031 | 0.747-1.425 | >=65/>65 | NS |
| Gender | 3.057 | 1,559-5.995 | Male/Female | 0.0011* |
| pT factor | 3.127 | 2.052-4.766 | T2 + T3/T1 | <0.0001* |
| pN factor | 3.976 | 2.759-6.203 | N1 + N2/N0 | <0.0001* |
| Multivariate | | | | |
| analysis | | | | |
| EPHA7 | 0.906 | 0.650-1.262 | Strong Positive/Weak | NS |
| | | | Positive or Negative | |
| Gender | 2.201 | 1.319-5.093 | Male/Female | 0.0057* |
| pT factor | 2.201 | 1.413-3.430 | T2 + T3/T1 | 0.0005* |
| pN factor | 3.220 | 2.104-4.927 | N1 + N2/N0 | <0.0001* |

ESCC, Esophageal sqamous-cell carcinoma
NS, no significance
*P < 0.05

### (3) Serum Levels of EPHA7 in Lung and Esophageal Cancer Patients.

Because the in vitro assay demonstrated that the N-terminal domain of EPHA7 protein in lung cancer cells were cleaved and secreted into extracellular space, the present inventors investigated whether the EPHA7 is secreted into sera of patients with lung or esophageal cancer or not. ELISA experiments detected EPHA7 protein in serological samples from the great majority of the 439 patients with lung or esophageal cancer. The mean (+/−1 SD) of serum EPHA7 in 343 lung cancer patients was 4.33+/−3.73 U/ml and those in 96 ESCC patients were 10.74+/−8.12 U/ml. In contrast, the mean (+/−1 SD) serum levels of EPHA7 in 127 healthy individuals were 1.69+/−0.80 U/ml. The difference was significant with P-value of <0.001 (Mann-Whitney U test).

According to histological types of lung cancer, the serum levels of EPHA7 were 4.40+/−3.54 U/ml in 205 ADC patients, 3.41+/−2.35 U/ml in 59 SCC patients, and 4.85+/−4.83 U/ml in 79 SCLC patients (FIG. 5A); the differences among the three histologic types were not significant. High levels of serum EPHA7 were detected even in patients with earlier-stage tumors (data not shown). Using receiver-operating characteristic (ROC) curves drawn with the data of these 439 cancer (NSCLC+SCLC+ESCC) patients and 127 healthy controls (FIG. 5B, left panel), the cut-off level in this assay was set to provide optimal diagnostic accuracy and likelihood ratios for EPHA7, i.e., 2.83 U/ml (with a sensitivity of 60.4% (265/439) and a specificity of 95.3% (121/127). According to tumor histology, the proportions of the serum EPHA7-positive cases was 58.5% for ADC (120 of 205), 49.2% for SCC (29 of 59), 44.3% for SCLC (35 of 79), and 84.4% for ESCC (81 of 96).

The present inventors then performed ELISA experiments using paired preoperative and postoperative (2 months after the surgery) serum samples from lung cancer patients to monitor the levels of serum EPHA7 in the same patients. The concentration of serum EPHA7 was dramatically reduced after surgical resection of primary tumors (FIG. 5B, right panel). The results independently support the high specificity and the use of serum EPHA7 as a biomarker for detection of cancer at an early stage and for monitoring of the relapse of the disease.

To evaluate the clinical usefulness of serum EPHA7 level as a tumor-detection biomarker, the present inventors also measured by ELISA the serum levels of two conventional tumor markers (CEA for NSCLC and ProGRP for SCLC patients), in the same set of serum samples from cancer patients and control individuals. ROC analyses determined the cut off value of CEA for NSCLC detection to be 2.5 ng/ml (with a sensitivity of 37.9% (88/232) and a specificity of 89.8% (114/127); FIG. 5C, upper panel). The correlation coefficient between serum EPHA7 and CEA values was not significant (Spearman rank correlation coefficient: ρ (rho)=−0.172, P=0.009), indicating that measuring both markers in serum can improve overall sensitivity for detection of NSCLC to 76.7% (178 of 232) (for diagnosing NSCLC, the sensitivity of CEA alone is 37.9% (88 of 232) and that of EPHA7 is 55.2% (128 of 232). False-positive rates for either of the two tumor markers among normal volunteers (control group) were 7.1% (9 of 127), although the false-positive rates for each of CEA and EPHA7 in the same control group were 2.4% (3 of 127) and 4.7% (6 of 127), respectively.

ROC analyses for the patients with SCLC determined the cut-off value of ProGRP as 46.0 pg/ml, with a sensitivity of 64.8% (46 of 71) and a specificity of 97.6% (120 of 123) (FIG. 5C, lower panel). The correlation coefficient between serum EPHA7 and ProGRP values was not significant (Spearman rank correlation coefficient: ρ (rho)=0.143, P=0.2325), also indicating that measurement of serum levels of both markers can improve overall sensitivity for detection of SCLC to 77.5% (55 of 71); for diagnosing SCLC, the sensitivity of ProGRP alone was 64.8% (46 of 71) and that of EPHA7 was 45.1% (32 of 71). False-positive cases for either of the two tumor markers among normal volunteers (control group) were 7.3% (9 of 123), although the false-positive rates for ProGRP and EPHA7 in the same control group were 2.4% (3 of 123) and 4.9% (6 of 123), respectively.

### (4) Cellular Growth and Invasive Effect of EPHA7 in Mammalian Cells.

Inhibition of growth of lung cancer cells by small interfering RNA against EPHA7. To assess whether EPHA7 is essential for growth or survival of lung cancer cells, the present inventors constructed siRNAs against EPHA7 (si-EPHA7s) as well as control plasmids (siRNAs for LUC/Luciferase and Scramble/SCR) and transfected them into NCI-H520 and SBC-5 cells. The mRNA levels in cells transfected with si-EPHA7-#2 were significantly decreased in comparison with cells transfected with either control siRNAs. We observed significant decreases in the number of colonies formed and in the numbers of viable cells measured by MTT assay (FIG. 6A, right and left panels). Transfection of si-EPHA7-#1 resulted in slight decreases in colony numbers and cell viability as well as the weak reduction of EPHA7 expression.

To determine the effect of EPHA7 on growth and transformation of mammalian cells, we carried out in vitro assays using COS-7 cells that transiently expressed EPHA7 (COS-7-EPHA7). Growth of the COS-7-EPHA7 cells was promoted in comparison with the empty vector controls, as determined by the MTT assay (FIG. 7B).

As the immunohistochemical and statistical analysis on tissue microarray had indicated that EPHA7 positivity was significantly associated with shorter cancer-specific survival period, we performed Matrigel invasion assays to determine whether EPHA7 plays a role in cellular invasive ability. Invasion of COS-7-EPHA7 cells or NIH3T3-EPHA7 cells through Matrigel was significantly enhanced, compared to the control cells transfected with mock plasmids, thus independently showing that EPHA7 also contributes to the highly malignant phenotype of lung-cancer cells (FIG. 7C).

(5) Identification of EGFR, p44/42 MAPK, and CDC25 as Downstream Targets for EPHA7.

To elucidate the function of EPHA7 kinase in carcinogenesis, the present inventors attempted to identify substrate and/or downstream target proteins that would be phosphorylated through EPHA7 signaling and activate cell-proliferation signaling. The present inventors performed immunoblot-screening of kinase substrates for EPHA7 using cell lysates of COS-7 cells transfected with EPHA7-expression vector and a series of antibodies specific for phospho-proteins related to cancer-cell signaling (see Table 2). The present inventors screened a total of 28 phosphoproteins and found that Tyr-845 of EGFR, Tyr-783 of PLCgamma, and Ser-216 of CDC25 were significantly phosphorylated in the cells transfected with the EPHA7-expression vector, compared with those with mock vector (FIG. 8A). The present inventors confirmed the cognate interaction between endogenous EGFR and exogenous EPHA7 by immunoprecipitation experiment (FIG. 7B).

### (6) Identification of EGFR and MET as Novel Substrates for EPHA7.

To elucidate the function of EPHA7 in carcinogenesis, we attempted to identify substrate proteins for EPHA7 kinase that would be directly phosphorylated by EPHA7 and activate cell-proliferation and/or survival signaling. We performed MALDI-TOF MS analysis using the immunoprecipitant of COS-7 cells expressing exogenous EPHA7, and identified that MET proto-oncogene precursors as candidate EPHA7-interacting proteins. We validated this interaction by immunoprecipitation using extracts of COS-7 exogenously expressed MET and EPHA7 (FIGS. 20A and 20B). Both EPHA7 and MET are members of receptor tyrosine kinase protein and recent report suggests that in cancer cells several receptor tyrosine kinase are activated and that they can play complementary role for activating downstream signal transduction (Reinmuth N et al. Int J Cancer. 2006 Aug. 15; 119(4):727-34). In fact, immunoblot-screening of kinase substrates for EPHA7 using cell lysates of COS-7 cells transfected with EPHA7-expression vector and a series of antibodies specific for phospho-proteins related to cancer-cell signaling identified EGFR and MET as proteins phosphorylated by EPHA7 overexpression (see below). On the basis of this finding we performed immunoprecipitation using extracts of COS-7 exogenously expressed EGFR and EPHA7 and confirmed that EPHA7 could bind to EGFR (FIGS. 20C and 20D). To evaluate the possibility of synergical activation of EPHA7 with EGFR and/or MET in cancer cells, we examined their expression by western blotting in lung cancer cells (FIG. 20E). Certain population of lung cancer cells expressed both EPHA7 and MET or both EPHA7 and EGFR, indicating that these heterodimer complexes could be present in lung cancer cells.

To evaluate kinase-substrate reaction between EPHA7 and EGFR/MET, we performed in vitro kinase assay using active recombinant proteins of cytoplasmic EPHA7, MET, EGFR, and also using three inactive partial-proteins covering cytoplasmic EGFR (FIG. 21A). As expected, we found that EPHA7 could directly phosphorylate EGFR under the existence of EGFR kinase inhibitor that had diminished autophosphorylation of EGFR (FIGS. 6B and 6C). Additional in vitro kinase assay using three partial cytoplasmic EGFR as substrates revealed that phosphorylated tyrosine residues on cytoplasmic EGFR could be present in COOH-terminal portion (codons 1046-1186; FIGS. 21B and 21C). This region contains several phosphorylated tyrosine residues and some of them such as Tyr1068 and Tyr1173 have important roles in activating downstream signals. We also performed in vitro kinase assay using EPHA7 and MET, and found that EPHA7 could directly phosphorylate MET (FIG. 21D). Interestingly, we could observe EPHA7 autophosphorylation by addition of ATP into EPHA7, but the level of EPHA7 phosphorylation was markedly elevated when MET was co-incubated in the presence MET kinase inhibitor, indicating that EPHA7 could be activated by interacting with MET (FIG. 21D). We next screened the EPHA7-dependent phosphorylation sites on EGFR/MET in mammalian cells. In this screening, although we examined all currently available antibodies for phospho-EGFR and phospho-MET that recognized various phospho-residues within the cytoplasmic domain of the EGFR (Tyr-992, Tyr-1045, Tyr-1068, Tyr-1086, Tyr-1148, and Tyr-1173 as well as phospho-Ser-1046/1047) and the MET (Tyr-1230/1234/1235, Tyr-1313, Tyr-1349, and Tyr-1365), we found the increased phosphorylation of Tyr-1068, Tyr-1086, and Tyr-1173 of EGFR and that of Tyr-1230/1234/1235, Tyr-1313, Tyr-1349, Tyr-1365 of MET (FIG. 21E). No significant increase in phosphorylation levels of other Tyr-residues were observed (data not shown). The data strongly suggest that EPHA7 expressed in mammalian cells could phosphorylate endogenous EGFR/MET.

### (7) Enhancement of Oncogenic Downstream Signaling by EPHA7.

Since there are evidences that EGFR/MET play pivotal role for cell proliferation, survival, or motility of cancer cells, we then focused on the possibility that enhancement of EGFR/MET activity by EPHA7 leads to activation of EGFR/MET downstream signaling. We performed immunoblot analyses using cell lysates of COS-7 cells transfected with EPHA7-expression vector and a series of antibodies specific for oncogenic phospho-proteins including proteins related to phosphorylated sites of EGFR/MET (MAPK, AKT, STAT1, 3, 5, and Shc; see also Table 2). Among these proteins we found that enhanced phosphorylation of Shc (GenBank Accession No.: NM_{—}001014431), STAT3 (GenBank Accession No.: NM_{—}139276), MAPK and AKT in COS-7 cells transfected with EPHA7 expressing vector, compared with mock transfected COS-7 (FIG. 22). We detected no significant enhancement of phosphorylation in STAT1 and -5 (data not shown). The data clearly suggest that EPHA7 expressed in mammalian cells could enhance specific downstream pathways of EGFR/MET that are important for cancer cell growth, survival, and/or invasion.

### (8) Discussion

In the last decade, little improvement has been achieved in prognosis of lung cancer patients and quality of life in spite of the daily progression in therapeutic drugs and radiotherapies, and imaging of tumors. The powerful diagnostic strategies and tools for example, tumor biomarkers for lung cancers are still desired all over the world, since the early detection of tumors is one of the most effective demand in lung cancer treatment. A few tumor-specific biomarkers detecting cancer specific transmembrane/secretory proteins for example, CYFRA or Pro-GRP are now available (Pujol J L, et al., Cancer Res. 1993 Jan. 1; 53(1): 61-6; Miyake Y, et al., Cancer Res. 1994 Apr. 15; 54(8): 2136-40). Tumor-specific transmembrane/secretory proteins find use as molecular targets because they are presented either on the cell surface or the extracellular space, making them easily accessible as molecular therapeutic targets. Rituximab (Rituxan), a humanized monoclonal antibody against CD20-positive lymphomas, provides proof that targeting specific cell surface proteins can result in significant clinical benefits (Hennessy B T, et al., Lancet Oncol. 2004 June; 5(6):341-53). Therefore, we have exploited the power of genome-wide cDNA microarray analysis to select such genes encoding tumor-specific transmembrane/secretory proteins that are overexpressed in cancer cells, and identified EPHA7 as a target for development of effective tools for diagnosis and treatment of lung cancer.

Of all the receptor tyrosine kinases (RTKs) that are found in the human genome, the Eph-receptor family which have 13 members constitutes the largest family. The EPH receptors are divided on the basis of sequence similarity and ligand affinity into an A-subclass, which contains eight members (EPHA1-EPHA8), and a B-subclass, which in mammals contains five members (EPHB1-EPHB4, EPHB6). Their ligands, the ephrins, are divided into two subclasses, the A-subclass (ephrinA1-ephrinA5), which are tethered to the cell membrane by a glycosylphosphatidylinositol (GPI) ANCHOR, and the B-subclass (ephrinB1-ephrinB3), members of which have a transmembrane domain that is followed by a short cytoplasmic region (Kullander K & Klein R. Nat Rev Mol Cell Biol. 2002 July; 3(7):475-86). Several signal transduction pathways are known about EPH/ephrin axis, for example EPHA4 was involved in the JAK/Stat pathway (Lai K O, et al., J Biol Chem. 2004 Apr. 2; 279(14):13383-92. Epub 2004 Jan. 15), and EPHB4 receptor signaling mediates endothelial cell migration and proliferation via the PI3K pathway (Steinle J J, et al., J Biol Chem. 2002 Nov. 15; 277(46):43830-5. Epub 2002 Sep. 13). Furthermore EPH/ephrin axis regulated the activities of Rho signalling or small GTPases of the Ras family (Lawrenson I D, et al., J Cell Sci. 2002 Mar. 1; 115(Pt 5):1059-72; Murai K K & Pasquale E B. J Cell Sci. 2003 Jul. 15; 116(Pt 14):2823-32).

In spite of several reports about the importance of EPH receptor family proteins in signaling pathways for cell proliferation and transformation, EPHA7 was only reported to be expressed during limb development and in nervous system (Salsi V & Zappavigna V. J Biol Chem. 2006 Jan. 27; 281(4):1992-9. Epub 2005 Nov. 28; Rogers J H, et al., Brain Res Mol Brain Res. 1999 Dec. 10; 74(1-2):225-30; Araujo M & Nieto M A. Mech Dev. 1997 November; 68(1-2):173-7).

Our treatment of lung-cancer cells with specific siRNA to reduce expression of EPHA7 resulted in growth suppression. The expression of EPHA7 also resulted in the significant promotion of the cell growth and invasion in in vitro assays. Moreover, clinicopathological evidence obtained through our tissue-microarray experiments demonstrated that NSCLC patients with tumors strongly expressing EPHA7 showed shorter cancer-specific survival periods than those with weak or absent EPHA7 expression. The results obtained by in vitro and in vivo assays are consistent with the conclusion that overexpressed EPHA7 is an important growth factor and is associated with cancer cell growth and invasion, inducing a highly malignant phenotype of lung-cancer cells.

Furthermore, as an intracellular target molecule of EPHA7 kinase, the present inventors found Tyr-845 of EGFR, Tyr-783 of PLCgamma, and Ser-216 of CDC25, whose pathway was well known to be involved in cellular proliferation and invasion. For example, Phosphorylation of EGFR at tyrosine 845 was reported in hepatocellular carcinomas (Kannangai R, et al., Mod Pathol. 2006 November; 19(11):1456-61. Epub 2006 Aug. 25). PLCgamma is the PLC isozyme that mediates PDGF-induced inositol phospholipid hydrolysis whose phosphorylation on Tyr-783 is essential for PLCgamma activation (Kim H K, et al., Cell. 1991 May 3; 65(3):435-41). PLCgamma phosphorylation at tyrosine 783 by PDGF plays an important role in cytoskeletal reorganization in addition to mitogenesis (Yu H, et al., Exp Cell Res. 1998 Aug. 25; 243(1):113-22). CDC25 is a protein phosphatase responsible for dephosphorylating and activating cdc2, a crucial step in regulating the entry of all eukaryotic cells into mitosis (Jessus C & Ozon R. Prog Cell Cycle Res. 1995; 1:215-28).

In vitro, p38 binds and phosphorylates CDC25B at serines 309 and 361, and CDC25C at serine-216; phosphorylation of these residues is required for binding to 14-3-3 proteins (Bulavin D V, et al., Nature. 2001 May 3; 411(6833):102-7), and the binding of 14-3-3 proteins and nuclear export regulate the intracellular localization of CDC25 (Kumagai A & Dunphy W G. Genes Dev. 1999 May 1; 13(9):1067-72).

We identified an interesting evidence that EPHA7 activation functions as a unique signaling in tumor proliferation and invasion by directly interacting with and phosphorylating EGFR and/or MET that possibly enhance the downstream oncogenic signaling pathway including MAPK, AKT, and STAT3 (Blume-Jensen P, et al. Nature 2001; 411:355-65., Birchmeier C, et al. Nat Rev Mol Cell Biol 2003; 4:915-25). A recent report suggested that RTKs could be synergically activated on cancer cell surface and thereby complementary might activate downstream signaling such as MAPK and AKT (Stommel J M, et al. Science 2007; 318:287-290), however there was no report describing the new types of RTK heterodimer formation between EGFR and Eph-RTKs or between MET and Eph-RTKs that could drastically enhance subsequent downstream signals. The new heterodimeric activation of EGFR or MET might confer complementary function in individual oncogenic signaling and cause the natural and/or acquired resistance of cancer cells to EGFR tyrosine kinase inhibitors (i.e. gefitinib and erlotinib) or MET inhibitors. We found that tyrosine residues of C-terminal portion of EGFR/MET could be directly phosphorylated by EPHA7, which might lead to downstream signal enhancement. Phosphorylation of EGFR Tyr1068 and Tyr1086 is considered to be docking site of several adaptor proteins (Batzer A G, et al. Mol Cell Biol 1994; 14:5192-201., Rodrigues G A, et al. Mol Cell Biol 2000; 20:1448-59). Grb2, Gab1 and p85 can bind such phosphorylated residues and activate downstream MAPK or AKT signaling. Phosphorylated Tyr1068 and Tyr1086 can activate STAT3 signaling directly and indirectly (Shao H, et al. Cancer Res 2003; 63:3923-30., Xi S, et al. J Biol Chem 2003; 278:31574-83). Phosphorylated Tyr1173 associates with Shc (GenBank Accession No.: NM_{—}001130041) which subsequently leads to MAPK signaling (Batzer A G, et al. Mol Cell Biol 1994; 14:5192-201). On the other hand, together with Tyr1356, phosphorylated MET Tyr1349 is known as docking site for adaptor proteins such as Grb2 and phosphatidylinositol 3-kinase (Ponzetto C et al. Cell 1994; 77:261-71., Ponzetto C, et al. Mol Cell Biol 1993; 13:4600-8., Nguyen L, et al. J Biol Chem 1997; 272:20811-9), whereas the function of phospho-MET-Tyr1313 and -Tyr1365 in carcinogenesis have not been elucidated. Although which RTKs are important for downstream signaling may vary among cancer cells and how such ‘dominant RTKs’ are determined still unclear, there may be certain population of lung and esophageal cancers in which EPHA7 plays key roles in cancer proliferation, survival, and invasion. Our data strongly suggest that EPHA7 could contribute to the oncogenic addiction of cancer cells whose EGFR/MET signals were up-regulated, and that regulating EPHA7 activity could be a promising therapeutic strategy for treatment of cancer patients.

It also found high levels of EPHA7 protein in serologic samples from lung cancer and ESCC patients. To examine the feasibility for applying EPHA7 as the diagnostic tool, we compared serum levels of EPHA7 with those of CEA or ProGRP, two conventional diagnostic markers for NSCLCs and SCLCs, regarding its sensitivity and specificity for diagnosis. An assay combining both markers (EPHA7+CEA or EPHA7+ProGRP) increased the sensitivity to more than 75% for lung cancer (NSCLC as well as SCLC), significantly higher than that of CEA or ProGRP alone, while around 7% of healthy volunteers were falsely diagnosed as positive. Our data presented here sufficiently demonstrate the clinical usefulness of EPHA7 as a serological marker for lung and esophageal cancers.

In conclusion, activation of EPHA7 has a functional role for growth and/or malignant phenotype of lung and esophageal cancer cells. The combination of serum EPHA7 and other tumor markers significantly improves the sensitivity of lung cancer diagnosis. Designing new anti-cancer drugs to specifically target the EPHA7 signal transduction is a promising therapeutic and diagnostic strategy for treatment of cancer patients.

### Example 4

### STK31

### (1) STK31 Expression in Lung and Esophageal Tumors, and Normal Tissues.

To identify molecules that can be applicable to treatments based on the biological characteristics of cancer cells, the present inventors expression profile analysis of lung carcinoma and ESCC using a cDNA microarray. Among 27,648 genes screened, we identified STK31 to be overexpressed in a large population of lung and esophageal cancers sample examined. The present inventors confirmed its overexpression by means of semiquantitative RT-PCR experiments in 8 of 15 lung cancer tissues, in 11 of 23 lung cancer cell lines (FIG. 9A), in 4 of 10 ESCC tissues, and in 7 of 10 ESCC cell lines (FIG. 9B). To determine the subcellular localization of endogenous STK31 protein in cancer cells, we did immunofluorescence analysis using anti-STK31 antibody and NCI-H2170 cells, and found that STK31 was located at cytoplasm and nucleus of tumor cells (FIG. 9C).

Northern blot analysis using a STK31 cDNA fragment as a probe identified a 3.6-kb transcript, only in the testis among 23 human tissues examined (FIG. 9D). Furthermore, we compared STK31 protein expressions in 5 normal tissues (heart, liver, kidney, lung, and testis) with those in lung cancers using anti-STK31 polyclonal antibodies by immunohistochemistry. STK31 expressed in testis (in cytoplasm and/or nucleus of cells) and lung cancers, but its expression was hardly detectable in the remaining four normal tissues (FIG. 10A).

(2) Association of STK31 Expression with Poor Prognosis.

To investigate the biological and clinicopathologic significance of STK31 in pulmonary carcinogenesis, the present inventors carried out immunohistochemical staining on tissue microarray containing tissue sections from 368 NSCLC cases that underwent curative surgical resection. STK31 staining with polyclonal antibody specific to STK31 was mainly observed at nucleus and cytoplasm of tumor cells but was not detected in normal cells (FIG. 10B). Of the 368 NSCLCs, STK31 was positively stained in 235 (63.9%) cases (score 1+) and not stained in 133 (36.1%) cases (score 0). The present inventors then examined a correlation of STK31 expression (positive vs negative) with various clinicopathologic parameters and found its significant correlation with histological type (higher in non-ADC; P=0.0033 by Fisher's exact test) and smoking history (higher in smokers; P=0.0446 by Fisher's exact test) (Table 5A). The median survival time of NSCLC patients was significantly shorter in accordance with the expression of STK31 (P=0.0178, log-rank test; FIG. 10C). The present inventors also applied univariate analysis to evaluate associations between patient prognosis and other factors, including age (<65 vs ≧65), gender (female vs male), pathologic tumor stage (tumor size; T1+T2 vs T3+T4), pathologic node stage (node status; N0+N1 vs N2), histological type (ADC vs non ADC), and smoking history (never smoker vs smoker). Among those parameters, STK31 status (P=0.0178), male (P=0.0005), advanced pT stage (P=0.0005), advanced pN stage (P<0.0001), non-ADC histological classification (P=0.0115), and smoking history (P=0.0297) were significantly associated with poor prognosis (Table 5B). In multivariate analysis of the prognostic factors, STK31 status did not reach the statistically significant level as independent prognostic factor for surgically treated NSCLC patients enrolled in this study (P=0.0829), while pT and pN stages as well as gender did so (P=0.0017, <0.0090, and <0.0001, respectively), demonstrating the relevance of STK31 expression to these clinicopathological factors in lung cancer (Table 5B).

TABLE 5A
Association between STK31-positivity in NSCLC tissues and
patients' characteristics (n = 368)

| | | STK31 | STK31 | | P-value |
|---|---|---|---|---|---|
| | Total | positive | absent | | positive |
| | n = 368 | n = 236 | n = 132 | Chi-square | vs absent |
| Gender | | | | | |
| Male | 259 | 171 | 88 | 1.326 | NS |
| Female | 109 | 65 | 44 | | |
| Age (years) | | | | | |
| <65 | 180 | 113 | 67 | 0.28 | NS |
| >=65 | 188 | 123 | 65 | | |
| Histological type | | | | | |
| ADC | 234 | 137 | 97 | 8.709 | 0.0033* |
| non-ADC | 134 | 99 | 35 | | |
| pT factor | | | | | |
| T1 + T2 | 254 | 159 | 95 | 0.837 | NS |
| T3 + T4 | 114 | 77 | 37 | | |
| pN factor | | | | | |
| N0 + N1 | 271 | 171 | 100 | 0.475 | NS |
| N2 | 97 | 65 | 32 | | |
| Smoking history | | | | | |
| Never smoker | 110 | 62 | 48 | 4.114 | 0.0446* |
| smoker | 258 | 174 | 84 | | |

ADC, adenocarcinoma
non-ADC, squamous-cell carcinoma plus large-cell carcinoma and adenosquamous-cell carcinoma
NS, no significance
*P < 0.05 (Fisher's exact test)

TABLE 5B
Cox's proportional hazards model analysis of prognostic factors in
patients with NSCLCs

| | Hazards | | Unfavorable/ | |
|---|---|---|---|---|
| Variables | ratio | 95% CI | Favorable | P-value |
| Univariate | | | | |
| analysis | | | | |
| STK31 | 1.465 | 1.068-2.010 | Positive/Negative | 0.0178* |
| Age (years) | 1.258 | 0.938-1.688 | >=65/65> | NS |
| Gender | 1.862 | 1.310-2.646 | Male/Female | 0.0005* |
| pT factor | 1.712 | 1.268-2.313 | T3 + T4/T1 + T2 | 0.0005* |
| pN factor | 2.742 | 2.031-3.701 | N2/N0 + N1 | <0.0001* |
| Histological | 1.461 | 1.089-1.959 | non-ADC/ADC | 0.0115* |
| type | | | | |
| Smoking | 1.450 | 1.037-2.206 | Smoker/ | 0.0297* |
| history | | | Never smoker | |
| Multivariate | | | | |
| analysis | | | | |
| STK31 | 1.180 | 0.854-1.630 | Positive/Negative | 0.0829 |
| Gender | 1.903 | 1.170-3.095 | Male/Female | 0.0017* |
| pT factor | 2.315 | 1.564-3.428 | T3 + T4/T1 + T2 | <0.0090* |
| pN factor | 2.301 | 1.702-3.111 | N2/N0 + N1 | <0.0001* |
| Histological | 1.060 | 0.764-1.471 | non-ADC/ADC | 0.1645 |
| type | | | | |
| smoking | 0.707 | 0.440-1.137 | smoker/ | 0.1777 |
| history | | | Never smoker | |

ADC, adenocarcinoma
non-ADC, squamous-cell carcinoma plus large-cell carcinoma and adenosquamous-cell carcinoma
NS, no significance
*P < 0.05

### (3) Growth Promoting Effects of STK31.

To assess whether STK31 is essential for growth or survival of lung cancer cells, we constructed plasmids to express siRNA against STK31 (si-STK31-#1 and si-STK31-#2). The siRNAs were transfected each of them or siRNAs for EGFP and Luciferase as controls into LC319 and NCI-H2170 cells (representative data of LC319 is shown in FIGS. 11A-C). A knockdown effect was confirmed by RT-PCR when we used si-STK31-#1 and si-STK31-#2 constructs (FIG. 11A). MTT assays and colony-formation assays using LC319 revealed a drastic reduction in the number of cells transfected with si-STK31-#1 and si-STK31-#2 (FIGS. 11B and 11C; P<0.001). The present inventors next examined a role of STK31 in promoting cell growth. The present inventors prepared plasmids designed to express STK31 (pCAGGSn-STK31-3xFlag) and transfected them into COS-7 cells. As shown in FIG. 11D, transfection of STK31 cDNA into COS-7 cells significantly enhanced the growth of COS-7 cells, compared with that of mock vector.

### (4) Kinase Activity of STK31 Recombinant Protein.

To examine the kinase activity of STK31, the present inventors did in vitro kinase assay using recombinant STK31 protein and MBP (as universal substrate), and detected 15 kDa of phosphorylated MBP protein, indicating that STK31 protein appeared to have kinase activity (FIG. 12A).

(5) Identification of EGFR (Ser1046/1047) and p44/42 MAPK (Thr202/Tyr204) as Downstream Targets for STK31.

To elucidate the function of STK31 kinase in carcinogenesis, the present inventors attempted to identify substrate and/or downstream target proteins that would be phosphorylated through STK31 signaling and activate cell-proliferation signaling. The present inventors performed immunoblot-screening of kinase substrates for STK31 using cell lysates of COS-7 cells transfected with STK31-expression vector and a series of antibodies specific for phospho-proteins related to cancer-cell signaling (see Table 2). The present inventors screened a total of 26 phosphoproteins and found that Ser1046/1047 of EGFR and Thr202/Tyr204 of ERK (p44/42 MAPK) were significantly phosphorylated in the cells transfected with the STK31-expression vector, compared with those with mock vector (FIG. 12B). We subsequently performed in vitro kinase assay by incubating recombinant STK31 with whole extracts prepared from COS-7 cells. Western-blot analyses using the phospho-specific antibodies for ERK (P44/42 MAPK) (Thr202/Tyr204) found that recombinant STK31 specifically induced phosphorylation of ERK (P44/42 MAPK) at Thr202/Tyr204 in a dose dependent manner. (FIG. 12C)

### (6) Involvement of STK31 in MAPK Pathway.

To determine the mechanism of ERK (ERK1/2) (Thr202/Tyr204) phosphorylation by STK31, attempt examined the activation of the upstream pathway of ERK in cells transfected with STK31-expressing vector. Expression of STK31 increased phosphorylation of MEK (MEK1/2) in COS-7 cells and SBC-5 cells (FIG. 12D). Additionally, phosphorylation of both ERK1/2 and MEK in SBC-5 cells was reduced in accordance with the suppression of STK31 expression by siRNA against (FIG. 12E). Furthermore, we confirmed by immunoprecipitation using lysates from COS-7 cells transfected with STK31-expressing vector that exogenous STK31 could bind to endogenous c-raf, MEK, and ERK1/2, suggesting possible activation of the MAPK signals by STK31 overexpression.

### (7) Discussion

Lung cancer and ESCC are considered to reveal the worst prognosis among malignant tumors in spite of modern surgical techniques and adjuvant chemotherapy. Through identification of molecules specifically expressed in cancer cells, molecular-targeting drugs for cancer therapy have been recently developed. However, the proportion of patients showing good response to presently available treatments is still very limited. Hence, it is urgent to develop effective therapeutic anti-cancer drugs with a minimum risk of adverse reactions. Towards this aim, we performed a genome-wide expression profile analysis of 101 lung cancers and 19 ESCC cells after enrichment of cancer cells by laser microdissection using a cDNA microarray containing 27,648 genes (Kikuchi T, et al., Oncogene. 2003 Apr. 10; 22(14):2192-205; Kakiuchi S, et al., Mol Cancer Res. 2003 May; 1(7):485-99; Kikuchi T, et al., Int J Oncol. 2006 April; 28(4):799-805; Taniwaki M, et al., Int J Oncol. 2006 September; 29(3):567-75; Yamabuki T, et al., Int J Oncol. 2006 June; 28(6):1375-84). Through the analyses, the present inventors identified several candidate molecular target genes that were significantly up-regulated in cancer samples, but scarcely expressed in normal tissues. The present inventors verified the targeted genes whether they are essential for survival/growth of lung cancer cells as well as tumor progression using siRNA technique and tissue microarray consisting of hundreds of archived NSCLC tissue samples (Suzuki C, et al., Cancer Res. 2003 Nov. 1; 63(21):7038-41; Cancer Res. 2005 Dec. 15; 65(24):11314-25; Mol Cancer Ther. 2007 February; 6(2):542-51; Ishikawa N, et al., Clin Cancer Res. 2004 Dec. 15; 10(24):8363-70; Cancer Res. 2005 Oct. 15; 65(20):9176-84; Cancer Sci. 2006 August; 97(8):737-45; Kato T, et al., Cancer Res. 2005 Jul. 1; 65(13):5638-46; Clin Cancer Res. 2007 Jan. 15; 13(2 Pt 1):434-42; Furukawa C, et al., Cancer Res. 2005 Aug. 15; 65(16):7102-10; Takahashi K, et al., Cancer Res. 2006 Oct. 1; 66(19):9408-19; Hayama S, et al., Cancer Res. 2006 Nov. 1; 66(21):10339-48; Cancer Res. 2007 May 1; 67(9):4113-22; Yamabuki T, et al., Cancer Res. 2007 Mar. 15; 67(6):2517-25). By this systematic approach, we identified that STK31 was overexpressed in the great majority of clinical lung cancer and ESCC samples and that this molecule is indispensable for growth and progression of cancer cells.

In a systematic search for genes expressed in mouse spermatogonia but not in somatic tissues, Wang et al. (Wang P J, et al., Nat Genet. 2001 April; 27(4):422-6) identified 25 genes, 19 of which were not previously known, that are expressed in only male germ cells; one of these genes was STK31. STK31 encodes a 115-kDa protein that contains a Tudor domain on its N-terminus, which was known to be involved in RNA binding, and Ser/Thr-kinase protein kinase domain on the C-terminus, however its physiological function remains unclear. STK31 is classified into a very unique category by the phylogenetic tree of Kinome (on the worldwide web at cellsignal.com/reference/kinase/kinome.jsp). PKR is considered as a structural homolog of STK31. PKR protein kinase, also binds to double-strand RNA with its N-terminal domain, and has a C-terminal Ser/Thr-kinase domain.

When bound to an activating RNA and ATP, PKR undergoes autophosphorylation reactions and phosphorylates the alpha-subunit of eukaryotic initiation factor 2 (elF2 alpha), inhibiting the function of the elF2 complex and continued initiation of translation (Manche L, et al., Mol Cell Biol. 1992 November; 12(11):5238-48; Jammi N V & Beal P A. Nucleic Acids Res. 2001 Jul. 15; 29(14):3020-9; Kwon H C, et al., Jpn J Clin Oncol. 2005 September; 35(9):545-50. Epub 2005 Sep. 7). Recently, several serine threonine kinases are considered to be a good therapeutic target for cancer. Protein kinase C beta (PKC beta), which belongs to the member of serine threonine kinases, was found to be overexpressed in fatal/refractory diffuse large B-cell lymphoma (DLBCL) and to be as a target for anti-tumor therapy (Goekjian P G & Jirousek M R. Expert Opin Investig Drugs. 2001 December; 10(12):2117-40).

A phase II study was conducted with the inhibitor of PKC beta, enzastaurin, in patients with relapsed or refractory DLBCL (Goekjian P G & Jirousek M R. Expert Opin Investig Drugs. 2001 December; 10(12):2117-40). In this study, it was found that is STK31 was overexpressed in lung and esophageal cancers, but not detected in normal tissues except the testis.

The present inventors also proved that STK31 has a growth promoting effect on mammalian cells and also has protein kinase activity, demonstrating that STK31 finds use as a therapeutic target. Interestingly, induction of STK31 in mammalian cells promoted the phosphorylation of EGFR (Ser1046/1047), ERK (p44/42 MAPK) (Thr202/Tyr204) and MEK (S217/221), and STK31 could interact with c-raf, MEK1/2, and ERK1/2. The data suggests that these molecules are the downstream targets of STK31. It was shown that Ser1046/1047 of EGFR is phosphorylated by Ca²⁺/calmodulin-dependant kinase II (CaM kinase II) and its phosphorylation attenuated EGFR kinase activity (Robertson M J, et al., J Clin Oncol. 2007 May 1; 25(13):1741-6. Epub 2007 Mar. 26; Feinmesser R L, et al., J Biol Chem. 1999 Jun. 4; 274(23):16168-73; Countaway J L, et al., J Biol Chem. 1992 Jan. 15; 267(2):1129-40). CaM kinase II was also reported to cause ERK (P44/42 MAPK) activation that regulates cell growth and differentiation (Ginnan R & Singer H A. Am J Physiol Cell Physiol. 2002 April; 282(4):C754-61). These results of the present invention also raise a hypothesis that STK31 is a scaffold protein as a positive modulator of MAPK cascade. Scaffold proteins provide one of the mechanisms contributing to specificity in kinase signaling cascades. These proteins ensure efficient and specific transduction of signals by physical binding and bringing together the upstream and downstream elements of signaling pathways. Kinase suppressor of RAS1 (KSR1) has a putative kinase-like domain, but it is reported that KSR1 lacks enzymatic activity and serves as a docking platform for the authentic kinase components of MAPK cascade (Erzsebet Szatmari et al. J. Neurosci. 2007 27: 11389-11400, Jürgen Müller et al. Molecular Cell 2001; 8:983-993., M Therrien, et al. Genes Dev. 1996 10: 2684-2695., Scott Stewart, et al. Mol. Cell. Biol. 1999 19: 5523-5534).

In summary, it was identified that a cancer-testis antigen STK31 was overexpressed in the great majority of lung and esophageal cancer tissues, and its functional role was associated with growth and/or survival of cancer cells. STK31 is useful as a prognostic biomarker for lung cancers, and as a therapeutic target for the development of anti-cancer agents and cancer vaccines.

### Example 5

### WDHD1

### (1) WDHD1 Expression in Lung and Esophageal Cancers and Normal Tissues.

To identify molecules useful to detect presence of cancer at an early stage and to develop treatments based on the biological characteristics of cancer cells, the present inventors performed genome-wide expression profile analysis of lung carcinoma and ESCC using a cDNA microarray (Kikuchi T, et al., Oncogene. 2003 Apr. 10; 22(14):2192-205; Int J Oncol. 2006 April; 28(4):799-805; Kakiuchi S, et al., Mol Cancer Res. 2003 May; 1(7):485-99; Hum Mol Genet. 2004 Dec. 15; 13(24):3029-43. Epub 2004 Oct. 20; Taniwaki M, et al., Int J Oncol. 2006 September; 29(3):567-75; Yamabuki T, et al., Int J Oncol. 2006 June; 28(6):1375-84).

Among 27,648 genes screened, the present inventors identified elevated expression (3-fold or higher) of WDHD1 transcript in cancer cells in the great majority of the lung and esophageal cancer samples examined. The present inventors confirmed its over-expression by means of semi-quantitative RT-PCR experiments in 14 of 15 lung cancer tissues, in 20 of 24 lung-cancer cell lines, in 6 of 10 ESCC tissues, and in 6 of 10 ESCC cell lines (FIGS. 13A and 13B). The present inventors subsequently confirmed by Western blotting analysis over-expression of 126-kDa WDHD1 protein in lung and esophageal cancer cell lines using anti-WDHD1 antibody (FIG. 13C). To examine the subcellular localization of endogenous WDHD1 in NSCLC cells, the present inventors performed immunofluorescence analysis using anti-WDHD1 antibody and LC319 cells. WDHD1 was localized abundantly in the nucleus and weakly in cytoplasm throughout the cell cycle, and it was detected on chromosomes during the mitotic phase. (FIG. 13D).

Northern blot analysis using a WDHD1 cDNA fragment as a probe identified about 5 kb transcript only in testis (FIG. 14A). Furthermore, the present inventors compared WDHD1 protein expressions in 5 normal tissues (liver, heart, kidney, lung, and testis) with those in lung cancers using anti-WDHD1 polyclonal antibodies by immunohistochemistry. WDHD1 expressed abundantly in testis (mainly in nucleus and/or cytoplasm of primary spermatocytes) and lung cancers, but its expression was hardly detectable in the remaining four normal tissues (FIG. 14B).

(2) Association of WDHD1 Expression with Poor Prognosis.

To investigate the biological and clinicopathological significance of WDHD1 in pulmonary and esophageal carcinogenesis, the present inventors carried out immunohistochemical staining on tissue microarray containing tissue sections from 264 NSCLC and 297 ESCC cases that underwent curative surgical resection. WDHD1 staining with polyclonal antibody specific to WDHD1 was mainly observed at nucleus and cytoplasm of tumor cells, but not detected in normal cells (FIG. 14C, left panels). Of the 264 NSCLCs, WDHD1 was highly stained in 134 cases (50.8%) and not stained in 130 cases (49.2%) (details are shown in Table 6A). The present inventors then examined the association of WDHD1 expression with clinical outcomes. The median survival time of NSCLC patients was significantly shorter in accordance with the higher expression levels of WDHD1 (P=0.0208 by log-rank test; FIG. 2C, right panel). The present inventors also applied univariate analysis to evaluate associations between patient prognosis and several factors including age, gender, pT stage (tumor size; T1 versus T2+T3+T4), pN stage (node status; N0 versus N1+N2), histological type (non-ADC versus ADC), and WDHD1 status (positive versus negative). All those parameters were significantly associated with poor prognosis (Table 6B). In multivariate analysis, WDHD1 status did not reach the statistically significant level as independent prognostic factor for surgically treated lung cancer patients enrolled in this study (P=0.8668), demonstrating the relevance of WDHD1 expression to these clinicopathological factors in lung cancer (Table 6B).

Of the 297 ESCC cases examined, WDHD1 was highly stained in 180 cases (60.6%) and not stained in 117 cases (39.4%) (FIG. 14D, left panels; details are shown in Table 7A). The median survival time of ESCC patients was significantly shorter in accordance with the highly expression levels of WDHD1 (P=0.0285 by log-rank test; FIG. 14D, right panel). The present inventors also applied univariate analysis to evaluate associations between ESCC patient prognosis and several factors including age, gender, pT stage (tumor depth; T1+T2 versus T3+T4), pN stage (node status; N0 versus N1), and WDHD1 status (positive versus negative). All those parameters except for age were significantly associated with poor prognosis (Table 7B). Multivariate analysis using a Cox proportional hazard factors determined that WDHD1 (P=0.0085) as well as other three factors (male gender, larger tumor size, and lymph node metastasis) were independent prognostic factors for surgically treated ESCC patients (Table 7B).

TABLE 6A
Association between WDHD1-positivity in NSCLC tissues and
patients' characteristics (n = 264)

| | | | | | P-value |
|---|---|---|---|---|---|
| | | WDHD-1 | WDHD-1 | | positive |
| | Total | positive | negative | | vs |
| | n = 264 | n = 134 | n = 130 | Chi-square | negative |
| Gender | | | | | |
| Female | 85 | 26 | 59 | 20.404 | <0.0001* |
| Male | 179 | 108 | 71 | | |
| Age (years) | | | | | |
| <65 | 128 | 54 | 74 | 7.301 | 0.0096* |
| >=65 | 136 | 80 | 56 | | |
| Histological | | | | | |
| type | | | | | |
| ADC | 155 | 58 | 97 | 26.722 | <0.0001* |
| non-ADC | 109 | 76 | 33 | | |
| pT factor | | | | | |
| T1 | 105 | 39 | 66 | 12.929 | 0.0004* |
| T2 + T3 + T4 | 159 | 95 | 64 | | |
| pN factor | | | | | |
| N0 | 200 | 95 | 105 | 3.503 | 0.0639 |
| N1 + N2 | 64 | 39 | 25 | | |

ADC, adenocarcinoma
non-ADC, squamous-cell carcinoma plus large-cell carcinoma and
adenosquamous-cell carcinoma
*P < 0.05 (Fisher's exact test)

TABLE 6B
Cox's proportional hazards model analysis of prognostic factors
in patients with NSCLCs

| | Hazards | | Unfavorable/ | |
|---|---|---|---|---|
| Variables | ratio | 95% CI | Favorable | P-value |
| Univariate | | | | |
| analysis | | | | |
| WDHD-1 | 1.757 | 1.083-2.852 | Positive/Negative | 0.0225* |
| Age (years) | 2.053 | 1.259-3.347 | >=65/65> | 0.0039* |
| Gender | 1.919 | 1.096-3.360 | Male/Female | 0.0226* |
| pT factor | 3.441 | 1.879-6.298 | T2 + T3 + T4/T1 | <0.0001* |
| pN factor | 4.136 | 2.564-6.672 | N1 + N2/N0 | <0.0001* |
| Histological | 2.459 | 1.511-4.002 | non-ADC/ADC | 0.0003* |
| type | | | | |
| Multivariate | | | | |
| analysis | | | | |
| WDHD-1 | 0.955 | 0.556-1.639 | Positive/Negative | 0.8668 |
| Age (years) | 1.787 | 1.085-2.944 | >=65/65> | 0.0226 |
| Gender | 1.328 | 0.696-2.537 | Male/Female | 0.3895 |
| pT factor | 2.014 | 1.069-3.796 | T2 + T3 + T4/T1 | 0.0303* |
| pN factor | 3.562 | 2.188-5.798 | N1 + N2/N0 | <0.0001* |
| Histological | 1.634 | 0.910-2.933 | non-ADC/ADC | 0.0999 |
| type | | | | |

ADC, adenocarcinoma
non-ADC, squamous-cell carcinoma plus large-cell carcinoma and adenosquamous-cell carcinoma
*P < 0.05

TABLE 7A
Association between WDHD-1-positivity in ESCC tissues
and patients' characteristics (n = 297)

| | | WDHD-1 | WDHD-1 | | P-value |
|---|---|---|---|---|---|
| | Total | positive | negative | | positive vs |
| | n = 297 | n = 180 | n = 117 | Chi-square | negative |
| Gender | | | | | |
| Female | 28 | 16 | 12 | 0.155 | 0.6898 |
| Male | 269 | 164 | 105 | | |
| Age (years) | | | | | |
| <65 | 183 | 118 | 65 | 2.998 | 0.887 |
| >=65 | 114 | 62 | 52 | | |
| pT factor | | | | | |
| T1 + T2 | 128 | 73 | 55 | 1.204 | 0.2829 |
| T3 + T4 | 169 | 107 | 62 | | |
| pN factor | | | | | |
| N0 | 93 | 58 | 35 | 0.176 | 0.7025 |
| N1 | 204 | 122 | 82 | | |

TABLE 7B
Cox's proportional hazards model analysis of prognostic
factors in patients with ESCCs

| | Hazards | | Unfavorable/ | |
|---|---|---|---|---|
| Variables | ratio | 95% CI | Favorable | P-value |
| Univariate | | | | |
| analysis | | | | |
| WDHD-1 | 1.393 | 1.034-1.877 | Positive/Negative | 0.0293* |
| Age (years) | 1.050 | 0.785-1.405 | >=65/65> | 0.7401 |
| Gender | 2.858 | 1.510-5.409 | Male/Female | 0.013* |
| pT factor | 2.407 | 1.773-3.267 | T3 + T4/T1 + T2 | <0.0001* |
| pN factor | 3.552 | 2.436-5.180 | N1/N0 | <0.0001* |
| Multivariate | | | | |
| analysis | | | | |
| WDHD-1 | 1.496 | 1.108-2.020 | Positive/Negative | 0.0085* |
| Gender | 2.849 | 1.501-5.408 | Male/Female | 0.0014* |
| pT factor | 1.914 | 1.395-2.625 | T3 + T4/T1 + T2 | <0.0001* |
| pN factor | 2.957 | 1.999-4.373 | N1 + N2/N0 | <0.0001* |

*P < 0.05

### (3) Effects of WDHD1 on Growth of Cancer Cells.

The present inventors constructed several siRNA expression oligonucleotides specific to WDHD1 sequences and transfected them into A549, LC319 and TE9 cell lines that endogenously expressed high levels of WDHD1. A knockdown effect was confirmed by RT-PCR when we used si-WDHD1-#1 and si-WDHD1-#2 constructs (FIGS. 15A and 15B, top panels). MTT assays and colony-formation assays revealed a drastic reduction in the number of cells transfected with WDHD1-si2 (FIGS. 15A and 15B, middle and bottom panels). Flow cytometric analysis revealed that 72 h after WDHD1 knockdown, the number of cells in sub G1 phase was increased, demonstrating that WDHD1 knockdown induced apoptosis (FIG. 15C). On the other hand, transfection of WDHD1-expression vectors to COS-7 cells increased the viability of cells, compared with that of mock vectors (FIG. 15D). Flowcytometric analysis revealed that 24˜72 hours after the transfection of si-WDHD1 to the lung cancer A549 cells, the number of cells in S phase was continuously decreased, while the proportion of the cells in G0/G1 phase were increased during 48˜72 hours after the transfection (FIG. 15E). To further investigate the effect of WDHD1 on the cell cycle, we synchronized A549 cells which had been transfected siRNA for si-WDHD1 30 minutes before, and monitored their cell cycle. The number of the cells in G0/G1 phase was increased and the progression of S phase was delayed, suggesting that one population was repressed its entry into S phase and remained in G0/G1 phase, while the other population that had been in S phase was repressed its entry into G2/M phase (FIG. 15F). To further investigate the effect of WDHD1 knock-down on cellular morphology, we examined the A549 cells treated with siRNA for WDHD1 using time-lapse microscopy. While the cell division was observed at about every 10 hours in control cells, the WDHD I knocked-down cells divided slowly and died shortly after cell division (FIG. 15G). Immunocytochemical analysis revealed that mitotic cells transfected with siRNA for WDHD1 had a relatively normal spindle, but their chromosomes failed to congress at the spindle midzone, and were dispersed over the spindle. In contrast, the control cells treated with si-LUC assembled like normal metaphase figures in which the chromosomes were well organized at the metaphase plate (FIG. 15H).

### (4) Phosphorylation of WDHD1.

WDHD1 protein was detected as double bands by Western blotting when they were separated for longer times by SDS-PAGE. Therefore, we first incubated extracts from A549 cells in the presence or absence of protein phosphatase (New England Biolabs, Beverly, Mass.) and analyzed the molecular weight of WDHD1 protein by Western blotting analysis. Expectedly, the measured weight of the majority of WDHD1 protein in the extracts treated with phosphatase was smaller than that in the untreated cells. The data indicated that WDHD1 was phosphorylated in lung cancer cells (FIG. 16A, left panels). Immunoprecipitation of WDHD1 with anti-WDHD1 antibody followed by immunoblotting with pan-phospho-specific antibodies indicated phosphorylation of WDHD1 at its serine and tyrosine residues (FIG. 16A, right panels).

### (5) Cell-Cycle Dependent Expression of WDHD1.

Since overexpression of WDHD1 promoted the growth of COS-7 cells, the present inventors examined the expression levels of WDHD1 during cell cycle. LC319 and A549 cells were synchronized using aphidicolin and the expression levels of WDHD1 protein were detected by Western blotting after the release from G0/G1 arrest. WDHD1 levels increased at a transition period from G1 to S phases, reaching the maximum level at S phase and then decreasing in G2 and M phases, demonstrating its functional role in cell cycle progression (FIG. 16B, C).

### (6) Involvement of WDHD1 in PI3K Signaling.

To elucidate the importance of WDHD1 phosphorylation, the present inventors next screen the phosphorylation sites on the WDHD1 protein, and found that one of them had consensus phosphorylation site for AKT kinase (R—X—R—X—X—S374; Olsen J V, et al., Cell. 2006 Nov. 3; 127(3):635-48). Phosphatidylinositol-3 kinase (PI3K)/AKT pathway is well known to be activated in a wide range of tumor types, and this triggers a cascade of responses, from cell growth and proliferation to survival, motility, epithelial-mesenchymal transition and angiogenesis (Krystal G W, et al., Mol Cancer Ther. 2002 September; 1(11): 913-22; Nguyen D M, et al., J Thorac Cardiovasc Surg. 2004 February; 127(2): 365-75; Kandel E S & Hay N. Exp Cell Res. 1999 Nov. 25; 253(1): 210-29; Roy H K, et al., Carcinogenesis. 2002 January; 23(1): 201-5; Altomare D A, et al., J Cell Biochem. 2003 Jan. 1; 88(1): 470-6; Tanno S, et al., Cancer Res. 2004 May 15; 64(10):3486-90).

The present inventors therefore examined whether WDHD1 was involved in the PI3K and/or AKT pathway. The level of WDHD1 protein was measured after treatment with various concentrations of LY294002 (0-40 μmol/L for 24 hours), a specific inhibitor of the catalytic subunit of PI3K, which is directed at the ATP-binding site of the kinase (Vlahos C J, et al., J Biol Chem. 1994 Feb. 18; 269(7):5241-8) and decreases AKT phosphorylation and induces the G1 arrest of cells (Suzuki C, et al., Cancer Res. 2005 Dec. 15; 65(24):11314-25). Total amount of WDHD1 as well as phosphorylated WDHD1 was significantly decreased by LY294002 treatment, indicating that WDHD1 is a downstream target for PI3K pathway (FIG. 16D). To examine whether WDHD1 was a target of AKT1 (GenBank Accession No.: NM_{—}001014431), the expression levels of WDHD1 protein in A549 cells treated with siRNA for AKT1 were examined, and expectedly the levels of WDHD1 protein were decreased (FIG. 16E). We next immunoblotted using phosphor-AKT substrate (PAS) antibody the immunoprecipitated WDHD1 that was exogenously expressed in COS-7 cells, and detected the positive band that represented possibly phosphorylated by endogenous AKT (FIG. 16F). In vitro kinase assay using the WDHD1 immunoprecipitant as a substrate and AKT1 recombinant protein (rhAKT) as a kinase with subsequent immunoblotting with PAS antibody also proved the direct phosphorylation of WDHD1 by AKT (FIG. 16G), suggesting that WDHD1 could be a substrate of AKT kinase. To investigate the phosphorylation site(s) on WDHD1 by AKT1, we constructed WDHD1-expression vectors whose consensus AKT phosphorylation sequence at serine 374 or 1058 on WDHD1 had been replaced with alanine (S374A, S1058A), and transfected either of them into COS-7 cells. Immunoblotting of immunoprecipitated WDHD1 or in vitro kinase assay using immunoprecipitated WDHD1 combined with subsequent immunoblotting with PAS antibody clearly indicated the reduced levels of WDHD1 phosphorylation in cells transfected with S374A mutant, suggesting that serine 374 is one of the major AKT1-dependent phosphorylation sites on WDHD1 (FIG. 16H, I).

### (7) Discussion

We performed a genome-wide expression profile analysis of 101 lung cancers and 19 ESCC cells after enrichment of cancer cells by laser microdissection, using a cDNA microarray containing 27,648 genes (Kikuchi T, et al., Oncogene. 2003 Apr. 10; 22(14): 2192-205; Int J Oncol. 2006 April; 28(4): 799-805; Kakiuchi S, et al., Mol Cancer Res. 2003 May; 1(7): 485-99; Hum Mol Genet. 2004 Dec. 15; 13(24): 3029-43. Epub 2004 Oct. 20; Taniwaki M, et al., Int J Oncol. 2006 September; 29(3): 567-75; Yamabuki T, et al., Int J Oncol. 2006 June; 28(6): 1375-84).

Through the analyses, we identified a number of genes that are good candidates for development of effective diagnostic markers, therapeutic drugs, and/or immunotherapy (Suzuki C, et al., Cancer Res. 2003 Nov. 1; 63(21): 7038-41; Cancer Res. 2005 Dec. 15; 65(24): 11314-25; Mol Cancer Ther. 2007 February; 6(2): 542-51; Ishikawa N, et al., Clin Cancer Res. 2004 Dec. 15; 10(24): 8363-70; Cancer Res. 2005 Oct. 15; 65(20): 9176-84; Cancer Sci. 2006 August; 97(8): 737-45; Kato T, et al., Cancer Res. 2005 Jul. 1; 65(13):5638-46; Clin Cancer Res. 2007 Jan. 15; 13(2 Pt 1):434-42; Furukawa C, et al., Cancer Res. 2005 Aug. 15; 65(16): 7102-10; Takahashi K, et al., Cancer Res. 2006 Oct. 1; 66(19): 9408-19; Hayama S, et al., Cancer Res. 2006 Nov. 1; 66(21): 10339-48; Cancer Res. 2007 May 1; 67(9): 4113-22; Yamabuki T, et al., Cancer Res. 2007 Mar. 15; 67(6): 2517-25). In this study, we selected a WDHD1 as good candidate for diagnostic and prognostic biomarker(s) for lung cancer and/or ESCC and therapeutic target, and provided evidence for its role in human pulmonary and esophageal carcinogenesis.

From the result of northern blot and immunohistochemical analyses, WDHD1 was expressed only in testis and cancer cells. Cancer-testis antigens (CTAs) have been recognized as a group of highly attractive targets for cancer vaccine (Li M, et al., Clin Cancer Res. 2005 Mar. 1; 11(5): 1809-14). Although other factors, for example, the in vivo spontaneous immunogenicity of the protein are also important (Wang Y, et al., Cancer Immun. 2004 Nov. 1; 4:11) WDHD1 is a good target for immunotherapy of lung cancer and ESCC.

WDHD1 encodes a 1129-amino acid protein with high-mobility-group (HMG) box domains and WD repeats domain. The HMG box is well conserved and consists of three alpha-helices arranged in an L-shape, which binds the DNA minor groove (Thomas J O & Travers A A. Trends Biochem Sci. 2001 March; 26(3):167-74). The HMG proteins bind DNA in a sequence-specific or non-sequence-specific way to induce DNA bending, and regulate chromatin function and gene expression (Sessa L & Bianchi M E. Gene. 2007 Jan. 31; 387(1-2):133-40. Epub 2006 Nov. 10). In general, HMG proteins have been known to bind nucleosomes, repress transcription by interacting with the basal transcriptional machinery, act as transcriptional coactivator, or determine whether a specific regulator functions as an activator or a repressor of transcription (Ge H & Roeder R G. J Biol Chem. 1994; 269:17136-40; Paranjape S M, et al., Genes Dev 1995; 9:1978-91; Sutrias-Grau M, et al., J Biol Chem. 1999; 274: 1628-34; Shykind B M, et al., Genes Dev 1995; 9:354-65; Lehming N, et al., Nature 1994; 371:175-79).

Herein it was described that WDHD1 was phosphorylated and stabilized by AKT1. This broad spectrum of functions may be achieved in part by protein-protein interaction in addition to DNA binding activity conferred by the HMG domain. In the case of WDHD1, the candidate domain for protein-protein interaction is the WD-repeats. WD repeat proteins contribute to cellular functions ranging from signal transduction to cell cycle control and are conserved across eukaryotes as well as prokaryotes (Li D & Roberts R. Cell Mol Life Sci. 2001; 58:2085-97). Structural analysis has clarified that WD-repeat proteins form a propeller-like structure with several blades that is composed of a four-stranded antiparallel beta-sheet. This beta-propeller-like structure serves as a platform to which proteins can bind either stably or reversibly (Li D & Roberts R. Cell Mol Life Sci. 2001; 58:2085-97). Evidence of interacting protein with WDHD1 may help the understanding of the WDHD1 function(s).

Cell signaling mechanisms often transmit information via posttranslational protein modifications, most important reversible protein phosphorylation. Some phosphorylation sites in WDHD1 sequence have been detected (Tanno S, et al., Cancer Res. 2004 May 15; 64(10):3486-90 39; Beausoleil S A, et al., Proc Natl Acad Sci USA. 2004 Aug. 17; 101(33):12130-5. Epub 2004 Aug. 9). In our experiment using immunoprecipitation with anti-WDHD1 antibody followed by immunoblotting with pan-phospho-specific antibodies indicated phosphorylation of WDHD1 at its serine and tyrosine residues. The GSK3, CaMK2, AKT, and ALK were predicted as the kinases of these residues using NetPhos 2.0 program (on the worldwide web at cbs.dtu.dk/services/NetPhos/; data not shown). One of the phosphorylated regions of WDHD1 has consensus phosphorylation site for AKT kinase (R—X—R—X—X—S374; Olsen J V, et al., Cell. 2006 Nov. 3; 127(3):635-48). PI3K/AKT signaling is important for cell proliferation and survival (Liang J & Slingerland J M. Cell Cycle. 2003 July-August; 2(4):339-45; Hanahan D, Weinberg R A. Cell. 2000 Jan. 7; 100(1):57-70; Bellacosa A, et al., Oncogene. 1998 Jul. 23; 17(3):313-25). In addition, AKT phosphorylation frequently occurs in various human cancers, and has been recognized as a risk factor for early disease recurrence and poor prognosis (Chen Y L, et al., Cancer Res. 2004 Dec. 1; 64(23):8723-30; Nicholson K M, et al., Breast Cancer Res Treat. 2003 September; 81(2):117-28; Xu X, et al., Oncol Rep. 2004 January; 11(1):25-32; Nakanishi K, et al., Cancer. 2005 Jan. 15; 103(2):307-12). Our data indicated that inhibition of PI3K/AKT pathway using LY294002 and siRNA for AKT1 decreased the expression level of total and phosphorylated WDHD1. This result indicates the possibility that WDHD1 plays a significant role in cancer cell growth/survival as one of the components of the PI3K/AKT pathway.

This result indicates that WDHD1 is one of the components of the PI3K/AKT pathway and is stabilized by phosphorylation. On the other hand, PI3K/AKT/mTOR/p70S6K1 signaling regulates G1 cell cycle progression through the increased expression of cyclins and CDKs. Thus, inhibition of PI3K activity using LY294002 decreased the cell proliferation and induced the G1 cell cycle arrest (Gao N, et al., Am J Physiol Cell Physiol. 2004 August; 287(2):C281-91. Epub 2004 Mar. 17). In our experiment, the expression level of WDHD1 was high in S-phase, so the decrease of WDHD1 expression by LY294002 is due to G1 cell cycle arrest.

In conclusion, WDHD1 was overexpressed in the great majority of lung and esophageal cancer tissues, and plays significant roles in cancer cell growth and/or survival. The data indicated WDHD1 to find use as a therapeutic target and prognostic biomarker for treating patients with lung and esophageal cancers.

### INDUSTRIAL APPLICABILITY

The present inventors have shown that the cell growth is suppressed by double-stranded molecules that specifically target the CDCA5, EPHA7, STK31 or WDHD1 gene. Thus, these double-stranded molecules are useful candidates for the development of anti-cancer pharmaceuticals. For example, agents that block the expression of CDCA5, EPHA7, STK31 or WDHD1 gene protein or prevent its activity may find therapeutic utility as anti-cancer agents, particularly anti-cancer agents for the treatment of lung or esophageal cancer.

The expression of human genes CDCA5, EPHA7, STK31 and WDHD1 are markedly elevated in lung or esophageal cancer. Accordingly, these genes can be conveniently used as diagnostic markers of cancers and the proteins encoded thereby may be used in diagnostic assays of cancers.

Also, EPHA7 is detected in blood sample from lung or esophageal cancer patient. Accordingly, EPHA7 can be used as serological diagnostic markers.

Furthermore, CDCA5, EPHA7, STK31 or WDHD1 polypeptide is a useful target for the development of anti-cancer pharmaceuticals or cancer diagnostic agent. For example, agents that bind CDCA5, EPHA7, STK31 or WDHD1, or block the expression of CDCA5, EPHA7, STK31 and WDHD1, or prevent phosphorylation activity of EPHA7 or STK31, or prevent the phosphorylation of WDHD1, or inhibit the binding between EPHA7 and EGFR may find therapeutic utility as anti-cancer or diagnostic agents, particularly anti-cancer agents for the treatment of lung or esophageal cancer.

1. An isolated double-stranded molecule, which, when introduced into a cell, inhibits in vivo expression of a gene selected from the group consisting of CDCA5, EPHA7, STK31 and WDHD1, and cell proliferation, wherein said double-stranded molecule acts at mRNA which matches a target sequence selected from the group consisting of SEQ ID NO: 38 (at the position of 1713-1732 nt of SEQ ID NO: 5) and SEQ ID NO: 39 (at the position of 2289-2308 nt of SEQ ID NO: 5) for STK31, SEQ ID NO: 40 (at the position of 808-827 nt of SEQ ID NO: 1) and SEQ ID NO: 41 (at the position of 470-488 nt of SEQ ID NO: 1) for CDCA5, SEQ ID NO: 42 (at the position of 2182-2200 nt of SEQ ID NO: 3) and SEQ ID NO: 43 (at the position of 1968-1987 nt of SEQ ID NO: 3) for EPHA7, SEQ ID NO: 44 (at the position of 577-596 nt of SEQ ID NO: 7) and SEQ ID NO: 45 (at the position of 2041-2060 nt of SEQ ID NO: 7) for WDHD1.

2. The double-stranded molecule of claim 1, which comprises a sense strand and an antisense strand complementary thereto, hybridized to each other to form a double strand, wherein said sense strand comprises an oligonucleotide corresponding to a sequence selected from the group consisting of SEQ ID NO: 40 and SEQ ID NO: 41 for CDCA5, SEQ ID NO: 42 and SEQ ID NO: 43 for EPHA7, SEQ ID NO: 38 and SEQ ID NO: 39 for STK31, SEQ ID NO: 44 and SEQ ID NO: 45 for WDHD1.

3. The double-stranded molecule of claim 2, which consists of a single oligonucleotide comprising both the sense and antisense strands linked by an intervening single-strand.

4. The double-stranded molecule of claim 3, which has a general formula 5′-[A]-[B]-[A′]-3′, wherein
[A] is the sense strand comprising an oligonucleotide corresponding to a sequence selected from the group consisting of SEQ ID NO: 40 and SEQ ID NO: 41 for CDCA5, SEQ ID NO: 42 and SEQ ID NO: 43 for EPHA7, SEQ ID NO: 38 and SEQ ID NO: 39 for STK31, SEQ ID NO: 44 and SEQ ID NO: 45 for WDHD1;
[B] is the intervening single-strand; and
[A′] is the antisense strand comprising an oligonucleotide corresponding to a sequence complementary to the sequence selected in [A].

5. The double-stranded molecule of claim 1, which contains 3′ overhang.

6. A vector expressing the double-stranded molecule of claim 1.

7. A method for inhibiting or reducing a growth of a cell expressing a gene selected from the group consisting of CDCA5, EPHA7, STK31 and WDHD1, wherein said method comprising the step of giving at least one double-stranded molecule or a vector expressing at least one double-stranded molecule, wherein said double-stranded molecule or vector is introduced into a cell, inhibits or reduces in vivo expression of said gene.

8. The method of claim 7, wherein said double-stranded molecule, when introduced into a cell, inhibits in vivo expression of a gene selected from the group consisting of CDCA5, EPHA7, STK31 and WDHD1, and cell proliferation, wherein said double-stranded molecule acts at mRNA which matches a target sequence selected from the group consisting of SEQ ID NO: 38 (at the position of 1713-1732 nt of SEQ ID NO: 5) and SEQ ID NO: 39 (at the position of 2289-2308 nt of SEQ ID NO: 5) for STK31, SEQ ID NO: 40 (at the position of 808-827 nt of SEQ ID NO: 1) and SEQ ID NO: 41 (at the position of 470-488 nt of SEQ ID NO: 1) for CDCA5, SEQ ID NO: 42 (at the position of 2182-2200 nt of SEQ ID NO: 3) and SEQ ID NO: 43 (at the position of 1968-1987 nt of SEQ ID NO: 3) for EPHA7, SEQ ID NO: 44 (at the position of 577-596 nt of SEQ ID NO: 7) and SEQ ID NO: 45 (at the position of 2041-2060 nt of SEQ ID NO: 7) for WDHD1.

9. A method for treating or preventing a cancer expressing a gene selected from the group consisting of CDCA5, EPHA7, STK31 and WDHD1, wherein said method comprising the step of administering at least one double-stranded molecule or vector expressing at least one double-stranded molecule, wherein said double-stranded molecule or vector is introduced into a cell, inhibits or reduces in vivo expression of said gene.

10. The method of claim 9, wherein said double-stranded molecule, when introduced into a cell, inhibits in vivo expression of a gene selected from the group consisting of CDCA5, EPHA7, STK31 and WDHD1, and cell proliferation, wherein said double-stranded molecule acts at mRNA which matches a target sequence selected from the group consisting of SEQ ID NO: 38 (at the position of 1713-1732 nt of SEQ ID NO: 5) and SEQ ID NO: 39 (at the position of 2289-2308 nt of SEQ ID NO: 5) for STK31, SEQ ID NO: 40 (at the position of 808-827 nt of SEQ ID NO: 1) and SEQ ID NO: 41 (at the position of 470-488 nt of SEQ ID NO: 1) for CDCA5, SEQ ID NO: 42 (at the position of 2182-2200 nt of SEQ ID NO: 3) and SEQ ID NO: 43 (at the position of 1968-1987 nt of SEQ ID NO: 3) for EPHA7, SEQ ID NO: 44 (at the position of 577-596 nt of SEQ ID NO: 7) and SEQ ID NO: 45 (at the position of 2041-2060 nt of SEQ ID NO: 7) for WDHD1.

11. The method of claim 9, wherein the cancer is lung cancer and/or esophageal cancer.

12. A composition for inhibiting or reducing a growth of a cell expressing a gene selected from the group consisting of CDCA5, EPHA7, STK31 and WDHD1, which comprises at least one double-stranded molecule or vector expressing at least one double-stranded molecule, wherein said double-stranded molecule or vector is introduced into a cell, inhibits or reduces in vivo expression of said gene.

13. The composition of claim 12, wherein said double-stranded molecule, when introduced into a cell, inhibits in vivo expression of a gene selected from the group consisting of CDCA5, EPHA7, STK31 and WDHD1, and cell proliferation, wherein said double-stranded molecule acts at mRNA which matches a target sequence selected from the group consisting of SEQ ID NO: 38 (at the position of 1713-1732 nt of SEQ ID NO: 5) and SEQ ID NO: 39 (at the position of 2289-2308 nt of SEQ ID NO: 5) for STK31, SEQ ID NO: 40 (at the position of 808-827 nt of SEQ ID NO: 1) and SEQ ID NO: 41 (at the position of 470-488 nt of SEQ ID NO: 1) for CDCA5, SEQ ID NO: 42 (at the position of 2182-2200 nt of SEQ ID NO: 3) and SEQ ID NO: 43 (at the position of 1968-1987 nt of SEQ ID NO: 3) for EPHA7, SEQ ID NO: 44 (at the position of 577-596 nt of SEQ ID NO: 7) and SEQ ID NO: 45 (at the position of 2041-2060 nt of SEQ ID NO: 7) for WDHD1.

14. A composition for treating or preventing a cancer expressing a gene selected from the group consisting of CDCA5, EPHA7, STK31 and WDHD1, wherein said method comprising the step of administering at least one double-stranded molecule or vector expressing at least one double-stranded molecule, wherein said double-stranded molecule or vector is introduced into a cell, inhibits or reduces in vivo expression of said gene and cell proliferation.

15. The composition of claim 14, wherein said double-stranded molecule, when introduced into a cell, inhibits in vivo expression of a gene selected from the group consisting of CDCA5, EPHA7, STK31 and WDHD1, and cell proliferation, wherein said double-stranded molecule acts at mRNA which matches a target sequence selected from the group consisting of SEQ ID NO: 38 (at the position of 1713-1732 nt of SEQ ID NO: 5) and SEQ ID NO: 39 (at the position of 2289-2308 nt of SEQ ID NO: 5) for STK31, SEQ ID NO: 40 (at the position of 808-827 nt of SEQ ID NO: 1) and SEQ ID NO: 41 (at the position of 470-488 nt of SEQ ID NO: 1) for CDCA5, SEQ ID NO: 42 (at the position of 2182-2200 nt of SEQ ID NO: 3) and SEQ ID NO: 43 (at the position of 1968-1987 nt of SEQ ID NO: 3) for EPHA7, SEQ ID NO: 44 (at the position of 577-596 nt of SEQ ID NO: 7) and SEQ ID NO: 45 (at the position of 2041-2060 nt of SEQ ID NO: 7) for WDHD1.

16. A method for diagnosing lung cancers and/or esophageal cancers, wherein said method comprising the steps of
(a) detecting the expression level of the gene selected from the group consisting of CDCA5, EPHA7, STK31 and WDHD1 in a biological sample; and
(b) relating an increase of the expression level compared to a normal control level of the gene to the disease.

17. The method of claim 16, wherein the expression level is at least 10% greater than normal control level.

18. The method of claim 16, wherein the expression level is detected by any one of the method selected from the group consisting of:
(a) detecting the mRNA encoding the polypeptide selected from the group consisting of CDCA5, EPHA7, STK31 and WDHD1;
(b) detecting the polypeptide selected from the group consisting of CDCA5, EPHA7, STK31 and WDHD1, and
(c) detecting the biological activity of the polypeptide selected from the group consisting of CDCA5, EPHA7, STK31 and WDHD1.

19. The method of claim 16, wherein the lung cancer is non-small cell lung cancer or small cell lung cancer.

20. A method for assessing the prognosis of a patient with lung cancers and/or esophageal cancer, which method comprises the steps of:
(a) detecting the expression level of the gene selected from the group consisting of EPHA7, STK31 and WDHD1 in a biological sample; and
(b) comparing the detected expression level to a control level; and
(c) determining the prognosis of the patient based on the comparison of (b).

21. The method of claim 20, wherein the control level is a good prognosis control level and an increase of the expression level compared to the control level is determined as poor prognosis.

22. The method of claim 21, wherein the increase is at least 10% greater than said control level.

23. The method of claim 20, wherein said expression level is determined by any one method selected from the group consisting of:
(a) detecting the mRNA encoding the polypeptide selected from the group consisting of EPHA7, STK31 and WDHD1;
(b) detecting the polypeptide selected from the group consisting of EPHA7, STK31 and WDHD1; and
(c) detecting the biological activity of the polypeptide selected from the group consisting of CDCA5, EPHA7, STK31 and WDHD1.

24. The method of claim 23, wherein the lung cancer is non-small cell lung cancer or small cell lung cancer.

25. A method for detecting EPHA7 polypeptide in a subject, comprising the steps of:
(a) collecting a body fluid from a subject to be diagnosed;
(b) determining a level of EPHA7 polypeptide or fragment thereof in the body fluid by immunoassay.

26. The method of claim 25, wherein the body fluid is selected from the group consisting of whole blood, serum and plasma.

27. The method of claim 25, wherein the immunoassay is an ELISA.

28. The method of claim 25, further comprising the steps of:
(d) determining a level of pro-GRP in the blood sample;
(e) comparing the pro-GRP level determined in step (d) with that of a normal control, wherein either or both of high EPHA7 and high pro-GRP levels in the blood sample, compared to the normal control, indicate that the subject suffers from a lung cancer.

29. The method of claim 25, further comprising the steps of:
(d) determining a level of CEA in the blood sample;
(e) comparing the CEA level determined in step (d) with that of a normal control, wherein either or both of high EPHA7 and high CEA levels in the blood sample, compared to the normal control, indicate that the subject suffers from a lung cancer.

30. A kit for detecting lung cancers and/or esophageal cancer, wherein the kit comprises:
(a) an immunoassay reagent for determining a level of EPHA7 in a blood sample; and
(b) a positive control sample for EPHA7.

31. The kit of claim 30, the kit further comprises reagents for detecting CEA and/or pro-GRP.

32. A method of screening for an agent useful in diagnosing, treating or preventing cancer expressing at least one gene selected from the group consisting of CDCA5, EPHA7, STK31 or WDHD1 gene, said method comprising the steps of:
(a) contacting a test agent with a polypeptide encoded by the gene, or fragment thereof;
(b) detecting binding between the polypeptide and said test agent;
(c) selecting the test agent that binds to said polypeptides of step (a).

33. A method of screening for an agent useful in treating or preventing cancer expressing CDCA5, EPHA7, STK31 or WDHD1 gene, said method comprising the steps of:
(a) contacting a test agent with a cell expressing a polynucleotide encoding a polypeptide selected from the group consisting of CDCA5, EPHA7, STK31 and WDHD1 polypeptide, or functional equivalent thereof;
(b) detecting an expression level of said polynucleotide or polypeptide of step (a);
(c) comparing said level detected in the step (b) with those detected in the absence of the test agent; and
(d) selecting the test agent that reduces or inhibits said level comparing with those detected in the absence of the test agent in step (c).

34. A method of screening for an agent useful in treating or preventing cancer expressing CDCA5, EPHA7, STK31 or WDHD1 gene, said method comprising the steps of:
(a) contacting a test agent with a cell expressing a polynucleotide encoding a polypeptide selected from the group consisting of CDCA5, EPHA7, STK31 and WDHD1 polypeptide, or functional equivalent thereof;
(b) detecting a biological activity of said polynucleotide or polypeptide of step (a);
(c) comparing said biological activity detected in the step (b) with those detected in the absence of the test agent; and
(d) selecting the test agent that reduces said biological activity comparing with those detected in the absence of the test agent in step (c).

35. The method of claim 34, wherein the biological activity is any one of the activity selected from the group consisting of:
(a) a proliferation activity;
(b) an invasive activity; and
(c) a kinase activity.

36. The method of claim 35, wherein the kinase activity is detected with phosphorylation level of gene selected from the group consisting of EGFR, PLCgamma, CDC25, MET, Shc, ERK1/2(p44/42 MAPK), Akt, STAT3 and MEK1/2.

37. The method of claim 36, wherein the phosphorylation level is detected at residues selected from the group consisting of;
(a) Y845, Y1068, Y1086, Y1173, S1046 or S1047 of EGFR;
(b) Y783 of PLCgamma;
(c) S216 of CDC25;
(d) Y1230, Y1234, Y1235, Y1349 or Y1365 of MET;
(e) Y317, Y239, Y240 of Shc;
(f) T202 or Y204 of ERK1/2(p44/42 MAPK);
(g) S473 of Akt;
(h) Y705 of STAT3; and
(i) S217 or S221 of MEK1/2

38. A method of screening for an agent useful in treating or preventing cancer expressing EPHA7 gene, said method comprising the steps of:
(a) contacting a EPHA7 polypeptide or functional equivalent thereof with an substrate selected from group consist of EGFR, PLCgamma, CDC25, MET, Shc, ERK1/2(p44/42 MAPK), Akt, STAT3 and functional equivalent thereof, in the presence of a test compound under a condition that allows phosphorylation of the substrate;
(b) detecting a level of phosphorylation of substrate;
(c) comparing said level detected in the step (b) with those detected in the absence of the test agent; and
(d) selecting the test agent that reduces or inhibits said level comparing with those detected in the absence of the test agent in step (c).

39. The method of claim 38, wherein the level of phosphorylation of the substrate is detected at residues selected from the group consisting of Y845, Y1068, Y1086 and/or Y1173 of EGFR, Y783 of PLCgamma, S216 of CDC25, Y1230, Y1234, Y1235, Y1313, Y1349 and/or Y1365 of MET, Y317, Y239 and/or Y240 of Shc, T202 and/or Y204 of ERK1/2(p44/42 MAPK), S473 of Akt, and Y705 of STAT3

40. The method of claim 39, wherein the functional equivalent of EGFR is a polypeptide fragment comprising amino acid sequence of SEQ ID NO: 75.

41. The method of claim 38, wherein the functional equivalent of MET is a polypeptide fragment comprising amino acid sequence of SEQ ID NO: 76.

42. The method of claim 38, wherein the cancer is lung cancers and/or esophageal cancer.

43. A method of screening for an agent interrupts a binding between an EPHA7 polypeptide and an EGFR polypeptide or MET, said method comprising the steps of:
(a) contacting EPHA7 polypeptide or functional equivalent thereof with a EGFR or MET polypeptide or functional equivalent thereof in the presence of a test agent;
(b) detecting a binding between the polypeptides;
(c) comparing the binding level detected in the step (b) with those detected in the absence of the test agent; and
(d) selecting the test agent that reduces or inhibits the binding level comparing with those detected in the absence of the test agent in step (c).

44. The method of claim 38, wherein the functional equivalent of EPHA7 comprises the EGFR-binding domain.

45. The method of claim 38, wherein the functional equivalent of EGFR is a polypeptide fragment comprising amino acid sequence of SEQ ID NO: 75.

46. The method of claim 38, wherein the functional equivalent of MET is a polypeptide fragment comprising amino acid sequence of SEQ ID NO: 76.

47.-64. (canceled)

65. A method of screening for an agent useful in preventing or treating cancers expressing CDCA5, wherein said method comprising the steps of:
(a) contacting a test agent with a cell expressing a gene encoding CDCA5 polypeptide or functional equivalent thereof;
(b) culturing under a condition that allows phosphorylation of said polypeptide of step (a);
(c) detecting phosphorylation level of said polypeptide of step (a);
(d) comparing the phosphorylation level detected in the step (c) with those detected in the absence of the test agent; and
(e) selecting the test agent that inhibits or reduces the phosphorylation level comparing with those detected in the absence of the test agent in step (c).

66. The method of claim 65, wherein the agent inhibits or reduces CDC2-mediated phosphorylation activity or ERK-mediated phosphorylation activity of CDCA5.

67. The method of claim 65, wherein the phosphorylation level is phospho-serine or phospho-threonine level.

68. The method of claim 67, wherein phospho-serine of CDCA5 is Serine-21, Serine-75, Serine-79 or Serine-209 of SEQ ID NO: 2 (CDCA5).

69. The method of claim 68, wherein phospho-threonine of CDCA5 is Threonine-48, Threonine-111 or Threonine-115 of SEQ ID NO: 2 (CDCA5).

70. The method of claim 65, wherein the cancer is selected from the group consisting of lung cancers and esophageal cancer.

71. A method of screening for an agent useful in treating or preventing cancer expressing CDCA5, EPHA7, STK31 or WDHD1 gene, said method comprising the steps of:
(a) contacting a test agent with a cell into which a vector comprising the transcriptional regulatory region of CDCA5, EPHA7, STK31 and/or WDHD1 genes and a reporter gene that is expressed under the control of the transcriptional regulatory region has been introduced;
(b) measuring the expression of activity of said reporter gene; and;
(c) selecting a compound that reduces the expression of activity level of said reporter gene, as compared to a level in the absence of the test compound.

72. The method of claim 71, wherein the cancer is selected from the group consisting of lung cancers and esophageal cancer.


SEQUENCE LISTING (76 sequences)

SEQ ID NO: 1 - DNA, 2507 bp, Homo sapiens
  CDS (74)..(832)
gcagcgagtggccttcccggttggcgcgcgcccggggcggcggcgctggaggagctcgagacggagcctagttatgtctgggaggcgaacgcggtccggaggagccgctcagcgctccgggccaagggccccatctcctactaagcctctgcggaggtcccagcggaaatcaggctctgaactcccgagcatcctccctgaaatctggccgaagacacccagtgcggctgcagtcagaaagcccatcgtcttaaagaggatcgtggcccatgctgtagaggtcccagctgtccaatcacctcgcaggagccctaggatttcctttttcttggagaaagaaaacgagccccctggcagggagcttactaaggaggaccttttcaagacacacagcgtccctgccacccccaccagcactcctgtgccgaaccctgaggccgagtccagctccaaggaaggagagctggacgccagagacttggaaatgtctaagaaagtcaggcgttcctacagccggctggagaccctgggctctgcctctacctccaccccaggccgccggtcctgctttggcttcgaggggctgctgggggcagaagacttgtccggagtctcgccagtggtgtgctccaaactcaccgaggtccccagggtttgtgcaaagccctgggccccagacatgactctccctggaatctccccaccacccgagaaacagaaacgtaagaagaagaaaatgccagagatcttgaaaacggagctggatgagtgggctgcggccatgaatgccgagtttgaagctgctgagcagtttgatctcctggttgaatgagatgcagtggggggtgcacctggccagactctccctcctgtcctgtacatagccacctccctgtggagaggacacttagggtcccctcccctggtcttgttacctgtgtgtgtgctggtgctgcgcatgaggactgtctgcctttgagggcttgggcagcagcggcagccatcttggttttaggaaatggggccgcctggcccagccactcactggtgtcctgtctcttgtcgtcctgtccttcctatctccccaaagtaccatagccagtttccagatgggccacagactggggaggagaatcagtggcccagccagaagttaaagggctgagggttgaggtgagaggcacctctgctcttgttgggaggggtggctgcttggaaataggcccaggggctctgccagcctcggcctctccctcctgagttgccttctgttggtggctttcttcttgaacccacctgtgtaaagaggttttcagttccgtgggtttcccctttgattctgtaaatagtcccagagagaattcgtgggctgagggcaattctgtcttggaggaagaagctggacattcagcctgtggagtctgagttttgaaggatgtagggagccttagttgggtctcagaccataagtgtgtactacacagaagctgtgttttctagttctggtctgctgttgagatgtttggtaaatgccaggttgatagggcgctggctgcttggagcaaagggtgcatttcagggtgtggccaccaggtgctgtgagtttctgtggctcatggcctctgggctggtcccttgcacagggcccacgctggagtcttaccactctgctgcaggggtggaaggtggcccctcttgtcacccatacccatttcttacaaaataagttacaccgagtctacttggccctagaagagaaagttgaagagtcccagacctactagcattttgcaactatgcttgtaaagtcctcggaaagtttcctcgcgtaccagacagcggcgggggctgatagcaattttagtttttggcctccctatcctctcacatgagaacactgcctggatgcatctcatgatctctggagaatttccccatctttctcttctttccatcgtgtggattcaatagtttggatttgaaggctgccctgcccccgactctcctgccgcacccctggccattgtaccttttgatgtttagaagttcgtggaagtagacgctgaggtgtgcagaggagctggtggataacagagaatgccagggaagatgagtgctgggtcagggtacttggatgaaacggtgcaggccaggcgggccctaataaaaccctctgccaggtctgggagtcccaggccatctgctcaacgctctgtggtttgtcagacctgcaagcaagccccctgctggggaagcctaggtgtccttgagctgaaccgcactgaagaactcttgtcctcactggctgatgcagcagaactcttgggaaatgtcttagtcctgcagaatcaggagtcaccagatgatgcagagttgagatcatcattgcaaagttctctgttcctgaggaactaaatttaaggaaaaaatgggattttgttttagagttggaaaaaaagcctgattaaagagtttctgcctgttaaaaaaaaaaaaaaaaaaaaaaa

SEQ ID NO: 2 - protein, 252 aa, Homo sapiens
MSGRRTRSGGAAQRSGPRAPSPTKPLRRSQRKSGSELPSILPEIWPKTPSAAAVRKPIVLKRIVAHAVEVPAVQSPRRSPRISFFLEKENEPPGRELTKEDLFKTHSVPATPTSTPVPNPEAESSSKEGELDARDLEMSKKVRRSYSRLETLGSASTSTPGRRSCFGFEGLLGAEDLSGVSPVVCSKLTEVPRVCAKPWAPDMTLPGISPPPEKQKRKKKKMPEILKTELDEWAAAMNAEFEAAEQFDLLVE

SEQ ID NO: 3 - DNA, 5229 bp, Homo sapiens
  CDS (214)..(3210)
gcagtcggagacttgcaggcagcaaacacggtgcgagcgaacaggagtgggggggaaattaaaaaaagctaaacgtggagcagccgatcggggaccgagaaggggaatcgatgcaaggagcacaataaaacaaaagctacttcggaacaaacagcatttaaaaatccacgactcaagataactgaaacctaaaataaaacctgctcatgcaccatggtttttcaaactcggtacccttcatggattattttatgctacatctggctgctccgctttgcacacacaggggaggcgcaggctgcgaaggaagtactactgctggattctaaagcacaacaaacagagttggagtggatttcctctccacccaatgggtgggaagaaattagtggtttggatgagaactataccccgatacgaacataccaggtgtgccaagtcatggagcccaaccaaaacaactggctgcggactaactggatttccaaaggcaatgcacaaaggatttttgtagaattgaaattcaccctgagggattgtaacagtcttcctggagtactgggaacttgcaaggaaacatttaatttgtactattatgaaacagactatgacactggcaggaatataagagaaaacctctatgtaaaaatagacaccattgctgcagatgaaagttttacccaaggtgaccttggtgaaagaaagatgaagcttaacactgaggtgagagagattggacctttgtccaaaaagggattctatcttgcctttcaggatgtaggggcttgcatagctttggtttctgtcaaagtgtactacaagaagtgctggtccattattgagaacttagctatctttccagatacagtgactggttcagaattttcctctttagtcgaggttcgagggacatgtgtcagcagtgcagaggaagaagcggaaaacgcccccaggatgcactgcagtgcagaaggagaatggttagtgcccattggaaaatgtatctgcaaagcaggctaccagcaaaaaggagacacttgtgaaccctgtggccgtgggttctacaagtcttcctctcaagatcttcagtgctctcgttgtccaactcacagtttttctgataaagaaggctcctccagatgtgaatgtgaagatgggtattacagggctccatctgacccaccatacgttgcatgcacaaggcctccatctgcaccacagaacctcattttcaacatcaaccaaaccacagtaagtttggaatggagtcctcctgcagacaatgggggaagaaacgatgtgacctacagaatattgtgtaagcggtgcagttgggagcagggcgaatgtgttccctgtgggagtaacattggatacatgccccagcagactggattagaggataactatgtcactgtcatggacctgctagcccacgctaattatacttttgaagttgaagctgtaaatggagtttctgacttaagccgatcccagaggctctttgctgctgtcagtatcaccactggtcaagcagctccctcgcaagtgagcggagtaatgaaggagagagtactgcagcggagtgtcgagctttcctggcaggaaccagagcatcccaatggagtcatcacagaatatgaaatcaagtattacgagaaagatcaaagggaacggacctactcaacagtaaaaaccaagtctacttcagcctccattaataatctgaaaccaggaacagtgtatgttttccagattcgggcttttactgctgctggttatggaaattacagtcccagacttgatgttgctacactagaggaagctacaggtaaaatgtttgaagctacagctgtctccagtgaacagaatcctgttattatcattgctgtggttgctgtagctgggaccatcattttggtgttcatggtctttggcttcatcattgggagaaggcactgtggttatagcaaagctgaccaagaaggcgatgaagagctttactttcattttaaatttccaggcaccaaaacctacattgaccctgaaacctatgaggacccaaatagagctgtccatcaattcgccaaggagctagatgcctcctgtattaaaattgagcgtgtgattggtgcaggagaattcggtgaagtctgcagtggccgtttgaaacttccagggaaaagagatgttgcagtagccataaaaaccctgaaagttggttacacagaaaaacaaaggagagactttttgtgtgaagcaagcatcatggggcagtttgaccacccaaatgttgtccatttggaaggggttgttacaagagggaaaccagtcatgatagtaatagagttcatggaaaatggagccctagatgcatttctcaggaaacatgatgggcaatttacagtcattcagttagtaggaatgctgagaggaattgctgctggaatgagatatttggctgatatgggatatgttcacagggaccttgcagctcgcaatattcttgtcaacagcaatctcgtttgtaaagtgtcagattttggcctgtcccgagttatagaggatgatccagaagctgtctatacaactactggtggaaaaattccagtaaggtggacagcacccgaagccatccagtaccggaaattcacatcagccagtgatgtatggagctatggaatagtcatgtgggaagttatgtcttatggagaaagaccttattgggacatgtcaaatcaagatgttataaaagcaatagaagaaggttatcgtttaccagcacccatggactgcccagctggccttcaccagctaatgttggattgttggcaaaaggagcgtgctgaaaggccaaaatttgaacagatagttggaattctagacaaaatgattcgaaacccaaatagtctgaaaactcccctgggaacttgtagtaggccaataagccctcttctggatcaaaacactcctgatttcactaccttttgttcagttggagaatggctacaagctattaagatggaaagatataaagataatttcacggcagctggctacaattcccttgaatcagtagccaggatgactattgaggatgtgatgagtttagggatcacactggttggtcatcaaaagaaaatcatgagcagcattcagactatgagagcacaaatgctacatttacatggaactggcattcaagtgtgatatgcatttctcccttttaagggagattacagactgcaagagaacagtactggccttcagtatatgcatagaatgctgctagaagacaagtgatgtcctgggtccttccaacagtgaagagaagatttaagaagcacctatagacttgaactcctaagtgccaccagaatatataaaaagggaatttaggatccaccatcggtggccaggaaaatagcagtgacaataaacaaagtactacctgaaaaacatccaaacaccttgagctctctaacctcctttttgtcttatagactttttaaaatgtacataaagaatttaagaaagaatatatttgtcaaataaaatcatgatcttattgttaaaattaatgaaatattttccttaaatatgtgatttcagactattcctttttaaaatcatttgtgtttattcttcataaggactttgttttagaaagctgtttatagctttggacctttttagtgttaaatctgtaacattactacactgggtacctttgaaagaatctcaaatttcaaaagaaatagcatgattgaagatacatctctgttagaacattggtatcctttttgtgccattttattctgtttaatcagtgctgttttgatattgtttgctaattggcaggtagtcaagaaaatgcaagttgccaagagctctgatattttttaaaaagaatttttttgtaaagatcagacaacacactatcttttcaatgaaaaaagcaataatgatccatacatactataaggcacttttaacagattgtttatagagtgattttactagaaagaatttaataaactcgaagtttaggtttatgagtatataaacaaatgaggcacttcatctgaagaatgttggtgaaggcaagtctctgaaagcagaactatccagtgttatctaaaaattaatctgagcacatcaagattttttcattctcgtgacattaggaaatttaggataaatagttgacatatattttatatcctcttctgttgaatgcagtccaaacatgaaaggaaataattgttttatattataactctgaagcatgataaaggggcagttcacaattttcaccatttaaacacaaatttgctgcacagaatatcaccattgcagttcaaaacaaaacaaaacaaaaagtcttttgtttgtgaacactgatgcaagaaacttgttaaatgaaaggactctttaccctagaaggaagaggtgaaggatctggcttgtttttaaagctttatttattaaaccatattatttgattactgtgttagaatttcataagcaataattaaatgtgtctttatagatattgcaggaatgtatacatattgtgattaatgctttcaaaacttatgaaaatcatgaactaccccagaattgaactgttgtacttccaaagagaattgggctgtttataatgattttaatagagaaagatcccagggatcggtcataattggtcttgtttgataatgtgggcatccacaaacaaacaaacaaataacagaaacaaaatctgtaaatgttcctttgtaaaacttgtaaattttatttatactgtcttgttttgtacacacatttctctgtagtgggctctgaatacattgaaaatgcactatatttttctattttacttgcagagcatcacaaaagaacaggtattttcagtgctacataatgtgttttcccacatttaggaccaaagacggctatagaaaaactcaaatggattgcttcccaaacccctccccaccctttttttttggttttaaatcactgtacagtgttatttgatattttaatttattttttgattgactagaaaaatcattttaatttcactaaaatgttttttgtccctaaggaaaagtaatctgtaaaaataattttaattagcataatacagtcacctagacacttccatttgtaatctttgtaatagactgtaaatatatttttggaactataaaaaaaaaaaaaaaa

SEQ ID NO: 4 - protein, 998 aa, Homo sapiens
MVFQTRYPSWIILCYIWLLRFAHTGEAQAAKEVLLLDSKAQQTELEWISSPPNGWEEISGLDENYTPIRTYQVCQVMEPNQNNWLRTNWISKGNAQRIFVELKFTLRDCNSLPGVLGTCKETFNLYYYETDYDTGRNIRENLYVKIDTIAADESFTQGDLGERKMKLNTEVREIGPLSKKGFYLAFQDVGACIALVSVKVYYKKCWSIIENLAIFPDTVTGSEFSSLVEVRGTCVSSAEEEAENAPRMHCSAEGEWLVPIGKCICKAGYQQKGDTCEPCGRGFYKSSSQDLQCSRCPTHSFSDKEGSSRCECEDGYYRAPSDPPYVACTRPPSAPQNLIFNINQTTVSLEWSPPADNGGRNDVTYRILCKRCSWEQGECVPCGSNIGYMPQQTGLEDNYVTVMDLLAHANYTFEVEAVNGVSDLSRSQRLFAAVSITTGQAAPSQVSGVMKERVLQRSVELSWQEPEHPNGVITEYEIKYYEKDQRERTYSTVKTKSTSASINNLKPGTVYVFQIRAFTAAGYGNYSPRLDVATLEEATGKMFEATAVSSEQNPVIIIAVVAVAGTIILVFMVFGFIIGRRHCGYSKADQEGDEELYFHFKFPGTKTYIDPETYEDPNRAVHQFAKELDASCIKIERVIGAGEFGEVCSGRLKLPGKRDVAVAIKTLKVGYTEKQRRDFLCEASIMGQFDHPNVVHLEGVVTRGKPVMIVIEFMENGALDAFLRKHDGQFTVIQLVGMLRGIAAGMRYLADMGYVHRDLAARNILVNSNLVCKVSDFGLSRVIEDDPEAVYTTTGGKIPVRWTAPEAIQYRKFTSASDVWSYGIVMWEVMSYGERPYWDMSNQDVIKAIEEGYRLPAPMDCPAGLHQLMLDCWQKERAERPKFEQIVGILDKMIRNPNSLKTPLGTCSRPISPLLDQNTPDFTTFCSVGEWLQAIKMERYKDNFTAAGYNSLESVARMTIEDVMSLGITLVGHQKKIMSSIQTMRAQMLHLHGTGIQV

SEQ ID NO: 5 - DNA, 3244 bp, Homo sapiens
  CDS (16)..(3075)
cggcgaaagtccagtatgtgggtccagggtcactcttctagagcttccgcaacggaaagtgtgagtttttcaggaattgttcagatggatgaagatacacattacgataaagtggaagatgtggttggaagtcacatagaagatgcagtaacattttgggcccagagtatcaatagaaataaggatatcatgaagattggttgctcactgtctgaagtttgcccccaggccagttcagttttggggaatcttgacccaaacaagatttatggtggattattttctgaagatcagtgttggtacagatgcaaagtactgaaaatcatcagcgttgaaaagtgtctggtgaggtacattgactatggaaatactgaaattctaaatcgatctgatatagttgaaattcctttggagctgcagttttctagtgttgccaaaaagtataaactttggggactacacattccttctgatcaagaagttacccagtttgatcagggcacaacctttttggggagcttgatttttgaaaaggaaataaaaatgagaattaaagcaacctctgaagatggaacagttattgctcaggctgagtatggcagtgtggatataggggaagaggtgcttaagaaaggatttgcagagaaatgcagacttgcttccagaactgacatctgtgaggaaaaaaaattggatcctggtcaacttgttctcaggaacctcaaaagccccattcctttgtgggggcatagatcaaaccagtcaaccttcagcaggcccaaggggcacttaagtgagaaaatgactcttgacttgaaggatgaaaatgatgcaggcaatcttataacatttccaaaggaaagtttggctgttggtgactttaatttagggtctaacgtcagcctggaaaaaattaagcaggaccagaaactgattgaagaaaatgaaaaacttaaaacagagaaggacgctcttcttgaaagttataaggcgttagaattgaaagtagagcagattgcccaggagctgcagcaagagaaggcagctgctgtggatttgactaaccacttagaatacactctgaagacctatatagataccagaatgaaaaatctggcagctaagatggaaatactgaaagaaatgaggcatgtcgacatcagtgtccgtttcggaaaagacctttcagatgctatacaagtgttggatgaagggtgctttactactccagcttctttgaatggattagagataatatgggcagaatacagtctggctcaggagaatattaaaacttgtgaatatgtgagtgaagggaatattttgattgcccaaagaaatgaaatgcagcagaagctgtacatgtcagtagaagattttattctggaagttgatgagtcatctcttaataaacgcttaaaaacattgcaggatttgtcagtctctttagaagcagtgtatggacaagccaaagaaggagcaaattctgatgaaatacttaaaaaattttatgactggaagtgtgataaaagagaggagttcaccagtgttagaagtgaaacagacgcttctctgcaccgtcttgtagcatggttccaaagaaccttaaaggtttttgacctatctgtggaaggatcactgatttcagaagacgcaatggataatattgatgaaatcctagagaagactgagtcaagtgtctgcaaagagctggagatagctctggttgatcaaggtgatgcagacaaggagataatttcaaatacatatagtcaagtactgcaaaagattcattcagaggaaaggctcattgccacagtacaagctaagtacaaggacagtattgagtttaaaaagcagcttattgaatatttaaataagagtcccagtgtggatcacttgctatccattaagaagacattgaaaagcttaaaagctctactcagatggaaattggttgaaaagagtaatttggaagagtcagatgatcctgatggctctcaaattgagaaaataaaagaagaaataactcagctgcgcaataatgtctttcaggaaatttatcatgagagagaggaatatgagatgctaactagtttggcacagaaatggttccctgagctgcctctgcttcatcctgaaataggattactcaaatacatgaactctggtggtctccttacaatgagcttggaacgagatcttcttgatgctgagcccatgaaggaacttagcagcaagcgtcctttggtacgttctgaggttaatgggcagataattctgttaaagggctattctgtggatgttgacacagaagccaaggtgattgagagagcagccacctaccatagagcttggagagaagctgaaggagactcagggttactgccattgatattcctgtttttatgtaagtctgatcctatggcttatctgatggtcccatactaccctagggcaaacctgaatgctgttcaagccaacatgcctttaaattcagaagaaactttaaaggtcatgaaaggtgttgcccagggtctgcatacattgcataaggctgacataattcatggatcacttcatcagaacaatgtatttgctttaaaccgtgaacaaggaattgttggagattttgacttcaccaaatctgtgagtcagcgagcctcggtgaacatgatggttggtgacttgagtttgatgtcacctgagttgaaaatgggaaaacctgcttctccaggttcagacttatatgcttatggctgcctcttattatggctttctgttcaaaatcaggagtttgagataaataaagatggaatccccaaagtggatcagtttcatctggatgataaagtcaaatccctcctctgtagcttgatatgttatagaagttcaatgactgctgaacaagttttaaatgctgaatgtttcttgatgccaaaggagcaatcagttccaaacccagaaaaagatactgaatacaccctatataaaaaggaagaagaaataaagacggagaacttggataaatgtatggagaagacaagaaatggtgaagccaactttgattgttaaattattattgttgttgttgcagaggttctttttaaaaactttgtttggtttggttaatacacagaaatatctagaaatgttctgggactagttgagttgtatctttagtattcaggttgtgaaaaataaagatgtttggctatgcaaaaaaaaaaaaaaaaaaaaaagg

SEQ ID NO: 6 - protein, 1019 aa, Homo sapiens
MWVQGHSSRASATESVSFSGIVQMDEDTHYDKVEDVVGSHIEDAVTFWAQSINRNKDIMKIGCSLSEVCPQASSVLGNLDPNKIYGGLFSEDQCWYRCKVLKIISVEKCLVRYIDYGNTEILNRSDIVEIPLELQFSSVAKKYKLWGLHIPSDQEVTQFDQGTTFLGSLIFEKEIKMRIKATSEDGTVIAQAEYGSVDIGEEVLKKGFAEKCRLASRTDICEEKKLDPGQLVLRNLKSPIPLWGHRSNQSTFSRPKGHLSEKMTLDLKDENDAGNLITFPKESLAVGDFNLGSNVSLEKIKQDQKLIEENEKLKTEKDALLESYKALELKVEQIAQELQQEKAAAVDLTNHLEYTLKTYIDTRMKNLAAKMEILKEMRHVDISVRFGKDLSDAIQVLDEGCFTTPASLNGLEIIWAEYSLAQENIKTCEYVSEGNILIAQRNEMQQKLYMSVEDFILEVDESSLNKRLKTLQDLSVSLEAVYGQAKEGANSDEILKKFYDWKCDKREEFTSVRSETDASLHRLVAWFQRTLKVFDLSVEGSLISEDAMDNIDEILEKTESSVCKELEIALVDQGDADKEIISNTYSQVLQKIHSEERLIATVQAKYKDSIEFKKQLIEYLNKSPSVDHLLSIKKTLKSLKALLRWKLVEKSNLEESDDPDGSQIEKIKEEITQLRNNVFQEIYHEREEYEMLTSLAQKWFPELPLLHPEIGLLKYMNSGGLLTMSLERDLLDAEPMKELSSKRPLVRSEVNGQIILLKGYSVDVDTEAKVIERAATYHRAWREAEGDSGLLPLIFLFLCKSDPMAYLMVPYYPRANLNAVQANMPLNSEETLKVMKGVAQGLHTLHKADIIHGSLHQNNVFALNREQGIVGDFDFTKSVSQRASVNMMVGDLSLMSPELKMGKPASPGSDLYAYGCLLLWLSVQNQEFEINKDGIPKVDQFHLDDKVKSLLCSLICYRSSMTAEQVLNAECFLMPKEQSVPNPEKDTEYTLYKKEEEIKTENLDKCMEKTRNGEANFDC

SEQ ID NO: 7 - DNA, 4734 bp, Homo sapiens
  CDS (79)..(3468)
gcgagccgaagcgcgggaagcagctcttgtggatcctcagtggcggaggctcggtcacccggataggtaaaggaaaacatgcctgccacacggaagccaatgagatatgggcatacagagggacacacggaggtctgttttgatgattctgggagttttattgtgacttgtggaagtgatggtgatgtgaggatttgggaagacttggatgatgatgatcctaagttcattaatgttggagaaaaggcatattcatgtgctttgaagagtggaaaactggtcactgcagtttctaataatactattcaagtccacacatttcctgaaggagttccagatggtatattgactcgcttcactacaaatgcaaaccatgtggtctttaatggggatggtactaaaattgctgctggatctagtgattttctagtcaaaattgtggatgtgatggatagcagccaacagaaaacatttcgaggacatgatgcccctgttttaagtctttcctttgatcctaaggacatctttctggcatcagctagttgtgatggatctgtcagagtgtggcaaatttcagatcagacatgtgctattagttggccactgctacaaaaatgcaacgatgtgataaatgcaaaatcaatctgcagacttgcttggcagccaaaaagtgggaagttactggcaattcctgtggaaaaatctgttaagctatatagaagagaatcttggagtcatcaatttgatctttcagataatttcatctctcagaccctcaatatagtaacctggtctccctgtgggcaatatttagctgcaggtagtattaatggtctaatcatagtttggaatgtggaaaccaaagactgcatggaaagggtgaaacatgagaaaggttatgcaatttgtggtctggcatggcatcctacttgtggtcgaatatcgtatactgatgcggaaggaaatctagggcttctagagaatgtttgtgaccccagtggaaagacatcaagcagtaaggtatctagcagagtggaaaaggattataatgatctttttgatggagatgatatgagtaatgctggtgattttctaaatgacaatgcagttgagatcccttctttttcaaaagggattataaatgatgatgaggatgatgaagacctcatgatggcttcaggtcgtcctagacagcgaagtcacatcctagaagatgatgaaaactcagttgatatttcaatgctaaaaactggttctagtcttctcaaagaggaggaggaagatggtcaagaaggcagcattcacaatctaccacttgtaacatcccaaaggccattttatgatggacccatgccaactccccggcaaaagccatttcagtcaggttctacaccgttgcatctcactcacagattcatggtgtggaactctattggaattattcgctgctataatgatgagcaagacaatgccatagatgtggagttccatgatacctccatacaccatgcaacacacttatcaaacactttgaattatacaatagcagatctttcccacgaagctattttgttggcatgtgaaagcactgatgaactagcaagcaagcttcactgcctgcactttagttcttgggattcaagcaaagagtggataatagacttgcctcagaatgaggatattgaagccatatgtctcggtcaaggatgggctgctgccgctactagtgccctgcttcttcgattgtttactattggaggggttcaaaaagaggtattcagccttgctggacctgtggtgtcaatggcaggacatggagaacagcttttcattgtttatcacagaggtacaggatttgatggggatcagtgccttggagttcaactgctagagctggggaaaaagaaaaaacaaattttgcatggtgaccctcttcctcttacaaggaaatcctaccttgcatggattgggttttcagctgaaggtaccccttgttacgtggattcagaaggaattgttcgaatgcttaacagaggacttggtaatacgtggactcctatatgtaatacaagagagcactgcaaaggaaaatctgatcactactgggtggttggtatccatgaaaatccccagcaactaaggtgcattccttgtaaaggttctcggtttcccccaacccttccacgccctgctgttgctatattatcctttaagcttccttactgtcagattgcaacagagaaaggacaaatggaggagcaattttggcgttcagttatatttcacaaccaccttgattatttagctaaaaatggttatgaatatgaagagagcactaaaaatcaagcaacaaaagagcaacaggaacttttaatgaaaatgcttgcgctttcttgtaaactggagcgagaattccgttgtgtggaacttgctgatctaatgactcaaaatgctgtgaatttagccattaaatatgcttctcgctctcggaaattaatactggctcaaaaactaagtgaactggctgtagagaaggcagccgaattgacagcaacccaggtggaagaggaagaagaagaagaagatttcagaaaaaagctgaatgctggttacagcaatactgctacagagtggagccaaccaaggttcagaaatcaagttgaagaagatgctgaggacagtggagaagctgatgatgaagaaaaaccagaaatacataagcctggacagaactcgttttccaaaagtacaaattcctctgatgtttcagctaagtcaggtgcagttacctttagcagccaaggacgagtaaatccctttaaggtatcagccagttccaaagaaccagccatgtcaatgaattcagcacgttcaactaatattttagacaatatgggcaaatcatccaagaaatccactgcacttagtcgaactacaaataatgaaaagtctcccattataaagcctctgattccaaagccgaagcctaagcaggcatctgcagcatcctatttccagaaaagaaattctcaaactaataaaactgaggaagtgaaagaagaaaatcttaaaaatgtattatctgaaaccccagctatatgtcctcctcaaaacactgaaaaccaaaggccaaagaccgggttccagatgtggttagaagaaaatagaagtaatattttgtctgacaatcctgacttttcagatgaagcagacataataaaagaaggaatgattcgatttagagtattgtcaactgaagaaagaaaggtgtgggctaacaaagccaaaggagaaacggcaagtgaaggaactgaagcaaagaagcgaaaacgtgtggttgatgaaagtgatgaaacagaaaaccaggaagaaaaagcaaaagagaacctgaatttgtctaaaaagcagaaacctttagatttttctacaaatcagaaactatcagcttttgcatttaagcaggagtaaaggaagaaagtgaccctagggaagtaatggatttttttttactcatctttgaatatagactcgagtctttgggaaactcattatatatatattttttaaagagtttgaagcaactgtttgtctttataagataatgtagtaattatattggtgtaggtaacaggacatatgtaaaaactatcatctttgcagattactctgcctccaaatgcagggcctttcagagatgcattgtgattgtaattactgagttgaagctccaaccaatttgaatttgtttcttaaccttgaaaaatcattaaagccaaggtattaaaacctttgtgcattaataccttctaggggtttggttcatttggtttttgtcatgtgcaaggaaggacaatagtcctctttccaagtgtgttagcatagacttctctatatgtttctactagacctaggggatgacgtcttttaataatactggccctaaacatgtaaataatcttgtaggtgagactttttcttttgtgtttcggaaatttcctatgtggctttcagttgtctgtttgtatagcctggatttttttgaggtaaatgaaactttctcatttgtatatttggcttgatatggtcttaatattatctttccacgaaatggatatatttctagaaaatatatatttactaccataatttctaccaccacccccattttgctctgcattatacacagtagagaagaactgaagacactgctgtgacagtattgcagtccaaggcatcatgtgctcttggtgggatactctgattatcagcatcaacagtactttactgagcaagactttgaaggcctgagaagagagcaaagttatggaaagtatttaactcttattttatattgaacaaacaaggtttaatcatgtcatacatttttggttttctaagcagagactaatacaaatgcagccacataaaggcagtgtactgggggtgggagggaaggaaacaatcacataaaatcagctgactcaaaattgaggatagttaataggttgaaagggaaaaagtatgttgaaaatttagacataaatgaaccaagaatattccttatctggtgatgattaaagttaggaaaacatacattttttattttttaattagtagctgctatcagagacattatagcacagtggttatgagcacagattccaaagccagattacctaggttcggagacccgcttctctacctactactaatagttgggtcatgttgggccgg

SEQ ID NO: 8 - protein, 1129 aa, Homo sapiens
MPATRKPMRYGHTEGHTEVCFDDSGSFIVTCGSDGDVRIWEDLDDDDPKFINVGEKAYSCALKSGKLVTAVSNNTIQVHTFPEGVPDGILTRFTTNANHVVFNGDGTKIAAGSSDFLVKIVDVMDSSQQKTFRGHDAPVLSLSFDPKDIFLASASCDGSVRVWQISDQTCAISWPLLQKCNDVINAKSICRLAWQPKSGKLLAIPVEKSVKLYRRESWSHQFDLSDNFISQTLNIVTWSPCGQYLAAGSINGLIIVWNVETKDCMERVKHEKGYAICGLAWHPTCGRISYTDAEGNLGLLENVCDPSGKTSSSKVSSRVEKDYNDLFDGDDMSNAGDFLNDNAVEIPSFSKGIINDDEDDEDLMMASGRPRQRSHILEDDENSVDISMLKTGSSLLKEEEEDGQEGSIHNLPLVTSQRPFYDGPMPTPRQKPFQSGSTPLHLTHRFMVWNSIGIIRCYNDEQDNAIDVEFHDTSIHHATHLSNTLNYTIADLSHEAILLACESTDELASKLHCLHFSSWDSSKEWIIDLPQNEDIEAICLGQGWAAAATSALLLRLFTIGGVQKEVFSLAGPVVSMAGHGEQLFIVYHRGTGFDGDQCLGVQLLELGKKKKQILHGDPLPLTRKSYLAWIGFSAEGTPCYVDSEGIVRMLNRGLGNTWTPICNTREHCKGKSDHYWVVGIHENPQQLRCIPCKGSRFPPTLPRPAVAILSFKLPYCQIATEKGQMEEQFWRSVIFHNHLDYLAKNGYEYEESTKNQATKEQQELLMKMLALSCKLEREFRCVELADLMTQNAVNLAIKYASRSRKLILAQKLSELAVEKAAELTATQVEEEEEEEDFRKKLNAGYSNTATEWSQPRFRNQVEEDAEDSGEADDEEKPEIHKPGQNSFSKSTNSSDVSAKSGAVTFSSQGRVNPFKVSASSKEPAMSMNSARSTNILDNMGKSSKKSTALSRTTNNEKSPIIKPLIPKPKPKQASAASYFQKRNSQTNKTEEVKEENLKNVLSETPAICPPQNTENQRPKTGFQMWLEENRSNILSDNPDFSDEADIIKEGMIRFRVLSTEERKVWANKAKGETASEGTEAKKRKRVVDESDETENQEEKAKENLNLSKKQKPLDFSTNQKLSAFAFKQE

SEQ ID NO: 9 - DNA, 21 bp, Artificial
  An artificially synthesized primer sequence for RT-PCR
gaggtgatagcattgctttcg

SEQ ID NO: 10 - DNA, 21 bp, Artificial
  An artificially synthesized primer sequence for RT-PCR
caagtcagtgtacaggtaagc

SEQ ID NO: 11 - DNA, 20 bp, Artificial
  primer ?RT-PCR?
cgccagagacttggaaatgt

SEQ ID NO: 12 - DNA, 20 bp, Artificial
  An artificially synthesized primer sequence for RT-PCR
gtttctgtttctcgggtggt

SEQ ID NO: 13 - DNA, 23 bp, Artificial
  An artificially synthesized primer sequence for RT-PCR
gcaggtagtcaagaaaatgcaag

SEQ ID NO: 14 - DNA, 23 bp, Artificial
  primer ?RT-PCR?
cagatccttcacctcttccttct

SEQ ID NO: 15 - DNA, 20 bp, Artificial
  primer ?RT-PCR?
aagccaaagaaggagcaaat

SEQ ID NO: 16 - DNA, 20 bp, Artificial
  primer ?RT-PCR?
caatgagcctttcctctgaa

SEQ ID NO: 17 - DNA, 24 bp, Artificial
  An artificially synthesized primer sequence for RT-PCR
agtgaaggaactgaagcaaagaag

SEQ ID NO: 18 - DNA, 23 bp, Artificial
  An artificially synthesized primer sequence for RT-PCR
atccattacttccctagggtcac

SEQ ID NO: 19 - DNA, 23 bp, Artificial
  An artificially synthesized primer sequence for RT-PCR
gcttgtaaagtcctcggaaagtt

SEQ ID NO: 20 - DNA, 23 bp, Artificial
  An artificially synthesized primer sequence for RT-PCR
atctcaactctgcatcatctggt

SEQ ID NO: 21 - DNA, 20 bp, Artificial
  An artificially synthesized primer sequence for RT-PCR
gaaaatgggaaaacctgctt

SEQ ID NO: 22 - DNA, 20 bp, Artificial
  An artificially synthesized primer sequence for RT-PCR
ctctgattccaaagccgaag

SEQ ID NO: 23 - RNA, 19 nt, Artificial
  An artificially synthesized oligonucleotide for siRNA
cguacgcggaauacuucga

SEQ ID NO: 24 - RNA, 19 nt, Artificial
  An artificially synthesized oligonucleotide for siRNA
ugguuuacaugucgacuaa

SEQ ID NO: 25 - RNA, 19 nt, Artificial
  An artificially synthesized oligonucleotide for siRNA
ugguuuacauguuuucuga

SEQ ID NO: 26 - RNA, 19 nt, Artificial
  An artificially synthesized oligonucleotide for siRNA
ugguuuacauguuuuccua

SEQ ID NO: 27 - RNA, 19 nt, Artificial
  An artificially synthesized oligonucleotide for siRNA
ugguuuacauguuguguga

SEQ ID NO: 28 - RNA, 19 nt, Artificial
  An artificially synthesized oligonucleotide for siRNA
gcgcgcuuuguaggauucg

SEQ ID NO: 29 - RNA, 19 nt, Artificial
  An artificially synthesized oligonucleotide for siRNA
gaagcagcacgacuucuuc

SEQ ID NO: 30 - RNA, 20 nt, Artificial
  An artificially synthesized oligonucleotide for siRNA
gcaguuugaucuccugguuu

SEQ ID NO: 31 - RNA, 21 nt, Artificial
  An artificially synthesized oligonucleotide for siRNA
gccagagacuuggaaauguuu

SEQ ID NO: 32 - RNA, 18 nt, Artificial
  An artificially synthesized oligonucleotide for siRNA
aaaagagauguugcagua

SEQ ID NO: 33 - RNA, 19 nt, Artificial
  An artificially synthesized oligonucleotide for siRNA
uagcaaagcugaccaagaa

SEQ ID NO: 34 - RNA, 21 nt, Artificial
  An artificially synthesized oligonucleotide for siRNA
gaucagacaugugcuauuauu

SEQ ID NO: 35 - RNA, 21 nt, Artificial
  An artificially synthesized oligonucleotide for siRNA
gguaauacguggacuccuauu

SEQ ID NO: 36 - DNA, 19 bp, Artificial
  A target sequence for siRNA
gaagcagcacgacttcttc

SEQ ID NO: 37 - DNA, 19 bp, Artificial
  A target sequence for siRNA
cgtacgcggaatacttcga

SEQ ID NO: 38 - DNA, 19 bp, Artificial
  A target sequence for siRNA
ggagatagctctggttgat

SEQ ID NO: 39 - DNA, 19 bp, Artificial
  A target sequence for siRNA
gggctattctgtggatgtt

SEQ ID NO: 40 - DNA, 19 bp, Artificial
  A target sequence for siRNA
gcagtttgatctcctggtt

SEQ ID NO: 41 - DNA, 19 bp, Artificial
  A target sequence for siRNA
gccagagacttggaaatgt

SEQ ID NO: 42 - DNA, 18 bp, Artificial
  A target sequence for siRNA
aaaagagatgttgcagta

SEQ ID NO: 43 - DNA, 19 bp, Artificial
  A target sequence for siRNA
tagcaaagctgaccaagaa

SEQ ID NO: 44 - DNA, 19 bp, Artificial
  A target sequence for siRNA
gatcagacatgtgctatta

SEQ ID NO: 45 - DNA, 19 bp, Artificial
  A target sequence for siRNA
ggtaatacgtggactccta

SEQ ID NO: 46 - protein, 6 aa, Artificial
  A phosphorylation site
RPRQRS

SEQ ID NO: 47 - DNA, 1200 bp, Homo sapiens
  CDS (130)..(1020)
ggggggggggggcacttggcttcaaagctggctcttggaaattgagcggagagcgacgcggttgttgtagctgccgctgcggccgccgcggaataataagccgggatctaccatacccattgactaactatggaagattataccaaaatagagaaaattggagaaggtacctatggagttgtgtataagggtagacacaaaactacaggtcaagtggtagccatgaaaaaaatcagactagaaagtgaagaggaaggggttcctagtactgcaattcgggaaatttctctattaaaggaacttcgtcatccaaatatagtcagtcttcaggatgtgcttatgcaggattccaggttatatctcatctttgagtttctttccatggatctgaagaaatacttggattctatccctcctggtcagtacatggattcttcacttgttaagagttatttataccaaatcctacaggggattgtgttttgtcactctagaagagttcttcacagagacttaaaacctcaaaatctcttgattgatgacaaaggaacaattaaactggctgattttggccttgccagagcttttggaatacctatcagagtatatacacatgaggtagtaacactctggtacagatctccagaagtattgctggggtcagctcgttactcaactccagttgacatttggagtataggcaccatatttgctgaactagcaactaagaaaccacttttccatggggattcagaaattgatcaactcttcaggattttcagagctttgggcactcccaataatgaagtgtggccagaagtggaatctttacaggactataagaatacatttcccaaatggaaaccaggaagcctagcatcccatgtcaaaaacttggatgaaaatggcttggatttgctctcgaaaatgttaatctatgatccagccaaacgaatttctggcaaaatggcactgaatcatccatattttaatgatttggacaatcagattaagaagatgtagctttctgacaaaaagtttccatatgttatgtcaacagatagttgtgtttttattgttaactcttgtctatttttgtcttatatatatttctttgttatcaaacttcagctgtacttcgtcttctaatttcaaaaatataacttaaaaatgtaaatattctatatgaatttaaatata

SEQ ID NO: 48 - protein, 297 aa, Homo sapiens
MEDYTKIEKIGEGTYGVVYKGRHKTTGQVVAMKKIRLESEEEGVPSTAIREISLLKELRHPNIVSLQDVLMQDSRLYLIFEFLSMDLKKYLDSIPPGQYMDSSLVKSYLYQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKM

SEQ ID NO: 49 - DNA, 2005 bp, Homo sapiens
  CDS (101)..(1174)
ctggcgcgcgcggccctgcgggtgacaggcaggcgggaaggggcggggcctcgggcggggccgccgtggggaggagggcggtgggaggggaggagtggagatggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgaggtgggccagtccccagcagcagtggggctgggggcaggggagcaggggggcacgtaggcatcccccatgccaggcctgagccttgctgtctctaccaccccagccagtggccgaggagcccttcaccttcgccatggagctggatgacctacctaaggagcggctgaaggagctcatcttccaggagacagcacgcttccagcccggagtgctggaggccccctagcccagacagacatctctgcaccctggggcctggacctgcctcctgcctgcccctctcccgccagactgttagaaaatggacactgtgcccagcccggaccttggcagcccaggccggggtggagcatgggcctggccacctctctcctttgctgaggcctccagcttcaggcaggccaaggccttctcctccccacccgccctccccacggggcctcgggacctcaggtggccccagttcaatctcccgctgctgctgctgcgcccttaccttccccagcgtcccagtctctggcagttctggaatggaagggttctggctgccccaacctgctgaagggcagaggtggagggtggggggcgctgagtagggactcagggccatgcctgcccccctcatctcattcaaaccccaccctagtttccctgaaggaacattccttagtctcaagggctagcatccctgaggagccaggccgggccgaatcccctccctgtcaaagctgtcacttcgcgtgccctcgctgcttctgtgtgtggtgagcagaagtggagctggggggcgtggagagcccggcgcccctgccacctccctgacccgtctaatatataaatatagagatgtgtctatggctgaaaaaaaaaaaaaaaaaaaaaaaaaaa

SEQ ID NO: 50 - protein, 357 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEVGQSPAAVGLGAGEQGGT

SEQ ID NO: 51 - DNA, 5205 bp, Homo sapiens
  CDS (122)..(3997)
gagccgccgccgggtcccgctcgtctgccgcctcagcctcagccccaacctcagccgccgccgttgcgcttgctcccgggcggtcctggcctgtgccgccgccgcccccagcgtcggagccatggcgggcgccgcgtccccttgcgccaacggctgcgggcccggcgcgccctcggacgccgaggtgctgcacctctgccgcagcctcgaggtgggcaccgtcatgactttgttctactccaagaagtcgcagcgacccgagcggaagaccttccaggtcaagctggagacgcgccagatcacgtggagccggggcgccgacaagatcgagggggccattgacattcgtgaaattaaggagatccgcccagggaagacctcacgggactttgatcgctatcaagaggacccagctttccggccggaccagtcacattgctttgtcattctctatggaatggaatttcgcctgaaaacgctgagcctgcaagccacatctgaggatgaagtgaacatgtggatcaagggcttaacttggctgatggaggatacattgcaggcacccacacccctgcagattgagaggtggctccggaagcagttttactcagtggatcggaatcgtgaggatcgtatatcagccaaggacctgaagaacatgctgtcccaggtcaactaccgggtccccaacatgcgcttcctccgagagcggctgacggacctggagcagcgcagcggggacatcacctacgggcagtttgctcagctgtaccgcagcctcatgtacagcgcccagaagacgatggacctccccttcttggaagccagtactctgagggctggggagcggccggagctttgccgagtgtcccttcctgagttccagcagttccttcttgactaccagggggagctgtgggctgttgatcgcctccaggtgcaggagttcatgctcagcttcctccgagaccccttacgagagatcgaggagccatacttcttcctggatgagtttgtcaccttcctgttctccaaagagaacagtgtgtggaactcgcagctggatgcagtatgcccggacaccatgaacaaccctctttcccactactggatctcctcctcgcacaacacgtacctgaccggggaccagttctccagtgagtcctccttggaagcctatgctcgctgcctgcggatgggctgtcgctgcattgagttggactgctgggacggcccggatgggatgccagttatttaccatgggcacacccttaccaccaagatcaagttctcagatgtcctgcacaccatcaaggagcatgcctttgtggcctcagagtacccagtcatcctgtccattgaggaccactgcagcattgcccagcagagaaacatggcccaatacttcaagaaggtgctgggggacacactcctcaccaagcccgtggagatctctgccgacgggctcccctcacccaaccagcttaagaggaagatcctcatcaagcacaagaagctggctgagggcagtgcctacgaggaggtgcctacatccatgatgtactctgagaacgacatcagcaactctatcaagaatggcatcctctacctggaggaccctgtgaaccacgaatggtatccccactactttgttctgaccagcagcaagatctactactctgaggagaccagcagtgaccagggcaacgaggatgaggaggagcccaaggaggtcagcagcagcacagagctgcactccaatgagaagtggttccatgggaagctaggggcagggcgtgacgggcgtcacatcgctgagcgcctgcttactgagtactgcatcgagaccggagcccctgacggctccttcctcgtgcgagagagtgagaccttcgtgggcgactacacgctctctttctggcggaacgggaaagtccagcactgccgtatccactcccggcaagatgctgggacccccaagttcttcttgacagacaacctcgtctttgactccctctatgacctcatcacgcactaccagcaggtgcccctgcgctgtaatgagtttgagatgcgactttcagagcctgtcccacagaccaacgcccacgagagcaaagagtggtaccacgcgagcctgaccagagcacaggctgagcacatgctaatgcgcgtccctcgtgatggggccttcctggtgcggaagcggaatgaacccaactcatatgccatctctttccgggctgagggcaagatcaagcattgccgtgtccagcaagagggccagacagtgatgctagggaactcggagttcgacagccttgttgacctcatcagctactatgagaaacacccgctataccgcaagatgaagctgcgctatcccatcaacgaggaggcactggagaagattggcacagctgagcctgactacggggccctgtatgagggacgcaaccctggcttctatgtagaggcaaaccctatgccaactttcaagtgtgcagtcaaagccctctttgactacaaggcccagagggaggacgagctgaccttcatcaagagcgccatcatccagaatgtggagaagcaagagggaggctggtggcgaggggactacggagggaagaagcagctgtggttcccatcaaactacgtggaagagatggtcaaccccgtggccctggagccggagagggagcacttggacgagaacagccccctaggggacttgctgcggggggtcttggatgtgccggcttgtcagattgccatccgtcctgagggcaagaacaaccggctcttcgtcttctccatcagcatggcgtcggtggcccactggtccctggatgttgctgccgactcacaggaggagctgcaggactgggtgaaaaagatccgtgaagtggcccagacagcagacgccaggctcactgaagggaagataatggaacggaggaagaagattgccctggagctctctgaacttgtcgtctactgccggcctgttccctttgatgaagagaagattggcacagaacgtgcttgctaccgggacatgtcatccttcccggaaaccaaggctgagaaatacgtgaacaaggccaaaggcaagaagttccttcagtacaatcgactgcagctctcccgcatctaccccaagggccagcgactggattcctccaactacgatcctttgcccatgtggatctgtggcagtcagcttgtggccctcaacttccagacccctgacaagcctatgcagatgaaccaggccctcttcatgacgggcaggcactgtggctacgtgctgcagccaagcaccatgcgggatgaggccttcgacccctttgacaagagcagcctccgcgggctggagccatgtgccatctctattgaggtgctgggggcccgacatctgccaaagaatggccgaggcattgtgtgtccttttgtggagattgaggtggctggagctgagtatgacagcaccaagcagaagacagagtttgtggtggacaatggactcaaccctgtatggccagccaagcccttccacttccagatcagtaaccctgaatttgcctttctgcgcttcgtggtgtatgaggaagacatgtttagtgaccagaatttcctggctcaggctactttcccagtaaaaggcctgaagacaggatacagagcagtgcctttgaagaacaactacagtgaggacctggagttggcctccctgctgatcaagattgacattttccctgccaagcaggagaatggtgacctcagtcccttcagtggtacgtccctgcgggagcggggctcagatgcctcaggccagctgtttcatggccgagcccgggaaggctcctttgaatcccgctaccagcagccgtttgaggacttccgcatctcccaggagcatctcgcagaccattttgacagtcgagaacgaagggccccaagaaggactcgggtcaatggagacaaccgcctctagttgtaccccagcctcgttggagagcagcaggtgctgtgcgccttgtagaatgccgcgaactgggttctttggaagcagccccctgtggcggccttccgggtctcgcagcctgaagcctggattccagcagtgaatgctagacagaaaccaagccattaatgagatgttattactgttttgggcctccatgccccagctctggatgaaggcaaaaactgtactgtgtttcgcattaagcacacacatctggccctgacttctggagatggatccttccatcttgtggggccaggaccatggccgaagccccttggagagagaggctgcctcagccagtggcacaggagactccaaggagctactgacattcctaagagtggaggaggaggaggagccttgctgggccagggaaacaaagtttacattgtcctgtagctttaaaaccacagctgggcagggtgagaagctagatgcccctgcagtttggccctggagccagggcagaggaatgtagggcctgcatggagaagggttctgccctgcctgaggaggaggacacagcacaagggcacattgcccatggctgggaacatgacccagcctgaaagatacaggggatcatgttaaaaatagcagtattatttttcgtctcaatggtattgtaactaagttatttactcctcctgctcctcacccctgtagggaaaccttggagaggagagtggcaggtgggctgcctgctgtgttaagaggacttagtttgtgatgtaaggcactgtcaggaatggggggcgggccagggtgggaagagaagaaatagcagagcctattttggtgaggttttttgtttttaagtcaaagaagactcagtatgctttccctgaggaatgaaaaagggattgaggagttgcctgactcctgggtgggtggggtacaggcagttaggtgctgaatgaagctgccatccttgctgcagcttctaactggtaaaaagatccagggatggagatgggaaggttagaaaggcagccctcacctctgaggacagaggccggggtccaggcccgtgggcgcaaaggtgcctcatagcatagccagcattcagcacacacaaacctactgcccacatttgggctcagggttggccatttgctagttctgctgccctcttaagatctgactgccaaataaatcatcctcatgtcctt

SEQ ID NO: 52 - protein, 1291 aa, Homo sapiens
MAGAASPCANGCGPGAPSDAEVLHLCRSLEVGTVMTLFYSKKSQRPERKTFQVKLETRQITWSRGADKIEGAIDIREIKEIRPGKTSRDFDRYQEDPAFRPDQSHCFVILYGMEFRLKTLSLQATSEDEVNMWIKGLTWLMEDTLQAPTPLQIERWLRKQFYSVDRNREDRISAKDLKNMLSQVNYRVPNMRFLRERLTDLEQRSGDITYGQFAQLYRSLMYSAQKTMDLPFLEASTLRAGERPELCRVSLPEFQQFLLDYQGELWAVDRLQVQEFMLSFLRDPLREIEEPYFFLDEFVTFLFSKENSVWNSQLDAVCPDTMNNPLSHYWISSSHNTYLTGDQFSSESSLEAYARCLRMGCRCIELDCWDGPDGMPVIYHGHTLTTKIKFSDVLHTIKEHAFVASEYPVILSIEDHCSIAQQRNMAQYFKKVLGDTLLTKPVEISADGLPSPNQLKRKILIKHKKLAEGSAYEEVPTSMMYSENDISNSIKNGILYLEDPVNHEWYPHYFVLTSSKIYYSEETSSDQGNEDEEEPKEVSSSTELHSNEKWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFIKSAIIQNVEKQEGGWWRGDYGGKKQLWFPSNYVEEMVNPVALEPEREHLDENSPLGDLLRGVLDVPACQIAIRPEGKNNRLFVFSISMASVAHWSLDVAADSQEELQDWVKKIREVAQTADARLTEGKIMERRKKIALELSELVVYCRPVPFDEEKIGTERACYRDMSSFPETKAEKYVNKAKGKKFLQYNRLQLSRIYPKGQRLDSSNYDPLPMWICGSQLVALNFQTPDKPMQMNQALFMTGRHCGYVLQPSTMRDEAFDPFDKSSLRGLEPCAISIEVLGARHLPKNGRGIVCPFVEIEVAGAEYDSTKQKTEFVVDNGLNPVWPAKPFHFQISNPEFAFLRFVVYEEDMFSDQNFLAQATFPVKGLKTGYRAVPLKNNYSEDLELASLLIKIDIFPAKQENGDLSPFSGTSLRERGSDASGQLFHGRAREGSFESRYQQPFEDFRISQEHLADHFDSRERRAPRRTRVNGDNRL

SEQ ID NO: 53 - DNA, 2115 bp, Homo sapiens
  CDS (272)..(1693)
ggtcaacgcctgcggctgttgatattcttgctcagaggccgtaactttggccttctgctcagggaagactctgagtccgacgttggcctacccagtcggaaggcagagctgcaatctagttaactacctcctttcccctagatttcctttcattctgctcaagtcttcgcctgtgtccgatccctatctactttctctcctcttgtaggcaagcctcagactccaggcttgagctaggttttgtttttctcctggtgagaattcgaagaccatgtctacggaactcttctcatccacaagagaggaaggaagctctggctcaggacccagttttaggtctaatcaaaggaaaatgttaaacctgctcctggagagagacacttcctttaccgtctgtccagatgtccctagaactccagtgggcaaatttcttggtgattctgcaaacctaagcattttgtctggaggaaccccaaaacgttgcctcgatctttcgaatcttagcagtggggagataactgccactcagcttaccacttctgcagaccttgatgaaactggtcacctggattcttcaggacttcaggaagtgcatttagctgggatgaatcatgaccagcacctaatgaaatgtagcccagcacagcttctttgtagcactccgaatggtttggaccgtggccatagaaagagagatgcaatgtgtagttcatctgcaaataaagaaaatgacaatggaaacttggtggacagtgaaatgaaatatttgggcagtcccattactactgttccaaaattggataaaaatccaaacctaggagaagaccaggcagaagagatttcagatgaattaatggagttttccctgaaagatcaagaagcaaaggtgagcagaagtggcctatatcgctccccgtcgatgccagagaacttgaacaggccaagactgaagcaggtggaaaaattcaaggacaacacaataccagataaagttaaaaaaaagtatttttctggccaaggaaagctcaggaagggcttatgtttaaagaagacagtctctctgtgtgacattactatcactcagatgctggaggaagattctaaccaggggcacctgattggtgatttttccaaggtatgtgcgctgccaaccgtgtcagggaaacaccaagatctgaagtatgtcaacccagaaacagtggctgccttactgtcggggaagttccagggtctgattgagaagttttatgtcattgattgtcgctatccatatgagtatctgggaggacacatccagggagccttaaacttatatagtcaggaagaactgtttaacttctttctgaagaagcccatcgtccctttggacacccagaagagaataatcatcgtgttccactgtgaattctcctcagagaggggcccccgaatgtgccgctgtctgcgtgaagaggacaggtctctgaaccagtatcctgcattgtactacccagagctatatatccttaaaggcggctacagagacttctttccagaatatatggaactgtgtgaaccacagagctactgccctatgcatcatcaggaccacaagactgagttgctgaggtgtcgaagccagagcaaagtgcaggaaggggagcggcagctgcgggagcagattgcccttctggtgaaggacatgagcccatgataacattccagccactggctgctaacaagtcaccaaaagacactgcagaaaccctgagcagaaagaggccttctggatggccaaacccaagattattaaaagatgtctctgcaaaccaacaggctaccaacttgtatccaggcctgggaatggattaggtttcagcagagctgaaagctggtggcagagtcctggagctggctctataaggcagccttgagttgcatagagatttgtattggttcagggaactctggcattccttttcccaactcctcatgtcttctcacaagccagccaactctttctctctgggcttcgggctatgcaagagcgttgtctaccttctttctttgtattttccttctttgtttccccctctttcttttttaaaaatggaaaaataaacactacagaatgag

SEQ ID NO: 54 - protein, 473 aa, Homo sapiens
MSTELFSSTREEGSSGSGPSFRSNQRKMLNLLLERDTSFTVCPDVPRTPVGKFLGDSANLSILSGGTPKRCLDLSNLSSGEITATQLTTSADLDETGHLDSSGLQEVHLAGMNHDQHLMKCSPAQLLCSTPNGLDRGHRKRDAMCSSSANKENDNGNLVDSEMKYLGSPITTVPKLDKNPNLGEDQAEEISDELMEFSLKDQEAKVSRSGLYRSPSMPENLNRPRLKQVEKFKDNTIPDKVKKKYFSGQGKLRKGLCLKKTVSLCDITITQMLEEDSNQGHLIGDFSKVCALPTVSGKHQDLKYVNPETVAALLSGKFQGLIEKFYVIDCRYPYEYLGGHIQGALNLYSQEELFNFFLKKPIVPLDTQKRIIIVFHCEFSSERGPRMCRCLREEDRSLNQYPALYYPELYILKGGYRDFFPEYMELCEPQSYCPMHHQDHKTELLRCRSQSKVQEGERQLREQIALLVKDMSP

SEQ ID NO: 55 - DNA, 6641 bp, Homo sapiens
  CDS (188)..(4360)
gccctcgccgcccgcggcgccccgagcgctttgtgagcagatgcggagccgagtggagggcgcgagccagatgcggggcgacagctgacttgctgagaggaggcggggaggcgcggagcgcgcgtgtggtccttgcgccgctgacttctccactggttcctgggcaccgaaagataaacctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctcttgccaaaattgcactattataggacttgtattgttatttaaattactggattctaaggaatttcttatctgacagagcatcagaaccagaggcttggtcccacaggccacggaccaatggcctgcagccgtgacaacactcctgtcatattggagtccaaaacttgaattctgggttgaattttttaaaaatcaggtaccacttgatttcatatgggaaattgaagcaggaaatattgagggcttcttgatcacagaaaactcagaagagatagtaatgctcaggacaggagcggcagccccagaacaggccactcatttagaattctagtgtttcaaaacacttttgtgtgttgtatggtcaataacatttttcattactgatggtgtcattcacccattaggtaaacattcccttttaaatgtttgtttgttttttgagacaggatctcactctgttgccagggctgtagtgcagtggtgtgatcatagctcactgcaacctccacctcccaggctcaagcctcccgaatagctgggactacaggcgcacaccaccatccccggctaatttttgtattttttgtagagacggggttttgccatgttgccaaggctggtttcaaactcctggactcaagaaatccacccacctcagcctcccaaagtgctaggattacaggcatgagccactgcgcccagcccttataaatttttgtatagacattcctttggttggaagaatatttataggcaatacagtcaaagtttcaaaatagcatcacacaaaacatgtttataaatgaacaggatgtaatgtacatagatgacattaagaaaatttgtatgaaataatttagtcatcatgaaatatttagttgtcatataaaaacccactgtttgagaatgatgctactctgatctaatgaatgtgaacatgtagatgttttgtgtgtatttttttaaatgaaaactcaaaataagacaagtaatttgttgataaatatttttaaagataactcagcatgtttgtaaagcaggatacattttactaaaaggttcattggttccaatcacagctcataggtagagcaaagaaagggtggatggattgaaaagattagcctctgtctcggtggcaggttcccacctcgcaagcaattggaaacaaaacttttggggagttttattttgcattagggtgtgttttatgttaagcaaaacatactttagaaacaaatgaaaaaggcaattgaaaatcccagctatttcacctagatggaatagccaccctgagcagaactttgtgatgcttcattctgtggaattttgtgcttgctactgtatagtgcatgtggtgtaggttactctaactggttttgtcgacgtaaacatttaaagtgttatattttttataaaaatgtttatttttaatgatatgagaaaaattttgttaggccacaaaaacactgcactgtgaacattttagaaaaggtatgtcagactgggattaatgacagcatgattttcaatgactgtaaattgcgataaggaaatgtactgattgccaatacaccccaccctcattacatcatcaggacttgaagccaagggttaacccagcaagctacaaagagggtgtgtcacactgaaactcaatagttgagtttggctgttgttgcaggaaaatgattataactaaaagctctctgatagtgcagagacttaccagaagacacaaggaattgtactgaagagctattacaatccaaatattgccgtttcataaatgtaataagtaatactaattcacagagtattgtaaatggtggatgacaaaagaaaatctgctctgtggaaagaaagaactgtctctaccagggtcaagagcatgaacgcatcaatagaaagaactcggggaaacatcccatcaacaggactacacacttgtatatacattcttgagaacactgcaatgtgaaaatcacgtttgctatttataaacttgtccttagattaatgtgtctggacagattgtgggagtaagtgattcttctaagaattagatacttgtcactgcctatacctgcagctgaactgaatggtacttcgtatgttaatagttgttctgataaatcatgcaattaaagtaaagtgatgcaacatcttgtaaaaaaaaaaaaaaaaaaaa

SEQ ID NO: 56 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 57 - DNA, 3192 bp, Homo sapiens
  CDS (240)..(1661)
actgcctttgtgcgcgatctcgcgctgccattggctaactcgggaaagtgggaagcgtgaaggagggaccctgaggtagagggtcaggggttagtgaggccggaagtgagtgtaataaagtttctccagggaggcagggcccggggagaaagttggagcggtaacctaagctggcagtggcgtgatccggcaccaaatcggcccgcggtgcggtgcggagactccatgaggccctggacatgaacaagctgagtggaggcggcgggcgcaggactcgggtggaagggggccagcttgggggcgaggagtggacccgccacgggagctttgtcaataagcccacgcggggctggctgcatcccaacgacaaagtcatgggacccggggtttcctacttggttcggtacatgggttgtgtggaggtcctccagtcaatgcgtgccctggacttcaacacccggactcaggtcaccagggaggccatcagtctggtgtgtgaggctgtgccgggtgctaagggggcgacaaggaggagaaagccctgtagccgcccgctcagctctatcctggggaggagtaacctgaaatttgctggaatgccaatcactctcaccgtctccaccagcagcctcaacctcatggccgcagactgcaaacagatcatcgccaaccaccacatgcaatctatctcatttgcatccggcggggatccggacacagccgagtatgtcgcctatgttgccaaagaccctgtgaatcagagagcctgccacattctggagtgtcccgaagggcttgcccaggatgtcatcagcaccattggccaggccttcgagttgcgcttcaaacaatacctcaggaacccacccaaactggtcacccctcatgacaggatggctggctttgatggctcagcatgggatgaggaggaggaagagccacctgaccatcagtactataatgacttcccggggaaggaaccccccttggggggggtggtagacatgaggcttcgggaaggagccgctccaggggctgctcgacccactgcacccaatgcccagacccccagccacttgggagctacattgcctgtaggacagcctgttgggggagatccagaagtccgcaaacagatgccacctccaccaccctgtccaggcagagagctttttgatgatccctcctatgtcaacgtccagaacctagacaaggcccggcaagcagtgggtggtgctgggccccccaatcctgctatcaatggcagtgcaccccgggacctgtttgacatgaagcccttcgaagatgctcttcgcgtgcctccacctccccagtcggtgtccatggctgagcagctccgaggggagccctggttccatgggaagctgagccggcgggaggctgaggcactgctgcagctcaatggggacttcctggtacgggagagcacgaccacacctggccagtatgtgctcactggcttgcagagtgggcagcctaagcatttgctactggtggaccctgagggtgtggttcggactaaggatcaccgctttgaaagtgtcagtcaccttatcagctaccacatggacaatcacttgcccatcatctctgcgggcagcgaactgtgtctacagcaacctgtggagcggaaactgtgatctgccctagcgctctcttccagaagatgccctccaatcctttccaccctattccctaactctcgggacctcgtttgggagtgttctgtgggcttggccttgtgtcagagctgggagtagcatggactctgggtttcatatccagctgagtgagagggtttgagtcaaaagcctgggtgagaatcctgcctctccccaaacattaatcaccaaagtattaatgtacagagtggcccctcacctgggcctttcctgtgccaacctgatgccccttccccaagaaggtgagtgcttgtcatggaaaatgtcctgtggtgacaggcccagtggaacagtcacccttctgggcaagggggaacaaatcacacctctgggcttcagggtatcccagacccctctcaacacccgccccccccatgtttaaactttgtgcctttgaccatctcttaggtctaatgatattttatgcaaacagttcttggacccctgaattcaatgacagggatgccaacaccttcttggcttctgggacctgtgttcttgctgagcaccctctccggtttgggttgggataacagaggcaggagtggcagctgtcccctctccctggggatatgcaacccttagagattgccccagagccccactcccggccaggcgggagatggacccctcccttgctcagtgcctcctggccggggcccctcaccccaaggggtctgtatatacatttcataaggcctgccctcccatgttgcatgcctatgtactctacgccaaagtgcagcccttcctcctgaagcctctgccctgcctccctttctgggagggcggggtgggggtgactgaatttgggcctcttgtacagttaactctcccaggtggattttgtggaggtgagaaaaggggcattgagactataaagcagtagacaatccccacataccatctgtagagttggaactgcattcttttaaagttttatatgcatatattttagggctgtagacttactttcctattttcttttccattgcttattcttgagcacaaaatgataatcaattattacatttatacatcacctttttgacttttccaagcccttttacagctcttggcattttcctcgcctaggcctgtgaggtaactgggatcgcaccttttataccagagacctgaggcagatgaaatttatttccatctaggactagaaaaacttgggtctcttaccgcgagactgagaggcagaagtcagcccgaatgcctgtcagtttcatggaggggaaacgcaaaacctgcagttcctgagtaccttctacaggcccggcccagcctaggcccggggtggccacaccacagcaagccggccccccctcttttggccttgtggataagggagagttgaccgttttcatcctggcctccttttgctgtttggatgtttccacgggtctcacttataccaaagggaaaactcttcattaaagtccgtatttcttctaaaaaaaaaaaaaaaaaaaaaaaaaaa

SEQ ID NO: 58 - protein, 473 aa, Homo sapiens
MNKLSGGGGRRTRVEGGQLGGEEWTRHGSFVNKPTRGWLHPNDKVMGPGVSYLVRYMGCVEVLQSMRALDFNTRTQVTREAISLVCEAVPGAKGATRRRKPCSRPLSSILGRSNLKFAGMPITLTVSTSSLNLMAADCKQIIANHHMQSISFASGGDPDTAEYVAYVAKDPVNQRACHILECPEGLAQDVISTIGQAFELRFKQYLRNPPKLVTPHDRMAGFDGSAWDEEEEEPPDHQYYNDFPGKEPPLGGVVDMRLREGAAPGAARPTAPNAQTPSHLGATLPVGQPVGGDPEVRKQMPPPPPCPGRELFDDPSYVNVQNLDKARQAVGGAGPPNPAINGSAPRDLFDMKPFEDALRVPPPPQSVSMAEQLRGEPWFHGKLSRREAEALLQLNGDFLVRESTTTPGQYVLTGLQSGQPKHLLLVDPEGVVRTKDHRFESVSHLISYHMDNHLPIISAGSELCLQQPVERKL

SEQ ID NO: 59 - DNA, 2794 bp, Homo sapiens
  CDS (341)..(1783)
cggcaggaccgagcgcggcaggcggctggcccagcgcagccagcgcggcccgaaggacgggagcaggcggccgagcaccgagcgctgggcaccgggcaccgagcggcggcggcacgcgaggcccggccccgagcagcgcccccgcccgccgcggcctccagcccggccccgcccagcgccggcccgcggggatgcggagcggcgggcgccggaggccgcggcccggctaggcccgcgctcgcgcccggacgcggcggcccgaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

SEQ ID NO: 60 - protein, 480 aa, Homo sapiens [sequence omitted]

SEQ ID NO: 61 - DNA, 4978 bp, Homo sapiens
  CDS (241)..(2553)
ggtttccggagctgcggcggcgcagactgggagggggagccgggggttccgacgtcgcagccgagggaacaagccccaaccggatcctggacaggcaccccggcttggcgctgtctctccccctcggctcggagaggcccttcggcctgagggagcctcgccgcccgtccccggcacacgcgcagccccggcctctcggcctctgccggagaaacagttgggacccctgattttagcaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtgaatgtgggtgatctgcttttatctaaatgcaaataaggatgtgttctctgagacccatgatcaggggatgtggcggggggtggctagagggagaaaaaggaaatgtcttgtgttgttttgttcccctgccctcctttctcagcagctttttgttattgttgttgttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctttttaaattaagaaataataacaattaaagggcaaaaaacactgtatcagcatagcctttctgtatttaagaaacttaagcagccgggcatggtggctcacgcctgtaatcccagcactttgggaggccgaggcggatcataaggtcaggagatcaagaccatcctggctaacacggtgaaaccccgtctctactaaaagtacaaaaaattagctgggtgtggtggtgggcgcctgtagtcccagctactcgggaggctgaggcaggagaatcgcttgaacctgagaggcggaggttgcagtgagccaaaattgcaccactgcacactgcactccatcctgggcgacagtctgagactctgtctcaaaaaaaaaaaaaaaaaaaagaaacttcagttaacagcctccttggtgctttaagcattcagcttccttcaggctggtaatttatataatccctgaaacgggcttcaggtcaaacccttaagacatctgaagctgcaacctggcctttggtgttgaaataggaaggtttaaggagaatctaagcattttagacttttttttataaatagacttattttcctttgtaatgtattggccttttagtgagtaaggctgggcagagggtgcttacaaccttgactccctttctccctggacttgatctgctgtttcagaggctaggttgtttctgtgggtgccttatcagggctgggatacttctgattctggcttccttcctgccccaccctcccgaccccagtccccctgatcctgctagaggcatgtctccttgcgtgtctaaaggtccctcatcctgtttgttttaggaatcctggtctcaggacctcatggaagaagagggggagagagttacaggttggacatgatgcacactatggggccccagcgacgtgtctggttgagctcagggaatatggttcttagccagtttcttggtgatatccagtggcacttgtaatggcgtcttcattcagttcatgcagggcaaaggcttactgataaacttgagtctgccctcgtatgagggtgtatacctggcctccctctgaggctggtgactcctccctgctggggccccacaggtgaggcagaacagctagagggcctccccgcctgcccgccttggctggctagctcgcctctcctgtgcgtatgggaacacctagcacgtgctggatgggctgcctctgactcagaggcatggccggatttggcaactcaaaaccaccttgcctcagctgatcagagtttctgtggaattctgtttgttaaatcaaattagctggtctctgaattaagggggagacgaccttctctaagatgaacagggttcgccccagtcctcctgcctggagacagttgatgtgtcatgcagagctcttacttctccagcaacactcttcagtacataataagcttaactgataaacagaatatttagaaaggtgagacttgggcttaccattgggtttaaatcatagggacctagggcgagggttcagggcttctctggagcagatattgtcaagttcatggccttaggtagcatgtatctggtcttaactctgattgtagcaaaagttctgagaggagctgagccctgttgtggcccattaaagaacagggtcctcaggccctgcccgcttcctgtccactgccccctccccatccccagcccagccgagggaatcccgtgggttgcttacctacctataaggtggtttataagctgctgtcctggccactgcattcaaattccaatgtgtacttcatagtgtaaaaatttatattattgtgaggttttttgtcttttttttttttttttttttttggtatattgctgtatctactttaacttccagaaataaacgttatataggaaccgtaaaaa

SEQ ID NO: 62 - protein, 770 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 63 - DNA, 3291 bp, Homo sapiens
  CDS (416)..(2362)
agaatcggagagccggtggcgtcgcaggtcgggaggacgagcaccgagtcgagggctcgctcgtctgggccgcccgagagtcttaatcgcgggcgcttgggccgccatcttagatggcgggagtaagaggaaaacgattgtgaggcgggaacggctttctgctgccttttttgggccccgaaaagggtcagctggccgggctttggggcgcgtgccctgaggcgcggagcgcgtttgctacgatgcgggggctgctcggggctccgtcccctgggctggggacgcgccgaatgtgaccgcctcccgctccctcacccgccgcggggaggaggagcgggcgagaagctgccgccgaacgacaggacgttggggcggcctggctccctcaggtttaagaattgtttaagctgcatcaatggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtcaatgtgcgaaatggaatgagcttgcatgactgccttatgaaagcactcaaggtgaggggcctgcaaccagagtgctgtgcagtgttcagacttctccacgaacacaaaggtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccaccctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctagttgactttgcacctgtcttcaggctgccaggggaggaggagaagccagcaggcaccacttttctgctccctttctccagaggcagaacacatgttttcagagaagctgctgctaaggaccttctagactgctcacagggccttaacttcatgttgccttcttttctatccctttgggccctgggagaaggaagccatttgcagtgctggtgtgtcctgctccctccccacattccccatgctcaaggcccagccttctgtagatgcgcaagtggatgttgatggtagtacaaaaagcaggggcccagccccagctgttggctacatgagtatttagaggaagtaaggtagcaggcagtccagccctgatgtggagacacatgggattttggaaatcagcttctggaggaatgcatgtcacaggcgggactttcttcagagagtggtgcagcgccagacattttgcacataaggcaccaaacagcccaggactgccgagactctggccgcccgaaggagcctgctttggtactatggaacttttcttaggggacacgtcctcctttcacagcttctaaggtgtccagtgcattgggatggttttccaggcaaggcactcggccaatccgcatctcagccctctcagggagcagtcttccatcatgctgaattttgtcttccaggagctgcccctatggggcggggccgcagggccagccttgtttctctaacaaacaaacaaacaaacagccttgtttctctagtcacatcatgtgtatacaaggaagccaggaatacaggttttcttgatgatttgggttttaattttgtttttattgcacctgacaaaatacagttatctgatggtccctcaattatgttattttaataaaataaattaaatttaggtgtaaaaaaaaaaaaaaaaaa

SEQ ID NO: 64 - protein, 648 aa, Homo sapiens [sequence omitted]

SEQ ID NO: 65 - DNA, 30 bp, Artificial
  An artificially synthesized primer sequence for RT-PCR
cgcggatcccaccatggtttttcaaactcg

SEQ ID NO: 66 - DNA, 33 bp, Artificial
  An artificially synthesized primer sequence for RT-PCR
ccgctcgagcacttgaatgccagttccatgtaa

SEQ ID NO: 67 - DNA, 34 bp, Artificial
  An artificially synthesized primer sequence for RT-PCR
ttgcggccgcaaatgaaggcccccgctgtgcttg

SEQ ID NO: 68 - DNA, 35 bp, Artificial
  An artificially synthesized primer sequence for RT-PCR
ccgctcgagcggtgatgtctcccagaaggaggctg

SEQ ID NO: 69 - DNA, 5616 bp, Homo sapiens
  CDS (247)..(3879)
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 70 - protein, 1210 aa, Homo sapiens [sequence omitted]

SEQ ID NO: 71 - DNA, 2603 bp, Homo sapiens
  CDS (476)..(1657)
aggcgaggcttccccttccccgcccctcccccggcctccagtccctcccagggccgcttcgcagagcggctaggagcacggcggcggcggcactttccccggcaggagctggagctgggctctggtgcgcgcgcggctgtgccgcccgagccggagggactggttggttgagagagagagaggaagggaatcccgggctgccgaaccgcacgttcagcccgctccgctcctgcagggcagcctttcggctctctgcgcgcgaagccgagtcccgggcgggtggggcgggggtccactgagaccgctaccggcccctcggcgctgacgggaccgcgcggggcgcacccgctgaaggcagccccggggcccgcggcccggacttggtcctgcgcagcgggcgcggggcagcgcagcgggaggaagcgagaggtgctgccctccccccggagttggaagcgcgttacccgggtccaaaatgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaagtgtttgggaagcaacaaagagcgagtcccctgcccggtggtttgccatgtcgcttttgggcctccttcccatgcctgtctctgttcagatgtgcatttcacctgtgacaaaggatgaagaacacagcatgtgccaagattctactcttgtcatttttaatattactgtctttattcttattactattattgttcccctaagtggattggctttgtgcttggggctatttgtgtgtatgctgatgatcaaaacctgtgccaggctgaattacagtgaaattttggtgaatgtgggtagtcattcttacaattgcactgctgttcctgctccatgactggctgtctgcctgtattttcgggattctttgacatttggtggtactttattcttgctgggcatactttctctctaggagggagccttgtgagatccttcacaggcagtgcatgtgaagcatgctttgctgctatgaaaatgagcatcagagagtgtacatcatgttattttattattattatttgcttttcatgtagaactcagcagttgacatccaaatctagccagagcccttcactgccatgatagctggggcttcaccagtctgtctactgtggtgatctgtagacttctggttgtatttctatatttattttcagtatactgtgtgggatacttagtggtatgtctctttaagttttgattaatgtttcttaaatggaattattttgaatgtcacaaattgatcaagatattaaaatgtcggatttatctttccccatatccaagtaccaatgctgttgtaaacaacgtgtatagtgcctaaaattgtatgaaaatccttttaaccattttaacctagatgtttaacaaatctaatctcttattctaataaatatactatgaaataaaaaaaaaaggatgaaagctaaaaaaaaaaaaaaaaaa

SEQ ID NO: 72 - protein, 393 aa, Homo sapiens [sequence omitted]

SEQ ID NO: 73 - DNA, 1759 bp, Homo sapiens
  CDS (255)..(1457)
cccctgcctctcggactcgggctgcggcgtcagccttcttcgggcctcggcagcggtagcggctcgctcgcctcagccccagcgcccctcggctaccctcggcccaggcccgcagcgccgcccgccctcggccgccccgacgccggcctgggccgcggccgcagccccgggctcgcgtaggcgccgaccgctcccggcccgccccctatgggccccggctagaggcgccgccgccgccggcccgcggagccccgatgctggcccggaggaagccggtgctgccggcgctcaccatcaaccctaccatcgccgagggcccatcccctaccagcgagggcgcctccgaggcaaacctggtggacctgcagaagaagctggaggagctggaacttgacgagcagcagaagaagcggctggaagcctttctcacccagaaagccaaggtcggcgaactcaaagacgatgacttcgaaaggatctcagagctgggcgcgggcaacggcggggtggtcaccaaagtccagcacagaccctcgggcctcatcatggccaggaagctgatccaccttgagatcaagccggccatccggaaccagatcatccgcgagctgcaggtcctgcacgaatgcaactcgccgtacatcgtgggcttctacggggccttctacagtgacggggagatcagcatttgcatggaacacatggacggcggctccctggaccaggtgctgaaagaggccaagaggattcccgaggagatcctggggaaagtcagcatcgcggttctccggggcttggcgtacctccgagagaagcaccagatcatgcaccgagatgtgaagccctccaacatcctcgtgaactctagaggggagatcaagctgtgtgacttcggggtgagcggccagctcatcgactccatggccaactccttcgtgggcacgcgctcctacatggctccggagcggttgcagggcacacattactcggtgcagtcggacatctggagcatgggcctgtccctggtggagctggccgtcggaaggtaccccatccccccgcccgacgccaaagagctggaggccatctttggccggcccgtggtcgacggggaagaaggagagcctcacagcatctcgcctcggccgaggccccccgggcgccccgtcagcggtcacgggatggatagccggcctgccatggccatctttgaactcctggactatattgtgaacgagccacctcctaagctgcccaacggtgtgttcacccccgacttccaggagtttgtcaataaatgcctcatcaagaacccagcggagcgggcggacctgaagatgctcacaaaccacaccttcatcaagcggtccgaggtggaagaagtggattttgccggctggttgtgtaaaaccctgcggctgaaccagcccggcacacccacgcgcaccgccgtgtgacagtggccgggctccctgcgtcccgctggtgacctgcccaccgtccctgtccatgccccgcccttccagctgaggacaggctggcgcctccacccaccctcctgcctcacccctgcggagagcaccgtggcggggcgacagcgcatgcaggaacgggggtctcctctcctgcccgtcctggccggggtgcctctggggacgggcgacgctgctgtgtgtggtctcagaggctctgcttccttaggttacaaaacaaaacagggagagaaaaagcaaaaaaaaaaaaaaaaaaaaaaaaaaaa

SEQ ID NO: 74 - protein, 400 aa, Homo sapiens [sequence omitted]

SEQ ID NO: 75 - protein, 141 aa, Artificial
  An artificially synthesized a polypeptidefragment
SSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 76 - protein, 420 aa, Artificial
  An artificially synthesized a polypeptidefragment
YDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS